SEQUENCE LISTING (242 sequences)

SEQ ID NO: 1 - protein, 288 aa, Artificial Sequence
  synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGADSLGREAKCYNELNGCTKIYDPVCGTDGNTYPNECVLCFENRKRQTSILIQKSGPC

SEQ ID NO: 2 - protein, 290 aa, Artificial Sequence
  synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGARPDFCLEPPYTGPCKARIIRYFYNAKAGLCQTFVYGGCRAKRNNFKSAEDCMRTCGGA

SEQ ID NO: 3 - protein, 58 aa, Artificial Sequence [sequence omitted]

SEQ ID NO: 4 - protein, 290 aa, Artificial Sequence
  synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGARPDFCLEPPYTGPCKHRIIRYFYNAKAGLCQTFVYGGCRAKRNNFKSAEDCMRTCGGA

SEQ ID NO: 5 - protein, 290 aa, Artificial Sequence
  synthetic polypeptide
EPKSCDKTHTCPPCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCPVKGFYPSDIAVEWESSGQPENNYNTTPPMLDSDGSFFLYSKLTVDKSRWQQGNIFSCSVMHEALHNHYTQKSLSLSPGARPDFCLEPPYTGPCKHRIIRYFYNAKAGLCQTFVYGGCRAKRNNFKSAEDCMRTCGGA

SEQ ID NO: 6 - protein, 288 aa, Artificial Sequence; identical to SEQ ID NO: 1
  synthetic polypeptide

SEQ ID NO: 7 - protein, 319 aa, Artificial Sequence
  synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGADSLGREAKCYNELNGCTKIYDPVCGTDGNTYPNECVLCFENRKRQTSILIQKSGPCGPEQKLISEEDLNSALVPRGSRLVDHHHHHH

SEQ ID NO: 8 - protein, 288 aa, Artificial Sequence
  synthetic polypeptide
EPKSSDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGADSLGREAKCYNELNGCTKIYDPVCGTDGNTYPNECVLCFENRKRQTSILIQKSGPC

SEQ ID NO: 9 - protein, 288 aa, Artificial Sequence
  synthetic polypeptide
EPEESDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGEDSLGREAKCYNELNGCTKIYDPVCGTDGNTYPNECVLCFENRKRQTSILIQKSGPC

SEQ ID NO: 10 - protein, 290 aa, Artificial Sequence
  synthetic polypeptide
  misc_feature (1)..(1): Xaa is independently aspartic acid (D) or glutamic acid (E), and each Xaa can independently range from 1 to up to 10 or more repeats
  misc_feature (4)..(5): Xaa is independently aspartic acid (D) or glutamic acid (E), and each Xaa can independently range from 1 to up to 10 or more repeats
  misc_feature (83)..(83): Xaa is independently aspartic acid (D) or glutamic acid (E), and each Xaa can independently range from 1 to up to 10 or more repeats
  misc_feature (233)..(233): Xaa is independently aspartic acid (D) or glutamic acid (E), and each Xaa can independently range from 1 to up to 10 or more repeats
  misc_feature (290)..(290): Xaa is independently aspartic acid (D) or glutamic acid (E), and each Xaa can independently range from 1 to up to 10 or more repeats
XEPXXSDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGXDSLGREAKCYNELNGCTKIYDPVCGTDGNTYPNECVLCFENRKRQTSILIQKSGPCX

SEQ ID NO: 11 - protein, 292 aa, Artificial Sequence
  synthetic polypeptide
  misc_feature (1)..(1): Xaa is independently aspartic acid (D) or glutamic acid (E), and each Xaa can independently range from 1 to up to 10 or more repeats
  misc_feature (234)..(234): Xaa is independently aspartic acid (D) or glutamic acid (E), and each Xaa can independently range from 1 to up to 10 or more repeats
  misc_feature (292)..(292): Xaa is independently aspartic acid (D) or glutamic acid (E), and each Xaa can independently range from 1 to up to 10 or more repeats
XEPEESDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGEXQDSLGREAKCYNELNGCTKIYDPVCGTDGNTYPNECVLCFENRKRQTSILIQKSGPCX

SEQ ID NO: 12 - DNA, 17 bp, Artificial Sequence
  synthetic polynucleotide
gggaggagcagtacaac

SEQ ID NO: 13 - DNA, 21 bp, Artificial Sequence
  synthetic polynucleotide
gaggctctgcacaaccactac

SEQ ID NO: 14 - protein, 56 aa, Artificial Sequence
  synthetic polypeptide
DSLGREAKCYNELNGCTKIYDPVCGTDGNTYPNECVLCFENRKRQTSILIQKSGPC

SEQ ID NO: 15 - protein, 87 aa, Artificial Sequence
  synthetic polypeptide
DSLGREAKCYNELNGCTKIYDPVCGTDGNTYPNECVLCFENRKRQTSILIQKSGPCGPEQKLISEEDLNSALVPRGSRLVDHHHHHH

SEQ ID NO: 16 - protein, 77 aa, Artificial Sequence
  synthetic polypeptide
MKVTGIFLLSALALLSLSGNTGADSLGREAKCYNELNGCTKIYDPVCGGNTYPNECVLCFENRKRQTSILIQKSGPC

SEQ ID NO: 17 - protein, 77 aa, Artificial Sequence
  synthetic polypeptide
MKVTGIFLLSALALLSLSGNTGADSLGREAKCYNELNGCTKIYNPVCGGNTYPNECVLCFENRKRQTSILIQKSGPC

SEQ ID NO: 18 - protein, 77 aa, Artificial Sequence
  synthetic polypeptide
MKVTGIFLLSALALLSLSGNTGADSLGREAKCYNELNGCTKIYDPVCGGDTYPNECVLCFENRKRQTSILIQKSGPC

SEQ ID NO: 19 - protein, 77 aa, Artificial Sequence
  synthetic polypeptide
MKVTGIFLLSALALLSLSGNTGADSLGREAKCYNELNGCTKIYDPVCGGNTYPNECVLCFEGRKRQTSILIQKSGPC

SEQ ID NO: 20 - protein, 77 aa, Artificial Sequence
  synthetic polypeptide
MKVTGIFLLSAFALLSLSGNTGADSLGREAKCYNELNGCTKIYDPVCGGNTYPNECVLCFENRKRQTSILIQKSGPC

SEQ ID NO: 21 - protein, 77 aa, Artificial Sequence
  synthetic polypeptide
MKVTGIFLLSALAPLSLSGNTGADSLGREAKCYSELNGCTKIYDPVCGGNTYPNECVLCFENRKRQTSILIQKSGPC

SEQ ID NO: 22 - protein, 77 aa, Artificial Sequence
  synthetic polypeptide
MKVTGIFLLSALALLSLSGNTGADSLGREAKCYNELNGCTKIYDPVCGGNTYSNECVLCFENRKHQTSILIQKSGPC

SEQ ID NO: 23 - protein, 58 aa, Artificial Sequence
  synthetic polypeptide
RPDFCLEPPYTGPCKHRIIRYFYNAKAGLCQTFVYGGCRAKRNNFKSAEDCMRTCGGA

SEQ ID NO: 24 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 231 aa, Homo sapiens
KTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 26 - protein, 264 aa, Homo sapiens
ELKTPLGDTTHTCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPWEEQYNSTFRVVSVLTVLHQDWLNGKEYKCKVSNKALPAPIEKTISKTKGQPREPQVYTLPPSREEMTKNQVSLTCLVKGFYPSDIAMEWESSGQPENNYKTTPPVLDSDGSFFLYSKLTVDKSRWQQGNIFSCSVMHEALHNHYTQKSLSLSPGK

SEQ ID NO: 27 - protein, 235 aa, Homo sapiens
KVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 238 aa, Homo sapiens
EPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 29 - protein, 12 aa, Artificial Sequence
  synthetic polypeptide
EPKSCDKTHTCP

SEQ ID NO: 30 - protein, 12 aa, Artificial Sequence
  synthetic polypeptide
EPKSSDKTHTCP

SEQ ID NO: 31 - protein, 20 aa, Artificial Sequence
  synthetic polypeptide
KTKPREEQYNSTYRVVSVLT

SEQ ID NO: 32 - protein, 20 aa, Artificial Sequence
  synthetic polypeptide
KTKPREEQYDSTYRVVSVLT

SEQ ID NO: 33 - protein, 20 aa, Artificial Sequence
  synthetic polypeptide
KTKPREEQYQSTYRVVSVLT

SEQ ID NO: 34 - protein, 10 aa, Artificial Sequence
  synthetic polypeptide
QKSLSLSLGK

SEQ ID NO: 35 - protein, 10 aa, Artificial Sequence
  synthetic polypeptide
QKSLSLSLGA

SEQ ID NO: 36 - protein, 232 aa, Artificial Sequence
  synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 37 - protein, 232 aa, Artificial Sequence
  synthetic polypeptide
EPKSSDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 38 - protein, 219 aa, Artificial Sequence
  synthetic polypeptide
CPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCPVKGFYPSDIAVEWESSGQPENNYNTTPPMLDSDGSFFLYSKLTVDKSRWQQGNIFSCSVMHEALHNHYTQKSLSLSPGA

SEQ ID NO: 39 - protein, 232 aa, Artificial Sequence
  synthetic polypeptide
EPKSCDKTHTCPPCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCPVKGFYPSDIAVEWESSGQPENNYNTTPPMLDSDGSFFLYSKLTVDKSRWQQGNIFSCSVMHEALHNHYTQKSLSLSPGA

SEQ ID NO: 40 - protein, 232 aa, Artificial Sequence
  synthetic polypeptide
EPEESDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGE

SEQ ID NO: 41 - protein, 4 aa, Artificial Sequence
  synthetic polypeptide
GGGS

SEQ ID NO: 42 - protein, 8 aa, Artificial Sequence
  synthetic polypeptide
GGGSGGGS

SEQ ID NO: 43 - protein, 12 aa, Artificial Sequence
  synthetic polypeptide
GGGSGGGSGGGS

SEQ ID NO: 44 - protein, 16 aa, Artificial Sequence
  synthetic polypeptide
GGGSGGGSGGGSGGGS

SEQ ID NO: 45 - protein, 20 aa, Artificial Sequence
  synthetic polypeptide
GGGSGGGSGGGSGGGSGGGS

SEQ ID NO: 46 - protein, 24 aa, Artificial Sequence
  synthetic polypeptide
GGGSGGGSGGGSGGGSGGGSGGGS

SEQ ID NO: 47 - protein, 28 aa, Artificial Sequence
  synthetic polypeptide
GGGSGGGSGGGSGGGSGGGSGGGSGGGS

SEQ ID NO: 48 - protein, 32 aa, Artificial Sequence
  synthetic polypeptide
GGGSGGGSGGGSGGGSGGGSGGGSGGGSGGGS

SEQ ID NO: 49 - protein, 36 aa, Artificial Sequence
  synthetic polypeptide
GGGSGGGSGGGSGGGSGGGSGGGSGGGSGGGSGGGS

SEQ ID NO: 50 - protein, 40 aa, Artificial Sequence
  synthetic polypeptide
GGGSGGGSGGGSGGGSGGGSGGGSGGGSGGGSGGGSGGGS

SEQ ID NO: 51 - protein, 5 aa, Artificial Sequence
  synthetic polypeptide
GGGGS

SEQ ID NO: 52 - protein, 10 aa, Artificial Sequence
  synthetic polypeptide
GGGGSGGGGS

SEQ ID NO: 53 - protein, 15 aa, Artificial Sequence
  synthetic polypeptide
GGGGSGGGGSGGGGS

SEQ ID NO: 54 - protein, 20 aa, Artificial Sequence
  synthetic polypeptide
GGGGSGGGGSGGGGSGGGGS

SEQ ID NO: 55 - protein, 25 aa, Artificial Sequence
  synthetic polypeptide
GGGGSGGGGSGGGGSGGGGSGGGGS

SEQ ID NO: 56 - protein, 30 aa, Artificial Sequence
  synthetic polypeptide
GGGGSGGGGSGGGGSGGGGSGGGGSGGGGS

SEQ ID NO: 57 - protein, 35 aa, Artificial Sequence
  synthetic polypeptide
GGGGSGGGGSGGGGSGGGGSGGGGSGGGGSGGGGS

SEQ ID NO: 58 - protein, 40 aa, Artificial Sequence
  synthetic polypeptide
GGGGSGGGGSGGGGSGGGGSGGGGSGGGGSGGGGSGGGGS

SEQ ID NO: 59 - protein, 45 aa, Artificial Sequence
  synthetic polypeptide
GGGGSGGGGSGGGGSGGGGSGGGGSGGGGSGGGGSGGGGSGGGGS

SEQ ID NO: 60 - protein, 50 aa, Artificial Sequence
  synthetic polypeptide
GGGGSGGGGSGGGGSGGGGSGGGGSGGGGSGGGGSGGGGSGGGGSGGGGS

SEQ ID NO: 61 - protein, 6 aa, Artificial Sequence
  synthetic polypeptide
GGGGGS

SEQ ID NO: 62 - protein, 12 aa, Artificial Sequence
  synthetic polypeptide
GGGGGSGGGGGS

SEQ ID NO: 63 - protein, 18 aa, Artificial Sequence
  synthetic polypeptide
GGGGGSGGGGGSGGGGGS

SEQ ID NO: 64 - protein, 24 aa, Artificial Sequence
  synthetic polypeptide
GGGGGSGGGGGSGGGGGSGGGGGS

SEQ ID NO: 65 - protein, 30 aa, Artificial Sequence
  synthetic polypeptide
GGGGGSGGGGGSGGGGGSGGGGGSGGGGGS

SEQ ID NO: 66 - protein, 36 aa, Artificial Sequence
  synthetic polypeptide
GGGGGSGGGGGSGGGGGSGGGGGSGGGGGSGGGGGS

SEQ ID NO: 67 - protein, 42 aa, Artificial Sequence
  synthetic polypeptide
GGGGGSGGGGGSGGGGGSGGGGGSGGGGGSGGGGGSGGGGGS

SEQ ID NO: 68 - protein, 48 aa, Artificial Sequence
  synthetic polypeptide
GGGGGSGGGGGSGGGGGSGGGGGSGGGGGSGGGGGSGGGGGSGGGGGS

SEQ ID NO: 69 - protein, 54 aa, Artificial Sequence
  synthetic polypeptide
GGGGGSGGGGGSGGGGGSGGGGGSGGGGGSGGGGGSGGGGGSGGGGGSGGGGGS

SEQ ID NO: 70 - protein, 60 aa, Artificial Sequence
  synthetic polypeptide
GGGGGSGGGGGSGGGGGSGGGGGSGGGGGSGGGGGSGGGGGSGGGGGSGGGGGSGGGGGS

SEQ ID NO: 71 - protein, 6 aa, Artificial Sequence
  synthetic polypeptide
SGGGGS

SEQ ID NO: 72 - protein, 11 aa, Artificial Sequence
  synthetic polypeptide
SGGGGSGGGGS

SEQ ID NO: 73 - protein, 16 aa, Artificial Sequence
  synthetic polypeptide
SGGGGSGGGGSGGGGS

SEQ ID NO: 74 - protein, 21 aa, Artificial Sequence
  synthetic polypeptide
SGGGGSGGGGSGGGGSGGGGS

SEQ ID NO: 75 - protein, 26 aa, Artificial Sequence
  synthetic polypeptide
SGGGGSGGGGSGGGGSGGGGSGGGGS

SEQ ID NO: 76 - protein, 31 aa, Artificial Sequence
  synthetic polypeptide
SGGGGSGGGGSGGGGSGGGGSGGGGSGGGGS

SEQ ID NO: 77 - protein, 36 aa, Artificial Sequence
  synthetic polypeptide
SGGGGSGGGGSGGGGSGGGGSGGGGSGGGGSGGGGS

SEQ ID NO: 78 - protein, 41 aa, Artificial Sequence
  synthetic polypeptide
SGGGGSGGGGSGGGGSGGGGSGGGGSGGGGSGGGGSGGGGS

SEQ ID NO: 79 - protein, 46 aa, Artificial Sequence
  synthetic polypeptide
SGGGGSGGGGSGGGGSGGGGSGGGGSGGGGSGGGGSGGGGSGGGGS

SEQ ID NO: 80 - protein, 51 aa, Artificial Sequence
  synthetic polypeptide
SGGGGSGGGGSGGGGSGGGGSGGGGSGGGGSGGGGSGGGGSGGGGSGGGGS

SEQ ID NO: 81 - protein, 16 aa, Artificial Sequence
  synthetic polypeptide
GSGGGSGGGGSGGGGS

SEQ ID NO: 82 - protein, 1 aa, Artificial Sequence
  synthetic polypeptide
  misc_feature (1)..(1): Xaa is aspartic acid (D) or glutamic acid (E), and Xaa can range from 1 to up to 10 or more repeats
X

SEQ ID NO: 83 - protein, 6 aa, Artificial Sequence
  synthetic polypeptide
HHHHHH

SEQ ID NO: 84 - protein, 10 aa, Artificial Sequence
  synthetic polypeptide
EQKLISEEDL

SEQ ID NO: 85 - protein, 288 aa, Artificial Sequence
  synthetic polypeptide
EPEESDKTHTCPPCPAPEAAGGPSVFLFPPKPKDTLYITREPEVTCVVVDVSHEDPEVKFNWYVDGVEVHNAKTKPREEQYDSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGEDSLGREAKCYNELNGCTKIYDPVCGTDGNTYPNECVLCFENRKRQTSILIQKSGPC

SEQ ID NO: 86 - protein, 319 aa, Artificial Sequence
  synthetic polypeptide
EPKSSDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGADSLGREAKCYNELNGCTKIYDPVCGTDGNTYPNECVLCFENRKRQTSILIQKSGPCGPEQKLISEEDLNSALVPRGSRLVDHHHHHH

SEQ ID NO: 87 - protein, 288 aa, Artificial Sequence
  synthetic polypeptide
DSLGREAKCYNELNGCTKIYDPVCGTDGNTYPNECVLCFENRKRQTSILIQKSGPCEPKSSDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 88 - protein, 440 aa, Artificial Sequence
  synthetic polypeptide
DSLGREAKCYNELNGCTKIYDPVCGTDGNTYPNECVLCFENRKRQTSILIQKSGPCEPKSSDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGARPSGRKSSKMQAFRIWDVNQKTFYLRNNQLVAGYLQGPNVNLEEKIDVVPIEPHALFLGIHGGKMCLSCVKSGDETRLQLEAVDITDLSENRKQDKRFAFIRSDSGPTTSFESAACPGWFLCTAMEADQPVSLTNMPDEGVMVTKFYFQEDE

SEQ ID NO: 89 - protein, 440 aa, Artificial Sequence
  synthetic polypeptide
RPSGRKSSKMQAFRIWDVNQKTFYLRNNQLVAGYLQGPNVNLEEKIDVVPIEPHALFLGIHGGKMCLSCVKSGDETRLQLEAVDITDLSENRKQDKRFAFIRSDSGPTTSFESAACPGWFLCTAMEADQPVSLTNMPDEGVMVTKFYFQEDEEPKSSDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGADSLGREAKCYNELNGCTKIYDPVCGTDGNTYPNECVLCFENRKRQTSILIQKSGPC

SEQ ID NO: 90 - protein, 288 aa, Artificial Sequence; identical to SEQ ID NO: 9
  synthetic polypeptide

SEQ ID NO: 91 - protein, 290 aa, Artificial Sequence
  synthetic polypeptide
EPRGPTIKPCPPCKCPAPNLLGGPSVFIFPPKIKDVLMISLSPIVTCVVVDVSEDDPDVQISWFVNNVEVHTAQTQTHREDYDSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKAKVTGKEASCHDAVAGCPRIYDPVCGTDGITYANECVLCFENRKRIEPVLIRKGGPC

SEQ ID NO: 92 - protein, 290 aa, Artificial Sequence
  synthetic polypeptide
AKVTGKEASCHDAVAGCPRIYDPVCGTDGITYANECVLCFENRKRIEPVLIRKGGPCEPRGPTIKPCPPCKCPAPNLLGGPSVFIFPPKIKDVLMISLSPIVTCVVVDVSEDDPDVQISWFVNNVEVHTAQTQTHREDYDSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 93 - protein, 442 aa, Artificial Sequence
  synthetic polypeptide
AKVTGKEASCHDAVAGCPRIYDPVCGTDGITYANECVLCFENRKRIEPVLIRKGGPCEPRGPTIKPCPPCKCPAPNLLGGPSVFIFPPKIKDVLMISLSPIVTCVVVDVSEDDPDVQISWFVNNVEVHTAQTQTHREDYDSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 94 - protein, 442 aa, Artificial Sequence
  synthetic polypeptide
AKVTGKEASCHDAVAGCPRIYDPVCGTDGITYANECVLCFENRKRIEPVLIRKGGPCEPRGPTIKPCPPCKCPAPNLLGGPSVFIFPPKIKDVLMISLSPIVTCVVVDVSEDDPDVQISWFVNNVEVHTAQTQTHREDYDSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKRPSGKRPCKMQAFRIWDTNQKTFYLRNNQLIAGYLQGPNIKLEEKIDMVPIDLHSVFLGIHGGKLCLSCAKSGDDIKLQLEEVDITDLSKNKEEDKRFTFIRSEKGPTTSFESAACPGWFLCTTLEADRPVSLTNTPEEPLIVTKFYFQEDQ

SEQ ID NO: 95 - protein, 440 aa, Artificial Sequence; identical to SEQ ID NO: 88
  synthetic polypeptide

SEQ ID NO: 96 - protein, 440 aa, Artificial Sequence; identical to SEQ ID NO: 88
  synthetic polypeptide

SEQ ID NO: 97 - protein, 440 aa, Artificial Sequence; identical to SEQ ID NO: 88
  synthetic polypeptide

SEQ ID NO: 98 - protein, 296 aa, Artificial Sequence
  synthetic polypeptide
EPSGPISTINPCPPCKECHKCPAPNLEGGPSVFIFPPNIKDVLMISLTPKVTCVVVDVSEDDPDVRISWFVNNVEVHTAQTQTHREDYDSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGAAKVTGKEASCHDAVAGCPRIYDPVCGTDGITYANECVLCFENRKRIEPVLIRKGGPC

SEQ ID NO: 99 - protein, 296 aa, Artificial Sequence
  synthetic polypeptide
EPSGPISTINPCPPCKECHKCPAPNLEGGPSVFIFPPNIKDVLYITLEPKVTCVVVDVSEDDPDVRISWFVNNVEVHTAQTQTHREDYDSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGAAKVTGKEASCHDAVAGCPRIYDPVCGTDGITYANECVLCFENRKRIEPVLIRKGGPC

SEQ ID NO: 100 - protein, 296 aa, Artificial Sequence
  synthetic polypeptide
EPSGPISTINPCPPCKECHKCPAPNAAGGPSVFIFPPNIKDVLYITLEPKVTCVVVDVSEDDPDVRISWFVNNVEVHTAQTQTHREDYDSTIRVVSALPIQHQDWMSGKEFKCKVNNKDLGSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGAAKVTGKEASCHDAVAGCPRIYDPVCGTDGITYANECVLCFENRKRIEPVLIRKGGPC

SEQ ID NO: 101 - protein, 296 aa, Artificial Sequence
  synthetic polypeptide
EPSGPISTINPCPPCKECHKCPAPNLEGGPSVFIFPPNIKDVLYITLEPKVTCVVEDVSEDDPDVRISWFVNNVEVHTAQTQTHREDYDSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGAAKVTGKEASCHDAVAGCPRIYDPVCGTDGITYANECVLCFENRKRIEPVLIRKGGPC

SEQ ID NO: 102 - protein, 296 aa, Artificial Sequence
  synthetic polypeptide
EPSGPISTINPCPPCKECHKCPAPNLEGGPSVFIFPPNIKDVLMISLTPKVTCVVVDVSEDDPDVRISWFVNNVEVHTAQTQTHREDYDSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVLHEGLKSYYLKKTISRSPGAAKVTGKEASCHDAVAGCPRIYDPVCGTDGITYANECVLCFENRKRIEPVLIRKGGPC

SEQ ID NO: 103 - protein, 296 aa, Artificial Sequence
  synthetic polypeptide
EPSGPISTINPCPPCKECHKCPAPNAAGGPSVFIFPPNIKDVLMISLTPKVTCVVVDVSEDDPDVRISWFVNNVEVHTAQTQTHREDYDSTIRVVSALPIQHQDWMSGKEFKCKVNNKDLGSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVLHEGLKSYYLKKTISRSPGAAKVTGKEASCHDAVAGCPRIYDPVCGTDGITYANECVLCFENRKRIEPVLIRKGGPC

SEQ ID NO: 104 - protein, 296 aa, Artificial Sequence
  synthetic polypeptide
EPSGPISTINPCPPCKECHKCPAPNLEGGPSVFIFPPNIKDVLMISLTPKVTCVVEDVSEDDPDVRISWFVNNVEVHTAQTQTHREDYDSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVLHEGLKSYYLKKTISRSPGAAKVTGKEASCHDAVAGCPRIYDPVCGTDGITYANECVLCFENRKRIEPVLIRKGGPC

SEQ ID NO: 105 - protein, 296 aa, Artificial Sequence
  synthetic polypeptide
EPSGPISTINPCPPCKECHKCPAPNLEGGPSVFIFPPNIKDVLMISLTPKVTCVVVDVSEDDPDVRISWFVNNVEVHTAQTQTHREDYDSTIRVVSALPIDHHDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKSYYLKKTISRSPGAAKVTGKEASCHDAVAGCPRIYDPVCGTDGITYANECVLCFENRKRIEPVLIRKGGPC

SEQ ID NO: 106 - protein, 296 aa, Artificial Sequence
  synthetic polypeptide
EPSGPISTINPCPPCKECHKCPAPNAAGGPSVFIFPPNIKDVLMISLTPKVTCVVVDVSEDDPDVRISWFVNNVEVHTAQTQTHREDYDSTIRVVSALPIDHHDWMSGKEFKCKVNNKDLGSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKSYYLKKTISRSPGAAKVTGKEASCHDAVAGCPRIYDPVCGTDGITYANECVLCFENRKRIEPVLIRKGGPC

SEQ ID NO: 107 - protein, 296 aa, Artificial Sequence
  synthetic polypeptide
EPSGPISTINPCPPCKECHKCPAPNLEGGPSVFIFPPNIKDVLMISLTPKVTCVVEDVSEDDPDVRISWFVNNVEVHTAQTQTHREDYDSTIRVVSALPIDHHDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKSYYLKKTISRSPGAAKVTGKEASCHDAVAGCPRIYDPVCGTDGITYANECVLCFENRKRIEPVLIRKGGPC

SEQ ID NO: 108 - protein, 289 aa, Artificial Sequence
  synthetic polypeptide
EPKSSPPCKECSIFPAPDAAGGPSVFIFPPKIKDVLYITLEPEVTCVVVDVSEDDPDVQISWFVNNVEVHTAQTQTHREDYDSTLRVVSALPIQHQDWMSGKEFKCKVNNRALPSPIEKTISKPRGPVRAPQVYVLPPPAEEMTKKEFSLTCMITDFLPAEIAVDWTSNGHKELNYKNTAPVLDTDGSYFMYSKLRVQKSTWEKGSLFACSVVHEGLHNHHTTKTISRSLGAAKVTGKEASCHDAVAGCPRIYDPVCGTDGITYANECVLCFENRKRIEPVLIRKGGPC

SEQ ID NO: 109 - protein, 289 aa, Artificial Sequence
  synthetic polypeptide
EPESEPPCKECSIFPAPDAAGGPSVFIFPPKIKDVLYITLEPEVTCVVVDVSEDDPDVQISWFVNNVEVHTAQTQTHREDYDSTLRVVSALPIQHQDWMSGKEFKCKVNNRALPSPIEKTISKPRGPVRAPQVYVLPPPAEEMTKKEFSLTCMITDFLPAEIAVDWTSNGHKELNYKNTAPVLDTDGSYFMYSKLRVQKSTWEKGSLFACSVVHEGLHNHHTTKTISRSLGEAKVTGKEASCHDAVAGCPRIYDPVCGTDGITYANECVLCFENRKRIEPVLIRKGGPC

SEQ ID NO: 110 - protein, 301 aa, Artificial Sequence
  synthetic polypeptide
YPYDVPDYAKPKEPESEPPCKECSIFPAPDAAGGPSVFIFPPKIKDVLYITLEPEVTCVVVDVSEDDPDVQISWFVNNVEVHTAQTQTHREDYDSTLRVVSALPIQHQDWMSGKEFKCKVNNRALPSPIEKTISKPRGPVRAPQVYVLPPPAEEMTKKEFSLTCMITDFLPAEIAVDWTSNGHKELNYKNTAPVLDTDGSYFMYSKLRVQKSTWEKGSLFACSVVHEGLHNHHTTKTISRSLGEAKVTGKEASCHDAVAGCPRIYDPVCGTDGITYANECVLCFENRKRIEPVLIRKGGPC

SEQ ID NO: 111 - protein, 288 aa, Artificial Sequence
  synthetic polypeptide
EPKSSDKTHTCPPCPAPEAAGGPSVFLFPPKPKDTLYITREPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGADSLGREAKCYNELNGCTKIYDPVCGTDGNTYPNECVLCFENRKRQTSILIQKSGPC

SEQ ID NO: 112 - protein, 288 aa, Artificial Sequence
  synthetic polypeptide
EPKSSDKTHTCPPCPAPEAAGGPSVFLFPPKPKDTLYITREPEVTCVVVDVSHEDPEVKFNWYVDGVEVHNAKTKPREEQYDSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGADSLGREAKCYNELNGCTKIYDPVCGTDGNTYPNECVLCFENRKRQTSILIQKSGPC

SEQ ID NO: 113 - protein, 288 aa, Artificial Sequence
  synthetic polypeptide
EPKSSDKTHTCPPCPAPEAAGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGADSLGREAKCYNELNGCTKIYDPVCGTDGNTYPNECVLCFENRKRQTSILIQKSGPC

SEQ ID NO: 114 - protein, 288 aa, Artificial Sequence
  synthetic polypeptide
EPKSSDKTHTCPPCPAPEAAGGPSVFLFPPKPKDTLMISRTPEVTCVVVDVSHEDPEVKFNWYVDGVEVHNAKTKPREEQYDSTYRVVSVLTVDHHDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGADSLGREAKCYNELNGCTKIYDPVCGTDGNTYPNECVLCFENRKRQTSILIQKSGPC

SEQ ID NO: 115 - DNA, 4327 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaaccagaagagtctgataagactcatacttgtccaccatgtccagctccagaagctgctggtggtccatctgtttttttgtttccaccaaagccaaaggatactttgtacattactagagagccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttttgcatcaagattggttgaacggtaaggaatacaagtgtaaggtttctaacaaggctttgggtgctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgaggattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgttgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 116 - DNA, 4420 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaaccaaagtcttctgataagacccatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgtggtccagaacaaaagttgatttctgaagaagatttgaactctgctttggttccaagaggttccagattggttgatcatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 117 - DNA, 4327 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaaccaaagtcttctgataagactcatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 118 - DNA, 4327 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgtgaaccaaagtcttctgataagactcatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgcttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 119 - DNA, 4783 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgtgaaccaaagtcttctgataagacacatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgattctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctaggccatccggtagaaaatcttccaaaatgcaagccttcaggatctgggatgttaaccaaaaaactttttacctgaggaacaatcaattggtggctggatatttacaaggaccaaatgttaacctcgaagagaaaatagatgtagtccctatcgagccacatgccttgtttttgggaatccatggtgggaaaatgtgtttatcgtgtgtcaagtctggagacgagacgcgtctccaattggaggccgtcgatattacggacttatctgagaaccgaaaacaagacaaacgttttgctttcatcaggtcagacagtggacccacaacttccttcgaatcagctgcctgtccaggatggtttctatgcacagcaatggaagctgatcagcctgtttcgttgactaatatgcccgatgagggtgtcatggtcactaaattctattttcaggaggacgagtaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 120 - DNA, 4783 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctaggccatccggtagaaaatcttccaaaatgcaagccttcaggatctgggatgttaaccaaaaaactttttacctgaggaacaatcaattggtggctggatatttacaaggaccaaatgttaacctcgaagagaaaatagatgtagtccctatcgagccacatgccttgtttttgggaatccatggtgggaaaatgtgtttatcgtgtgtcaagtctggagacgagacgcgtctccaattggaggccgtcgatattacggacttatctgagaaccgaaaacaagacaaacgttttgctttcatcaggtcagacagtggacccacaacttccttcgaatcagctgcctgtccaggatggtttctatgcacagcaatggaagctgatcagcctgtttcgttgactaatatgcccgatgagggtgtcatggtcactaaattctattttcaggaggacgaggaaccaaagtcttctgataagacacatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgattctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 121 - DNA, 4327 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaaccagaagaatctgataagacccatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgattctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgaagattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 122 - DNA, 4333 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaacctagaggtccaacaattaagccttgtcctccatgcaagtgtccagctccaaatttgttgggaggtccatctgtttttatcttcccacctaagatcaaagacgttttgatgatctctttgtctcctatcgttacatgtgttgtcgttgatgtctctgaggacgatccagacgttcagatttcttggtttgttaataacgttgaagttcacactgcccaaactcagacccacagagaggattatgactctactttgagagtcgtttctgccttgcctatccaacaccaagactggatgtctggaaaagaatttaaatgtaaagttaacaataaggacttgcctgcaccaattgaaagaactatttctaagcctaagggttctgttagagcaccacaggtttacgttttgcctccacctgaggaagagatgactaaaaagcaggtcaccttgacctgcatggttaccgactttatgccagaagacatctacgttgaatggacaaacaatggtaaaacagaattgaactacaaaaacactgaacctgtcttggattctgatggttcttatttcatgtactctaaattgagagtcgagaaaaagaattgggttgaaagaaactcttactcttgttctgttgtccacgaaggtttgcataaccaccatactaccaagtctttctccagaaccccaggtaaagctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 123 - DNA, 4333 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgtgaacctagaggtccaacaattaagccttgtcctccatgcaagtgtccagctccaaatttgttgggaggtccatctgtttttatcttcccacctaagatcaaagacgttttgatgatctctttgtctcctatcgttacatgtgttgtcgttgatgtctctgaggacgatccagacgttcagatttcttggtttgttaataacgttgaagttcacactgcccaaactcagacccacagagaggattatgactctactttgagagtcgtttctgccttgcctatccaacaccaagactggatgtctggaaaagaatttaaatgtaaagttaacaataaggacttgcctgcaccaattgaaagaactatttctaagcctaagggttctgttagagcaccacaggtttacgttttgcctccacctgaggaagagatgactaaaaagcaggtcaccttgacctgcatggttaccgactttatgccagaagacatctacgttgaatggacaaacaatggtaaaacagaattgaactacaaaaacactgaacctgtcttggattctgatggttcttatttcatgtactctaaattgagagtcgagaaaaagaattgggttgaaagaaactcttactcttgttctgttgtccacgaaggtttgcataaccaccatactaccaagtctttctccagaaccccaggtaaataagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 124 - DNA, 4789 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgtgaacctagaggtccaacaattaagccttgtcctccatgcaagtgtccagctccaaatttgttgggaggtccatctgtttttatcttcccacctaagatcaaagacgttttgatgatctctttgtctcctatcgttacatgtgttgtcgttgatgtctctgaggacgatccagacgttcagatttcttggtttgttaataacgttgaagttcacactgcccaaactcagacccacagagaggattatgactctactttgagagtcgtttctgccttgcctatccaacaccaagactggatgtctggaaaagaatttaaatgtaaagttaacaataaggacttgcctgcaccaattgaaagaactatttctaagcctaagggttctgttagagcaccacaggtttacgttttgcctccacctgaggaagagatgactaaaaagcaggtcaccttgacctgcatggttaccgactttatgccagaagacatctacgttgaatggacaaacaatggtaaaacagaattgaactacaaaaacactgaacctgtcttggattctgatggttcttatttcatgtactctaaattgagagtcgagaaaaagaattgggttgaaagaaactcttactcttgttctgttgtccacgaaggtttgcataaccaccatactaccaagtctttctccagaaccccaggtaaaagaccttctggaaaaagaccttgtaagatgcaagcctttagaatctgggatactaaccaaaagacattctatttgagaaataaccaattgattgccggatatttgcagggacctaatattaaattggaggaaaagattgatatggttccaatcgatttgcactctgtctttttgggaatccatggaggtaagttgtgtttgtcttgcgccaagtctggtgacgatattaagttgcagttggaagaggttaacatcactgacttgtctaagaacaaagaagaggataaaagattcacttttatcagatctgaaaagggtcctactacatctttcgagtctgccgcttgtccaggttggtttttgtgcaccactttggaggctgatagaccagtctctttgactaacactcctgaggaaccattgatcgttactaagttctatttccaggaagatcaataagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 125 - DNA, 4789 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgtgaacctagaggtccaacaattaagccttgtcctccatgcaagtgtccagctccaaatttgttgggaggtccatctgtttttatcttcccacctaagatcaaagacgttttgatgatctctttgtctcctatcgttacatgtgttgtcgttgatgtctctgaggacgatccagacgttcagatttcttggtttgttaataacgttgaagttcacactgcccaaactcagacccacagagaggattatgactctactttgagagtcgtttctgccttgcctatccaacaccaagactggatgtctggaaaagaatttaaatgtaaagttaacaataaggacttgcctgcaccaattgaaagaactatttctaagcctaagggttctgttagagcaccacaggtttacgttttgcctccacctgaggaagagatgactaaaaagcaggtcaccttgacctgcatggttaccgactttatgccagaagacatctacgttgaatggacaaacaatggtaaaacagaattgaactacaaaaacactgaacctgtcttggattctgatggttcttatttcatgtactctaaattgagagtcgagaaaaagaattgggttgaaagaaactcttactcttgttctgttgtccacgaaggtttgcataaccaccatactaccaagtctttctccagaaccccaggtaaaagaccttctggaaaaagaccttgtaagatgcaagcctttagaatctgggatactaaccaaaagacattctatttgagaaataaccaattgattgccggatatttgcagggacctaatattaaattggaggaaaagattgatatggttccaatcgatttgcactctgtctttttgggaatccatggaggtaagttgtgtttgtcttgcgccaagtctggtgacgatattaagttgcagttggaagaggttgatatcactgacttgtctaagaacaaagaagaggataaaagattcacttttatcagatctgaaaagggtcctactacatctttcgagtctgccgcttgtccaggttggtttttgtgcaccactttggaggctgatagaccagtctctttgactaacactcctgaggaaccattgatcgttactaagttctatttccaggaagatcaataagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 126 - DNA, 4792 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgaggcaggtttggttctcttggattgtgggattgttcctatgttttttcaacgtgtcttctgctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgtgaaccaaagtcttctgataagacacatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgattctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctaggccatccggtagaaaatcttccaaaatgcaagccttcaggatctgggatgttaaccaaaaaactttttacctgaggaacaatcaattggtggctggatatttacaaggaccaaatgttaacctcgaagagaaaatagatgtagtccctatcgagccacatgccttgtttttgggaatccatggtgggaaaatgtgtttatcgtgtgtcaagtctggagacgagacgcgtctccaattggaggccgtcgatattacggacttatctgagaaccgaaaacaagacaaacgttttgctttcatcaggtcagacagtggacccacaacttccttcgaatcagctgcctgtccaggatggtttctatgcacagcaatggaagctgatcagcctgtttcgttgactaatatgcccgatgagggtgtcatggtcactaaattctattttcaggaggacgagtaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 127 - DNA, 4741 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccgccagcattgctgctaaagaagaaggggtatctctcgagaaaagagaggctgaagctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgtgaaccaaagtcttctgataagacacatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgattctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctaggccatccggtagaaaatcttccaaaatgcaagccttcaggatctgggatgttaaccaaaaaactttttacctgaggaacaatcaattggtggctggatatttacaaggaccaaatgttaacctcgaagagaaaatagatgtagtccctatcgagccacatgccttgtttttgggaatccatggtgggaaaatgtgtttatcgtgtgtcaagtctggagacgagacgcgtctccaattggaggccgtcgatattacggacttatctgagaaccgaaaacaagacaaacgttttgctttcatcaggtcagacagtggacccacaacttccttcgaatcagctgcctgtccaggatggtttctatgcacagcaatggaagctgatcagcctgtttcgttgactaatatgcccgatgagggtgtcatggtcactaaattctattttcaggaggacgagtaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 128 - DNA, 4585 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgaggctgaagctaggccatccggtagaaaatcttccaaaatgcaagccttcaggatctgggatgttaaccaaaaaactttttacctgaggaacaatcaattggtggctggatatttacaaggaccaaatgttaacctcgaagagaaaatagatgtagtccctatcgagccacatgccttgtttttgggaatccatggtgggaaaatgtgtttatcgtgtgtcaagtctggagacgagacgcgtctccaattggaggccgtcgatattacggacttatctgagaaccgaaaacaagacaaacgttttgctttcatcaggtcagacagtggacccacaacttccttcgaatcagctgcctgtccaggatggtttctatgcacagcaatggaagctgatcagcctgtttcgttgactaatatgcccgatgagggtgtcatggtcactaaattctattttcaggaggacgaggaaccaaagtcttctgataagacacatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgattctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 129 - DNA, 4351 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgagcctagtggaccgattagtaccatcaacccatgtcctccatgcaaagagtgccataaatgccctgcccctaatttggaagggggaccatcggtgtttatttttccacccaatattaaggacgttttgatgatttctctaactccaaaggttacctgcgttgtcgtggatgtttcagaggacgatcctgatgtacgtatctcttggtttgtgaacaatgtcgaagttcacaccgcacaaacgcagacacacagagaagattacgattctaccatcagagtggtatcagctctaccaatccaacatcaagattggatgagtggaaaggaatttaaatgtaaagttaacaataaagacctaccgtctcctattgaaaggacgatatcgaaaattaagggtctggtgagagcacctcaagtgtatattcttccacctccggcagagcagttgtcaagaaaggatgtgtcattgacttgcttagtggtcggatttaacccgggtgatataagcgtggagtggacatctaatggtcatactgaggaaaattataaagacacggctccagtcctcgactctgacgggtcctattttatatactcgaagctcgatatcaagacctctaagtgggaaaaaaccgactccttcagttgcaatgtaagacatgaaggactgaagaattactatctgaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 130 - DNA, 4351 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgagcctagtggaccgattagtaccatcaacccttgccctccatgtaaggaatgccataaatgtcctgcacctaacttagagggtggaccgtcggtcttcattttcccacctaatataaaagatgtcttgtacataacactggaaccaaaagttacatgtgtggtcgtggatgtttccgaagatgacccagatgttagaattagctggttcgtaaacaatgtagaagtccacaccgctcaaacccaaacacatcgtgaggattatgactccaccattagagtcgtttccgctctccctatacaacaccaggactggatgtcagggaaagagtttaaatgtaaagttaataacaaggatctcccatctccaattgagaggactatctccaagattaaaggtctggtaagagcaccccaagtttatattctaccacctccagctgaacaactttctagaaaggatgtttctcttacatgtttagtggttggcttcaaccccggagacatatctgttgagtggacgtcaaacggtcatactgaagagaattataaagataccgcccccgtgttggacagtgacggaagttacttcatctactccaagttggatataaagacctcaaagtgggagaaaaccgattccttctcctgcaatgttcgtcacgagggtttaaaaaactattacctgaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 131 - DNA, 4351 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgagcctagtggaccgattagtaccatcaacccatgtcccccttgtaaagaatgtcataagtgtcctgctccgaacgctgccggcggtccttcggtgtttatttttccccctaatataaaggacgttctttacattactctggagccaaaggtcacttgcgtagttgtggacgtatcagaagatgacccagatgttagaatatcttggttcgtgaacaatgtggaagtgcatacagcgcagacacaaacccatcgagaggactacgactcaaccatccgtgtagtttcagctcttcccattcaacatcaagactggatgtcaggtaaggaattcaagtgtaaggtaaataacaaggatcttggatcaccaattgaacgtaccattagcaagattaagggtcttgtaagagcccctcaagtgtacattcttcctccacctgctgaacaattatctcgtaaagatgtttcattaacctgtctggtcgttggttttaatcccggtgatatttcagttgaatggacctcaaacggtcatacagaggaaaattacaaagacactgccccagtgttagattctgatgggtcttactttatctactcaaagttagatatcaagacttctaagtgggaaaagactgactctttcagctgcaacgttcgacacgaaggactgaagaattattacttgaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 132 - DNA, 4351 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgagcctagtggaccgattagtaccatcaacccttgccctccatgtaaggaatgccataaatgtcctgcacctaacttagagggtggaccgtcggtcttcattttcccacctaatataaaagatgtcttgtacataacactggaaccaaaagttacatgtgtggtcgaggatgtttccgaagatgacccagatgttagaattagctggttcgtaaacaatgtagaagtccacaccgctcaaacccaaacacatcgtgaggattatgactccaccattagagtcgtttccgctctccctatacaacaccaggactggatgtcagggaaagagtttaaatgtaaagttaataacaaggatctcccatctccaattgagaggactatctccaagattaaaggtctggtaagagcaccccaagtttatattctaccacctccagctgaacaactttctagaaaggatgtttctcttacatgtttagtggttggcttcaaccccggagacatatctgttgagtggacgtcaaacggtcatactgaagagaattataaagataccgcccccgtgttggacagtgacggaagttacttcatctactccaagttggatataaagacctcaaagtgggagaaaaccgattccttctcctgcaatgttcgtcacgagggtttaaaaaactattacctgaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 133 - DNA, 4351 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgagcctagtggaccgattagtaccatcaacccatgtcctccctgtaaggaatgtcacaaatgtccagctcctaatctggagggagggccatccgtgtttatcttcccacctaacattaaagatgttttgatgatttccttgactccaaaagtgacttgcgtcgtagtggacgtttctgaagacgatccagatgtgagaattagctggttcgttaacaatgtcgaggttcatacagcacaaacccaaacccacagagaggattacgatagcactatcagggtcgtttccgctcttcctattcagcaccaagattggatgagcggaaaagaatttaaatgtaaggttaataacaaagacctgccatccccaattgaaagaacaatttctaagattaaaggtttggtccgtgcacctcaagtttatatcttgccacccccagcagaacaattgtcaagaaaagatgtctctcttacatgcttagtcgttggtttcaatcctggggatatttctgtagagtggacaagtaatggacatacagaggaaaattataaagataccgctccagtacttgattccgatggttcctacttcatctattctaagctggacattaaaacctcaaagtgggaaaaaactgactcttttagttgtaacgttctgcatgagggcttgaaatcttattaccttaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 134 - DNA, 4351 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgagcctagtggaccgattagtaccatcaacccgtgtccaccttgcaaagaatgtcataaatgtcctgcccccaatgccgctggaggtccttcggtatttatttttcccccaaatattaaagacgtccttatgatttctttaactcccaaagtcacttgtgttgtagttgacgtgtcagaggacgatcccgacgtaagaatttcctggtttgtaaataacgttgaagtacacacagcacaaactcaaactcacagagaggactacgattctactattagagtggtatctgctttgccaattcaacatcaggactggatgtcaggtaaggagtttaaatgtaaagttaacaataaggaccttggttcacccatcgaaagaactattagtaaaatcaagggattagtccgtgctcctcaggtgtacattcttccacctccagccgagcagctgagtcgaaaggatgttagtctaacctgcctggtggtaggtttcaaccctggagatatttcggtggagtggacatccaacggtcatactgaagagaattacaaggataccgcccccgtcctagattcagatggttcttacttcatttactcgaaactagacattaaaaccagcaagtgggagaagactgacagtttctcctgcaatgtcttgcatgaaggattgaagtcttattaccttaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 135 - DNA, 4351 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgagcctagtggaccgattagtaccatcaacccatgtcctccctgtaaggaatgtcacaaatgtccagctcctaatctggagggagggccatccgtgtttatcttcccacctaacattaaagatgttttgatgatttccttgactccaaaagtgacttgcgtcgtagaggacgtttctgaagacgatccagatgtgagaattagctggttcgttaacaatgtcgaggttcatacagcacaaacccaaacccacagagaggattacgatagcactatcagggtcgtttccgctcttcctattcagcaccaagattggatgagcggaaaagaatttaaatgtaaggttaataacaaagacctgccatccccaattgaaagaacaatttctaagattaaaggtttggtccgtgcacctcaagtttatatcttgccacccccagcagaacaattgtcaagaaaagatgtctctcttacatgcttagtcgttggtttcaatcctggggatatttctgtagagtggacaagtaatggacatacagaggaaaattataaagataccgctccagtacttgattccgatggttcctacttcatctattctaagctggacattaaaacctcaaagtgggaaaaaactgactcttttagttgtaacgttctgcatgagggcttgaaatcttattaccttaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 136 - DNA, 4351 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgagcctagtggaccgattagtaccatcaacccttgccctccatgcaaagaatgtcataaatgtcctgccccgaaccttgagggtggaccgtctgttttcatctttcccccaaacattaaggacgttttgatgatcagcctgactccaaaagtgacatgtgttgtggttgatgtttcagaagatgacccagacgtgcggatctcttggtttgtgaataacgtcgaggtccatacagcgcagacccaaacccaccgagaggattacgactcgacaattcgcgtggtctctgcacttcccattgatcaccatgactggatgtccggtaaggaatttaagtgcaaagttaacaataaggacttgccttctcctattgagagaacaatttctaagattaaaggccttgtaagggctccacaagtttacattttgccacccccagcggagcaactgtctagaaaggatgtcagtcttacctgcctagtagtggggtttaatcctggtgatatctctgtcgaatggacgagcaacggacacactgaagagaattataaggatacagcacctgtcttagattcagatggaagttactttatttactctaaattggacattaaaacctctaaatgggaaaagactgattcatttagttgtaatgtccgccatgaggggttaaaatcctattaccttaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 137 - DNA, 4351 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgagcctagtggaccgattagtaccatcaacccttgcccaccttgtaaagaatgtcataaatgccctgctccgaatgctgcaggcggtccaagtgtattcattttcccgccaaacataaaagatgtcttaatgatttctctgactcctaaggtgacatgtgtagtcgtggatgtttcggaggacgatcccgacgttagaattagttggttcgttaataacgtcgaagtgcacactgctcaaactcagacacacagagaagactatgattctactattagagtcgtttctgcactacctatcgaccaccatgattggatgagtggaaaggagtttaaatgtaaagtcaacaataaggatcttggatctccaattgagcgtacgatttctaagatcaaaggtctggttcgtgcgccacaggtctatatactacccccacctgcagagcaactgtccagaaaagacgtttctcttacttgcttggtagtggggttcaatcctggtgatatttctgtcgagtggacaagtaacggtcacactgaggaaaattacaaggatactgcacctgtcctagattctgatggttcctactttatttacagtaaattagacatcaaaacgtctaagtgggaaaagacagactcattcagctgtaatgttcgtcacgagggtctaaaatcatactatttgaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 138 - DNA, 4351 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgagcctagtggaccgattagtaccatcaacccatgtccaccctgtaaagagtgccataagtgccctgcacctaatttagagggcggtccatccgtgttcatttttccacccaacatcaaggatgtcctcatgatctcactgacacctaaagttacctgtgtagttgaggatgtttcagaagacgatcccgatgtccggatttcttggtttgtaaacaatgttgaagtgcacacggcacaaacacaaactcatagagaagattatgatagcacaatcagggtcgtttccgcccttccaattgatcatcacgattggatgtccggaaaagagtttaaatgtaaagttaacaataaagatctgccctcgccaattgaaagaaccatctctaagattaagggattagttcgtgccccgcaagtttacattcttccacctccagctgaacagcttagtagaaaggacgttagtttgacctgtttggtagttggtttcaatcccggtgatataagtgtggaatggacctctaatggtcacactgaagagaattacaaggataccgctccagtgttggatagcgatggttcttacttcatttactctaagttggacattaagacgtctaaatgggagaaaactgacagtttttcttgtaacgttagacatgaaggcctaaagtcttattacttgaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 139 - DNA, 4330 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgagcccaaatcaagtcccccttgcaaagagtgtagcatttttccagcacccgatgcagccggaggtccttccgtgttcattttccctcccaaaatcaaagatgttctatacatcacgctggagccagaagtcacgtgtgtggttgtcgatgttagtgaagatgaccccgatgtccaaattagttggttcgttaacaatgtagaagtccatacggctcagactcagactcacagagaggattatgactcgactttaagagtcgtttctgcattaccaattcaacaccaagactggatgtctggtaaagaatttaaatgtaaagtaaataacagggcccttccatctccgattgaaaagactatttccaagccaagaggtcccgtacgtgctccacaggtctacgttctgccgccacctgccgaggaaatgactaaaaaggaattttctttgacctgtatgattactgatttcttgccagctgaaattgctgttgattggacctcaaatggtcacaaagagttgaactataagaataccgctccagtcttggacacggatggttcttattttatgtatagcaagcttcgagttcaaaagagtacttgggaaaaagggtcactatttgcatgttcagttgtgcatgaggggcttcataaccaccataccactaagacaatttctagatcgttgggtgcagctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 140 - DNA, 4330 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaacctgagtctgagcctccctgtaaagaatgctccatttttccagctccagatgccgcagggggtccttccgttttcatatttccgccaaagataaaagatgtcctgtacattacactggagcctgaagtgacttgtgttgtagttgatgtttcagaagatgaccctgacgtgcagatctcttggtttgttaacaatgttgaagtgcataccgctcaaactcagacccacagagaggactacgactctactttacgtgttgtgtctgcactgcctattcagcaccaagattggatgtctggtaaagagtttaaatgtaaggtgaataaccgtgctttgccttctccaattgagaaaacaataagtaagccaagaggaccggttagagcccctcaggtttatgtcttacctccacctgctgaggaaatgacgaaaaaggagttttcactgacatgtatgatcacagattttctgcctgccgagattgcagttgactggacgagtaacggacataaagagttaaactacaaaaataccgcacctgtcctggataccgatggatcttattttatgtactcaaagcttagagtccaaaagtctacatgggagaagggatctttgttcgcttgtagcgtggttcatgaaggtcttcacaatcaccatactacaaagacaatcagtcggtcgctaggagaagctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 141 - DNA, 4366 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcttacccctacgatgtcccagactacgccaaacctaaagagcccgaatctgagccaccttgtaaggagtgttccatctttcctgccccagatgcagccggtggacctagtgtctttatcttccctccaaaaatcaaggatgttctgtatattacgttagaaccagaagttacttgtgtcgttgtagacgtttcagaagacgatcctgacgtccaaatttcgtggtttgttaataacgtcgaagtacatactgcacagactcaaacccatcgcgaggactacgactcaaccttgagagttgtctcagccctgcctatccaacatcaagactggatgtctggtaaggagtttaaatgtaaggttaataacagagcccttccatctcctatagagaaaactatttctaagccgcgaggtcctgttcgggctccgcaggtttatgtcctgcccccacctgctgaggaaatgactaagaaagaattctctttaacatgtatgattacggactttctacccgcagagatcgccgtcgactggacttcaaacggtcacaaagaactaaattacaaaaacactgctcctgtattagatacggacggtagttattttatgtattccaagttgagagttcaaaagtccacttgggagaagggttctctgttcgcttgttcagttgtccacgaaggattgcataaccatcacacgactaagaccatttcaagatctctgggggaggctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 142 - DNA, 4327 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaaccaaagtcttctgataagactcatacttgtccaccatgtccagctccagaagctgctggtggtccatctgtttttttgtttccaccaaagccaaaggatactttgtacattactagagagccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 143 - DNA, 4327 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaaccaaagtcttctgataagactcatacttgtccaccatgtccagctccagaagctgctggtggtccatctgtttttttgtttccaccaaagccaaaggatactttgtacattactagagagccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttttgcatcaagattggttgaacggtaaggaatacaagtgtaaggtttctaacaaggctttgggtgctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 144 - DNA, 4327 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaaccaaagtcttctgataagactcatacttgtccaccatgtccagctccagaagctgctggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttttgcatcaagattggttgaacggtaaggaatacaagtgtaaggtttctaacaaggctttgggtgctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttttgcatgaagctttgcattctcattacactcaaaagtctttgtctttgtctccaggtgctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 145 - DNA, 4327 bp, Artificial Sequence
  synthetic vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaaccaaagtcttctgataagactcatacttgtccaccatgtccagctccagaagctgctggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttgatcatcatgattggttgaacggtaaggaatacaagtgtaaggtttctaacaaggctttgggtgctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcattctcattacactcaaaagtctttgtctttgtctccaggtgctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgttaagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 146 - DNA, 957 bp, Artificial Sequence
  synthetic polynucleotide
gaaccaaagtcttctgataagacccatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgtggtccagaacaaaagttgatttctgaagaagatttgaactctgctttggttccaagaggttccagattggttgatcatcatcatcatcatcat

SEQ ID NO: 147 - DNA, 864 bp, Artificial Sequence
  synthetic polynucleotide
gaaccaaagtcttctgataagactcatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgt

SEQ ID NO: 148 - DNA, 864 bp, Artificial Sequence
  synthetic polynucleotide
gattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgtgaaccaaagtcttctgataagactcatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgct

SEQ ID NO: 149 - DNA, 1320 bp, Artificial Sequence
  synthetic polynucleotide
gattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgtgaaccaaagtcttctgataagacacatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgattctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctaggccatccggtagaaaatcttccaaaatgcaagccttcaggatctgggatgttaaccaaaaaactttttacctgaggaacaatcaattggtggctggatatttacaaggaccaaatgttaacctcgaagagaaaatagatgtagtccctatcgagccacatgccttgtttttgggaatccatggtgggaaaatgtgtttatcgtgtgtcaagtctggagacgagacgcgtctccaattggaggccgtcgatattacggacttatctgagaaccgaaaacaagacaaacgttttgctttcatcaggtcagacagtggacccacaacttccttcgaatcagctgcctgtccaggatggtttctatgcacagcaatggaagctgatcagcctgtttcgttgactaatatgcccgatgagggtgtcatggtcactaaattctattttcaggaggacgag

SEQ ID NO: 150 - DNA, 1320 bp, Artificial Sequence
  synthetic polynucleotide
aggccatccggtagaaaatcttccaaaatgcaagccttcaggatctgggatgttaaccaaaaaactttttacctgaggaacaatcaattggtggctggatatttacaaggaccaaatgttaacctcgaagagaaaatagatgtagtccctatcgagccacatgccttgtttttgggaatccatggtgggaaaatgtgtttatcgtgtgtcaagtctggagacgagacgcgtctccaattggaggccgtcgatattacggacttatctgagaaccgaaaacaagacaaacgttttgctttcatcaggtcagacagtggacccacaacttccttcgaatcagctgcctgtccaggatggtttctatgcacagcaatggaagctgatcagcctgtttcgttgactaatatgcccgatgagggtgtcatggtcactaaattctattttcaggaggacgaggaaccaaagtcttctgataagacacatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgattctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgt

SEQ ID NO: 151 - DNA, 870 bp, Artificial Sequence
  synthetic polynucleotide
gaacctagaggtccaacaattaagccttgtcctccatgcaagtgtccagctccaaatttgttgggaggtccatctgtttttatcttcccacctaagatcaaagacgttttgatgatctctttgtctcctatcgttacatgtgttgtcgttgatgtctctgaggacgatccagacgttcagatttcttggtttgttaataacgttgaagttcacactgcccaaactcagacccacagagaggattatgactctactttgagagtcgtttctgccttgcctatccaacaccaagactggatgtctggaaaagaatttaaatgtaaagttaacaataaggacttgcctgcaccaattgaaagaactatttctaagcctaagggttctgttagagcaccacaggtttacgttttgcctccacctgaggaagagatgactaaaaagcaggtcaccttgacctgcatggttaccgactttatgccagaagacatctacgttgaatggacaaacaatggtaaaacagaattgaactacaaaaacactgaacctgtcttggattctgatggttcttatttcatgtactctaaattgagagtcgagaaaaagaattgggttgaaagaaactcttactcttgttctgttgtccacgaaggtttgcataaccaccatactaccaagtctttctccagaaccccaggtaaagctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgt

SEQ ID NO: 152 - DNA, 870 bp, Artificial Sequence
  synthetic polynucleotide
gctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgtgaacctagaggtccaacaattaagccttgtcctccatgcaagtgtccagctccaaatttgttgggaggtccatctgtttttatcttcccacctaagatcaaagacgttttgatgatctctttgtctcctatcgttacatgtgttgtcgttgatgtctctgaggacgatccagacgttcagatttcttggtttgttaataacgttgaagttcacactgcccaaactcagacccacagagaggattatgactctactttgagagtcgtttctgccttgcctatccaacaccaagactggatgtctggaaaagaatttaaatgtaaagttaacaataaggacttgcctgcaccaattgaaagaactatttctaagcctaagggttctgttagagcaccacaggtttacgttttgcctccacctgaggaagagatgactaaaaagcaggtcaccttgacctgcatggttaccgactttatgccagaagacatctacgttgaatggacaaacaatggtaaaacagaattgaactacaaaaacactgaacctgtcttggattctgatggttcttatttcatgtactctaaattgagagtcgagaaaaagaattgggttgaaagaaactcttactcttgttctgttgtccacgaaggtttgcataaccaccatactaccaagtctttctccagaaccccaggtaaa

SEQ ID NO: 153 - DNA, 1326 bp, Artificial Sequence
  synthetic polynucleotide
gctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgtgaacctagaggtccaacaattaagccttgtcctccatgcaagtgtccagctccaaatttgttgggaggtccatctgtttttatcttcccacctaagatcaaagacgttttgatgatctctttgtctcctatcgttacatgtgttgtcgttgatgtctctgaggacgatccagacgttcagatttcttggtttgttaataacgttgaagttcacactgcccaaactcagacccacagagaggattatgactctactttgagagtcgtttctgccttgcctatccaacaccaagactggatgtctggaaaagaatttaaatgtaaagttaacaataaggacttgcctgcaccaattgaaagaactatttctaagcctaagggttctgttagagcaccacaggtttacgttttgcctccacctgaggaagagatgactaaaaagcaggtcaccttgacctgcatggttaccgactttatgccagaagacatctacgttgaatggacaaacaatggtaaaacagaattgaactacaaaaacactgaacctgtcttggattctgatggttcttatttcatgtactctaaattgagagtcgagaaaaagaattgggttgaaagaaactcttactcttgttctgttgtccacgaaggtttgcataaccaccatactaccaagtctttctccagaaccccaggtaaaagaccttctggaaaaagaccttgtaagatgcaagcctttagaatctgggatactaaccaaaagacattctatttgagaaataaccaattgattgccggatatttgcagggacctaatattaaattggaggaaaagattgatatggttccaatcgatttgcactctgtctttttgggaatccatggaggtaagttgtgtttgtcttgcgccaagtctggtgacgatattaagttgcagttggaagaggttaacatcactgacttgtctaagaacaaagaagaggataaaagattcacttttatcagatctgaaaagggtcctactacatctttcgagtctgccgcttgtccaggttggtttttgtgcaccactttggaggctgatagaccagtctctttgactaacactcctgaggaaccattgatcgttactaagttctatttccaggaagatcaa

SEQ ID NO: 154 - DNA, 1326 bp, Artificial Sequence
  synthetic polynucleotide
gctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgtgaacctagaggtccaacaattaagccttgtcctccatgcaagtgtccagctccaaatttgttgggaggtccatctgtttttatcttcccacctaagatcaaagacgttttgatgatctctttgtctcctatcgttacatgtgttgtcgttgatgtctctgaggacgatccagacgttcagatttcttggtttgttaataacgttgaagttcacactgcccaaactcagacccacagagaggattatgactctactttgagagtcgtttctgccttgcctatccaacaccaagactggatgtctggaaaagaatttaaatgtaaagttaacaataaggacttgcctgcaccaattgaaagaactatttctaagcctaagggttctgttagagcaccacaggtttacgttttgcctccacctgaggaagagatgactaaaaagcaggtcaccttgacctgcatggttaccgactttatgccagaagacatctacgttgaatggacaaacaatggtaaaacagaattgaactacaaaaacactgaacctgtcttggattctgatggttcttatttcatgtactctaaattgagagtcgagaaaaagaattgggttgaaagaaactcttactcttgttctgttgtccacgaaggtttgcataaccaccatactaccaagtctttctccagaaccccaggtaaaagaccttctggaaaaagaccttgtaagatgcaagcctttagaatctgggatactaaccaaaagacattctatttgagaaataaccaattgattgccggatatttgcagggacctaatattaaattggaggaaaagattgatatggttccaatcgatttgcactctgtctttttgggaatccatggaggtaagttgtgtttgtcttgcgccaagtctggtgacgatattaagttgcagttggaagaggttgatatcactgacttgtctaagaacaaagaagaggataaaagattcacttttatcagatctgaaaagggtcctactacatctttcgagtctgccgcttgtccaggttggtttttgtgcaccactttggaggctgatagaccagtctctttgactaacactcctgaggaaccattgatcgttactaagttctatttccaggaagatcaa

SEQ ID NO: 155 - DNA, 1326 bp, Artificial Sequence; identical to SEQ ID NO: 154
  synthetic polynucleotide

SEQ ID NO: 156 - DNA, 1320 bp, Artificial Sequence; identical to SEQ ID NO: 149
  synthetic polynucleotide

SEQ ID NO: 157 - DNA, 1320 bp, Artificial Sequence; identical to SEQ ID NO: 149
  synthetic polynucleotide

SEQ ID NO: 158 - DNA, 1320 bp, Artificial Sequence; identical to SEQ ID NO: 149
  synthetic polynucleotide

SEQ ID NO: 159 - DNA, 888 bp, Artificial Sequence
  synthetic polynucleotide
gagcctagtggaccgattagtaccatcaacccatgtcctccatgcaaagagtgccataaatgccctgcccctaatttggaagggggaccatcggtgtttatttttccacccaatattaaggacgttttgatgatttctctaactccaaaggttacctgcgttgtcgtggatgtttcagaggacgatcctgatgtacgtatctcttggtttgtgaacaatgtcgaagttcacaccgcacaaacgcagacacacagagaagattacgattctaccatcagagtggtatcagctctaccaatccaacatcaagattggatgagtggaaaggaatttaaatgtaaagttaacaataaagacctaccgtctcctattgaaaggacgatatcgaaaattaagggtctggtgagagcacctcaagtgtatattcttccacctccggcagagcagttgtcaagaaaggatgtgtcattgacttgcttagtggtcggatttaacccgggtgatataagcgtggagtggacatctaatggtcatactgaggaaaattataaagacacggctccagtcctcgactctgacgggtcctattttatatactcgaagctcgatatcaagacctctaagtgggaaaaaaccgactccttcagttgcaatgtaagacatgaaggactgaagaattactatctgaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgt

SEQ ID NO: 160 - DNA, 888 bp, Artificial Sequence
  synthetic polynucleotide
gagcctagtggaccgattagtaccatcaacccttgccctccatgtaaggaatgccataaatgtcctgcacctaacttagagggtggaccgtcggtcttcattttcccacctaatataaaagatgtcttgtacataacactggaaccaaaagttacatgtgtggtcgtggatgtttccgaagatgacccagatgttagaattagctggttcgtaaacaatgtagaagtccacaccgctcaaacccaaacacatcgtgaggattatgactccaccattagagtcgtttccgctctccctatacaacaccaggactggatgtcagggaaagagtttaaatgtaaagttaataacaaggatctcccatctccaattgagaggactatctccaagattaaaggtctggtaagagcaccccaagtttatattctaccacctccagctgaacaactttctagaaaggatgtttctcttacatgtttagtggttggcttcaaccccggagacatatctgttgagtggacgtcaaacggtcatactgaagagaattataaagataccgcccccgtgttggacagtgacggaagttacttcatctactccaagttggatataaagacctcaaagtgggagaaaaccgattccttctcctgcaatgttcgtcacgagggtttaaaaaactattacctgaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgt

SEQ ID NO: 161 - DNA, 888 bp, Artificial Sequence
  synthetic polynucleotide
gagcctagtggaccgattagtaccatcaacccatgtcccccttgtaaagaatgtcataagtgtcctgctccgaacgctgccggcggtccttcggtgtttatttttccccctaatataaaggacgttctttacattactctggagccaaaggtcacttgcgtagttgtggacgtatcagaagatgacccagatgttagaatatcttggttcgtgaacaatgtggaagtgcatacagcgcagacacaaacccatcgagaggactacgactcaaccatccgtgtagtttcagctcttcccattcaacatcaagactggatgtcaggtaaggaattcaagtgtaaggtaaataacaaggatcttggatcaccaattgaacgtaccattagcaagattaagggtcttgtaagagcccctcaagtgtacattcttcctccacctgctgaacaattatctcgtaaagatgtttcattaacctgtctggtcgttggttttaatcccggtgatatttcagttgaatggacctcaaacggtcatacagaggaaaattacaaagacactgccccagtgttagattctgatgggtcttactttatctactcaaagttagatatcaagacttctaagtgggaaaagactgactctttcagctgcaacgttcgacacgaaggactgaagaattattacttgaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgt

SEQ ID NO: 162 - DNA, 888 bp, Artificial Sequence
  synthetic polynucleotide
gagcctagtggaccgattagtaccatcaacccttgccctccatgtaaggaatgccataaatgtcctgcacctaacttagagggtggaccgtcggtcttcattttcccacctaatataaaagatgtcttgtacataacactggaaccaaaagttacatgtgtggtcgaggatgtttccgaagatgacccagatgttagaattagctggttcgtaaacaatgtagaagtccacaccgctcaaacccaaacacatcgtgaggattatgactccaccattagagtcgtttccgctctccctatacaacaccaggactggatgtcagggaaagagtttaaatgtaaagttaataacaaggatctcccatctccaattgagaggactatctccaagattaaaggtctggtaagagcaccccaagtttatattctaccacctccagctgaacaactttctagaaaggatgtttctcttacatgtttagtggttggcttcaaccccggagacatatctgttgagtggacgtcaaacggtcatactgaagagaattataaagataccgcccccgtgttggacagtgacggaagttacttcatctactccaagttggatataaagacctcaaagtgggagaaaaccgattccttctcctgcaatgttcgtcacgagggtttaaaaaactattacctgaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgt

SEQ ID NO: 163 - DNA, 888 bp, Artificial Sequence
  synthetic polynucleotide
gagcctagtggaccgattagtaccatcaacccatgtcctccctgtaaggaatgtcacaaatgtccagctcctaatctggagggagggccatccgtgtttatcttcccacctaacattaaagatgttttgatgatttccttgactccaaaagtgacttgcgtcgtagtggacgtttctgaagacgatccagatgtgagaattagctggttcgttaacaatgtcgaggttcatacagcacaaacccaaacccacagagaggattacgatagcactatcagggtcgtttccgctcttcctattcagcaccaagattggatgagcggaaaagaatttaaatgtaaggttaataacaaagacctgccatccccaattgaaagaacaatttctaagattaaaggtttggtccgtgcacctcaagtttatatcttgccacccccagcagaacaattgtcaagaaaagatgtctctcttacatgcttagtcgttggtttcaatcctggggatatttctgtagagtggacaagtaatggacatacagaggaaaattataaagataccgctccagtacttgattccgatggttcctacttcatctattctaagctggacattaaaacctcaaagtgggaaaaaactgactcttttagttgtaacgttctgcatgagggcttgaaatcttattaccttaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgt

SEQ ID NO: 164 - DNA, 888 bp, Artificial Sequence
  synthetic polynucleotide
gagcctagtggaccgattagtaccatcaacccgtgtccaccttgcaaagaatgtcataaatgtcctgcccccaatgccgctggaggtccttcggtatttatttttcccccaaatattaaagacgtccttatgatttctttaactcccaaagtcacttgtgttgtagttgacgtgtcagaggacgatcccgacgtaagaatttcctggtttgtaaataacgttgaagtacacacagcacaaactcaaactcacagagaggactacgattctactattagagtggtatctgctttgccaattcaacatcaggactggatgtcaggtaaggagtttaaatgtaaagttaacaataaggaccttggttcacccatcgaaagaactattagtaaaatcaagggattagtccgtgctcctcaggtgtacattcttccacctccagccgagcagctgagtcgaaaggatgttagtctaacctgcctggtggtaggtttcaaccctggagatatttcggtggagtggacatccaacggtcatactgaagagaattacaaggataccgcccccgtcctagattcagatggttcttacttcatttactcgaaactagacattaaaaccagcaagtgggagaagactgacagtttctcctgcaatgtcttgcatgaaggattgaagtcttattaccttaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgt

SEQ ID NO: 165 - DNA, 888 bp, Artificial Sequence
  synthetic polynucleotide
gagcctagtggaccgattagtaccatcaacccatgtcctccctgtaaggaatgtcacaaatgtccagctcctaatctggagggagggccatccgtgtttatcttcccacctaacattaaagatgttttgatgatttccttgactccaaaagtgacttgcgtcgtagaggacgtttctgaagacgatccagatgtgagaattagctggttcgttaacaatgtcgaggttcatacagcacaaacccaaacccacagagaggattacgatagcactatcagggtcgtttccgctcttcctattcagcaccaagattggatgagcggaaaagaatttaaatgtaaggttaataacaaagacctgccatccccaattgaaagaacaatttctaagattaaaggtttggtccgtgcacctcaagtttatatcttgccacccccagcagaacaattgtcaagaaaagatgtctctcttacatgcttagtcgttggtttcaatcctggggatatttctgtagagtggacaagtaatggacatacagaggaaaattataaagataccgctccagtacttgattccgatggttcctacttcatctattctaagctggacattaaaacctcaaagtgggaaaaaactgactcttttagttgtaacgttctgcatgagggcttgaaatcttattaccttaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgt

SEQ ID NO: 166 - DNA, 888 bp, Artificial Sequence
  synthetic polynucleotide
gagcctagtggaccgattagtaccatcaacccttgccctccatgcaaagaatgtcataaatgtcctgccccgaaccttgagggtggaccgtctgttttcatctttcccccaaacattaaggacgttttgatgatcagcctgactccaaaagtgacatgtgttgtggttgatgtttcagaagatgacccagacgtgcggatctcttggtttgtgaataacgtcgaggtccatacagcgcagacccaaacccaccgagaggattacgactcgacaattcgcgtggtctctgcacttcccattgatcaccatgactggatgtccggtaaggaatttaagtgcaaagttaacaataaggacttgccttctcctattgagagaacaatttctaagattaaaggccttgtaagggctccacaagtttacattttgccacccccagcggagcaactgtctagaaaggatgtcagtcttacctgcctagtagtggggtttaatcctggtgatatctctgtcgaatggacgagcaacggacacactgaagagaattataaggatacagcacctgtcttagattcagatggaagttactttatttactctaaattggacattaaaacctctaaatgggaaaagactgattcatttagttgtaatgtccgccatgaggggttaaaatcctattaccttaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgt

SEQ ID NO: 167 - DNA, 888 bp, Artificial Sequence
  synthetic polynucleotide
gagcctagtggaccgattagtaccatcaacccttgcccaccttgtaaagaatgtcataaatgccctgctccgaatgctgcaggcggtccaagtgtattcattttcccgccaaacataaaagatgtcttaatgatttctctgactcctaaggtgacatgtgtagtcgtggatgtttcggaggacgatcccgacgttagaattagttggttcgttaataacgtcgaagtgcacactgctcaaactcagacacacagagaagactatgattctactattagagtcgtttctgcactacctatcgaccaccatgattggatgagtggaaaggagtttaaatgtaaagtcaacaataaggatcttggatctccaattgagcgtacgatttctaagatcaaaggtctggttcgtgcgccacaggtctatatactacccccacctgcagagcaactgtccagaaaagacgtttctcttacttgcttggtagtggggttcaatcctggtgatatttctgtcgagtggacaagtaacggtcacactgaggaaaattacaaggatactgcacctgtcctagattctgatggttcctactttatttacagtaaattagacatcaaaacgtctaagtgggaaaagacagactcattcagctgtaatgttcgtcacgagggtctaaaatcatactatttgaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgt

SEQ ID NO: 168 - DNA, 888 bp, Artificial Sequence
  synthetic polynucleotide
gagcctagtggaccgattagtaccatcaacccatgtccaccctgtaaagagtgccataagtgccctgcacctaatttagagggcggtccatccgtgttcatttttccacccaacatcaaggatgtcctcatgatctcactgacacctaaagttacctgtgtagttgaggatgtttcagaagacgatcccgatgtccggatttcttggtttgtaaacaatgttgaagtgcacacggcacaaacacaaactcatagagaagattatgatagcacaatcagggtcgtttccgcccttccaattgatcatcacgattggatgtccggaaaagagtttaaatgtaaagttaacaataaagatctgccctcgccaattgaaagaaccatctctaagattaagggattagttcgtgccccgcaagtttacattcttccacctccagctgaacagcttagtagaaaggacgttagtttgacctgtttggtagttggtttcaatcccggtgatataagtgtggaatggacctctaatggtcacactgaagagaattacaaggataccgctccagtgttggatagcgatggttcttacttcatttactctaagttggacattaagacgtctaaatgggagaaaactgacagtttttcttgtaacgttagacatgaaggcctaaagtcttattacttgaaaaagacaatctcacgttcacctggcgccgctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgt

SEQ ID NO: 169 - DNA, 867 bp, Artificial Sequence
  synthetic polynucleotide
gagcccaaatcaagtcccccttgcaaagagtgtagcatttttccagcacccgatgcagccggaggtccttccgtgttcattttccctcccaaaatcaaagatgttctatacatcacgctggagccagaagtcacgtgtgtggttgtcgatgttagtgaagatgaccccgatgtccaaattagttggttcgttaacaatgtagaagtccatacggctcagactcagactcacagagaggattatgactcgactttaagagtcgtttctgcattaccaattcaacaccaagactggatgtctggtaaagaatttaaatgtaaagtaaataacagggcccttccatctccgattgaaaagactatttccaagccaagaggtcccgtacgtgctccacaggtctacgttctgccgccacctgccgaggaaatgactaaaaaggaattttctttgacctgtatgattactgatttcttgccagctgaaattgctgttgattggacctcaaatggtcacaaagagttgaactataagaataccgctccagtcttggacacggatggttcttattttatgtatagcaagcttcgagttcaaaagagtacttgggaaaaagggtcactatttgcatgttcagttgtgcatgaggggcttcataaccaccataccactaagacaatttctagatcgttgggtgcagctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgt

SEQ ID NO: 170 - DNA, 867 bp, Artificial Sequence
  synthetic polynucleotide
gaacctgagtctgagcctccctgtaaagaatgctccatttttccagctccagatgccgcagggggtccttccgttttcatatttccgccaaagataaaagatgtcctgtacattacactggagcctgaagtgacttgtgttgtagttgatgtttcagaagatgaccctgacgtgcagatctcttggtttgttaacaatgttgaagtgcataccgctcaaactcagacccacagagaggactacgactctactttacgtgttgtgtctgcactgcctattcagcaccaagattggatgtctggtaaagagtttaaatgtaaggtgaataaccgtgctttgccttctccaattgagaaaacaataagtaagccaagaggaccggttagagcccctcaggtttatgtcttacctccacctgctgaggaaatgacgaaaaaggagttttcactgacatgtatgatcacagattttctgcctgccgagattgcagttgactggacgagtaacggacataaagagttaaactacaaaaataccgcacctgtcctggataccgatggatcttattttatgtactcaaagcttagagtccaaaagtctacatgggagaagggatctttgttcgcttgtagcgtggttcatgaaggtcttcacaatcaccatactacaaagacaatcagtcggtcgctaggagaagctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgt

SEQ ID NO: 171 - DNA, 903 bp, Artificial Sequence
  synthetic polynucleotide
tacccctacgatgtcccagactacgccaaacctaaagagcccgaatctgagccaccttgtaaggagtgttccatctttcctgccccagatgcagccggtggacctagtgtctttatcttccctccaaaaatcaaggatgttctgtatattacgttagaaccagaagttacttgtgtcgttgtagacgtttcagaagacgatcctgacgtccaaatttcgtggtttgttaataacgtcgaagtacatactgcacagactcaaacccatcgcgaggactacgactcaaccttgagagttgtctcagccctgcctatccaacatcaagactggatgtctggtaaggagtttaaatgtaaggttaataacagagcccttccatctcctatagagaaaactatttctaagccgcgaggtcctgttcgggctccgcaggtttatgtcctgcccccacctgctgaggaaatgactaagaaagaattctctttaacatgtatgattacggactttctacccgcagagatcgccgtcgactggacttcaaacggtcacaaagaactaaattacaaaaacactgctcctgtattagatacggacggtagttattttatgtattccaagttgagagttcaaaagtccacttgggagaagggttctctgttcgcttgttcagttgtccacgaaggattgcataaccatcacacgactaagaccatttcaagatctctgggggaggctaaagttacaggaaaagaagcctcttgtcatgatgctgttgctggatgtccaagaatctatgacccagtctgtggtactgatggaattacatacgctaatgaatgtgttttgtgttttgaaaacagaaagagaattgaaccagttttgatcagaaaaggaggtccttgt

SEQ ID NO: 172 - DNA, 864 bp, Artificial Sequence
  synthetic polynucleotide
gaaccaaagtcttctgataagactcatacttgtccaccatgtccagctccagaagctgctggtggtccatctgtttttttgtttccaccaaagccaaaggatactttgtacattactagagagccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgt

SEQ ID NO: 173 - DNA, 864 bp, Artificial Sequence
  synthetic polynucleotide
gaaccaaagtcttctgataagactcatacttgtccaccatgtccagctccagaagctgctggtggtccatctgtttttttgtttccaccaaagccaaaggatactttgtacattactagagagccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttttgcatcaagattggttgaacggtaaggaatacaagtgtaaggtttctaacaaggctttgggtgctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgt

SEQ ID NO: 174 - DNA, 864 bp, Artificial Sequence
  synthetic polynucleotide
gaaccaaagtcttctgataagactcatacttgtccaccatgtccagctccagaagctgctggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttttgcatcaagattggttgaacggtaaggaatacaagtgtaaggtttctaacaaggctttgggtgctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttttgcatgaagctttgcattctcattacactcaaaagtctttgtctttgtctccaggtgctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgt

SEQ ID NO: 175 - DNA, 864 bp, Artificial Sequence
  synthetic polynucleotide
gaaccaaagtcttctgataagactcatacttgtccaccatgtccagctccagaagctgctggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttgatcatcatgattggttgaacggtaaggaatacaagtgtaaggtttctaacaaggctttgggtgctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcattctcattacactcaaaagtctttgtctttgtctccaggtgctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgt

SEQ ID NO: 176 - DNA, 1596 bp, Artificial Sequence
  synthetic polynucleotide
atgaggcaggtttggttctcttggattgtgggattgttcctatgttttttcaacgtgtcttctgctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgtgaaccaaagtcttctgataagacacatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgattctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctaggccatccggtagaaaatcttccaaaatgcaagccttcaggatctgggatgttaaccaaaaaactttttacctgaggaacaatcaattggtggctggatatttacaaggaccaaatgttaacctcgaagagaaaatagatgtagtccctatcgagccacatgccttgtttttgggaatccatggtgggaaaatgtgtttatcgtgtgtcaagtctggagacgagacgcgtctccaattggaggccgtcgatattacggacttatctgagaaccgaaaacaagacaaacgttttgctttcatcaggtcagacagtggacccacaacttccttcgaatcagctgcctgtccaggatggtttctatgcacagcaatggaagctgatcagcctgtttcgttgactaatatgcccgatgagggtgtcatggtcactaaattctattttcaggaggacgag

SEQ ID NO: 177 - protein, 532 aa, Artificial Sequence
  synthetic polypeptide
MRQVWFSWIVGLFLCFFNVSSAAPVNTTTEDETAQIPAEAVIGYSDLEGDFDVAVLPFSNSTNNGLLFINTTIASIAAKEEGVSLEKREAEADSLGREAKCYNELNGCTKIYDPVCGTDGNTYPNECVLCFENRKRQTSILIQKSGPCEPKSSDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGARPSGRKSSKMQAFRIWDVNQKTFYLRNNQLVAGYLQGPNVNLEEKIDVVPIEPHALFLGIHGGKMCLSCVKSGDETRLQLEAVDITDLSENRKQDKRFAFIRSDSGPTTSFESAACPGWFLCTAMEADQPVSLTNMPDEGVMVTKFYFQEDE

SEQ ID NO: 178 - DNA, 1545 bp, Artificial Sequence
  synthetic polynucleotide
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccgccagcattgctgctaaagaagaaggggtatctctcgagaaaagagaggctgaagctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgtgaaccaaagtcttctgataagacacatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgattctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctaggccatccggtagaaaatcttccaaaatgcaagccttcaggatctgggatgttaaccaaaaaactttttacctgaggaacaatcaattggtggctggatatttacaaggaccaaatgttaacctcgaagagaaaatagatgtagtccctatcgagccacatgccttgtttttgggaatccatggtgggaaaatgtgtttatcgtgtgtcaagtctggagacgagacgcgtctccaattggaggccgtcgatattacggacttatctgagaaccgaaaacaagacaaacgttttgctttcatcaggtcagacagtggacccacaacttccttcgaatcagctgcctgtccaggatggtttctatgcacagcaatggaagctgatcagcctgtttcgttgactaatatgcccgatgagggtgtcatggtcactaaattctattttcaggaggacgag

SEQ ID NO: 179 - protein, 515 aa, Artificial Sequence
  synthetic polypeptide
MRFPSIFTAVLFAASSALAAPVNTTTEDETAQIPAEAVIGYSDLEGDFDVAVLPFSASIAAKEEGVSLEKREAEADSLGREAKCYNELNGCTKIYDPVCGTDGNTYPNECVLCFENRKRQTSILIQKSGPCEPKSSDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGARPSGRKSSKMQAFRIWDVNQKTFYLRNNQLVAGYLQGPNVNLEEKIDVVPIEPHALFLGIHGGKMCLSCVKSGDETRLQLEAVDITDLSENRKQDKRFAFIRSDSGPTTSFESAACPGWFLCTAMEADQPVSLTNMPDEGVMVTKFYFQEDE

SEQ ID NO: 180 - DNA, 1389 bp, Artificial Sequence
  synthetic polynucleotide
atgagatttccttcaatttttactgctgttttattcgcagcatcctccgcattagctgaggctgaagctgattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgtgaaccaaagtcttctgataagacacatacttgtccaccatgtccagctccagaattgttgggtggtccatctgtttttttgtttccaccaaagccaaaggatactttgatgatttccagaactccagaagttacttgtgttgttgttgatgtttctcatgaagatccagaagttaagtttaactggtacgttgatggtgttgaagttcataacgctaagactaagccaagagaagaacaatacgattctacttacagagttgtttctgttttgactgttttgcatcaagattggttgaacggtaaggaatacaagtgtaaggtttctaacaaggctttgccagctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgctaggccatccggtagaaaatcttccaaaatgcaagccttcaggatctgggatgttaaccaaaaaactttttacctgaggaacaatcaattggtggctggatatttacaaggaccaaatgttaacctcgaagagaaaatagatgtagtccctatcgagccacatgccttgtttttgggaatccatggtgggaaaatgtgtttatcgtgtgtcaagtctggagacgagacgcgtctccaattggaggccgtcgatattacggacttatctgagaaccgaaaacaagacaaacgttttgctttcatcaggtcagacagtggacccacaacttccttcgaatcagctgcctgtccaggatggtttctatgcacagcaatggaagctgatcagcctgtttcgttgactaatatgcccgatgagggtgtcatggtcactaaattctattttcaggaggacgag

SEQ ID NO: 181 - protein, 463 aa, Artificial Sequence
  synthetic polypeptide
MRFPSIFTAVLFAASSALAEAEADSLGREAKCYNELNGCTKIYDPVCGTDGNTYPNECVLCFENRKRQTSILIQKSGPCEPKSSDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGARPSGRKSSKMQAFRIWDVNQKTFYLRNNQLVAGYLQGPNVNLEEKIDVVPIEPHALFLGIHGGKMCLSCVKSGDETRLQLEAVDITDLSENRKQDKRFAFIRSDSGPTTSFESAACPGWFLCTAMEADQPVSLTNMPDEGVMVTKFYFQEDE

SEQ ID NO: 182 - protein, 22 aa, Artificial Sequence
  synthetic polypeptide
TCPPCPAPELLGGPSVFLFPPK

SEQ ID NO: 183 - protein, 22 aa, Artificial Sequence
  synthetic polypeptide
TCPPCPAPEAAGGPSVFLFPPK

SEQ ID NO: 184 - protein, 17 aa, Artificial Sequence
  synthetic polypeptide
CKVSNKALPAPIEKTIS

SEQ ID NO: 185 - protein, 17 aa, Artificial Sequence
  synthetic polypeptide
CKVSNKALGAPIEKTIS

SEQ ID NO: 186 - protein, 57 aa, Artificial Sequence
  synthetic polypeptide
AKVTGKEASCHDAVAGCPRIYDPVCGTDGITYANECVLCFENRKRIEPVLIRKGGPC

SEQ ID NO: 187 - protein, 232 aa, Artificial Sequence
  synthetic polypeptide
EPEESDKTHTCPPCPAPEAAGGPSVFLFPPKPKDTLYITREPEVTCVVVDVSHEDPEVKFNWYVDGVEVHNAKTKPREEQYDSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGE

SEQ ID NO: 188 - protein, 233 aa, Artificial Sequence
  synthetic polypeptide
EPRGPTIKPCPPCKCPAPNLLGGPSVFIFPPKIKDVLMISLSPIVTCVVVDVSEDDPDVQISWFVNNVEVHTAQTQTHREDYDSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 189 - protein, 239 aa, Artificial Sequence
  synthetic polypeptide
EPSGPISTINPCPPCKECHKCPAPNLEGGPSVFIFPPNIKDVLMISLTPKVTCVVVDVSEDDPDVRISWFVNNVEVHTAQTQTHREDYDSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGA

SEQ ID NO: 190 - protein, 239 aa, Artificial Sequence
  synthetic polypeptide
EPSGPISTINPCPPCKECHKCPAPNLEGGPSVFIFPPNIKDVLYITLEPKVTCVVVDVSEDDPDVRISWFVNNVEVHTAQTQTHREDYDSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGA

SEQ ID NO: 191 - protein, 239 aa, Artificial Sequence
  synthetic polypeptide
EPSGPISTINPCPPCKECHKCPAPNAAGGPSVFIFPPNIKDVLYITLEPKVTCVVVDVSEDDPDVRISWFVNNVEVHTAQTQTHREDYDSTIRVVSALPIQHQDWMSGKEFKCKVNNKDLGSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGA

SEQ ID NO: 192 - protein, 239 aa, Artificial Sequence
  synthetic polypeptide
EPSGPISTINPCPPCKECHKCPAPNLEGGPSVFIFPPNIKDVLYITLEPKVTCVVEDVSEDDPDVRISWFVNNVEVHTAQTQTHREDYDSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGA

SEQ ID NO: 193 - protein, 239 aa, Artificial Sequence
  synthetic polypeptide
EPSGPISTINPCPPCKECHKCPAPNLEGGPSVFIFPPNIKDVLMISLTPKVTCVVVDVSEDDPDVRISWFVNNVEVHTAQTQTHREDYDSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVLHEGLKSYYLKKTISRSPGA

SEQ ID NO: 194 - protein, 239 aa, Artificial Sequence
  synthetic polypeptide
EPSGPISTINPCPPCKECHKCPAPNAAGGPSVFIFPPNIKDVLMISLTPKVTCVVVDVSEDDPDVRISWFVNNVEVHTAQTQTHREDYDSTIRVVSALPIQHQDWMSGKEFKCKVNNKDLGSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVLHEGLKSYYLKKTISRSPGA

SEQ ID NO: 195 - protein, 239 aa, Artificial Sequence
  synthetic polypeptide
EPSGPISTINPCPPCKECHKCPAPNLEGGPSVFIFPPNIKDVLMISLTPKVTCVVEDVSEDDPDVRISWFVNNVEVHTAQTQTHREDYDSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVLHEGLKSYYLKKTISRSPGA

SEQ ID NO: 196 - protein, 239 aa, Artificial Sequence
  synthetic polypeptide
EPSGPISTINPCPPCKECHKCPAPNLEGGPSVFIFPPNIKDVLMISLTPKVTCVVVDVSEDDPDVRISWFVNNVEVHTAQTQTHREDYDSTIRVVSALPIDHHDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKSYYLKKTISRSPGA

SEQ ID NO: 197 - protein, 239 aa, Artificial Sequence
  synthetic polypeptide
EPSGPISTINPCPPCKECHKCPAPNAAGGPSVFIFPPNIKDVLMISLTPKVTCVVVDVSEDDPDVRISWFVNNVEVHTAQTQTHREDYDSTIRVVSALPIDHHDWMSGKEFKCKVNNKDLGSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKSYYLKKTISRSPGA

SEQ ID NO: 198 - protein, 239 aa, Artificial Sequence
  synthetic polypeptide
EPSGPISTINPCPPCKECHKCPAPNLEGGPSVFIFPPNIKDVLMISLTPKVTCVVEDVSEDDPDVRISWFVNNVEVHTAQTQTHREDYDSTIRVVSALPIDHHDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKSYYLKKTISRSPGA

SEQ ID NO: 199 - protein, 232 aa, Artificial Sequence
  synthetic polypeptide
EPKSSPPCKECSIFPAPDAAGGPSVFIFPPKIKDVLYITLEPEVTCVVVDVSEDDPDVQISWFVNNVEVHTAQTQTHREDYDSTLRVVSALPIQHQDWMSGKEFKCKVNNRALPSPIEKTISKPRGPVRAPQVYVLPPPAEEMTKKEFSLTCMITDFLPAEIAVDWTSNGHKELNYKNTAPVLDTDGSYFMYSKLRVQKSTWEKGSLFACSVVHEGLHNHHTTKTISRSLGA

SEQ ID NO: 200 - protein, 232 aa, Artificial Sequence
  synthetic polypeptide
EPESEPPCKECSIFPAPDAAGGPSVFIFPPKIKDVLYITLEPEVTCVVVDVSEDDPDVQISWFVNNVEVHTAQTQTHREDYDSTLRVVSALPIQHQDWMSGKEFKCKVNNRALPSPIEKTISKPRGPVRAPQVYVLPPPAEEMTKKEFSLTCMITDFLPAEIAVDWTSNGHKELNYKNTAPVLDTDGSYFMYSKLRVQKSTWEKGSLFACSVVHEGLHNHHTTKTISRSLGE

SEQ ID NO: 201 - protein, 232 aa, Artificial Sequence
  synthetic polypeptide
EPKSSDKTHTCPPCPAPEAAGGPSVFLFPPKPKDTLYITREPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 202 - protein, 232 aa, Artificial Sequence
  synthetic polypeptide
EPKSSDKTHTCPPCPAPEAAGGPSVFLFPPKPKDTLYITREPEVTCVVVDVSHEDPEVKFNWYVDGVEVHNAKTKPREEQYDSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 203 - protein, 232 aa, Artificial Sequence
  synthetic polypeptide
EPKSSDKTHTCPPCPAPEAAGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGA

SEQ ID NO: 204 - protein, 232 aa, Artificial Sequence
  synthetic polypeptide
EPKSSDKTHTCPPCPAPEAAGGPSVFLFPPKPKDTLMISRTPEVTCVVVDVSHEDPEVKFNWYVDGVEVHNAKTKPREEQYDSTYRVVSVLTVDHHDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGA

SEQ ID NO: 205 - protein, 152 aa, Artificial Sequence
  synthetic polypeptide
RPSGRKSSKMQAFRIWDVNQKTFYLRNNQLVAGYLQGPNVNLEEKIDVVPIEPHALFLGIHGGKMCLSCVKSGDETRLQLEAVDITDLSENRKQDKRFAFIRSDSGPTTSFESAACPGWFLCTAMEADQPVSLTNMPDEGVMVTKFYFQEDE

SEQ ID NO: 206 - protein, 152 aa, Artificial Sequence
  synthetic polypeptide
RPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 207 - protein, 152 aa, Artificial Sequence
  synthetic polypeptide
RPSGKRPCKMQAFRIWDTNQKTFYLRNNQLIAGYLQGPNIKLEEKIDMVPIDLHSVFLGIHGGKLCLSCAKSGDDIKLQLEEVDITDLSKNKEEDKRFTFIRSEKGPTTSFESAACPGWFLCTTLEADRPVSLTNTPEEPLIVTKFYFQEDQ

SEQ ID NO: 208 - protein, 92 aa, Artificial Sequence
  synthetic polypeptide
MRQVWFSWIVGLFLCFFNVSSAAPVNTTTEDETAQIPAEAVIGYSDLEGDFDVAVLPFSNSTNNGLLFINTTIASIAAKEEGVSLEKREAEA

SEQ ID NO: 209 - protein, 75 aa, Artificial Sequence
  synthetic polypeptide
MRFPSIFTAVLFAASSALAAPVNTTTEDETAQIPAEAVIGYSDLEGDFDVAVLPFSASIAAKEEGVSLEKREAEA

SEQ ID NO: 210 - protein, 23 aa, Artificial Sequence
  synthetic polypeptide
MRFPSIFTAVLFAASSALAEAEA

SEQ ID NO: 211 - protein, 22 aa, Artificial Sequence
  synthetic polypeptide
ECHKCPAPNLEGGPSVFIFPPN

SEQ ID NO: 212 - protein, 22 aa, Artificial Sequence
  synthetic polypeptide
ECHKCPAPNAAGGPSVFIFPPN

SEQ ID NO: 213 - protein, 17 aa, Artificial Sequence
  synthetic polypeptide
CKVNNKDLPSPIERTIS

SEQ ID NO: 214 - protein, 17 aa, Artificial Sequence
  synthetic polypeptide
CKVNNKDLGSPIERTIS

SEQ ID NO: 215 - protein, 9 aa, Artificial Sequence
  synthetic polypeptide
VTCVVVDVS

SEQ ID NO: 216 - protein, 9 aa, Artificial Sequence
  synthetic polypeptide
VTCVVEDVS

SEQ ID NO: 217 - protein, 10 aa, Artificial Sequence
  synthetic polypeptide
DTLMISRTPE

SEQ ID NO: 218 - protein, 10 aa, Artificial Sequence
  synthetic polypeptide
DTLYITREPE

SEQ ID NO: 219 - protein, 10 aa, Artificial Sequence
  synthetic polypeptide
DVLMISLTPK

SEQ ID NO: 220 - protein, 10 aa, Artificial Sequence
  synthetic polypeptide
DVLYITLEPK

SEQ ID NO: 221 - protein, 15 aa, Artificial Sequence
  synthetic polypeptide
FSCSVMHEALHNHYT

SEQ ID NO: 222 - protein, 15 aa, Artificial Sequence
  synthetic polypeptide
FSCSVLHEALHSHYT

SEQ ID NO: 223 - protein, 15 aa, Artificial Sequence
  synthetic polypeptide
FSCNVRHEGLKNYYL

SEQ ID NO: 224 - protein, 15 aa, Artificial Sequence
  synthetic polypeptide
FSCNVLHEGLKSYYL

SEQ ID NO: 225 - protein, 10 aa, Artificial Sequence
  synthetic polypeptide
LTVLHQDWLN

SEQ ID NO: 226 - protein, 10 aa, Artificial Sequence
  synthetic polypeptide
LTVDHHDWLN

SEQ ID NO: 227 - protein, 10 aa, Artificial Sequence
  synthetic polypeptide
EALHNHYTQK

SEQ ID NO: 228 - protein, 10 aa, Artificial Sequence
  synthetic polypeptide
EALHSHYTQK

SEQ ID NO: 229 - protein, 10 aa, Artificial Sequence
  synthetic polypeptide
LPIQHQDWMS

SEQ ID NO: 230 - protein, 10 aa, Artificial Sequence
  synthetic polypeptide
LPIDHHDWMS

SEQ ID NO: 231 - protein, 10 aa, Artificial Sequence
  synthetic polypeptide
EGLKNYYLKK

SEQ ID NO: 232 - protein, 10 aa, Artificial Sequence
  synthetic polypeptide
EGLKSYYLKK

SEQ ID NO: 233 - protein, 10 aa, Artificial Sequence
  synthetic polypeptide
EPKSSDKTHT

SEQ ID NO: 234 - protein, 10 aa, Artificial Sequence
  synthetic polypeptide
EPEESDKTHT

SEQ ID NO: 235 - protein, 10 aa, Artificial Sequence
  synthetic polypeptide
QKSLSLSPGA

SEQ ID NO: 236 - protein, 10 aa, Artificial Sequence
  synthetic polypeptide
QKSLSLSPGE

SEQ ID NO: 237 - protein, 10 aa, Artificial Sequence
  synthetic polypeptide
EPKSSPPCKE

SEQ ID NO: 238 - protein, 10 aa, Artificial Sequence
  synthetic polypeptide
EPESEPPCKE

SEQ ID NO: 239 - protein, 10 aa, Artificial Sequence
  synthetic polypeptide
TKTISRSLGA

SEQ ID NO: 240 - protein, 10 aa, Artificial Sequence
  synthetic polypeptide
TKTISRSLGE

SEQ ID NO: 241 - DNA, 864 bp, Artificial Sequence
  synthetic polynucleotide
gaaccagaagagtctgataagactcatacttgtccaccatgtccagctccagaagctgctggtggtccatctgtttttttgtttccaccaaagccaaaggatactttgtacattactagagagccagaagttacttgtgttgttgttgatgtttctcatgaagatccagaagttaagtttaactggtacgttgatggtgttgaagttcataacgctaagactaagccaagagaagaacaatacgactctacttacagagttgtttctgttttgactgttttgcatcaagattggttgaacggtaaggaatacaagtgtaaggtttctaacaaggctttgggtgctccaattgaaaagactatttctaaggctaagggtcaaccaagagaaccacaagtttacactttgccaccatccagagatgaattgactaagaaccaagtttctttgacttgtttggttaagggtttttacccatctgatattgctgttgaatgggaatctaacggtcaaccagaaaacaactacaagactactccaccagttttggattctgatggttctttttttttgtactctaagttgactgttgataagtccagatggcaacaaggtaacgttttttcttgttctgttatgcatgaagctttgcataaccattacactcaaaagtctttgtctttgtctccaggtgaggattctttgggtagagaagctaagtgttacaacgaattgaacggttgtactaagatttacgatccagtttgtggtactgatggtaacacttacccaaacgaatgtgttttgtgttttgaaaacagaaagagacaaacttctattttgattcaaaagtctggtccatgt

SEQ ID NO: 242 - protein, 32 aa, Artificial Sequence
  synthetic polypeptide
GGGGSGGGGSGGGGSGGGGSGGGGSGGGGSGG

(19) United States
(12) Patent Application Publication
(10) Pub. No.: US 2024/0228585 A1
(43) Pub. Date: Jul. 11, 2024

(54) FC-FUSION PROTEIN THERAPEUTIC FOR THE TREATMENT OF PANCREATITIS

(71) Applicant: PRESIDENT AND FELLOWS OF HARVARD COLLEGE, Cambridge, MA (US)

(72) Inventors: Jeffrey Charles WAY, Cambridge, MA (US); Katherine REDFIELD CHAN, Cambridge, MA (US); Jungmin LEE, Cambridge, MA (US); Florian BIEBERICH, Cambridge, MA (US); Daniel HEID, Cambridge, MA (US); Dominik NIOPEK, Cambridge, MA (US)

(73) Assignee: PRESIDENT AND FELLOWS OF HARVARD COLLEGE, Cambridge, MA (US)

(21) Appl. No.: 18/558,640

(22) PCT Filed: May 2, 2022
(86) PCT No.: PCT/US22/27247

§ 371 Date: Nov. 2, 2023

Related U.S. Application Data
(60) Provisional application No. 63/183,318, filed on May 3, 2021.

(51) Int. Cl. C07K 14/715; A61K 38/00; A61P 1/18; C07K 14/47

(52) U.S. Cl. CPC C07K 14/7155; A61P 1/18; C07K 14/4703; A61K 38/00; C07K 2319/30

(57) ABSTRACT

The disclosure provides compositions, methods, and kits for treatment of a condition, disease or disorder characterized by elevated trypsin activity or level, e.g., pancreatitis.

### CROSS-REFERENCE TO RELATED APPLICATIONS

This application claims benefit under 35 U.S.C. § 119(e) of U.S. Provisional Application No. 63/183,318 filed May 3, 2021, the contents of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on May 2, 2022, is named 002806-07920WOPT_SL.txt and is 462,968 bytes in size.

### TECHNICAL FIELD

The technology described herein relates to compositions, methods, and kits for treatment of condition, disease or disorder characterized by elevated trypsin activity or level, e.g., pancreatitis.

### BACKGROUND

The pancreas is responsible for producing proteolytic enzymes, such as trypsin, which the body relies upon for food digestion. Pancreatitis is a painful, life-threatening condition that affects nearly 9 million people world-wide and kills over 100,000 people annually. [¹] In pancreatitis, these proteases become prematurely activated resulting in autodigestion. Patients with this disease can expect prolonged hospital stays, severe pain, and risk of death with little therapeutic recourse. Acute pancreatitis is the most frequent gastrointestinal cause of US hospitalizations (275,000 in the US in 2009 alone).[²]Treatment today is largely unchanged from 5 decades ago, focusing on fluids and pain management. This disease remains lethal in 3-16% of cases. [³] The primary treatment modalities are supportive measures-that is, treatment is designed to sustain the patient as they heal as opposed to treating the disease directly.

The triggers leading to pancreatitis are diverse: gallstones, alcohol, trauma, genetic predisposition and scorpion bites to name a few. Furthermore, the disease trigger is frequently unidentifiable, with approximately 30% of cases deemed idiopathic. It is thought that many or most of the idiopathic cases are due to genomic mutations affecting proteins that control trypsin activity, such as mutations in trypsin itself, in SPINK1 (a trypsin inhibitor expressed in the pancreas), or chymotrypsin (which can cleave and inactivate trypsin). Downstream of these instigating events, patients with pancreatitis appear to share a common pathway. The pancreas manufactures digestive proenzymes-premature activation, and failure of inactivation, of these digestive proenzymes is believed to be a major mechanism driving acute pancreatitis. [⁴]Bovine protein trypsin inhibitor (BPTI) is a 58AA peptide that inhibits the proteases implicated in pancreatitis.

This inhibitor showed promise as a treatment in experimental models of acute pancreatitis.[⁴][⁵]However, over two decades of clinical trials, researchers found no statistically significant treatment impact and use of BPTI as a treatment for pancreatitis was largely abandoned.[⁴][⁶]

In addition, BPTI (also termed aprotinin) inhibits a number of other proteases, such as the blood-clotting modulators plasmin and kallikrein. BPTI/aprotinin has been marketed as the drug Trasylol™, which was used after cardiac surgery to control blood loss. However, Trasylol has been withdrawn from the market in some countries, and its safety remains controversial. The rationale for use of BPTI/aprotinin after cardiac surgery relates to its inhibition of plasmin and not due to its inhibition of trypsin

Thus, there remains a need in the art for compositions and methods for treating pancreatitis. The present disclosure addresses some of these needs.

### SUMMARY

The invention is focused on forms of protease inhibitors that particularly inhibit trypsin, and that have superior properties with respect to frequency of dosing, reduced side effects, and manufacturability relative to other trypsin inhibitors that have been described previously.

According to the invention, an ideal pancreatitis treatment should be designed to not only inhibit trypsin, but to also enjoy minimal renal clearance, exhibit specific binding to trypsin as opposed to the generic inhibition of all proteases, and have the ability to penetrate pancreatic tissue. Inventors have found inter alia that it is possible to address the current weaknesses of BPTI as a human pancreatitis treatment through rationally designed modifications based on insights of the invention.

Thus, in one aspect, the disclosure provides a Fc fusion protein. Generally, the Fc fusion protein comprises a pancreatic trypsin inhibitor (PTI) domain linked to an Fc domain. The PTI domain is a serine protease inhibitor Kazal-type 1 (SPINK1) domain or a bovine pancreatic trypsin inhibitor (BPTI) domain. Generally, Fc domain comprises a hinge region, an IgG CH2 domain and an IgG CH3 domain.

Inventors have discovered inter alia that orientation of the Fc fusion protein effects properties of the Fc fusion properties. Accordingly, in some embodiments, the N-terminus of the Fc domain is linked to the C-terminus of the PTI domain. In some preferred embodiments, the C-terminus of the Fc domain is linked to the N-terminus of the PTI domain.

In some embodiments, the invention provides a SPINK-based Fc fusion protein comprising the amino acid sequence:

(SEQ ID NO: 1)
EPKSSDKTHTCPPCPAPELLGGPSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPGADSLGREAKCYNELNGCTKIYDPVCGTDGNTYPNECVLC
FENRKRQTSILIQKSGPC

This sequence in particular includes an N-linked glycosylation site in the Fc region. This protein product is preferentially expressed in mammalian cells, such that the N-linked oligosaccharide in the protein has human or at least mammalian characteristics.

In some embodiments, the invention provides a SPINK-based Fc fusion protein comprising the amino acid sequence:

(SEQ ID NO: 85)
EPEESDKTHTCPPCPAPEaaGGPSVFLFPPKPKDTLyItRePEVT
CVVVDVSHEDPEVKFNWYVDGVEVHNAKTKPREEQYDSTYRVVSV
LTVLHQDWLNGKEYKCKVSNKALgAPIEKTISKAKGQPREPQVYT
LPPSRDELTKNQVSLTCLVKGFYPSDIAVEWESNGQPENNYKTT
PPVLDSDGSFFLYSKLTVDKSRWQQGNVFSCSVMHEALHNHYTQK
SLSLSPGEDSLGREAKCYNELNGCTKIYDPVCGTDGNTYPNECVL
CFENRKRQTSILIQKSGPC

This sequence in particular includes an N-linked glycosylation site in the Fc region and additional modifications in the Fc region that enhance FcRn-based recycling, minimize Fc(gamma) receptor binding, and minimize passage across the kidney basement membrane.

In some embodiments, the invention provides a tripartite SPINK-based Fc fusion protein further comprising an inflammatory cytokine inhibitor, such as an interleukin1 receptor antagonist (IL-1RA) moiety.

Also provided herein is a pharmaceutical composition. The pharmaceutical composition comprises a Fc fusion protein described herein and a pharmaceutically acceptable carrier or excipient.

In another aspect, the disclosure provides a nucleic acid comprising a nucleotide sequence encoding a Fc fusion protein described herein. The nucleic acid can comprise a codon optimized nucleic acid sequence. Further, the nucleic acid can be present in a cell, e.g., a host cell for expressing the Fc fusion protein.

In still another aspect, the disclosure provides a kit. The kit comprises a Fc fusion protein described herein. In some embodiments, the kit comprises a nucleic acid comprising a nucleic acid sequence encoding a Fc fusion protein described herein.

Also provided herein is a method for treating a condition, disease or disorder characterized by elevated trypsin activity or level. Generally, the method comprising administering a Fc fusion protein described herein to a subject in need thereof, such as a human patient suffering from pancreatitis without gallstone involvement. In some embodiments, the condition, disease or disorder characterized by elevated trypsin activity or level is pancreatitis.

In some embodiments, the method can comprise a step of diagnosing or selecting the subject for treatment.

In some embodiments, the method can comprise a step of determining trypsin activity or level in the subject prior to administering the Fc fusion protein. An increased activity and/or level indicates a subject is in need of treatment.

In some embodiments, the method can comprise a step of obtaining or receiving results of an assay indicating determining trypsin activity or level prior to onset of administration.

In some administering is intravenous (IV) or intraperitoneal (IP) administration.

In a preferred embodiment, a human patient is treated with an Fc-protease inhibitor fusion protein at a dose of 500 mgs per day by intravenous infusion over 1 hour, 4 hours, or longer periods of time. In a preferred embodiment, a human patient is treated with an Fc-protease inhibitor fusion protein at a dose of at least 500 mgs per day. In another preferred embodiment, a human patient is treated with an Fc-protease inhibitor fusion protein at a dose of at least 1 gram per day. In another preferred embodiment, a human patient is treated with an Fc-protease inhibitor fusion protein at a dose of at least 2.5 grams per day.

Also provided herein is a method for minimizing bleeding during cardiac surgery, such as coronary artery bypass grafting (CABG). Generally, the method comprising administering an Fc-BPTI fusion protein described herein to a subject in need thereof.

Also provided herein is a method comprising inhibition of plasmin without kidney toxicity

### BRIEF DESCRIPTION OF THE DRAWINGS

FIG. 1 is a schematic representation of reasons for failure of BPTI to treat pancreatitis.

FIG. 2 is a schematic representation of general antibody structure.

FIG. 3 is a schematic representation of cycling of Fc-fusion proteins.

FIGS. 4A and 4B are schematic representations of cycling of free BPTI and Fc-fusion protein (FIG. 4B).

FIGS. 5 and 6 are depictions of the highly conserved active site of BPTI.

FIG. 7 is a schematic representation of BPTI enzymatic activity.

FIG. 8 is a schematic representation of PK/biodistribution of a drug after IV administration.

FIG. 9 shows design of a BPTI mutant that binds trypsin in a way that is both relatively less strong than the wildtype affinity but still able to inhibit trypsin in an objectively potent fashion.

FIGS. 10A-10C depict structures of Fc fusion proteins. The Fc fusion protein comprises a fusion of the IgG Fc chain (e.g., human IgG Fc chain) to either the bovine or human trypsin inhibitor, BPTI and SPINK1, respectively. The Fc domain can improve blood half-life time of the protein while avoiding renal clearance.

FIG. 11 is a schematic representation of some exemplary properties of the Fc fusion proteins of the invention.

FIG. 12 shows design of an exemplary IgG1/IgG3 hybrid Fc.

FIGS. 13A and 13B show screening of yeast (Pichia pastoris) clones expressing Fc-SPINK (FIG. 13A) and Fc-BPTI (FIG. 13B). Six clones for each construct were used to inoculate liquid cultures, grown for 24 h. 5 μl of culture supernatant was then run on a 4-20 % SOS gel followed by Western blot using an antibody targeting the Fc chain.

FIGS. 14A and 14B show protein expression time-course. Yeast (Pichia pastoris) cultures expressing Fc-BPTI or Fc-SPINK were grown for 24-90 h. Then, the culture supernatant was analyzed by Western blot using an antibody targeting the Fc chain (FIG. 14A) and Coomassie staining (FIG. 14B).

FIGS. 15A and 15B show protein production pipeline. Yeast cultures expressing Fc-BPTI or Fc-SPINK were grown for 72 h. Culture supernatants were sterile-filtered, concentrated and the Fc-fusion protein using protein A plus coated agarose beads. Shown is a Coomassie stain of the protein at the different purification steps (FIG. 15A). Following elution with low pH elution buffer (see 15A), the eluate was dialyzed against PBS and concentrated using ultrafiltration spin columns with a cutoff molecular weight of 10 kDa. The final protein was then analyzed on Coomassie gel (FIG. 15B).

FIGS. 16A-16C show effect of Fc fusion protein orientation.

FIG. 17 shows Fc fusion proteins retain trypsin inhibiting activity.

FIGS. 18-21 show Fc fusion proteins have improved affinity for trypsin over others.

FIG. 22 shows Fc fusion proteins exhibit unchanged affinity over time.

FIGS. 23 and 24 show Fc fusion proteins have a 10× improvement in FcRn binding from pH 7.4 to pH 6.5.

FIG. 25 shows that substitution at position 16 of the BPTI alters kinetics of binding with trypsin.

FIGS. 26A and 26B are bar graphs showing Fc-SPINK (FIG. 26A) and Fc-BPTI (FIG. 26B) potently inhibit Trypsin in vitro.

FIG. 27 shows pharmacokinetics of Fc-SPINK after intravenous (IV) and intraperitoneal (IP) administration. Fc-SPINK was injected intravenously and intraperitoneal and the amount of drug was subsequently.

FIG. 28 shows biodistribution of Fc-BPTI after intravenous (IV) and intraperitoneal (IP) administration.

FIG. 29 is a schematic representation of experimental workflow for Caerulein-mediated induction of acute pancreatitis. (Caerulein can also be referred to herein as cerulein or ceruletide.)

FIG. 30 shows analysis of pancreas morphology.

FIG. 31 shows FACS analysis of immune cells in pancreatic tissue.

FIG. 32 shows analysis of amylase activity in blood samples.

FIGS. 33A and 33B show analysis of cytokines in blood samples indicating caerulein-induced inflammation.

FIG. 34 is a schematic representation of experimental setup to evaluate drug efficacy in vivo.

FIGS. 35 and 36 show analysis of pancreas morphology (FIG. 35) and histology (FIG. 36).

FIG. 37 shows analysis of amylase activity in blood samples demonstrating efficacy of Fc-SPINK treatment.

FIG. 38 is a schematic representation of an exemplary pharmacodynamic model of Fc fusion protein.

FIG. 39 shows a tissue sections of pancreases that have been stained using an anti-CD11 antibody with a horseradish peroxidase (HRP)-coupled secondary antibody. The HRP staining gives a brown residue, such that the infiltrating immune cells are stained dark brown. The left panels show pancreases from untreated mice showing no pancreatitis; essentially no brown cells are seen. The central panels show pancreases from mice treated with caerulein, which induces pancreatitis; a significant levels of brown cells, most likely macrophages, are seen. The right panels show pancreases from mice treated with caerulein and also with a single dose of Fc-SPINK, which have dramatically reduced levels of CD11-positive cells compared to the mice treated with only caerulein.

FIG. 40 is a series of dot plots showing the pancreas weights of engineered knock-in mice expressing a mutant, hyperactivatable form of cationic trypsin. Open circles indicate non-engineered, wild-type mice pancreas weights. Open squares indicate pancreas weights of mutant, engineered mice. Filled squares indicate pancreas weights of mutant, engineered mice treated three times per week with 5 mgs of Fc-SPINK1.

### DETAILED DESCRIPTION

Aspects of the technology described herein comprise compositions, methods, and kits for treatment of pancreatitis.

The general concept of the invention is as follows. Without wishing to be bound by theory, the enzyme trypsin is thought to be a major driver of pancreatitis. While an initial insult to the pancreas may occur by a variety of mechanisms, such as prolonged consumption of alcohol, trauma, exposure to toxic chemicals or idiopathic causes, once pancreatitis is initiated trypsin becomes trapped in the pancreas and leads to autodigestion of this organ due to autoactivation of itself, chymotrypsin, and a number of other enzymes that are normally ejected from the pancreas into the small intestine to help digest food. Trypsin is thought to be the primary driver of protease activation during this process.

Experiments dating to the 1960s have shown that Bovine Pancreatic Trypsin Inhibitor (BPTI) is capable of ameliorating the course of disease in animal models. However, after repeated failures in human trials between 1960 and 1980, BPTI was largely abandoned as a possible treatment option.

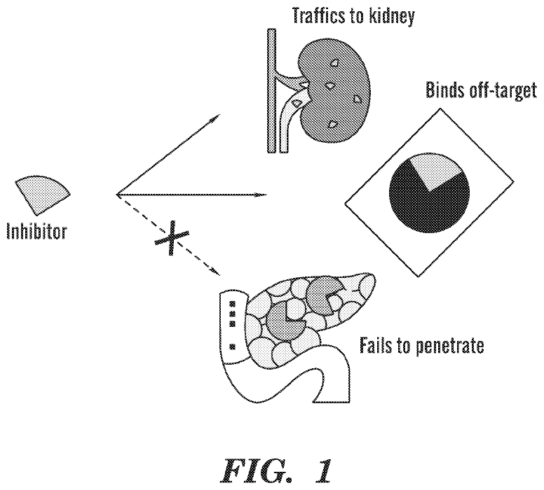

A review of the successful BPTI-pancreatitis animal studies, and subsequent failed human trials, suggests that humans were inadequately dosed. These disappointing trial results were likely due to three main issues: the small BPTI protein localizing to the kidneys, promiscuous off-target binding to serum proteases which then acted as a drug sink, and a failure to achieve adequate pancreatic distribution (FIG. 1). ^{[4][6]}

As the field has matured, so has the nuanced understanding of protein properties. BPTI is a naturally occurring protein with known folding pattern and DNA sequence. The interactions between BPTI and Trypsin, as well as other proteases, have been the subject of detailed study outside the realm of pancreatitis treatment possibilities. This vast foundation of knowledge regarding structure and binding functionality of BPTI makes it an ideal candidate for rapid but informed rational modification.

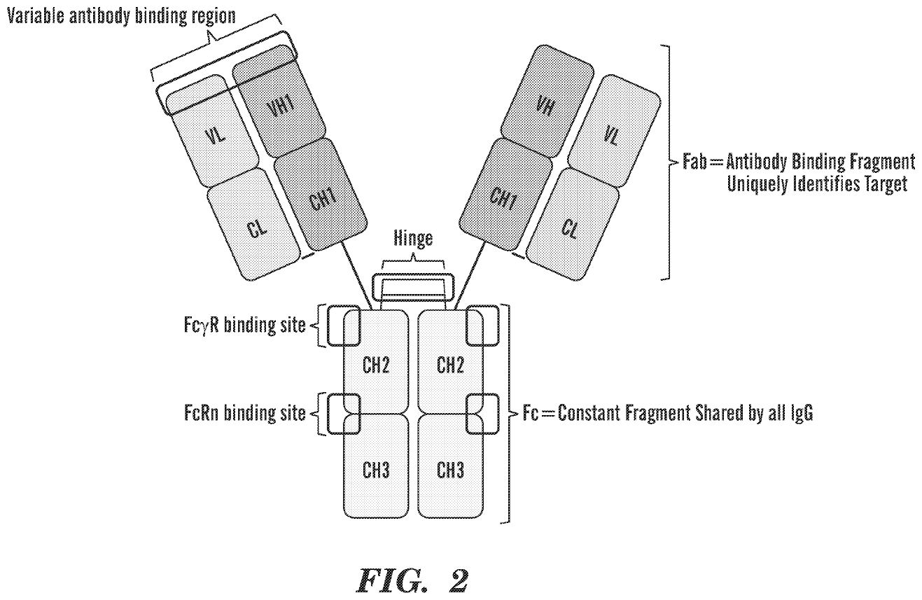

Antibodies make up another class of proteins that has been subject to extensive scientific scrutiny. In part because it is the only antibody class capable of passing into the womb, IgG and its receptors have been a source of particularly robust study in the scientific literature. IgG antibodies all share a general structure despite their ability to recognize different targets with high specificity (FIG. 2).

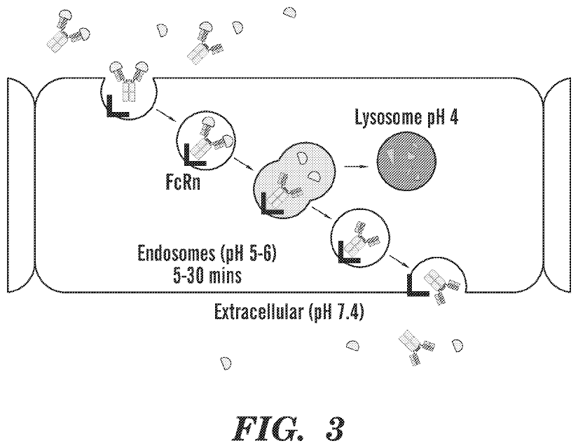

IgG antibodies enjoy a long half-life in the human body, typically 21 days. Due to a small hinge region and large number of disulfide bonds, IgG enjoys continued structural integrity and functionality despite long exposure to body temperatures and is unusually resistant to enzyme degradation. When an IgG is endocytosed by phagocytic cells, a stabilizing endosomal receptor causes the protein to be recycled to circulation instead of trafficked to the lysosome.^{[10]} Additionally, IgG is well over the size threshold for free kidney filtration, and should it get into the kidney tubules there are receptors in the tubule walls which rescue these antibodies from disposal. These properties are primarily due to the binding of sites found in the constant region of the protein, which is known as the fragment crystalizable (Fc). The stabilizing, transcytosis supporting receptor is known as the neonatal Fc receptor (FcRn). See, FIG. 3. Fusion of other protein domains to an Fc region is a routine tool for protein engineering, but an extremely large number of protein domain configurations, in combination with mutations that might be made in the Fc region, are possible and it is difficult to predict which configurations are optimal for a given application. In particular, levels of protein expression, secretion, aggregation of the resulting product, pharmacokinetic behavior, and steric hindrance of a fusion partner by an Fc element can all affect with fusion protein utility.

Considered as a drug target, trypsin presents a number of challenges. One issue is that trypsin is produced in large amounts-up to 100 mgs per day is synthesized and then normally deposited into the small intestine. This is consistent with its function as a major digestive enzyme. Moreover, typically only a small fraction of an injected protein distribute into the pancreas. Taken at face value, this means that a systemically administered protease inhibitor drug will need to be given in inconveniently large amounts. Moreover, as such a drug enters the blood compartment before entering the pancreas, it may cause side effects by binding to other proteases with similar structures but unrelated functions. For example, BPTI, also known as aprotinin, has been previously approved as Trasylol for local use after surgery to enhance blood clotting due to its inhibition of plasmin. This molecule was subsequently withdrawn from the market in the much of the world, and its safety remains controversial.

The invention provides forms of protease inhibitors that address limitations of previously protease inhibitors. For example, the invention provides forms of BPTI that are fused to an Fc region of an antibody. Both BPTI-Fc and Fc-BPTI (N-terminal to C-terminal) are provided, but the Fc-BPTI configuration is preferred. In particular, when mammalian expression vectors expressing secreted forms of BPTI-Fc and Fc-BPTI are placed in mammalian cells by transient transfection, the production of Fc-BPTI is at least 100-fold higher than BPTI-Fc, even though all of the other features of the expression vector are identical, including the promoter elements, ribosome binding site, signal sequence for secretion, and 3′ end elements. Without wishing to be bound by theory, this effect may be due to the fact that BPTI is normally synthesized with an N-terminal pro-sequence that is presumably required for correct folding; the BPTI element may not be able to fold correctly when it is at the N-terminus of the Fc. The sequence of an illustrative Fc-BPTI fusion protein in its mature form is as follows:

(SEQ ID NO: 2)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGARPDFCLEPPYTGPCKARI
IRYFYNAKAGLCQTFVYGGCRAKRNNFKSAEDCMRTCGGA-

A space is inserted between the Fc element and the BPTI element.

The invention further provides Fc-BPTI fusion proteins with mutations in the BPTI element. Mutations at alanine 16 (Ala16) in the BPTI domain sequence

(SEQ ID NO: 3)
RPDFCLEPPYTGPCKARIIRYFYNAKAGLCQTFVYGGCRAKRNNFKSAED
CMRTCGGA

are useful. For example, the mutations Ala16Ser, Ala16Val, and Ala16His are particularly useful. The mutations Ala16Ser and Ala16Val have the property of reducing affinity for trypsin, chymotrypsin, plasmin and kallikrein by several orders of magnitude across the board. Each mutation reduces binding and inhibition of each target protease by roughly the same proportion, but since the dissociation constant of BPTI for trypsin is about 10-100 femtomolar, a reduction of affinity of even 100,000-fold would still result in a pharmaceutically acceptable affinity for trypsin, but the affinity for kallikrein and plasmin is then so low that it does not occur at concentrations achieved in the body after a typical dose of Fc-BPTI. Thus, Fc-BPTI(Ala16Ser) and Fc-BPTI(Ala16Val) have reduced clotting-related side effects relative to wild-type BPTI itself and relative to Fc-BPTI(wild-type).

The mutation Ala16His has a surprising property that makes it particularly useful in some contexts, such as in an Fc-BPTI fusion protein: this mutant protein shows pH-dependent binding to trypsin as well as reduced overall binding. Specifically, the dissociation constant (K_{D}) of trypsin binding to Fc-BPTI(Ala16His) is about 7.1×10⁻⁶ at pH6.5, and about 5.5×10⁻⁷ at pH7.4. This characteristic is useful because the Fc-BPTI first binds to trypsin in the extracellular space in the pancreas at a pH above 7, but when the Fc-BPTI(Ala16His) is internalized into a cell and enters the endosome, the pH is lowered and dissociation occurs. The Fc-BPTI(Ala16His) is then recycled back out of the cell due to FcRn binding, while the trypsin is preferentially transported to the lysosome and degraded. It should be noted that to obtain the full advantage of Fc-BPTI(Ala16His), the Fc region should have an intact N-linked glycosylation site and be capable of binding to an Fc receptor. Forms of Fc with CH2 and CH3 domains based on IgG1 and IgG3 are particularly useful in this regard, and expression of these proteins in mammalian cells is preferred.

The full sequence of IgG1-based Fc-BPTI(Ala16His) is:

EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGARPDFCLEPPYTGPCKHRI
IRYFYNAKAGLCQTFVYGGCRAKRNNFKSAEDCMRTCGGA-
(SEQ ID NO: 4, BPTI domain underlined and
Ala16His substitution in bold).

The full sequence of Fc-BPTI(Ala16His) in which the CH2 and CH3 domains are based primarily on IgG3 is:

epkscdkthtcppCPAPELLGGPSVFLFPPKPKDTLMISRTPEVTCVVVD
VSHEDPEVQFKWYVDGVEVHNAKTKPREEQYNSTFRVVSVLTVLHQDWLN
GKEYKCKVSNKALPAPIEKTISKTKGQPREPQVYTLPPSREEMTKNQVSL
TCPVKGFYPSDIAVEWESSGQPENNYNTTPPMLDSDGSFFLYSKLTVDKS
RWQQGNIFSCSVMHEALHNhyTQKSLSLSPGaRPDFCLEPPYTGPCKHRI
IRYFYNAKAGLCQTFVYGGCRAKRNNFKSAEDCMRTCGGA
(SEQ ID NO: 5, BPTI domain underlined and
Ala16His substitution in bold).

In some embodiments, the Fc-BPTI fusion protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to one of SEQ ID NOs: 2-5.

Without wishing to be bound by theory, the mechanism by which Fc-BPTI(Ala16His) optimally functions is that the BPTI element first binds to trypsin in the extracellular space, secondly binds to an Fc receptor (FcR) on and FcR-bearing cell, thirdly is internalized into the endosomal compartment of the FcR-bearing cell, fourthly dissociation of trypsin from the BPTI element as a result of the reduced pH in the endosome, and fifthly a significant fraction of the free trypsin is transported to the lysosome while the Fc-BPTI element binds to the antibody recycling receptor FcRn and is returned to the extracellular space. To optimally achieve this mechanism, the invention provides the primarily IgG3-based sequence above, which includes the following features: (a) the upper hinge region is from IgG1 instead of IgG3 to improve molecule-to-molecule consistency of the manufactured protein product; (b) the majority of the CH2 and CH3 sequence is based on IgG3; this feature enhances FcR binding; (c) Arg435 and Phe436 of IgG3 are changed to His and Tyr, respectively; this enhances the plasma half-life of the fusion protein; (d) Lys447 is mutated to Alanine to reduce proteolytic cleavage at the junction between the CH3 domain and the BPTI domain; and (e) the BPTI element has the mutation Ala16His.

The invention further provides Fc-SPINK fusion proteins. The SPINK family of proteins are protease inhibitors with specificity for different sets of proteases. These proteins are small, with about the same size as BPTI but with an unrelated structure. SPINK1 is relatively specific for trypsin and is preferred for use in the context of pancreatitis treatment. The protein configuration Fc-SPINK1 is particularly preferred.

In one embodiment, the invention provides Fc-SPINK1 fusion proteins with the following sequences:

(SEQ ID NO: 6)
EPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFS
CSVMHEALHNHYTQKSLSLSPGADSLGREAKCYNELNGCTKIYDPVCGTDG
NTYPNECVLCFENRKRQTSILIQKSGPC
(SEQ ID NO: 7)
EPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFS
CSVMHEALHNHYTQKSLSLSPGADSLGREAKCYNELNGCTKIYDPVCGTDG
NTYPNECVLCFENRKRQTSILIQKSGPCGPEQKLISEEDLNSALVPRGSRLVDH
HHHHH
(SEQ ID NO: 8)
EPKSSDKTHTCPPCPAPELLGGPSVFLFPPKPKDTLMISRTPEVTCVVVDVSHE
DPEVKFNWYVDGVEVHNAKTKPREEQYDSTYRVVSVLTVLHQDWLNGKEY
KCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFS
CSVMHEALHNHYTQKSLSLSPGADSLGREAKCYNELNGCTKIYDPVCGTDG
NTYPNECVLCFENRKRQTSILIQKSGPC.

These three Fc-SPINK1 sequences constitute a mature form of the protein as it would be secreted from a cell after cleavage and removal of a signal sequence. The first contains a typical human IgG1-based Fc sequence, with an N-linked glycosylation site at Asn297, and should ideally be expressed in mammalian cells in secreted form. The second has the same initial sequence as the first, but with a C-terminal cMYC epitope tag and His₆ purification tag. The third protein has a mutated N-linked glycosylation site (Asn297Asp) and is well-suited for expression and secretion in the yeast Pichia pastoris.

Another type of Fc-SPINK1 sequence is illustrated by the following specific embodiment:

(SEQ ID NO: 9)
EPeeSDKTHTCPPCPAPELLGGPSVFLFPPKPKDTLMISRTPEVTCVVVD
VSHEDPEVKFNWYVDGVEVHNAKTKPREEQYdSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGeDSLGREAKCYNELNGCTK
IYDPVCGTDGNTYPNECVLCFENRKRQTSILIQKSGPC.

This protein includes the mutation of several amino acids to a more negatively charged form, which confers a superior pharmacokinetic profile. According to the invention, it is possible to convert neutral or positively charged amino acids to negatively charged amino acids. In addition, it is generally possible to insert negatively charged amino acids at the N-terminus, C-terminus, or at the junction between the Fc element and the SPINK element. The following generalized sequence illustrates this principle:

(SEQ ID NO: 10)
(X)ₗEPxxSDKTHTCPPCPAPELLGGPSVFLFPPKPKDTLMISRTPEVTCV
VVDVSHEDPEVKFNWYVDGVEVHNAKTKPREEQYxSTYRVVSVLTVLHQDW
LNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKS
RWQQGNVFSCSVMHEALHNHYTQKSLSLSPG(X)ₘDSLGREAKCYNELNGC
TKIYDPVCGTDGNTYPNECVLCFENRKRQTSILIQKSGPC(X)ₙ,,

where each X is independently aspartic acid (D) or glutamic acid (E), and the subscripts ‘ ’1′, ‘m’ and ‘n’ and N independently range from 1 to up to 10 or more.

Another exemplary sequence illustrating this principle is as follows:

(SEQ ID NO: 11)
(X)ₚEPeeSDKTHTCPPCPAPELLGGPSVFLFPPKPKDTLMISRTPEVTCV
VVDVSHEDPEVKFNWYVDGVEVHNAKTKPREEQYdSTYRVVSVLTVLHQD
WLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTV
DKSRWQQGNVFSCSVMHEALHNHYTQKSLSLSPGe(X)_{q}DSLGREAKCYN
ELNGCTKIYDPVCGTDGNTYPNECVLCFENRKRQTSILIQKSGPC(X)ᵣ,
where each X is independently aspartic acid (D)
or glutamic acid (E), and the subscripts
'‘p’, ‘p’ and ‘r’ independently range from 1
to up to 10 or more

In some embodiments, the Fc-SPINK fusion protein is selected from Table 2. In some embodiments, the Fc-SPINK fusion protein is encoded by a vector comprising one of SEQ ID NOs: 115-145 or a nucleic acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to one of SEQ ID NOs: 115-145. In some embodiments, the Fc-SPINK vector is pPICZαA, which is an expression vector for the yeast Pichia pastoris.

In some embodiments, the Fc-SPINK fusion protein is encoded by a polynucleotide comprising one of SEQ ID NOs: 146-175 and 241 or a nucleic acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to one of SEQ ID NOs: 146-175. SEQ ID NOs: 146-175 and 241 represent coding sequences; since SEQ ID NOs: 146-175 and 241 encode for secreted proteins that are preceded by a signal sequence, they include no ATG start codon. In addition, SEQ ID NOs: 146-175 and 241 include no stop codon at the end of the sequence. In some embodiments, at least one of SEQ ID NOs: 146-175 and 241 further comprises a start codon at the 5′ end of the sequence and/or a stop codon at the 3′ end of the sequence.

In some embodiments, the Fc-SPINK fusion protein comprises SEQ ID NO: 85 or an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 85, that maintains the same function (see e.g., Example 14).

In some embodiments, the Fc-SPINK fusion protein comprises an amino acid sequence selected from SEQ ID NOs: 1, 6-11 and 85-114 or an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to one of SEQ ID NOs: 1, 6-11 and 85-114, that maintains the same function.

TABLE 2
Exemplary Fc-SPINK Sequences; SID = SEQ ID NO

| | Fusion | | Fusion | Fusion Protein Sequence (SPINK domain bolded, |
|---|---|---|---|---|
| | Protein | | Protein | see e.g., SEQ ID NO: 14 or 186; Fc domain |
| | Amino | | Nucleic | double-underlined, see e.g., SEQ ID NOs: |
| Fusion Protein | Acid | Plasmid | Acid | 37. 40. 187-204; IL-1ra domain, if present, |
| Name | SID | SID | SID | italicized, see e.g., SEQ ID NO: 205-207) |
| Fc(N82D; | 85 | 115 | 241 | EPEESDKTHTCPPCPAPEaaGGPSVFLFPPKPKDTLyItR |
| YTE;LALA-PG)- | | | | ePEVTCVVVDVSHEDPEVKFNWYVDGVEVHNAKTK |
| SPINK_neg- | | | | PREEQYDSTYRVVSVLTVLHQDWLNGKEYKCKVSN |
| charge_noTag | | | | KALgAPIEKTISKAKGQPREPQVYTLPPSRDELTKNQ |
| | | | | VSLTCLVKGFYPSDIAVEWESNGQPENNYKTTPPVL |
| | | | | DSDGSFFLYSKLTVDKSRWQQGNVFSCSVMHEALH |
| | | | | NHYTQKSLSLSPGEDSLGREAKCYNELNGCTKIYD |
| | | | | PVCGTDGNTYPNECVLCFENRKRQTSILIQKSGPC |
| Fc(N82D)- | 86 | 116 | 146 | EPKSSDKTHTCPPCPAPELLGGPSVFLFPPKPKDTLMI |
| SPINK_Myc-6xHis | | | | SRTPEVTCVVVDVSHEDPEVKFNWYVDGVEVHNAK |
| (original | | | | TKPREEQYDSTYRVVSVLTVLHQDWLNGKEYKCKV |
| construct) | | | | SNKALPAPIEKTISKAKGQPREPQVYTLPPSRDELTK |
| | | | | NQVSLTCLVKGFYPSDIAVEWESNGOPENNYKTTPP |
| | | | | VLDSDGSFFLYSKLTVDKSRWQQGNVFSCSVMHEA |
| | | | | LHNHYTQKSLSLSPGADSLGREAKCYNELNGCTKI |
| | | | | YDPVCGTDGNTYPNECVLCFENRKRQTSILIQKSG |
| | | | | PCGPEQKLISEEDLNSALVPRGSRLVDHHHHHH |
| Fc(N82D)- | 8 | 117 | 147 | EPKSSDKTHTCPPCPAPELLGGPSVFLFPPKPKDTLMI |
| SPINK_noTag | | | | SRTPEVTCVVVDVSHEDPEVKFNWYVDGVEVHNAK |
| | | | | TKPREEQYDSTYRVVSVLTVLHQDWLNGKEYKCKV |
| | | | | SNKALPAPIEKTISKAKGOPREPQVYTLPPSRDELTK |
| | | | | NQVSLTCLVKGFYPSDIAVEWESNGQPENNYKTTPP |
| | | | | VLDSDGSFFLYSKLTVDKSRWQQGNVFSCSVMHEA |
| | | | | LHNHYTQKSLSLSPGADSLGREAKCYNELNGCTKI |
| | | | | YDPVCGTDGNTYPNECVLCFENRKRQTSILIQKSG |
| | | | | PC |
| SPINK- | 87 | 118 | 148 | DSLGREAKCYNELNGCTKIYDPVCGTDGNTYPNE |
| Fc(N82D)_noTag | | | | CVLCFENRKRQTSILIQKSGPCEPKSSDKTHTCPPC |
| | | | | PAPELLGGPSVFLFPPKPKDTLMISRTPEVTCVVVDV |
| | | | | SHEDPEVKFNWYVDGVEVHNAKTKPREEQYDSTYR |
| | | | | VVSVLTVLHODWLNGKEYKCKVSNKALPAPIEKTIS |
| | | | | KAKGQPREPQVYTLPPSRDELTKNQVSLTCLVKGFY |
| | | | | PSDIAVEWESNGQPENNYKTTPPVLDSDGSFFLYSKL |
| | | | | TVDKSRWQQGNVESCSVMHEALHNHYTQKSLSLSP |
| | | | | GA |
| SPINK-Fc(N82D)- | 88 | 119 | 149 | DSLGREAKCYNELNGCTKIYDPVCGTDGNTYPNE |
| Il1Ra_noTag | | | | CVLCFENRKRQTSILIQKSGPCEPKSSDKTHTCPPC |
| | | | | PAPELLGGPSVFLFPPKPKDTLMISRTPEVTCVVVDV |
| | | | | SHEDPEVKFNWYVDGVEVHNAKTKPREEQYDSTYR |
| | | | | VVSVLTVLHQDWLNGKEYKCKVSNKALPAPIEKTIS |
| | | | | KAKGQPREPQVYTLPPSRDELTKNQVSLTCLVKGFY |
| | | | | PSDIAVEWESNGOPENNYKTTPPVLDSDGSFFLYSKL |
| | | | | TVDKSRWQQGNVFSCSVMHEALHNHYTQKSLSLSP |
| | | | | GARPSGRKSSKMQAFRIWDVNQKTFYLRNNQLVAGYL |
| | | | | QGPNVNLEEKIDVVPIEPHALFLGIHGGKMCLSCVKSG |
| | | | | DETRLQLEAVDITDLSENRKQDKRFAFIRSDSGPTTSFE |
| | | | | SAACPGWFLCTAMEADQPVSLTNMPDEGVMVTKFYFQ |
| | | | | EDE |
| IL1Ra-Fc(N82D)- | 89 | 120 | 150 | RPSGRKSSKMQAFRIWDVNQKTFYLRNNQLVAGYLQGP |
| SPINK_noTag | | | | NVNLEEKIDVVPIEPHALFLGIHGGKMCLSCVKSGDET |
| | | | | RLQLEAVDITDLSENRKQDKRFAFIRSDSGPTTSFESAA |
| | | | | CPGWFLCTAMEADQPVSLTNMPDEGVMVTKFYFQED |
| | | | | EEPKSSDKTHTCPPCPAPELLGGPSVFLFPPKPKDTL |
| | | | | MISRTPEVTCVVVDVSHEDPEVKFNWYVDGVEVHN |
| | | | | AKTKPREEQYDSTYRVVSVLTVLHODWLNGKEYKC |
| | | | | KVSNKALPAPIEKTISKAKGQPREPQVYTLPPSRDEL |
| | | | | TKNQVSLTCLVKGFYPSDIAVEWESNGQPENNYKTT |
| | | | | PPVLDSDGSFFLYSKLTVDKSRWQQGNVFSCSVMHE |
| | | | | ALHNHYTQKSLSLSPGADSLGREAKCYNELNGCTK |
| | | | | IYDPVCGTDGNTYPNECVLCFENRKRQTSILIQKS |
| | | | | GPC |
| Fc(N82D)- | 90 | 121 | 151 | EPEESDKTHTCPPCPAPELLGGPSVFLFPPKPKDTLMI |
| SPINK_neg- | | | | SRTPEVTCVVVDVSHEDPEVKFNWYVDGVEVHNAK |
| charge_noTag | | | | TKPREEQYDSTYRVVSVLTVLHODWLNGKEYKCKV |
| | | | | SNKALPAPIEKTISKAKGOPREPOVYTLPPSRDELTK |
| | | | | NQVSLTCLVKGFYPSDIAVEWESNGOPENNYKTTPP |
| | | | | VLDSDGSFFLYSKLTVDKSRWQQGNVFSCSVMHEA |
| | | | | LHNHYTQKSLSLSPGEDSLGREAKCYNELNGCTKI |
| | | | | YDPVCGTDGNTYPNECVLCFENRKRQTSILIQKSG |
| | | | | PC |
| murine_Fc(N83D)- | 91 | 122 | 152 | EPRGPTIKPCPPCKCPAPNLLGGPSVFIFPPKIKDVLMI |
| SPINK1_noTag | | | | SLSPIVTCVVVDVSEDDPDVQISWFVNNVEVHTAQT |
| | | | | QTHREDYDSTLRVVSALPIQHQDWMSGKEFKCKVN |
| | | | | NKDLPAPIERTISKPKGSVRAPQVYVLPPPEEEMTKK |
| | | | | QVTLTCMVTDFMPEDIYVEWTNNGKTELNYKNTEP |
| | | | | VLDSDGSYFMYSKLRVEKKNWVERNSYSCSVVHEG |
| | | | | LHNHHTTKSFSRTPGKAKVTGKEASCHDAVAGCP |
| | | | | RIYDPVCGTDGITYANECVLCFENRKRIEPVLIRK |
| | | | | GGPC |
| murine_SPINK1- | 92 | 123 | 153 | AKVTGKEASCHDAVAGCPRIYDPVCGTDGITYAN |
| Fc(N83D)_noTag | | | | ECVLCFENRKRIEPVLIRKGGPCEPRGPTIKPCPPC |
| | | | | KCPAPNLLGGPSVFIFPPKIKDVLMISLSPIVTCVVVD |
| | | | | VSEDDPDVQISWFVNNVEVHTAQTQTHREDYDSTL |
| | | | | RVVSALPIQHQDWMSGKEFKCKVNNKDLPAPIERTI |
| | | | | SKPKGSVRAPQVYVLPPPEEEMTKKQVTLTCMVTDE |
| | | | | MPEDIYVEWTNNGKTELNYKNTEPVLDSDGSYFMY |
| | | | | SKLRVEKKNWVERNSYSCSVVHEGLHNHHTTKSES |
| | | | | RTPGK |
| murine SPINK1- | 93 | 124 | 154 | AKVTGKEASCHDAVAGCPRIYDPVCGTDGITYAN |
| Fc(N83D)_IL- | | | | ECVLCFENRKRIEPVLIRKGGPCEPRGPTIKPCPPC |
| 1Ra_noTag | | | | KCPAPNLLGGPSVFIFPPKIKDVLMISLSPIVTCVVVD |
| | | | | VSEDDPDVQISWFVNNVEVHTAQTQTHREDYDSTL |
| | | | | RVVSALPIQHQDWMSGKEFKCKVNNKDLPAPIERTI |
| | | | | SKPKGSVRAPQVYVLPPPEEEMTKKQVTLTCMVTDE |
| | | | | MPEDIYVEWTNNGKTELNYKNTEPVLDSDGSYFMY |
| | | | | SKLRVEKKNWVERNSYSCSVVHEGLHNHHTTKSFS |
| | | | | RTPGKRPSGKRPCKMQAFRIWDTNQKTFYLRNNQLIA |
| | | | | GYLQGPNIKLEEKIDMVPIDLHSVFLGIHGGKLCLSCAK |
| | | | | SGDDIKLQLEEVNITDLSKNKEEDKRFTFIRSEKGPTTSF |
| | | | | ESAACPGWFLCTTLEADRPVSLTNTPEEPLIVTKFYFQE |
| | | | | DQ |
| murine_SPINK1- | 94 | 125 | 155 | AKVTGKEASCHDAVAGCPRIYDPVCGTDGITYAN |
| Fc(N83D)_IL- | | | | ECVLCFENRKRIEPVLIRKGGPCEPRGPTIKPCPPC |
| 1Ra(N84D)_noTag | | | | KCPAPNLLGGPSVFIFPPKIKDVLMISLSPIVTCVVVD |
| | | | | VSEDDPDVQISWFVNNVEVHTAQTQTHREDYDSTL |
| | | | | RVVSALPIQHQDWMSGKEFKCKVNNKDLPAPIERTI |
| | | | | SKPKGSVRAPQVYVLPPPEEEMTKKQVTLTCMVTDF |
| | | | | MPEDIYVEWTNNGKTELNYKNTEPVLDSDGSYFMY |
| | | | | SKLRVEKKNWVERNSYSCSVVHEGLHNHHTTKSES |
| | | | | RTPGKRPSGKRPCKMQAFRIWDTNQKTFYLRNNQLIA |
| | | | | GYLQGPNIKLEEKIDMVPIDLHSVFLGIHGGKLCLSCAK |
| | | | | SGDDIKLQLEEVDITDLSKNKEEDKRFTFIRSEKGPTTS |
| | | | | FESAACPGWFLCTTLEADRPVSLTNTPEEPLIVTKFYFQ |
| | | | | EDQ |
| (pOst1)_SPINK- | 95 | 126 | 156 | DSLGREAKCYNELNGCTKIYDPVCGTDGNTYPNE |
| Fc(N82D)- | | | | CVLCFENRKRQTSILIQKSGPCEPKSSDKTHTCPPC |
| Il1Ra_noTag | | | | PAPELLGGPSVFLFPPKPKDTLMISRTPEVTCVVVDV |
| | | | | SHEDPEVKFNWYVDGVEVHNAKTKPREEQYDSTYR |
| | | | | VVSVLTVLHQDWLNGKEYKCKVSNKALPAPIEKTIS |
| | | | | KAKGQPREPQVYTLPPSRDELTKNQVSLTCLVKGFY |
| | | | | PSDIAVEWESNGQPENNYKTTPPVLDSDGSFFLYSKL |
| | | | | TVDKSRWQQGNVFSCSVMHEALHNHYTQKSLSLSP |
| | | | | GARPSGRKSSKMQAFRIWDVNQKTFYLRNNQLVAGYL |
| | | | | QGPNVNLEEKIDVVPIEPHALFLGIHGGKMCLSCVKSG |
| | | | | DETRLQLEAVDITDLSENRKQDKRFAFIRSDSGPTTSFE |
| | | | | SAACPGWFLCTAMEADQPVSLTNMPDEGVMVTKFYFQ |
| | | | | EDE |
| (pro-d57-70) | 96 | 127 | 157 | DSLGREAKCYNELNGCTKIYDPVCGTDGNTYPNE |
| SPINK- | | | | CVLCFENRKRQTSILIQKSGPCEPKSSDKTHTCPPC |
| Fc(N82D)- | | | | PAPELLGGPSVFLFPPKPKDTLMISRTPEVTCVVVDV |
| Il1Ra_noTag | | | | SHEDPEVKFNWYVDGVEVHNAKTKPREEQYDSTYR |
| | | | | VVSVLTVLHQDWLNGKEYKCKVSNKALPAPIEKTIS |
| | | | | KAKGQPREPQVYTLPPSRDELTKNQVSLTCLVKGFY |
| | | | | PSDIAVEWESNGQPENNYKTTPPVLDSDGSFFLYSKL |
| | | | | TVDKSRWQQGNVFSCSVMHEALHNHYTQKSLSLSP |
| | | | | GARPSGRKSSKMQAFRIWDVNQKTFYLRNNQLVAGYL |
| | | | | QGPNVNLEEKIDVVPIEPHALFLGIHGGKMCLSCVKSG |
| | | | | DETRLQLEAVDITDLSENRKQDKRFAFIRSDSGPTTSFE |
| | | | | SAACPGWFLCTAMEADQPVSLTNMPDEGVMVTKFYFQ |
| | | | | EDE |
| (rm_pro)_ SPINK- | 97 | 128 | 158 | DSLGREAKCYNELNGCTKIYDPVCGTDGNTYPNE |
| Fc(N82D)- | | | | CVLCFENRKRQTSILIQKSGPCEPKSSDKTHTCPPC |
| IL1Ra_noTag | | | | PAPELLGGPSVFLFPPKPKDTLMISRTPEVTCVVVDV |
| | | | | SHEDPEVKFNWYVDGVEVHNAKTKPREEQYDSTYR |
| | | | | VVSVLTVLHQDWLNGKEYKCKVSNKALPAPIEKTIS |
| | | | | KAKGQPREPQVYTLPPSRDELTKNQVSLTCLVKGFY |
| | | | | PSDIAVEWESNGQPENNYKTTPPVLDSDGSFFLYSKL |
| | | | | TVDKSRWQQGNVFSCSVMHEALHNHYTQKSLSLSP |
| | | | | GARPSGRKSSKMQAFRIWDVNQKTFYLRNNQLVAGYL |
| | | | | QGPNVNLEEKIDVVPIEPHALFLGIHGGKMCLSCVKSG |
| | | | | DETRLQLEAVDITDLSENRKQDKRFAFIRSDSGPTTSFE |
| | | | | SAACPGWFLCTAMEADQPVSLTNMPDEGVMVTKFYFQ |
| | | | | EDE |
| murine_Fc(N89D)- | 98 | 129 | 159 | EPSGPISTINPCPPCKECHKCPAPNLEGGPSVFIFPPNI |
| SPINK1_noTag - | | | | KDVLMISLTPKVTCVVVDVSEDDPDVRISWFVNNVE |
| IgG2b | | | | VHTAQTQTHREDYDSTIRVVSALPIQHQDWMSGKEF |
| Fc(N89D; K239A) | | | | KCKVNNKDLPSPIERTISKIKGLVRAPQVYILPPPAEQ |
| | | | | LSRKDVSLTCLVVGFNPGDISVEWTSNGHTEENYKD |
| | | | | TAPVLDSDGSYFIYSKLDIKTSKWEKTDSFSCNVRHE |
| | | | | GLKNYYLKKTISRSPGAAKVTGKEASCHDAVAGCP |
| | | | | RIYDPVCGTDGITYANECVLCFENRKRIEPVLIRK |
| | | | | GGPC |
| murine_Fc(N89D)- | 99 | 130 | 160 | EPSGPISTINPCPPCKECHKCPAPNLEGGPSVFIFPPNI |
| SPINK1_noTag - | | | | KDVLYITLEPKVTCVVVDVSEDDPDVRISWFVNNVE |
| IgG2b Fc | | | | VHTAQTQTHREDYDSTIRVVSALPIQHQDWMSGKEF |
| (N89D; K239A; | | | | KCKVNNKDLPSPIERTISKIKGLVRAPQVYILPPPAEQ |
| YTE) | | | | LSRKDVSLTCLVVGFNPGDISVEWTSNGHTEENYKD |
| | | | | TAPVLDSDGSYFIYSKLDIKTSKWEKTDSFSCNVRHE |
| | | | | GLKNYYLKKTISRSPGAAKVTGKEASCHDAVAGCP |
| | | | | RIYDPVCGTDGITYANECVLCFENRKRIEPVLIRK |
| | | | | GGPC |
| murine_Fc(N89D)- | 100 | 131 | 161 | EPSGPISTINPCPPCKECHKCPAPNAAGGPSVFIFPPNI |
| SPINK1_noTag - | | | | KDVLYITLEPKVTCVVVDVSEDDPDVRISWFVNNVE |
| IgG2b | | | | VHTAQTQTHREDYDSTIRVVSALPIQHQDWMSGKEF |
| Fc(N89D; K239A; | | | | KCKVNNKDLGSPIERTISKIKGLVRAPQVYILPPPAEQ |
| YTE; LALA-PG) | | | | LSRKDVSLTCLVVGFNPGDISVEWTSNGHTEENYKD |
| | | | | TAPVLDSDGSYFIYSKLDIKTSKWEKTDSFSCNVRHE |
| | | | | GLKNYYLKKTISRSPGAAKVTGKEASCHDAVAGCP |
| | | | | RIYDPVCGTDGITYANECVLCFENRKRIEPVLIRK |
| | | | | GGPC |
| murine_Fc(N89D)- | 101 | 132 | 162 | EPSGPISTINPCPPCKECHKCPAPNLEGGPSVFIFPPNI |
| SPINK1_noTag - | | | | KDVLYITLEPKVTCVVEDVSEDDPDVRISWFVNNVE |
| IgG2b | | | | VHTAQTQTHREDYDSTIRVVSALPIQHQDWMSGKEF |
| Fc(N89D; K239A; | | | | KCKVNNKDLPSPIERTISKIKGLVRAPQVYILPPPAEQ |
| YTE; V264E) | | | | LSRKDVSLTCLVVGFNPGDISVEWTSNGHTEENYKD |
| | | | | TAPVLDSDGSYFIYSKLDIKTSKWEKTDSFSCNVRHE |
| | | | | GLKNYYLKKTISRSPGAAKVTGKEASCHDAVAGCP |
| | | | | RIYDPVCGTDGITYANECVLCFENRKRIEPVLIRK |
| | | | | GGPC |
| murine_Fc(N89D)- | 102 | 133 | 163 | EPSGPISTINPCPPCKECHKCPAPNLEGGPSVFIFPPNI |
| SPINK1_noTag - | | | | KDVLMISLTPKVTCVVVDVSEDDPDVRISWFVNNVE |
| IgG2b | | | | VHTAQTQTHREDYDSTIRVVSALPIQHQDWMSGKEF |
| Fc(N89D; K239A; | | | | KCKVNNKDLPSPIERTISKIKGLVRAPQVYILPPPAEQ |
| LS) | | | | LSRKDVSLTCLVVGFNPGDISVEWTSNGHTEENYKD |
| | | | | TAPVLDSDGSYFIYSKLDIKTSKWEKTDSFSCNVLHE |
| | | | | GLKSYYLKKTISRSPGAAKVTGKEASCHDAVAGCP |
| | | | | RIYDPVCGTDGITYANECVLCFENRKRIEPVLIRK |
| | | | | GGPC |
| murine_Fc(N89D)- | 103 | 134 | 164 | EPSGPISTINPCPPCKECHKCPAPNAAGGPSVFIFPPNI |
| SPINK1_noTag - | | | | KDVLMISLTPKVTCVVVDVSEDDPDVRISWFVNNVE |
| IgG2b | | | | VHTAQTQTHREDYDSTIRVVSALPIQHQDWMSGKEF |
| Fc(N89D; K239A; | | | | KCKVNNKDLGSPIERTISKIKGLVRAPQVYILPPPAEQ |
| LS; LALA-PG) | | | | LSRKDVSLTCLVVGFNPGDISVEWTSNGHTEENYKD |
| | | | | TAPVLDSDGSYFIYSKLDIKTSKWEKTDSFSCNVLHE |
| | | | | GLKSYYLKKTISRSPGAAKVTGKEASCHDAVAGCP |
| | | | | RIYDPVCGTDGITYANECVLCFENRKRIEPVLIRK |
| | | | | GGPC |
| murine_Fc(N89D)- | 104 | 135 | 165 | EPSGPISTINPCPPCKECHKCPAPNLEGGPSVFIFPPNI |
| SPINK1_noTag - | | | | KDVLMISLTPKVTCVVEDVSEDDPDVRISWFVNNVE |
| IgG2b | | | | VHTAQTQTHREDYDSTIRVVSALPIQHQDWMSGKEE |
| Fc(N89D; K239A; | | | | KCKVNNKDLPSPIERTISKIKGLVRAPQVYILPPPAEQ |
| LS; V264E) | | | | LSRKDVSLTCLVVGFNPGDISVEWTSNGHTEENYKD |
| | | | | TAPVLDSDGSYFIYSKLDIKTSKWEKTDSFSCNVLHE |
| | | | | GLKSYYLKKTISRSPGAAKVTGKEASCHDAVAGCP |
| | | | | RIYDPVCGTDGITYANECVLCFENRKRIEPVLIRK |
| | | | | GGPC |
| murine_Fc(N89D)- | 105 | 136 | 166 | EPSGPISTINPCPPCKECHKCPAPNLEGGPSVFIFPPNI |
| SPINK1_noTag - | | | | KDVLMISLTPKVTCVVVDVSEDDPDVRISWFVNNVE |
| IgG2b | | | | VHTAQTQTHREDYDSTIRVVSALPIDHHDWMSGKEF |
| Fc(N89D; K239A; | | | | KCKVNNKDLPSPIERTISKIKGLVRAPQVYILPPPAEQ |
| DHS) | | | | LSRKDVSLTCLVVGFNPGDISVEWTSNGHTEENYKD |
| | | | | TAPVLDSDGSYFIYSKLDIKTSKWEKTDSFSCNVRHE |
| | | | | GLKSYYLKKTISRSPGAAKVTGKEASCHDAVAGCP |
| | | | | RIYDPVCGTDGITYANECVLCFENRKRIEPVLIRK |
| | | | | GGPC |
| murine_Fc(N89D)- | 106 | 137 | 167 | EPSGPISTINPCPPCKECHKCPAPNAAGGPSVFIFPPNI |
| SPINK1_noTag - | | | | KDVLMISLTPKVTCVVVDVSEDDPDVRISWFVNNVE |
| IgG2b | | | | VHTAQTQTHREDYDSTIRVVSALPIDHHDWMSGKEF |
| Fc(N89D; K239A; | | | | KCKVNNKDLGSPIERTISKIKGLVRAPQVYILPPPAEQ |
| DHS; LALA-PG) | | | | LSRKDVSLTCLVVGFNPGDISVEWTSNGHTEENYKD |
| | | | | TAPVLDSDGSYFIYSKLDIKTSKWEKTDSFSCNVRHE |
| | | | | GLKSYYLKKTISRSPGAAKVTGKEASCHDAVAGCP |
| | | | | RIYDPVCGTDGITYANECVLCFENRKRIEPVLIRK |
| | | | | GGPC |
| murine_Fc(N89D)- | 107 | 138 | 168 | EPSGPISTINPCPPCKECHKCPAPNLEGGPSVFIFPPNI |
| SPINK1_noTag- | | | | KDVLMISLTPKVTCVVEDVSEDDPDVRISWFVNNVE |
| IgG2b | | | | VHTAQTQTHREDYDSTIRVVSALPIDHHDWMSGKEF |
| Fc(N89D; K239A; | | | | KCKVNNKDLPSPIERTISKIKGLVRAPQVYILPPPAEQ |
| EDHS) | | | | LSRKDVSLTCLVVGFNPGDISVEWTSNGHTEENYKD |
| | | | | TAPVLDSDGSYFIYSKLDIKTSKWEKTDSFSCNVRHE |
| | | | | GLKSYYLKKTISRSPGAAKVTGKEASCHDAVAGCP |
| | | | | RIYDPVCGTDGITYANECVLCFENRKRIEPVLIRK |
| | | | | GGPC |
| murine_Fc(N89D)- | 108 | 139 | 169 | EPKSSPPCKECSIFPAPDAAGGPSVFIFPPKIKDVLYIT |
| SPINK1_noTag- | | | | LEPEVTCVVVDVSEDDPDVQISWFVNNVEVHTAQT |
| IgG2c Fc(YTE & | | | | QTHREDYDSTLRVVSALPIQHQDWMSGKEFKCKVN |
| LALA) | | | | NRALPSPIEKTISKPRGPVRAPQVYVLPPPAEEMTKK |
| | | | | EFSLTCMITDFLPAEIAVDWTSNGHKELNYKNTAPV |
| | | | | LDTDGSYFMYSKLRVQKSTWEKGSLFACSVVHEGL |
| | | | | HNHHTTKTISRSLGAAKVTGKEASCHDAVAGCPRI |
| | | | | YDPVCGTDGITYANECVLCFENRKRIEPVLIRKGG |
| | | | | PC |
| murine_Fc(N89D)- | 109 | 140 | 170 | EPESEPPCKECSIFPAPDAAGGPSVFIFPPKIKDVLYIT |
| SPINK1_noTag- | | | | LEPEVTCVVVDVSEDDPDVQISWFVNNVEVHTAQT |
| IgG2c Fc(YTE & | | | | QTHREDYDSTLRVVSALPIQHQDWMSGKEFKCKVN |
| LALA; Neg. | | | | NRALPSPIEKTISKPRGPVRAPQVYVLPPPAEEMTKK |
| Charge) | | | | EFSLTCMITDFLPAEIAVDWTSNGHKELNYKNTAPV |
| | | | | LDTDGSYFMYSKLRVQKSTWEKGSLFACSVVHEGL |
| | | | | HNHHTTKTISRSLGEAKVTGKEASCHDAVAGCPRI |
| | | | | YDPVCGTDGITYANECVLCFENRKRIEPVLIRKGG |
| | | | | PC |
| murine_Fc(N89D)- | 110 | 141 | 171 | YPYDVPDYAKPKEPESEPPCKECSIFPAPDAAGGPSV |
| SPINK1_noTag- | | | | FIFPPKIKDVLYITLEPEVTCVVVDVSEDDPDVQISWF |
| IgG2c Fc(YTE & | | | | VNNVEVHTAQTQTHREDYDSTLRVVSALPIQHQDW |
| LALA; Neg. | | | | MSGKEFKCKVNNRALPSPIEKTISKPRGPVRAPQVYV |
| Charge; HA Tag + | | | | LPPPAEEMTKKEFSLTCMITDELPAEIAVDWTSNGHK |
| KPK) | | | | ELNYKNTAPVLDTDGSYFMYSKLRVQKSTWEKGSL |
| | | | | FACSVVHEGLHNHHTTKTISRSLGEAKVTGKEASC |
| | | | | HDAVAGCPRIYDPVCGTDGITYANECVLCFENRK |
| | | | | RIEPVLIRKGGPC |
| Fc(N82D; YTE; | 111 | 142 | 172 | EPKSSDKTHTCPPCPAPEAAGGPSVFLFPPKPKDTLYI |
| LALA) | | | | TREPEVTCVVVDVSHEDPEVKFNWYVDGVEVHNAK |
| SPINK_noTag | | | | TKPREEQYDSTYRVVSVLTVLHQDWLNGKEYKCKV |
| | | | | SNKALPAPIEKTISKAKGQPREPQVYTLPPSRDELTK |
| | | | | NQVSLTCLVKGFYPSDIAVEWESNGQPENNYKTTPP |
| | | | | VLDSDGSFFLYSKLTVDKSRWQQGNVFSCSVMHEA |
| | | | | LHNHYTQKSLSLSPGADSLGREAKCYNELNGCTKI |
| | | | | YDPVCGTDGNTYPNECVLCFENRKRQTSILIQKSG |
| | | | | PC |
| Fc(N82D; YTE; | 112 | 143 | 173 | EPKSSDKTHTCPPCPAPEAAGGPSVFLFPPKPKDTLYI |
| LALA-PG) | | | | TREPEVTCVVVDVSHEDPEVKFNWYVDGVEVHNAK |
| SPINK_noTag | | | | TKPREEQYDSTYRVVSVLTVLHQDWLNGKEYKCKV |
| | | | | SNKALGAPIEKTISKAKGQPREPQVYTLPPSRDELTK |
| | | | | NQVSLTCLVKGFYPSDIAVEWESNGQPENNYKTTPP |
| | | | | VLDSDGSFFLYSKLTVDKSRWQQGNVFSCSVMHEA |
| | | | | LHNHYTQKSLSLSPGADSLGREAKCYNELNGCTKI |
| | | | | YDPVCGTDGNTYPNECVLCFENRKRQTSILIQKSG |
| | | | | PC |
| Fc(N82D; LS; | 113 | 144 | 174 | EPKSSDKTHTCPPCPAPEAAGGPSVFLFPPKPKDTLM |
| LALA-PG) | | | | ISRTPEVTCVVVDVSHEDPEVKFNWYVDGVEVHNA |
| SPINK_noTag | | | | KTKPREEQYDSTYRVVSVLTVLHQDWLNGKEYKCK |
| | | | | VSNKALGAPIEKTISKAKGQPREPQVYTLPPSRDELT |
| | | | | KNQVSLTCLVKGFYPSDIAVEWESNGQPENNYKTTP |
| | | | | PVLDSDGSFFLYSKLTVDKSRWQQGNVFSCSVLHEA |
| | | | | LHSHYTQKSLSLSPGADSLGREAKCYNELNGCTKI |
| | | | | YDPVCGTDGNTYPNECVLCFENRKRQTSILIQKSG |
| | | | | PC |
| Fc(N82D; DHS; | 114 | 145 | 175 | EPKSSDKTHTCPPCPAPEAAGGPSVFLFPPKPKDTLM |
| LALA-PG) | | | | ISRTPEVTCVVVDVSHEDPEVKENWYVDGVEVHNA |
| SPINK_noTag | | | | KTKPREEQYDSTYRVVSVLTVDHHDWLNGKEYKCK |
| | | | | VSNKALGAPIEKTISKAKGQPREPQVYTLPPSRDELT |
| | | | | KNQVSLTCLVKGFYPSDIAVEWESNGQPENNYKTTP |
| | | | | PVLDSDGSFFLYSKLTVDKSRWQQGNVESCSVMHE |
| | | | | ALHSHYTQKSLSLSPGADSLGREAKCYNELNGCTK |
| | | | | IYDPVCGTDGNTYPNECVLCFENRKRQTSILIQKS |
| | | | | GPC |

Fc-SPINK proteins such as the particular Fc-SPINK1 proteins described herein, as well as the Fc-BPTI fusion proteins, are effective in treating pancreatitis. Required doses and duration of treatment vary with the specific fusion protein. The Fc-SPINK1 protein, for example, was used to treat cerulean-induced pancreatitis in a mouse model, and treatment with Fc-SPINK1 ameliorated the release of pancreatic amylase into plasma, the appearance of necrotic tissue in the pancreas, and the infiltration of immune cells into the pancreas. The details of the experiments and results are given in the Examples and in the Figures. The important point is that treatment with fusion proteins of the invention had a beneficial effect on all of these sequelae, even though they are significantly downstream of trypsin in the disease process. These experiments further validate trypsin as a drug target and indicate that the entire disease can be reversed by inhibiting this enzyme.

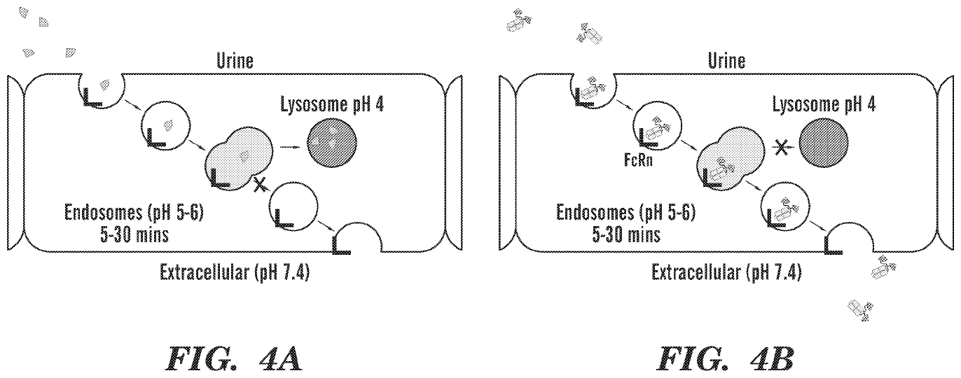

Upon IV injection, BPTI experiences a rapid distribution phase half-life of 0.3 to 0.7 hours, followed by an elimination phase half-life of 2-3 hours. Its small size and neutral charge result in free kidney filtration, where-due to protein conserving receptors in the kidney tubules-BPTI is then actively reabsorbed from the proximal tubule without significant loss into the urine (FIG. 4). Despite this reabsorption, instead of being redistributing to the plasma or broken down into component amino acids, the filtered BPTI remains inside the kidneys trapped by phagolysosomes that struggle to degrade the drug due to its protease inhibitory effects. As a result, the majority of BPTI injected in an attempt to treat pancreatitis instead localizes permanently to an off-target organ. Such an accumulation may contribute to renal side effects; an advantage of the molecules of the invention is that they do not enter the phagolysosomes in the first place, thus avoiding toxic side effects that occur as a result of inhibition of protease activity in kidney phagolysosomes. In addition, the molecules of the invention are quantitatively tuned so that they do not cause inhibition of other proteases.

It should be noted that the marketed form of BPTI/aprotinin, namely TRASYLOL(TM) was withdrawn from the market in the United States due to renal side effects, and in clinical studies, renal side effects of Trasylol treatment were observed and were thought to be the cause of increased mortality (Mangano et al. N Engl J Med 354; 4 Jan. 26, 2006; Schloss et al. Journal of Cardiothoracic Surgery 2011, 6:10). Trasylol is still approved in other countries. It should also be noted that the concentration of BPTI/aprotinin in formulated Trasylol is 1.4 mg/ml (10,000 Units) and typically several hundred milliliters are given during cardiac surgery, with total doses up to 7,000,000 Units (980 milligrams) but typically around 4-5,000,000 Units. It should be noted that, considered in terms of moles of protease inhibitor administered, these doses are comparable to or higher than the doses described here for treatment of pancreatitis (www.rxlist.com/trasylol-drug.htm #description). Thus, an additional use of the BPTI-based molecules of the invention, preferably Fc-BPTI wherein BPTI does not contain weakening mutations, is during cardiac surgery.

Considered broadly, given the small size of BPTI and the high incidence of phagolysosome trapping, the Fc-BPTI fusion proteins of the invention create a much improved profile for the treatment of pancreatitis.

In addition, Fc-BPTI, Fc-SPINK, and the general Fc-proteinase inhibitors of the invention are recycled from the endosomal compartment of Antigen Presenting Cells such as dendritic cells and macrophages, avoiding proteolysis like unfused BPTI. This trait is particularly desirable because degradation is the first step in creating peptides for Antigen Presenting Cells. The fusion proteins of the invention thus have reduced risk of immunogenicity.

The Fc fusion proteins described herein comprise N-linked glycosylation site of the Fc region: GGGAGGAGCAGTA [symbol] (SEQ ID NO: 12, codon for N297 highlighted)). When tissues become inflamed, the resulting release of chemokines draws these cells to areas of injury. An Fc-fusion can then gain access to the injured pancreas while carried inside of such immune cells.

The Fc fusion proteins described herein also comprise the FcRN histidine binding site ILE254 SER254 His435 TYR436: GAGGCTCTGCACAAC [symbol] (SEQ ID NO: 13). Researchers have confirmed that the complex of residues I253, S254, H435, and Y436 is critical for the interaction. See, for example, Firan et al., Immunol., 2001, 13).

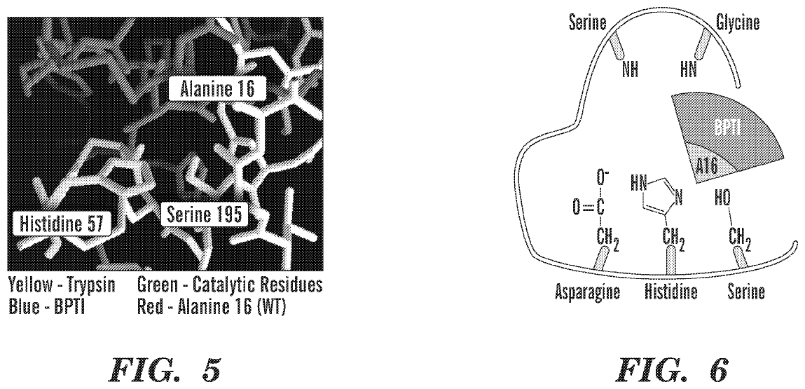

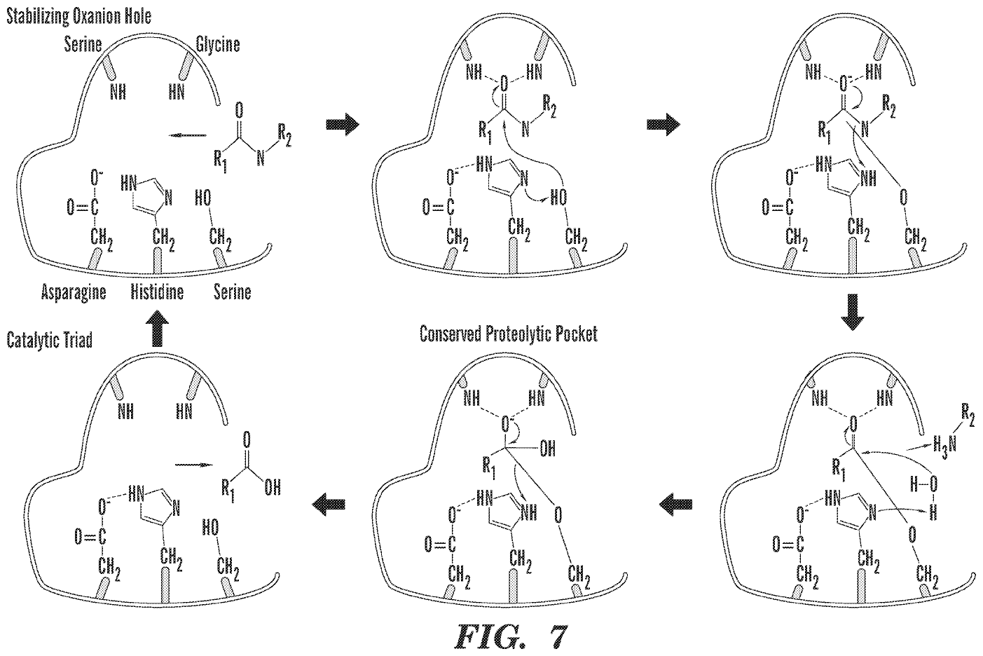

Despite the name of Bovine Pancreatic “Trypsin” Inhibitor, BPTI would be more accurately be described as a general serine protease inhibitor. This class of proteases share a highly conserved active site (FIGS. 5 and 6). BPTI is a competitive inhibitor, blocking this shared pocket (FIG. 7).

BPTI inhibits trypsin most potently, with a K_{D} in the femtomolar range, however this conserved active site results in binding of BPTI across a spectrum of human and animal proteases that is remarkably robustly. For example, the binding between BPTI and plasmin has a K_{D} in the nanomolar range and results in a complex with a half-life on the scale of months. Accordingly, Wild type BPTI can inhibit several other enzymes at the doses needed to titrate trypsin in the pancreas.

Serine proteases are prevalent throughout the body, in both serum and tissue. In the blood, they are found abundantly as the proteases which make up the sequentially activated elements in the coagulation cascade. BPTI is so effective at preventing the destruction of fibrin clots by the serine protease plasmin that it is currently in use, under the trade name TRASYLOL®, as a component of surgical glue throughout Europe.

The abundance of strong, silencing, off-target proteases limits the use of BPTI as a pancreatitis treatment due to narrowing of the therapeutic index via unintended binding side effects. For example, the inhibition of plasmin which makes BPTI useful in surgical glue can lead to unwanted blood clots in pancreatitis patients, resulting in life threatening thrombotic events such as stroke, heart attack, and deep vein thrombosis.

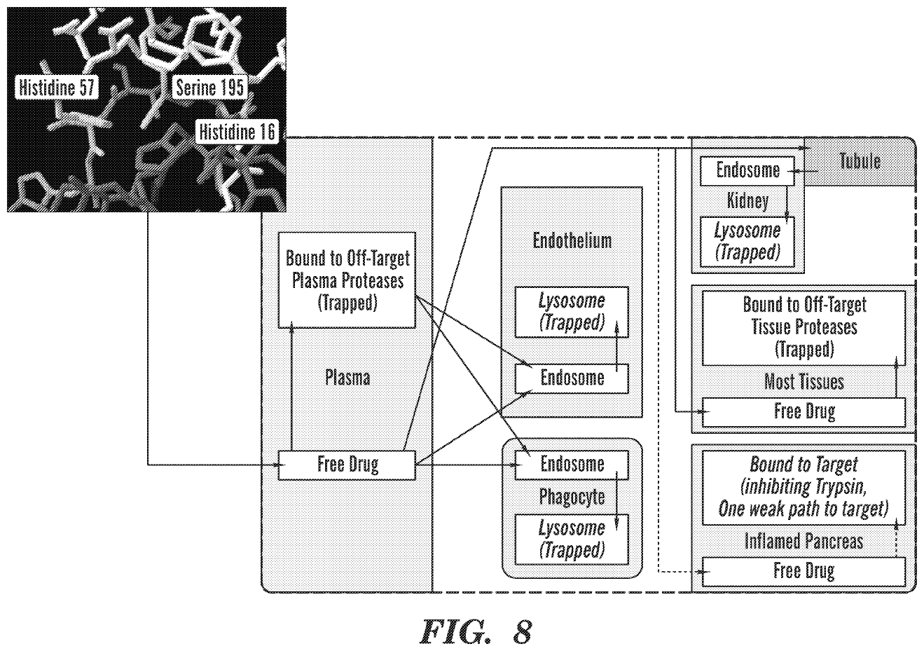

Such binding promiscuity, in addition to risking severe side effects, creates a complex environment for appropriate pharmacokinetic modeling (FIG. 8). Even calculations of half-life provide a misleadingly high number, given that functionally irreversible BPTI binding to off-target serum proteases will inherently increase the molecule's half-life, but ignores the resulting loss of the desired inhibitory functionality. The ability of BPTI to inhibit lysosomal proteases also means that tissue sample testing for the drug would be expected to overestimate the quantity of active drug in the pancreas, because phagocytosed BPTI would be sequestered inside lysosomes without digestion. These situations result in increasingly complex dose calculations, with these issues having sufficiently large effect sizes that they cannot be abstracted away.

Historically, maximizing the affinity of a drug to its target has been a primary goal in drug design. However, in the case of BPTI and its use in treating premature pancreatic trypsin activation, strong target affinity is instead a hindrance to efficacy because affinity to trypsin is tightly coupled to potent and costly off-target binding. This creates a situation where, counterintuitively, reduced potency has the potential to improve drug efficacy by creating a molecule that is more specific to the target of interest despite being more weakly bound in absolute measurements.

Theoretically, an ideal pancreatitis treatment should be designed to not only inhibit trypsin, but to also enjoy minimal renal clearance, exhibit specific binding to trypsin as opposed to the generic inhibition of all proteases, and have the ability to penetrate pancreatic tissue.

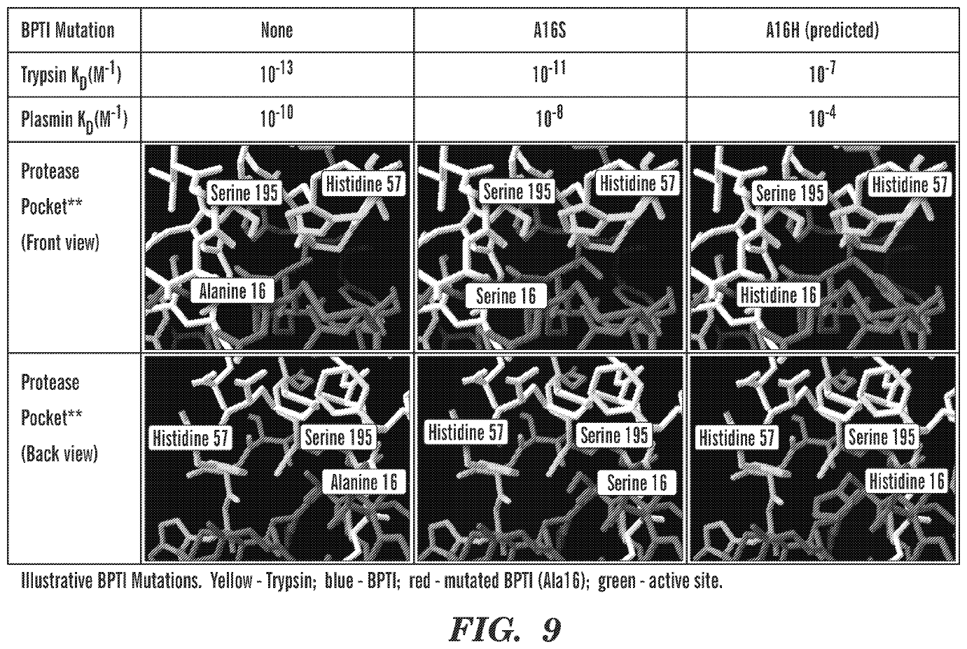

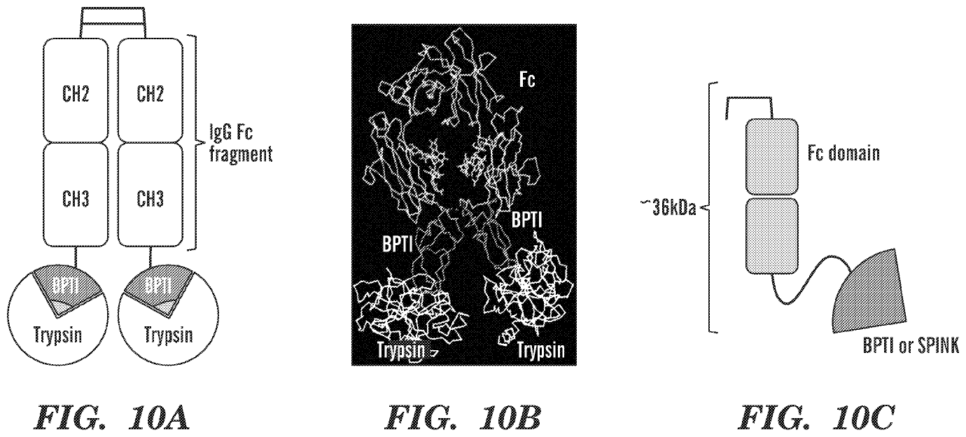

It has been shown that mutations of BPTI that hinder binding to the catalytic pocket are able to decrease binding affinities by a shared order of magnitude across the entire class of serine proteases. Wild type BPTI binds strongly to all serine proteases, with complex half-lives on the scale of months in the case of plasmin to centuries in the case of trypsin. The primary difference is that among these strongly bound complexes, the affinity between BPTI and trypsin is several orders of magnitude more potent than with any other protease. As such, by weakening binding affinity across all proteases, it is possible to design a BPTI mutant that binds trypsin in a way that is both relatively less strong than the wildtype affinity but still able to inhibit trypsin in an objectively potent fashion (FIG. 9). Meanwhile, because all other wildtype-protease binding is less strong to begin with, the same relative drop in affinity can render off-target protease binding into an objectively weak range. Such a mutant can be expected to have improved specificity to trypsin in vivo by avoiding issues related to off-target drug sequestration.

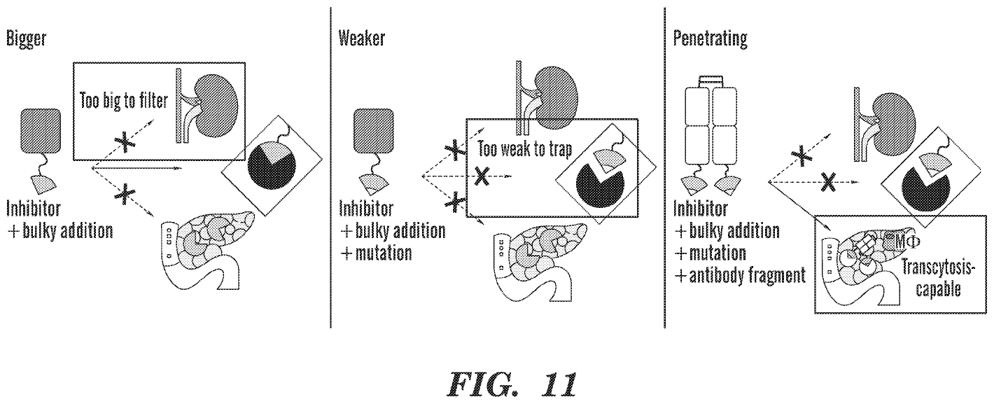

In this study, the inventors designed several novel trypsin inhibitors to address the therapeutic shortcomings of BPTI. Several Fc fusion proteins were designed with the following goals: improved likelihood of tissue penetration, decreased probability of renal clearance, and targeted inhibition of trypsin. In vitro testing indicates that these candidate molecules enjoy robust expression, stability and reflect the intended size and specificity properties. The Fc fusion proteins of the invention are too large to freely filter into the kidney, inhibitory against trypsin, with a stronger affinity for the target than for plasmin, and able to bind FcRn at both physiologic and endosomal pH (FIG. 11).

In addition to modifying the in vivo half-life and phagocyte binding affinity, Fc fusion proteins of the invention can have altered affinity for Protein A-a derivative of the pathogen Staphylococcus Aureus that is frequently used in kits for IgG and Fc protein purification. This change can affect final protein yields.

Fc fusion proteins of the invention can inhibit trypsin, bind reversibly with FcRn at physiological pH, have improved affinity for FcRN at early endosomal pH, avoid kidney filtration and/or support phagocytic recycling-easily released at physiologic pH but stably bound at endosomal pH. In addition, exemplary Fc fusion proteins of the invention, either via steric hindrance or intentional mutation binding to trypsin, can have an affinity range that demonstrates stable complexes with trypsin but have sufficiently weak affinity to plasma proteases to avoid off-target sequestration.

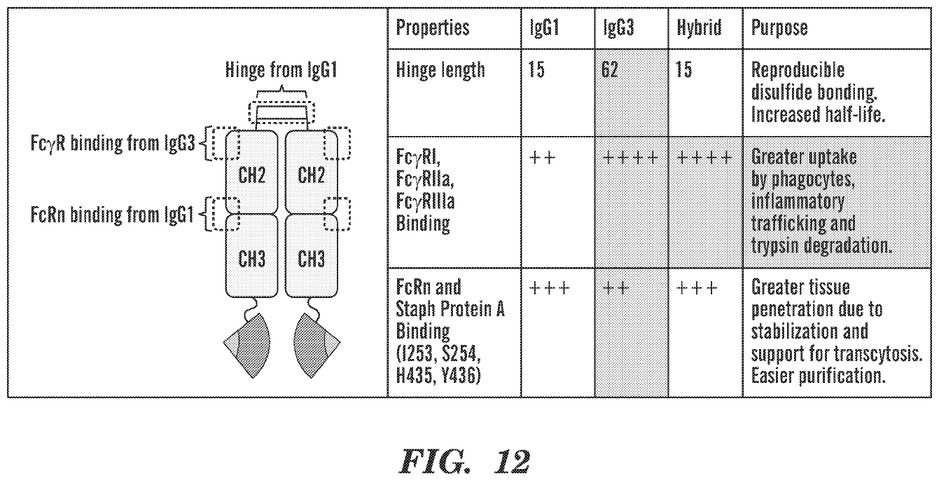

The class of IgG antibodies includes several subclasses of molecules. Specifically, IgG1 and IgG3 express a mixture of properties that them attractive as a potential source for the Fc fragment of a fusion therapeutic. In addition to creating fusions from wildtype IgG1 and IgG3 Fc, a hybrid Fc can be used to capitalize on ideal properties from each subclass (FIG. 12).

These specific illustrations of molecules of the invention are provided to enable users to more fully understand the invention, but are not intended to be limiting. The following text illustrates more fully the breadth of the invention and provides additional scientific information relevant to making and using the invention.

As described herein, the Fc fusion protein comprises linking a serine protease inhibitor Kazal-type 1 (SPINK1) domain or a bovine pancreatic trypsin inhibitor (BPTI) domain to the N—or C—terminus of an Fc domain of an immunoglobulin. The Fc fusion protein formed has an increased in vivo half-life compared to the corresponding SPINK1 domain or BPTI domain which has not been linked to a Fc domain.

In some embodiments, the Fc fusion protein comprises a SPINK1 domain. The SPINK1 domain is a protein or polypeptide that mimics the activity of SPINK1. The Kazal-type serine protease inhibitor family is one of the numerous families of serine protease inhibitors. Many proteins from different species have been described. Serine protease inhibitor Kazal-type 1 is a trypsin inhibitor, which is secreted from pancreatic acinar cells into pancreatic juice. SPINK1 is also known as pancreatic secretory trypsin inhibitor (PSTI) or tumor-associated trypsin inhibitor (TATI) in the art.

SPINK1 is thought to function in the prevention of trypsin-catalyzed premature activation of zymogens within the pancreas and the pancreatic duct. Mutations in this gene are associated with hereditary pancreatitis and tropical calcific pancreatitis. The terms “serine protease inhibitor Kazal-type 1” and “SPINK1,” as used herein, refers to any native SPINK1 from any vertebrate source, including mammals such as primates (e.g., humans) and rodents (e.g., human, mice and rats), unless otherwise indicated. The term encompasses “full-length,” unprocessed SPINK1 as well as any form of SPINK1 that result from processing in the cell. The term also encompasses naturally occurring variants of SPINK1, e.g., splice variants or allelic variants.

It is understood that the SPINK1 domain can be altered such that they vary sequences from the naturally occurring or native sequences from which they were derived, while retaining the desired activity of the native sequence. Preferable the SPINK1 domain is native SPINK1, analogs, and variants thereof. Variants of SPINK1 include replacing or modifying one or more amino acids of native SPINK1 that are not a required structural feature or provide functional activity, including conservative substitutions. Variants of SPINK1 include removing or inserting one or more amino acids in native SPINK1 that are not a required structural feature or provide functional activity. Variants of SPINK1 include replacing or modifying one or more amino acids of native SPINK1 to modify one or more properties or activities. Variants of SPINK1 include removing or inserting one or more amino acids in native SPINK1 to modify one or more SPINK1 properties or activities. Variants of SPINK1 include removing or altering glycosylation sites in native SPINK1. Variants of SPINK1 can be introduced by standard techniques, such as site-directed mutagenesis and PCR-mediated mutagenesis. In some embodiment, the SPINK1 domain is a native SPINK1 domain. In other words, the SPINK1 domain is not an analog or variant of the native SPINK1. For example, the SPINK1 domain comprises a wild-type SPINK1 amino acid sequence.

In some embodiments, the SPINK domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to an amino acid sequence selected from the group consisting of:

(SEQ ID NO: 14)
DSLGREAKCYNELNGCTKIYDPVCGTDGNTYPNECVLCFENRKRQTSILIQKS
GPC;
(SEQ ID NO: 15)
DSLGREAKCYNELNGCTKIYDPVCGTDGNTYPNECVLCFENRKRQTSILIQKS
GPCGPEQKLISEEDLNSALVPRGSRLVDHHHHHH;
(SEQ ID NO: 16)
MKVTGIFLLSALALLSLSGNTGADSLGREAKCYNELNGCTKIYDPVCGGNTY
PNECVLCFENRKRQTSILIQKSGPC;
(SEQ ID NO: 17)
MKVTGIFLLSALALLSLSGNTGADSLGREAKCYNELNGCTKIYNPVCGGNTY
PNECVLCFENRKRQTSILIQKSGPC;
(SEQ ID NO: 18)
MKVTGIFLLSALALLSLSGNTGADSLGREAKCYNELNGCTKIYDPVCGGDTY
PNECVLCFENRKRQTSILIQKSGPC;
(SEQ ID NO: 19)
MKVTGIFLLSALALLSLSGNTGADSLGREAKCYNELNGCTKIYDPVCGGNTY
PNECVLCFEGRKRQTSILIQKSGPC;
(SEQ ID NO: 20)
MKVTGIFLLSAFALLSLSGNTGADSLGREAKCYNELNGCTKIYDPVCGGNTY
PNECVLCFENRKRQTSILIQKSGPC;
(SEQ ID NO: 21)
MKVTGIFLLSALAPLSLSGNTGADSLGREAKCYSELNGCTKIYDPVCGGNTYP
NECVLCFENRKRQTSILIQKSGPC;
and
(SEQ ID NO: 22)
MKVTGIFLLSALALLSLSGNTGADSLGREAKCYNELNGCTKIYDPVCGGNTY
SNECVLCFENRKHQTSILIQKSGPC.

In some embodiments, the SPINK1 domain comprises an amino acid sequence having at least 90%, 91%, 92%, 93%, 94%, 95%, 96%, 97%, 98% or 99% identity to an amino acid sequence selected from the group consisting of SEQ ID NO: 14-22. In some embodiments, the SPINK1 domain comprises an amino acid sequence having at least 95%, 96%, 97%, 98% or 99% identity to an amino acid sequence selected from the group consisting of SEQ ID NO: 14-22. In some embodiments, the SPINK1 domain comprises an amino acid sequence having at least 97%, 98% or 99% identity to an amino acid sequence selected from the group consisting of SEQ ID NO: 14-22. In some embodiments, the SPINK1 domain comprises an amino acid sequence having 100% identity to an amino acid sequence selected from the group consisting of SEQ ID NO: 14-22.

In some preferred embodiments, the SPINK1 domain comprises the amino acid sequence of SEQ ID NO: 14 or 15.

In some embodiments, the SPINK domain comprises a murine SPINK1 domain. In some embodiments, the murine SPINK1 domain comprises SEQ ID NO: 186 or an amino acid sequence having at least 80%, 81%, 82%, 83%, 84%, 85%, 86%, 87%, 88%, 89%, 90%, 91%, 92%, 93%. 94%. 95%. 96%, 97%. 98% or 99% identity to SEQ ID NO: 186.

AKVTGKEASCHDAVAGCPRIYDPVCGTDGITYANECVLCFENRKRIEPVLI RKGGPC (SEQ ID NO: 186).

In some embodiments, the Fc fusion protein comprises a bovine pancreatic trypsin inhibitor domain. Bovine pancreatic trypsin inhibitor, also referred to as aprotinin, is a protease inhibitor which affects known serine proteases such as trypsin, chymotrypsin, plasmin and kallikrein. The terms “bovine pancreatic trypsin inhibitor” and “BPTI,” as used herein, refers to any native BPTI. The term encompasses “full-length,” unprocessed BPTI as well as any form of BPTI that result from processing in the cell. The term also encompasses naturally occurring variants of BPTI e.g., splice variants or allelic variants.

It is understood that the BPTI domain can be altered such that they vary sequences from the naturally occurring or native sequences from which they were derived, while retaining the desired activity of the native sequence. Preferable the BPTI domain is native BPTI, analogs, and variants thereof. Variants of BPTI include replacing or modifying one or more amino acids of native BPTI that are not a required structural feature or provide functional activity, including conservative substitutions. Variants of BPTI include removing or inserting one or more amino acids in native BPTI that are not a required structural feature or provide functional activity. Variants of BPTI include replacing or modifying one or more amino acids of native BPTI to modify one or more properties or activities. Variants of BPTI include removing or inserting one or more amino acids in native BPTI to modify one or more BPTI properties or activities. Variants of BPTI include removing or altering glycosylation sites in native BPTI. Variants of BPTI can be introduced by standard techniques, such as site-directed mutagenesis and PCR-mediated mutagenesis. In some embodiment, the BPTI domain is a native BPTI domain. In other words, the BPTI domain is not an analog or variant of the native SPINK1. For example, the BPTI domain comprises a wild-type BPTI amino acid sequence

In some embodiments, the BPTI domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to the amino acid sequence:

(SEQ ID NO: 3)
RPDFCLEPPYTGPCKARIIRYFYNAKAGLCQTFVYGGCRAKRNNFKSAE
DCMRTCGGA

In some embodiments, the BPTI domain comprises an amino acid sequence having at least 90%, 91%, 92%, 93%, 94%, 95%, 96%, 97%, 98% or 99% identity to SEQ ID NO: 3. In some embodiments, the BPTI domain comprises an amino acid sequence having 100% identity to SEQ ID NO: 3. In some embodiments, the BPTI domain comprises an amino acid sequence having at least 95%, 96%, 97%, 98% or 99% identity to SEQ ID NO: 3. In some embodiments, the BPTI domain comprises an amino acid sequence having at least 97%, 98% or 99% identity to SEQ ID NO: 3. In some embodiments, the BPTI domain comprises an amino acid sequence having 100% identity to SEQ ID NO: 3.

In some embodiments, the BPTI domain comprises a substitution at position 16 of SEQ ID NO: 3. For example, the BPTI domain comprises an A to H or an A to S substitution at position 16 of SEQ ID NO: 3. Accordingly, in some embodiments, the BPTI domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to the amino acid sequence:

(SEQ ID NO: 23)
RPDFCLEPPYTGPCKHRIIRYFYNAKAGLCQTFVYGGCRAKRNNFKSAE
DCMRTCGGA

In some embodiments, the BPTI domain comprises an amino acid sequence having at least 90%, 91%, 92%, 93%, 94%, 95%, 96%, 97%, 98% or 99% identity to SEQ ID NO: 23. In some embodiments, the BPTI domain comprises an amino acid sequence having 100% identity to SEQ ID NO: 23. In some embodiments, the BPTI domain comprises an amino acid sequence having at least 95%, 96%, 97%, 98% or 99% identity to SEQ ID NO: 23. In some embodiments, the BPTI domain comprises an amino acid sequence having at least 97%, 98% or 99% identity to SEQ ID NO: 23. In some embodiments, the BPTI domain comprises an amino acid sequence having 100% identity to SEQ ID NO: 23.

In some preferred embodiments, the BPTI domain comprises an amino acid sequence of SEQ ID NO: 3 or 23.

As used herein, an “Fc region” or “Fc element” is a protein sequence that comprises at least a CH3 domain of an IgG antibody element, and more preferably a CH2 and CH3 domain, and in some cases a hinge region as well. The following are typical Fc regions that are useful in constructing fusion proteins and that consist of a few amino acids from the CH1 domain, a hinge region, a CH2 domain, and a CH3 domain. Fc regions are related to each other through sequence similarity, and are defined herein as sequences that can be aligned using BLAST (https://blast.ncbi.nlm.nih.gov/Blast.cgi) with one of the sequences below to give an “Expect” score of at most 1e-100 (with lower scores indicating greater similarity).

Generally, the “Fc domain” is the polypeptide comprising the constant region of an antibody excluding the first constant region immunoglobulin domain and in some cases, part or all of the hinge. Thus, an Fc domain refers to the non-antigen binding portion of an antibody, whether in monomeric or multimeric form. The Fe domain can be from any vertebrate source, including mammals such as primates (e.g., humans), non-human primates (e.g. Chimpanzee, Macaque) and rodents (e.g. a mouse, rat, rabbit, guinea pig). Preferably, the antibody from which the Fc domain arises is of human origin.

An Fc domain includes the hinge region of the heavy chain. By “hinge” or “hinge region” or “antibody hinge region” or “immunoglobulin hinge region” herein is meant the flexible polypeptide comprising the amino acids between the first and second constant domains of an antibody, just upstream of the papain cleavage. Accordingly, for IgG, an Fc domain comprises immunoglobulin domains CH2 and CH3 and the hinge region between CH1 and CH2. Although the boundaries of the Fc region may vary, the human IgG heavy chain Fc region is usually defined to include residues C226 or P230 to its carboxyl-terminus, wherein the numbering is according to the EU index and in Kabat. In some embodiments, as is more fully described below, amino acid modifications are made to the Fc domain, for example to alter binding to one or more FcγR receptors or to the FcRn receptor.

Accordingly, in certain embodiments, the term Fc domain includes the hinge region which may be truncated, modified by replacement, deletion and/or insertion and further the modified or unmodified hinge region may be the site of attachment of a linker domain.

An “analog of an Fc domain” refers to a molecule or sequence that is modified from the native Fc but still comprises a binding site for the salvage receptor. The term analog of an Fc domain includes a molecule or sequence that is humanized from a non-human native Fc. The term analog of an Fc domain also includes a molecule or sequence that lacks, or has modifications of, one or more native Fc residues that affect or are involved in disulfide formation, incompatibility with a host cell, N-terminal heterogeneity upon expression, stability, glycosylation, interaction with a complement, binding to an Fc salvage receptor and/or interaction with an Fcγ receptor.

The terms “fragments of the Fc domain” or “fragment of the Fc domain” refers to a native Fc from which one or more sites have been removed where the removed site(s) does not constitute structural features or functional activity that is required by the fusion proteins of the present invention. Fragments of the Fc domain include deleting residues from the native Fc or truncating the native Fc and may include substitutions of the remaining residues. The inserted or altered residues (e.g., the substituted residues) may be natural amino acids or altered amino acids, peptidomimetics, unnatural amino acids, or D-amino acids.

Generally, the Fe domain includes a sequence at least 80%, 81%, 82%, 83%, 84%, 85%, 86%, 87%, 88%, 89%, 90%, 91%, 92%, 93%, 94%, 95%, 96%, 97%, 98%, or 99% identical to IgG1, IgG2, IgG3, IgG4, IgD, IgA, IgE, or IgM, in particular human IgG1 or IgG3.

The term Fc domain encompasses native Fc and analogs of Fc and includes monomeric and multimeric forms whether prepared by a digest of an intact antibody or produced by other means.

In some embodiments, the Fc domain comprises at least a hinge domain (upper, middle, and/or lower hinge region), a CH2 domain (or a variant or fragment thereof), and a CH3 domain (or a variant or fragment thereof). In some embodiments, the Fc domain consists of a hinge domain (upper, middle, and/or lower hinge region), a CH2 domain (or a variant or fragment thereof), and a CH3 domain (or a variant or fragment thereof). In some embodiments, the Fc domain consists of a hinge domain (upper, middle, and/or lower hinge region), a CH2 domain (or a variant or fragment thereof), a CH3 domain (or a variant or fragment thereof), and a CH4 domain (or a variant or fragment thereof). In some embodiments, the Fc domain consists of a hinge domain (upper, middle, and/or lower hinge region) and a CH2 domain. In some embodiments, the Fc domain consists of a hinge domain (upper, middle, and/or lower hinge region) and a CH3 domain (or a variant or fragment thereof). In some embodiments, the Fc domain consists of a CH2 domain (or a variant or fragment thereof), and a CH3 domain (or a variant or fragment thereof). In some embodiments, the Fc domain consists of a complete CH2 domain and a complete CH3 domain. In some embodiments, the Fc domain consists of a complete CH2 domain and a complete CH3 domain. In some embodiments, the Fc domain comprises at least the portion of an Fc molecule known in the art to be required for FcRn binding. In some embodiments, the Fc domain comprises at least the portion of an Fc molecule known in the art to be required for FcγR binding. In some embodiments, the Fc domain comprises at least the portion of an Fc molecule known in the art to be required for Protein A binding. In some embodiments, the Fc domain comprises at least the portion of an Fc molecule known in the art to be required for Protein G binding.

As described herein, an Fc domain generally refers to a polypeptide comprising all or part of the Fc domain of an immunoglobulin heavy-chain. As discussed above, this includes, but is not limited to polypeptides comprising the entire hinge region, CH1, CH2, and/or CH3 domains as well as fragments of such peptides comprising, for example, the hinge, CH2 and CH3 domains. The Fc domain may be derived from any immunoglobulin of any species and/or subtype, including but not limited to, a human IgG1, IgG2, IgG3, IgG4, IgD, IgA, IgE, or IgM antibody. The Fc domain includes the last two constant region immunoglobulin domains of IgA, IgD, and IgG, the last three constant region immunoglobulin domains of IgE and IgM, and the flexible hinge N-terminal to these domains. For IgA and IgM, Fc may include the J chain.

The Fc domain as used herein encompasses native Fc and Fc variant molecules. As with the Fc variants and native Fc proteins, the term Fc domain includes molecules in monomeric and multimeric form, whether digested from an antibody or produced by other means.

It is noted that any Fc domain may be modified such that it varies in amino acid sequence from the native Fc domain of a naturally occurring immunoglobulin molecule. In some embodiments, the Fc domain retains an effector function, for example, FcRN and/or FcβR binding.

It is noted that the Fc domain can be derived from different immunoglobulin molecules. For example, the Fc domain can comprise a CH2 and/or CH3 domain derived from one type or subtype of immunoglobulin and a hinge region from a different type or subtype of immunoglobulin, such as a CH2 and/or CH3 domain derived from IgG1 and a hinge region derived from IgG3 or vice-versa.

In some embodiments of the various aspects described herein, the Fc domain comprises a hinge region, an IgG CH2 domain and an IgG CH3 domain.

In some embodiments, the hinge region of the Fc domain has a length of about 10 to about 20 amino acids.

In some embodiments of the various aspects described herein, the Fc domain is of human origin. In some embodiments, the Fc region extends from Cys226, or from Pro230, to the carboxyl-terminus of the heavy chain of a human IgG.

In some embodiments of the various aspects described herein, the Fc domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to an amino acid sequence selected from the group consisting of:

(SEQ ID NO: 24, human IgG1)
EPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFS
CSVMHEALHNHYTQKSLSLSPGK;
(SEQ ID NO: 25, human IgG2)
KTVERKCCVECPPCPAPPVAGPSVFLFPPKPKDTLMISRTPEVTCVVVDVSHE
DPEVQFNWYVDGVEVHNAKTKPREEQFNSTFRVVSVLTVVHQDWLNGKEY
KCKVSNKGLPAPIEKTISKTKGQPREPQVYTLPPSREEMTKNQVSLTCLVKGF
YPSDIAVEWESNGQPENNYKTTPPMLDSDGSFFLYSKLTVDKSRWQQGNVFS
CSVMHEALHNHYTQKSLSLSPGK;
(SEQ ID NO: 26, human IgG3)
ELKTPLGDTTHTCPRCPEPKSCDTPPPCPRCPEPKSCDTPPPCPRCPAPELLGGP
SVFLFPPKPKDTLMISRTPEVTCVVVDVSHEDPEVQFKWYVDGVEVHNAKTK
PWEEQYNSTFRVVSVLTVLHQDWLNGKEYKCKVSNKALPAPIEKTISKTKGQ
PREPQVYTLPPSREEMTKNQVSLTCLVKGFYPSDIAMEWESSGQPENNYKTTP
PVLDSDGSFFLYSKLTVDKSRWQQGNIFSCSVMHEALHNHYTQKSLSLSPGK;
(SEQ ID NO: 27, human IgG4)
KVDKRVESKYGPPCPSCPAPEFLGGPSVFLFPPKPKDTLMISRTPEVTCVVVD
VSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQEG
NVFSCSVMHEALHNHYTQKSLSLSLGK;
(SEQ ID NO: 28, mouse IgA2 heavy chain)
EPRVPITQNPCPPLKECPPCAAPDLLGGPSVFIFPPKIKDVLMISLSPMVTCVVV
DVSEDDPDVQISWFVNNVEVHTAQTQTHREDYNSTLRVVSALPIQHQDWMS
GKEFKCKVNNRALPSPIEKTISKPRGPVRAPQVYVLPPPAEEMTKKEFSLTCMI
TGFLPAEIAVDWTSNGRTEQNYKNTATVLDSDGSYFMYSKLRVQKSTWERG
SLFACSVVHEGLHNHLTTKTISRSLGK

It is often useful to introduce mutations in the Fc region, such as the conversion of the sequence EPKSCDKTHTCP . . . . (SEQ ID NO: 29) to EPKSSDKTHTCP . . . . (SEQ ID NO: 30, Cys220Ser); the conversion of . . . . KTKPREEQYNSTYRVVSVLT . . . . (SEQ ID NO: 31) to . . . . KTKPREEQYDSTYRVVSVLT . . . . (SEQ ID NO: 32, Asn297Asp) or KTKPREEQYQSTYRVVSVLT . . . . (SEQ ID NO: 33, Asn297Gln); and/or . . . . QKSLSLSLGK (SEQ ID NO: 34) to . . . . QKSLSLSLGA (SEQ ID NO: 35, Lys447Ala).

Some exemplary Fc domain amino acid sequences are as follows:

(SEQ ID NO: 36)
EPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFS
CSVMHEALHNHYTQKSLSLSPGA;
(SEQ ID NO: 37)
EPKSSDKTHTCPPCPAPELLGGPSVFLFPPKPKDTLMISRTPEVTCVVVDVSHE
DPEVKFNWYVDGVEVHNAKTKPREEQYDSTYRVVSVLTVLHQDWLNGKEY
KCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFS
CSVMHEALHNHYTQKSLSLSPGA (Fc(N82D); see e.g., SEQ ID
NOs: 8, 86-89, 95-97, 177, 179, 181);
(SEQ ID NO: 38)
CPAPELLGGPSVFLFPPKPKDTLMISRTPEVTCVVVDVSHEDPEVQFKWYVDG
VEVHNAKTKPREEQYNSTFRVVSVLTVLHQDWLNGKEYKCKVSNKALPAPI
EKTISKTKGQPREPQVYTLPPSREEMTKNQVSLTCPVKGFYPSDIAVEWESSG
QPENNYNTTPPMLDSDGSFFLYSKLTVDKSRWQQGNIFSCSVMHEALHNhyT
QKSLSLSPGA;
(SEQ ID NO: 39)
epkscdkthtcppCPAPELLGGPSVFLFPPKPKDTLMISRTPEVTCVVVDVSHEDPEV
QFKWYVDGVEVHNAKTKPREEQYNSTFRVVSVLTVLHQDWLNGKEYKCKV
SNKALPAPIEKTISKTKGQPREPQVYTLPPSREEMTKNQVSLTCPVKGFYPSDI
AVEWESSGQPENNYNTTPPMLDSDGSFFLYSKLTVDKSRWQQGNIFSCSVMH
EALHNhyTQKSLSLSPGa;
and
(SEQ ID NO: 40)
EPeeSDKTHTCPPCPAPELLGGPSVFLFPPKPKDTLMISRTPEVTCVVVDVSHE
DPEVKFNWYVDGVEVHNAKTKPREEQYdSTYRVVSVLTVLHQDWLNGKEY
KCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFS
CSVMHEALHNHYTQKSLSLSPGe.

In some embodiments, the Fe domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a sequence selected from the group consisting of SEQ ID NO: 24-40. In some embodiments, the Fc domain comprises an amino acid sequence having at least 95%, 96%, 97%, 98% or 99% identity to a sequence selected from the group consisting of SEQ ID NO: 24-40. In some embodiments, the Fe domain comprises an amino acid sequence having at least 97%, 98% or 99% identity to a sequence selected from the group consisting of SEQ ID NO: 24-40.

In some embodiments, the Fc domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a sequence selected from the group consisting of SEQ ID NO: 24-40 and 187-204. In some embodiments, the Fc domain comprises an amino acid sequence having at least 95%, 96%, 97%, 98% or 99% identity to a sequence selected from the group consisting of SEQ ID NO: 24-40 and 187-204. In some embodiments, the Fc domain comprises an amino acid sequence having at least 97%, 98% or 99% identity to a sequence selected from the group consisting of SEQ ID NO: 24-40 and 187-204.

In some preferred embodiments, the Fc domain comprises an amino acid sequence selected from the group consisting of SEQ ID NO: 36, SEQ ID NO: 37, SEQ ID NO: 38, SEQ ID NO: 39, and SEQ ID NO: 40.

The hinge-region between the CH2 and CH3 domains of IgG is able to bind several proteins beyond protein A, such as the neonatal Fc receptor (FcRn). Without wishing to be bound by a theory, FcRn functions to salvage IgG from the lysosomal degradation pathway, resulting in reduced clearance and increased half-life. The FcRn is a heterodimeric protein consisting of two polypeptides: a 50 kDa class I major histocompatibility complex-like protein (a-FcRn) and a 15 kDa B2-microglobulin (β2m). FcRn binds with high affinity to the CH2-CH3 portion of the Fc-region of IgG. The interaction between IgG and FcRn is strictly pH dependent and occurs in a 1:2 stoichiometry, with one IgG binding to two FcRn molecules via its two heavy chains (Huber, A. H., et al., J. Mol. Biol. 230 (1993) 1077-1083). FcRn binding occurs in the endosome at acidic pH (pH<6.5) and IgG is released at the neutral cell surface (pH of about 7.4). The pH-sensitive nature of the interaction facilitates the FcRn mediated protection of IgGs pinocytosed into cells from intracellular degradation by binding to the receptor within the acidic environment of endosomes. FcRn then facilitates the recycling of IgG to the cell surface and subsequent release into the blood stream upon exposure of the FcRn IgG complex to the neutral pH environment outside the cell.

Accordingly, in some embodiments, the Fc fusion protein includes a wild-type Fc domain that can allow the fusion protein to undergo endocytosis after binding FcRn. For example, the Fc fusion protein described herein comprises the FcRn binding portion of the Fc of an immunoglobulin. The term “FcRn binding portion of an Fc region” denotes the part of an antibody heavy chain polypeptide that extends approximately from EU position 243 to EU position 261, and approximately from EU position 275 to EU position 293, and approximately from EU position 302 to EU position 319, and approximately from EU position 336 to EU position 348, and approximately from EU position 367 to EU position 393, and EU position 408, and approximately from EU position 424 to EU position 440.

In some embodiments of the various aspects described herein, one or more of the following amino acid residues, in the Fc domain, according to the EU numbering of Kabat are altered F243, P244, P245, K246, P247, K248, D249, T250, L251, M252, 1253, S254, R255, T256, P257, E258, V259, 1260, C261, F275, N276, W277, Y278, V279, D280, V282, E283, V284, H285, N286, A287, K288, T289, K290, P291, R292, E293, V302, V303, S304, V305, L306, T307, V308, L309, H310, 0311, D312, W313, L314, N315, G316, K317, E318, Y319, 1336, S337, K338, A339, K340, G341, Q342, P343, R344, E345, P346, Q347, V348, C367, V369, F372, Y373, P374, S375, D376, 1377, A378, V379, E380, W381, E382, S383, N384, G385, Q386, P387, E388, N389, Y391, T393, S408, S424, C425, S426, V427, M428, H429, E430, A431, L432, H433, N434, H435, Y436, T437, Q438, K439, and S440 (EU numbering).

In some embodiments, the Fc domain comprises a sequence where the following amino acid residues according to the EU numbering of Kabat are present 1253, S254, H435 and Y436 (EU numbering).

In some embodiments, the Fc domain comprises one or more of the following amino acid substitutions M252Y, S254T and T256E (EU numbering of Kabat).

In some embodiments, the Fc domain comprises at least one modification selected from the group consisting of: LALA; PG; VE; YTE; LS; DHS; and negative charge modifications. In some embodiments, a human Fc domain (e.g., IgG1; e.g., SEQ ID NO: 36) comprises at least one modification selected from the group consisting of: L19A, L20A, P114G, V49E, M37Y, S39T, T41E, M213L, N219S, L94D, Q96H, N219S, K3E, S4E, and A232E. In some embodiments, a murine Fc domain (e.g., IgG2b; e.g., SEQ ID NO: 188) comprises at least one modification selected from the group consisting of: L26A, E27A, P121G, V56E, M44Y, S46T, T48E, R220L, N226S, Q101D, Q103H, N226S, K3E, S5E, and A232E.

In some embodiments, a “LALA” modification comprises converting the sequence “ . . . . TCPPCPAPELLGGPSVFLFPPK”(SEQ ID NO: 182) to “ . . . . TCPPCPAPEAAGGPSVFLFPPK . . . . ” (SEQ ID NO: 183; e.g., L19A, L20A) near the N-terminus of the Fc region. In a murine IgG2b Fc, a “LALA” modification comprises converting the sequence“ . . . . ECHKCPAPNLEGGPSVFIFPPN . . . . ” (SEQ ID NO: 211) to “ . . . . ECHKCPAPNAAGGPSVFIFPPN . . . . ” (SEQ ID NO: 212; e.g., L26A, E27A) near the N-terminus of the Fc region.

In some embodiments, a “PG” modification comprises converting the sequence” . . . . CKVSNKALPAPIEKTIS . . . . ” (SEQ ID NO: 184) to “ . . . . CKVSNKALGAPIEKTIS . . . . ” (SEQ ID NO: 185; e.g., P114G) in the human Fc region. In a murine IgG2b Fc, a “PG” modification comprises converting the sequence” . . . . CKVNNKDLPSPIERTIS . . . . ” (SEQ ID NO: 213) to “ . . . . CKVNNKDLGSPIERTIS . . . . ” (SEQ ID NO: 214; e.g., P121G)

In some embodiments, for example, a “VE” modification comprises converting the sequence” . . . . VTCVVVDVS . . . . ” (SEQ ID NO: 215) to “ . . . . VTCVVEDVS . . . . ” (SEQ ID NO: 216; e.g., V49E in a human IgG1; V56E in a murine IgG2b Fc) near the N-terminus of the Fc region.

In some embodiments, a “YTE” modification comprises converting the sequence” . . . . DTLMISRTPE . . . . ” (SEQ ID NO: 217) to “ . . . . DTLYITREPE . . . . ” (SEQ ID NO: 218; e.g., M37Y, S39T, T41E) near the N-terminus of the Fc region. In a murine IgG2b Fc, a “YTE” modification comprises converting the sequence” . . . . DVLMISLTPK . . . . ” (SEQ ID NO: 219) to “ . . . . DVLYITLEPK . . . . ” (SEQ ID NO: 220; e.g., M44Y, S46T, T48E) near the N-terminus of the Fc region.

In some embodiments, a “LS” modification comprises converting the sequence” . . . . FSCSVMHEALHNHYT . . . . ” (SEQ ID NO: 221) to “ . . . . FSCSVLHEALHSHYT . . . . ” (SEQ ID NO: 222; e.g., M213L, N219S) near the C-terminus of the Fc region. In a murine IgG2b Fc, a “LS” modification comprises converting the sequence” . . . . FSCNVRHEGLKNYYL . . . . ” (SEQ ID NO: 223) to “ . . . . FSCNVLHEGLKSYYL . . . . ” (SEQ ID NO: 224; e.g., R220L, N226S) near the C-terminus of the Fc region.

In some embodiments, a “DHS”modification comprises converting the sequence” . . . . LTVLHQDWLN . . . . ” (SEQ ID NO: 225) to “ . . . . LTVDHHDWLN . . . . ” (SEQ ID NO: 226; e.g., L94D, Q96H) near the middle of the Fc region and/or converting the sequence “ . . . . EALHNHYTQK . . . . ” (SEQ ID NO: 227) to “ . . . . EALHSHYTQK . . . . ” (SEQ ID NO: 228; e.g., N219S) near the C-terminus of the Fc region. In a murine IgG2b Fc, a “DHS” modification comprises converting the sequence” . . . . LPIQHQDWMS . . . . ” (SEQ ID NO: 229) to “ . . . . LPIDHHDWMS . . . . ” (SEQ ID NO: 230; e.g., Q101D, Q103H) near the middle of the Fc region and/or converting the sequence “ . . . . EGLKNYYLKK . . . . ” (SEQ ID NO: 231) to “ . . . . EGLKSYYLKK . . . . ” (SEQ ID NO: 232; e.g., N226S) near the C-terminus of the Fc region. An “EDHS” modification comprises a “VE” modification combined with a “DHS” modification.

In some embodiments, a “negative charge” modification comprises modifying at least one amino acid to a negatively charged amino acid, such as aspartic acid (Asp, D) or glutamic acid (Glu, E) (acidic side chains). For example, a “negative charge” modification comprises converting the sequence “ . . . . EPKSSDKTHT . . . . ” (SEQ ID NO: 233) to “ . . . . EPEESDKTHT . . . . ” (SEQ ID NO: 234; e.g., K3E, S4E) at the N-terminus of the Fc region, and/or converting the sequence “ . . . . QKSLSLSPGA . . . . ” (SEQ ID NO: 235) to “ . . . . QKSLSLSPGE . . . . ” (SEQ ID NO: 236; e.g., A232E) at the C-terminus of the Fc region. In a murine IgG2b Fc, a “negative charge” modification comprises converting the sequence “ . . . . EPKSSPPCKE . . . . ” (SEQ ID NO: 237) to “ . . . . EPESEPPCKE . . . . ” (SEQ ID NO: 238; e.g., K3E, S5E) at the N-terminus of the Fc region, and/or converting the sequence “ . . . . TKTISRSLGA . . . . ” (SEQ ID NO: 239) to “ . . . . TKTISRSLGE . . . . ” (SEQ ID NO: 240; e.g., A232E) at the C-terminus of the Fc region.

Some exemplary Fc domain amino acid sequences are as follows:

EPEESDKTHTCPPCPAPEaaGGPSVFLFPPKPKDTLyItRePEVTCVVVDVSHEDP
EVKFNWYVDGVEVHNAKTKPREEQYDSTYRVVSVLTVLHQDWLNGKEYKCKVSN
KALgAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQ
KSLSLSPGE (SEQ ID NO: 187, Fc(N82D; YTE;LALA-PG)-neg-charge;
see e.g., SEQ ID NO: 85);
EPEESDKTHTCPPCPAPELLGGPSVFLFPPKPKDTLMISRTPEVTCVVVDVSHE
DPEVKFNWYVDGVEVHNAKTKPREEQYDSTYRVVSVLTVLHQDWLNGKEYKCKV
SNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYT
QKSLSLSPGE (SEQ ID NO: 40, Fc(N82D) neg. charge; see e.g.,
SEQ ID NO: 90);
EPRGPTIKPCPPCKCPAPNLLGGPSVFIFPPKIKDVLMISLSPIVTCVVVDVSED
DPDVQISWFVNNVEVHTAQTQTHREDYDSTLRVVSALPIQHQDWMSGKEFKCKVN
NKDLPAPIERTISKPKGSVRAPQVYVLPPPEEEMTKKQVTLTCMVTDFMPEDIYVEW
TNNGKTELNYKNTEPVLDSDGSYFMYSKLRVEKKNWVERNSYSCSVVHEGLHNHH
TTKSFSRTPGK (SEQ ID NO: 188, murine Fc; see e.g., SEQ ID
NOs: 91-94);
EPSGPISTINPCPPCKECHKCPAPNLEGGPSVFIFPPNIKDVLMISLTPKVTCVVV
DVSEDDPDVRISWFVNNVEVHTAQTQTHREDYDSTIRVVSALPIQHQDWMSGKEFK
CKVNNKDLPSPIERTISKIKGLVRAPQVYILPPPAEQLSRKDVSLTCLVVGFNPGDISV
EWTSNGHTEENYKDTAPVLDSDGSYFIYSKLDIKTSKWEKTDSFSCNVRHEGLKNY
YLKKTISRSPGA (SEQ ID NO: 189, IgG2b Fc(N89D;K239A);
see e.g., SEQ ID NO: 98);
EPSGPISTINPCPPCKECHKCPAPNLEGGPSVFIFPPNIKDVLYITLEPKVTCVVV
DVSEDDPDVRISWFVNNVEVHTAQTQTHREDYDSTIRVVSALPIQHQDWMSGKEFK
CKVNNKDLPSPIERTISKIKGLVRAPQVYILPPPAEQLSRKDVSLTCL VVGFNPGDISV
EWTSNGHTEENYKDTAPVLDSDGSYFIYSKLDIKTSKWEKTDSFSCNVRHEGLKNY
YLKKTISRSPGA (SEQ ID NO: 190, murine IgG2b Fc(N89D;K239A; YTE);
see e.g., SEQ ID NO: 99);
EPSGPISTINPCPPCKECHKCPAPNAAGGPSVFIFPPNIKDVLYITLEPKVTCVV
VDVSEDDPDVRISWFVNNVEVHTAQTQTHREDYDSTIRVVSALPIQHQDWMSGKEF
KCKVNNKDLGSPIERTISKIKGLVRAPQVYILPPPAEQLSRKDVSLTCLVVGFNPGDIS
VEWTSNGHTEENYKDTAPVLDSDGSYFIYSKLDIKTSKWEKTDSFSCNVRHEGLKN
YYLKKTISRSPGA (SEQ ID NO: 191, murine IgG2b Fc(N89D;K239A;
YTE;LALA-PG); see e.g., SEQ ID NO: 100);
EPSGPISTINPCPPCKECHKCPAPNLEGGPSVFIFPPNIKDVLYITLEPKVTCVVE
DVSEDDPDVRISWFVNNVEVHTAQTQTHREDYDSTIRVVSALPIQHQDWMSGKEFK
CKVNNKDLPSPIERTISKIKGLVRAPQVYILPPPAEQLSRKDVSLTCLVVGFNPGDISV
EWTSNGHTEENYKDTAPVLDSDGSYFIYSKLDIKTSKWEKTDSFSCNVRHEGLKNY
YLKKTISRSPGA (SEQ ID NO: 192, murine IgG2b Fc(N89D;K239A;
YTE; V264E); see e.g., SEQ ID NO: 101);
EPSGPISTINPCPPCKECHKCPAPNLEGGPSVFIFPPNIKDVLMISLTPKVTCVVV
DVSEDDPDVRISWFVNNVEVHTAQTQTHREDYDSTIRVVSALPIQHQDWMSGKEFK
CKVNNKDLPSPIERTISKIKGLVRAPQVYILPPPAEQLSRKDVSLTCLVVGFNPGDISV
EWTSNGHTEENYKDTAPVLDSDGSYFIYSKLDIKTSKWEKTDSFSCNVLHEGLKSYY
LKKTISRSPGA (SEQ ID NO: 193, murine IgG2b Fc(N89D;K239A;LS);
see e.g., SEQ ID NO: 102);
EPSGPISTINPCPPCKECHKCPAPNAAGGPSVFIFPPNIKDVLMISLTPKVTCVV
VDVSEDDPDVRISWFVNNVEVHTAQTQTHREDYDSTIRVVSALPIQHQDWMSGKEF
KCKVNNKDLGSPIERTISKIKGLVRAPQVYILPPPAEQLSRKDVSLTCLVVGFNPGDIS
VEWTSNGHTEENYKDTAPVLDSDGSYFIYSKLDIKTSKWEKTDSFSCNVLHEGLKSY
YLKKTISRSPGA (SEQ ID NO: 194, murine IgG2b Fc(N89D; K239A;
LS; LALA-PG); see e.g., SEQ ID NO: 103);
EPSGPISTINPCPPCKECHKCPAPNLEGGPSVFIFPPNIKDVLMISLTPKVTCVVE
DVSEDDPDVRISWFVNNVEVHTAQTQTHREDYDSTIRVVSALPIQHQDWMSGKEFK
CKVNNKDLPSPIERTISKIKGLVRAPQVYILPPPAEQLSRKDVSLTCLVVGFNPGDISV
EWTSNGHTEENYKDTAPVLDSDGSYFIYSKLDIKTSKWEKTDSFSCNVLHEGLKSYY
LKKTISRSPGA (SEQ ID NO: 195, murine IgG2b Fc(N89D; K239A;
LS; V264E); see e.g., SEQ ID NO: 104);
EPSGPISTINPCPPCKECHKCPAPNLEGGPSVFIFPPNIKDVLMISLTPKVTCVVV
DVSEDDPDVRISWFVNNVEVHTAQTQTHREDYDSTIRVVSALPIDHHDWMSGKEFK
CKVNNKDLPSPIERTISKIKGLVRAPQVYILPPPAEQLSRKDVSLTCLVVGFNPGDISV
EWTSNGHTEENYKDTAPVLDSDGSYFIYSKLDIKTSKWEKTDSFSCNVRHEGLKSYY
LKKTISRSPGA (SEQ ID NO: 196, murine IgG2b Fc(N89D; K239A;
DHS); see e.g., SEQ ID NO: 105);
EPSGPISTINPCPPCKECHKCPAPNAAGGPSVFIFPPNIKDVLMISLTPKVTCVV
VDVSEDDPDVRISWFVNNVEVHTAQTQTHREDYDSTIRVVSALPIDHHDWMSGKEF
KCKVNNKDLGSPIERTISKIKGLVRAPQVYILPPPAEQLSRKDVSLTCLVVGFNPGDIS
VEWTSNGHTEENYKDTAPVLDSDGSYFIYSKLDIKTSKWEKTDSFSCNVRHEGLKSY
YLKKTISRSPGA (SEQ ID NO: 197, murine IgG2b Fc(N89D; K239A;
DHS; LALA-PG); see e.g., SEQ ID NO: 106);
EPSGPISTINPCPPCKECHKCPAPNLEGGPSVFIFPPNIKDVLMISLTPKVTCVVE
DVSEDDPDVRISWFVNNVEVHTAQTQTHREDYDSTIRVVSALPIDHHDWMSGKEFK
CKVNNKDLPSPIERTISKIKGLVRAPQVYILPPPAEQLSRKDVSLTCLVVGFNPGDISV
EWTSNGHTEENYKDTAPVLDSDGSYFIYSKLDIKTSKWEKTDSFSCNVRHEGLKSYY
LKKTISRSPGA (SEQ ID NO: 198, murine IgG2b Fc(N89D; K239A;
EDHS); see e.g., SEQ ID NO: 107);
EPKSSPPCKECSIFPAPDAAGGPSVFIFPPKIKDVLYITLEPEVTCVVVDVSEDD
PDVQISWFVNNVEVHTAQTQTHREDYDSTLRVVSALPIQHQDWMSGKEFKCKVNN
RALPSPIEKTISKPRGPVRAPQVYVLPPPAEEMTKKEFSLTCMITDFLPAEIAVDWTSN
GHKELNYKNTAPVLDTDGSYFMYSKLRVQKSTWEKGSLFACSVVHEGLHNHHTTK
TISRSLGA (SEQ ID NO: 199, murine IgG2b Fc(YTE & LALA);
see e.g., SEQ ID NO: 108);
EPESEPPCKECSIFPAPDAAGGPSVFIFPPKIKDVLYITLEPEVTCVVVDVSEDD
PDVQISWFVNNVEVHTAQTQTHREDYDSTLRVVSALPIQHQDWMSGKEFKCKVNN
RALPSPIEKTISKPRGPVRAPQVYVLPPPAEEMTKKEFSLTCMITDFLPAEIAVDWTSN
GHKELNYKNTAPVLDTDGSYFMYSKLRVQKSTWEKGSLFACSVVHEGLHNHHTTK
TISRSLGE (SEQ ID NO: 200, murine IgG2b Fc(YTE & LALA; Neg.
Charge); see e.g., SEQ ID NO: 109 or 110);
EPKSSDKTHTCPPCPAPEAAGGPSVFLFPPKPKDTLYITREPEVTCVVVDVSHE
DPEVKFNWYVDGVEVHNAKTKPREEQYDSTYRVVSVLTVLHQDWLNGKEYKCKV
SNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYT
QKSLSLSPGA (SEQ ID NO: 201, Fc(N82D; YTE; LALA); see e.g.,
SEQ ID NO: 111);
EPKSSDKTHTCPPCPAPEAAGGPSVFLFPPKPKDTLYITREPEVTCVVVDVSHE
DPEVKFNWYVDGVEVHNAKTKPREEQYDSTYRVVSVLTVLHQDWLNGKEYKCKV
SNKALGAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYT
QKSLSLSPGA (SEQ ID NO: 202, Fc(N82D; YTE; LALA-PG); see e.g.,
SEQ ID NO: 112);
EPKSSDKTHTCPPCPAPEAAGGPSVFLFPPKPKDTLMISRTPEVTCVVVDVSHE
DPEVKFNWYVDGVEVHNAKTKPREEQYDSTYRVVSVLTVLHQDWLNGKEYKCKV
SNKALGAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVLHEALHSHYT
QKSLSLSPGA (SEQ ID NO: 203, Fc(N82D; LS; LALA-PG); see e.g.,
SEQ ID NO: 113);
or
EPKSSDKTHTCPPCPAPEAAGGPSVFLFPPKPKDTLMISRTPEVTCVVVDVSHE
DPEVKFNWYVDGVEVHNAKTKPREEQYDSTYRVVSVLTVDHHDWLNGKEYKCKV
SNKALGAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHSHYT
QKSLSLSPGA (SEQ ID NO: 204, Fc(N82D;DHS;LALA-PG); see e.g.,
SEQ ID NO: 114).

In some embodiments, the Fe domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a sequence selected from the group consisting of SEQ ID NO: 187-204. In some embodiments, the Fc domain comprises an amino acid sequence having at least 95%, 96%, 97%, 98% or 99% identity to a sequence selected from the group consisting of SEQ ID NO: 187-204. In some embodiments, the Fc domain comprises an amino acid sequence having at least 97%, 98% or 99% identity to a sequence selected from the group consisting of SEQ ID NO: 187-204.

In embodiments, the Fc domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a sequence selected from the group consisting of SEQ ID NO: 187, 189, and 201-204. In some embodiments, the Fc domain comprises an amino acid sequence having at least 95%, 96%, 97%, 98% or 99% identity to a sequence selected from the group consisting of SEQ ID NO: 187, 189, and 201-204. In some embodiments, the Fc domain comprises an amino acid sequence having at least 97%, 98% or 99% identity to a sequence selected from the group consisting of SEQ ID NO: 187, 189, and 201-204.

In embodiments, the Fe domain is a murine Fe domain comprising an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a sequence selected from the group consisting of SEQ ID NO: 188 and 190-200. In some embodiments, the Fc domain comprises an amino acid sequence having at least 95%, 96%, 97%, 98% or 99% identity to a sequence selected from the group consisting of SEQ ID NO: 188 and 190-200. In some embodiments, the Fc domain comprises an amino acid sequence having at least 97%, 98% or 99% identity to a sequence selected from the group consisting of SEQ ID NO: 188 and 190-200.

In some embodiments of the various aspects described herein, the Fc domain is an asymmetric Fc domain. The term “asymmetric Fc domain” denotes a pair of Fc region polypeptides that have different amino acid residues at corresponding positions according to the Kabat EU index numbering system.

In some embodiments of the various aspects described herein, the fusion protein further comprises an inflammatory cytokine inhibitor. Non-limiting examples of inflammatory cytokines include interleukin1 (IL-1), IL-6, IL-12, IL-18, tumor necrosis factor alpha (TNF-α), interferon gamma (IFNγ), and granulocyte-macrophage colony stimulating factor (GM-CSF). In some embodiments, the fusion protein further comprises an inflammatory cytokine receptor antagonist. In some embodiments, the fusion protein further comprises an interleukin-1 receptor antagonist (IL-1Ra) domain (see e.g., SEQ ID NOs: 88, 89, 93-97, 177, 179, 181). Some exemplary IL-1Ra domain amino acid sequences are as follows:

RPSGRKSSKMQAFRIWDVNQKTFYLRNNQLVAGYLQGPNVNLEEK
IDVVPIEPHALFLGIHGGKMCLSCVKSGDETRLQLEAVDITDLSE
NRKQDKRFAFIRSDSGPTTSFESAACPGWFLCTAMEADQPVSLTN
MPDEGVMVTKFYFQEDE
(SEQ ID NO: 205; human IL-1Ra; see e.g.,
SEQ ID NOs: 88, 89, 95-97, 177, 179, 181);
RPSGKRPCKMQAFRIWDTNQKTFYLRNNQLIAGYLQGPNIKLEEK
IDMVPIDLHSVFLGIHGGKLCLSCAKSGDDIKLQLEEVNITDLSK
NKEEDKRFTFIRSEKGPTTSFESAACPGWFLCTTLEADRPVSLTN
TPEEPLIVTKFYFQEDQ
(SEQ ID NO: 206; murine
IL-1Ra; see e.g., SEQ ID NO: 93);
or
RPSGKRPCKMQAFRIWDTNQKTFYLRNNQLIAGYLQGPNIKLEEK
IDMVPIDLHSVFLGIHGGKLCLSCAKSGDDIKLQLEEVDITDLSK
NKEEDKRFTFIRSEKGPTTSFESAACPGWFLCTTLEADRPVSLTN
TPEEPLIVTKFYFQEDQ
(SEQ ID NO: 207; murine IL-1Ra (N84D);
see e.g., SEQ ID NO: 94).

In some embodiments, the IL-1Ra domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a sequence selected from the group consisting of SEQ ID NO: 205-207. In some embodiments, the IL-1Ra domain comprises an amino acid sequence having at least 95%, 96%, 97%, 98% or 99% identity to a sequence selected from the group consisting of SEQ ID NO: 205-207. In some embodiments, the IL-1Ra domain comprises an amino acid sequence having at least 97%, 98% or 99% identity to a sequence selected from the group consisting of SEQ ID NO: 205-207.

In some embodiments, the fusion protein can comprise any order of the Fc, SPINK, and IL-1Ra domains. Non-limiting examples of the N terminus to C terminus order include: Fc-SPINK-IL1Ra; Fc-IL1Ra-SPINK; SPINK-Fc-IL1Ra; SPINK-IL1Ra-Fc; IL1Ra-Fc-SPINK; or IL1Ra-SPINK-Fc. In some embodiments, the fusion protein comprises from N terminus to C terminus: SPINK-Fc-IL1Ra.

In some embodiments, the fusion protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a sequence selected from the group consisting of SEQ ID NO: 88, 89, 93-97. In some embodiments, the fusion protein comprises an amino acid sequence having at least 95%, 96%, 97%, 98% or 99% identity to a sequence selected from the group consisting of SEQ ID NO: 88, 89, 93-97. In some embodiments, the fusion protein comprises an amino acid sequence having at least 97%, 98% or 99% identity to a sequence selected from the group consisting of SEQ ID NO: 88, 89, 93-97.

It is noted the Fc domain and the SPINK1 domain, BPTI domain, and/or IL-1Ra domain can be linked directly via a linker. The linker can be a chemical linker, a single peptide bond (e.g., linked directly to each other) or a peptide linker containing one or more amino acid residues (e.g. with an intervening amino acid or amino acid sequence between the Fc domain and the SPINK1 or BPTI domain).

In some embodiments, the Fc domain and the SPINK1 domain, BPTI domain, and/or IL-1Ra domain are linked via a peptide linker. The term “peptide linker” as used herein denotes a peptide with amino acid sequences, which is in some embodiments of synthetic origin. It is noted that peptide linkers may affect folding of a given fusion protein, and may also react/bind with other proteins, and these properties can be screened for by known techniques.

Exemplary peptide linkers include those that consist of glycine and serine residues, the so-called Gly-Ser polypeptide linkers. As used herein, the term “Gly-Ser polypeptide linker” refers to a peptide that consists of glycine and serine residues. In some embodiments, the peptide linker comprises the amino acid sequence (GxS)n with G is glycine, S is serine, where x is 3, 4 or 5 and n is 1, 2, 3, 4, 5, 6, 7, 8, 9 or 10, SEQ ID NO: 41-70. In some embodiments, x is 3 and, n is 8, 9 or 10. In some embodiments, x is 4 and n is 6, 7, or 8. In some embodiments, x is 4 and n is 6 or 7. In some embodiments, x is 4 and n is 7. In some embodiments, the peptide linker comprises the amino acid sequence Ser(Gly₄Ser)ₙ wherein n is an integer 1 to 10, SEQ ID NO: 71-80, respectively. In some embodiments, the peptide linker comprises the amino acid sequence (G₄S)₆G₂ (SEQ ID NO: 242). In some embodiments, the peptide linker comprises the amino acid sequence GSGGGSGGGGSGGGGS (SEQ ID NO: 81).

In some embodiments, the peptide linker comprises the amino acid sequence (X)ₙ, where each X is independently aspartic acid (D) or glutamic acid (E), and n ranges from 1 to up to 10 or more (SEQ ID NO: 82).

Exemplary linkers, in addition to those described herein, include a string of histidine residues, e.g., His6 (SEQ ID NO: 83); sequences made up of Ala and Pro, varying the number of Ala-Pro pairs to modulate the flexibility of the linker; and sequences made up of charged amino acid residues e.g., mixing Glu and Lys. Flexibility can be controlled by the types and numbers of residues in the linker. See, e.g., Perham, 30 Biochem. 8501 (1991); Wriggers et al., 80 Biopolymers 736 (2005).

Chemical linkers can comprise a direct bond or an atom such as oxygen or sulfur, a unit such as NH, C(O), C(O)NH, SO, SO₂, SO₂NH, or a chain of atoms, such as substituted or unsubstituted C₁-C₆ alkyl, substituted or unsubstituted C₂-C₆ alkenyl, substituted or unsubstituted C₂-C₆ alkynyl, substituted or unsubstituted C₆-C₁₂ aryl, substituted or unsubstituted C₅-C₁₂heteroaryl, substituted or unsubstituted C5-C12 heterocyclyl, substituted or unsubstituted C₃-C₁₂ cycloalkyl, where one or more methylenes can be interrupted or terminated by 0, S, S (0), SO₂, NH, or C(O). The linker can be 1 amino acid or more, 5 amino acids or more, 10 amino acids or more, 15 amino acids or more, 20 amino acids or more, 25 amino acids or more, 30 amino acids or more, 35 amino acids or more, 40 amino acids or more, 45 amino acids or more, 50 amino acids or more and beyond.

In some embodiments, the linker can be a cleavable linker. For example, the linker comprises a cleavable group. A cleavable group is one which is sufficiently stable under a first set of conditions and can be cleaved to release the two parts the cleavable group is holding together. In a preferred embodiment, the cleavable group is cleaved at least 10 times or more, preferably at least 100 times faster under a first reference condition (which can, e.g., be selected to mimic or represent intracellular conditions) than under a second reference condition (which can, e.g., be selected to mimic or represent conditions found in the blood or serum).

Cleavable groups are susceptible to cleavage agents, e.g., pH, redox potential or the presence of degradative molecules. Generally, cleavage agents are more prevalent or found at higher levels or activities at the desired site of action of the molecule comprising the cleavable group. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases.

Exemplary cleavable groups include, but are not limited to peptide-based cleavable groups, (e.g., groups that are cleaved by enzymes such as peptidases and proteases, e.g., —NHCHR^{A}C(O)NHCHR^{B}C(O)—, where R^{A} and R^{B} are the R groups of the two adjacent amino acids); redox cleavable groups (e.g., —S—S— and —C(R)₂—S—S—, wherein R is H or C1-C6 alkyl and at least one R is C₁-C₆ alkyl such as CH3 or CH2CH3); phosphate-based cleavable linking groups (e.g., —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(ORk)—S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—, wherein R is optionally substituted linear or branched C₁-C₁₀ alkyl); acid cleavable groups (e.g., hydrazones, esters, and esters of amino acids, —C═NN— and —OC(O)—); and ester-based cleavable groups (e.g., —C(O)O—).

In some embodiments, the linker comprises a peptide based cleavable group comprises two or more amino acids. In some embodiments, the peptide-based cleavable group comprises the amino acid sequence that is the substrate for a peptidase or a protease found in pancreas.

The Fc fusion protein described herein can be used for treating a condition, disease or disorder characterized by an elevated trypsin, e.g., pancreatic trypsin activity or level. Generally, the method comprises administering a therapeutically effective amount of a Fc fusion protein described herein to a subject in need thereof.

As used herein terms “condition,” “disorder,” and “disease” relate to any unhealthy or abnormal state. The term “condition characterized by an elevated trypsin activity or level” includes conditions, disorders, and diseases in which inhibiting trypsin, e.g., pancreatic trypsin activity provides a therapeutic benefit. Conditions characterized by an elevated trypsin activity include conditions characterized by an immunomodulatory or an inflammatory effect. In particular, the such conditions include pancreatitis, including acute pancreatitis and chronic pancreatitis, systemic inflammatory response syndrome, acute circulatory failure (e.g., caused by shock), disseminated intravascular coagulation, and multiple organ dysfunction syndrome. The conditions characterized by an elevated trypsin activity also include use in high-risk surgical patients. The conditions characterized by an elevated trypsin activity also includes infections of the lung, liver, heart, or kidney. The conditions characterized by an elevated trypsin activity also includes severe sepsis, acute lung injury (ALI) caused by SARS viruses or acute respiratory distress syndrome (ARDS).

In some embodiments, the condition characterized by an elevated trypsin activity is pancreatitis. Pancreatitis is characterized by damage to the pancreas and surrounding tissues which arises from autodigestion of the cells by the various digestive enzymes activated by trypsin. Animal studies of chemically-induced pancreatitis suggest that the disorder is rooted in the inability of pancreatic acinar cells to excrete the digestive proenzymes. This results in the activation of trypsinogen to trypsin by lysosomal hydrolases within the cell, with the amount produced exceeding protective levels of protease inhibitor normally available. This results in the subsequent activation of the other digestive enzymes co-localized with trypsin in the lysosome. These activated digestive enzymes cause edema, interstitial hemorrhage, vascular damage, coagulation necrosis, fat necrosis and parenchymal cell necrosis. The activated digestive enzymes may subsequently enter the blood and the peritoneal cavity and can lead to secondary multiple organ damage.

Pancreatitis is generally divided into acute pancreatitis and chronic pancreatitis. Acute pancreatitis is characterized by acute inflammation in the pancreas accompanied by necrosis of the parenchymal cells, i.e. acinar cells, and duct cells. The most common causes of acute pancreatitis are alcoholism and gallstone disease. Alcohol sensitizes the pancreas to the inflammatory response and inhibition of this inflammatory response results in improvement in the severity of the pancreatitis. Gallstones cause pancreatitis by both obstructing the pancreatic duct and causing reflux of bile into the pancreatic duct as the stones migrate from the gallbladder to the common bile duct, ampulla of Vater and duodenum. Bile reflux-induced pancreatitis can also be ameliorated by inhibition of the inflammatory response. In some embodiments, acute pancreatitis includes disease post ERCP (endoscopic retrograde cholangiopancreatography). Chronic pancreatitis results from continued episodes of acute pancreatitis and is most commonly caused by alcohol abuse. Development of chronic pancreatitis includes chronic inflammation as well fibrosis and loss of parenchymal tissue.

In some embodiments of the various aspects described herein, pancreatitis is acute pancreatitis.

In some other embodiments of the various aspects described herein, pancreatitis is chronic pancreatitis.

In some embodiments, the condition characterized by an elevated trypsin activity is an inflammatory bowel disease.

In some embodiments, the method can comprise a step of assaying a sample from the subject for determining trypsin activity and/or level prior to onset of treatment. The activity or level can be compared to a reference, e.g., trypsin activity or level in a healthy subject. A subject having elevated trypsin activity or level can be selected for treatment. Methods for determining trypsin activity and level are well known in the art and available to one of skill in the art.

In some embodiments, the method can comprise a step of obtaining or receiving results of an assay determining trypsin activity or level in a subject prior to administration.

It is noted that the terms “administered” and “subjected” are used interchangeably in the context of treatment of a disease or disorder. In jurisdictions that forbid the patenting of methods that are practiced on the human body, the meaning of “administering” of a composition to a human subject shall be restricted to prescribing a controlled substance that a human subject will be administer to the subject by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “administer” refers to the placement of the Fc fusion protein or a composition comprising the same into a subject by a method or route which results in at least partial localization of the composition at a desired site such that desired effect is produced. A Fc fusion protein or a composition comprising the same can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, administration will generally be local rather than systemic. In some embodiments, administering is intravenous (IV) or intraperitoneal (IP) administration

The phrase “therapeutically effective amount” as used herein means that amount of a Fc fusion protein described herein which is effective for producing some desired therapeutic effect in at least a sub-population of cells, e.g., modulate or inhibit activity of trypsin (such as pancreatic trypsin) in a subject at a reasonable benefit/risk ratio applicable to any medical treatment. Thus, “therapeutically effective amount” means that amount which, when administered to a subject for treating pancreatitis, is sufficient to affect such treatment for pancreatitis.

Depending on the route of administration, effective doses can be calculated according to the body weight, body surface area, or organ size of the subject to be treated. Optimization of the appropriate dosages can readily be made by one skilled in the art in light of pharmacokinetic data observed in human clinical trials. Alternatively, or additionally, the dosage to be administered can be determined from studies using animal models for the particular type of condition to be treated, and/or from animal or human data obtained from agents which are known to exhibit similar pharmacological activities. The final dosage regimen will be determined by the attending surgeon or physician, considering various factors which modify the action of active agent, e.g., the agent's specific activity, the agent's specific half-life in vivo, the severity of the condition and the responsiveness of the patient, the age, condition, body weight, sex and diet of the patient, the severity of any present infection, time of administration, the use (or not) of other concomitant therapies, and other clinical factors.

Determination of an effective amount is well within the capability of those skilled in the art. Generally, the actual effective amount can vary with the specific compound, the use or application technique, the desired effect, the duration of the effect and side effects, the subject's history, age, condition, sex, as well as the severity and type of the medical condition in the subject, and administration of other pharmaceutically active agents. Accordingly, an effective dose of compound described herein is an amount sufficient to produce at least some desired therapeutic effect in a subject.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of use or administration utilized.

The effective dose can be estimated initially from cell culture assays. A dose can be formulated in animal models to achieve a circulating plasma concentration range that includes the IC₅₀ (i.e., the concentration of the therapeutic which achieves a half-maximal inhibition of symptoms) as determined in cell culture. Levels in plasma can be measured, for example, by high performance liquid chromatography. The effects of any particular dosage can be monitored by a suitable bioassay. The effective plasma concentration for Fc fusion protein or a fragment thereof can be about 0.01 μM to about 10 μM, about 0.2 μM to about 5 μM, or about 0.8 to about 3 μM in a subject, such as a rat, dog, or human.

Generally, the compositions are administered so that a Fc fusion protein described herein is used or given at a dose from 50 μg/kg to 1000 mg/kg; 1 μg/kg to 500 mg/kg; 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. Further contemplated is a dose (either as a bolus or continuous infusion) of about 0.1 mg/kg to about 10 mg/kg, about 0.3 mg/kg to about 5 mg/kg, or 0.5 mg/kg to about 3 mg/kg. It is to be further understood that the ranges intermediate to those given above are also within the scope of this disclosure, for example, in the range 1 mg/kg to 10 mg/kg, for example use or dose ranges such as 2 mg/kg to 8 mg/kg, 3 mg/kg to 7 mg/kg, 4 mg/kg to 6 mg/kg, and the like.

Typically, a dose of 500 mgs, 1 gram, 2.5 grams or more is used for a human subject. For example, about 200 mgs to about 3 mgs, or more of the Fc-fusion protein can be administered to the subject, e.g., a human patient per day. In some embodiments, a subject, e.g., a human patient is treated with an Fc-fusion protein described herein at a dose of at least 500 mgs per day. For example, a subject, e.g., a human patient can be treated with an Fc-fusion protein described herein at a dose of at least 1 gram per day. In some embodiments, a subject, e.g., a human patient can be treated with an Fc-fusion protein described herein at a dose of at least 2.5 grams per day.

In some embodiments, about 200 to 500 milligrams and more preferably about 320 milligrams of the Fc-fusion protein can be administered to a 70 kg subject, e.g., a human patient.

The Fc fusion protein described herein can be administered at once, or can be divided into a number of smaller doses to be administered at intervals of time. It is understood that the precise dosage and duration of treatment will be a function of the location of where the Fc fusion protein is administered, the carrier and other variables that can be determined empirically using known testing protocols or by extrapolation from in vivo or in vitro test data. It is to be noted that concentrations and dosage values can also vary with the age of the individual treated. It is to be further understood that for any particular subject, specific dosage regimens can need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The Fc fusion protein can be administered as a single bolus or multiple boluses, as a continuous infusion, or a combination thereof. For example, the Fc fusion protein can be administered as a single bolus initially, and then administered as a continuous infusion following the bolus. The rate of the infusion can be any rate sufficient to maintain effective concentration, for example, to maintain effective plasma concentration. Some contemplated infusion rates include from 1 μg/kg/min to 100 mg/kg/min, or from 1 μg/kg/hr to 1000 mg/kg/hr. Rates of infusion can include 0.2 to 1.5 mg/kg/min, or more specifically 0.25 to 1 mg/kg/min, or even more specifically 0.25 to 0.5 mg/kg/min. It will be appreciated that the rate of infusion can be determined based upon the dose necessary to maintain effective plasma concentration and the rate of elimination of the compound, such that the compound is administered via infusion at a rate sufficient to safely maintain a sufficient effective plasma concentration of compound in the bloodstream.

It will be appreciated that methods of treatment of the present invention can be employed in combination with additional therapies. For example, a treatment according to the present disclosure can be co-administered with one or more desired therapeutics or medical procedures for treating pancreatitis.

The terms “co-administration” or the like, as used herein, are meant to encompass administration of the selected therapeutic agents to a single patient and are intended to include treatment regimens in which the agents are administered by the same or different route of administration or at the same or different time. The particular combination of therapies (therapeutics or procedures) to employ in such a combination regimen will take into account compatibility of the desired therapeutics and/or procedures and the desired therapeutic effect to be achieved.

In some embodiments of the various aspects described herein, the subject methods include monitoring the patient for efficacy of treatment. Monitoring may measure weight loss, colon thickening, soft/loose stool (e.g., diarrhea, watery diarrhea, etc.), rectal bleeding (e.g., bloody stool), abdominal cramps, abdominal pain, vomiting, acute right lower quadrant pain, malaise, fatigue, fever, and/or anemia; etc.) and/or monitoring for the presence or absence (either quantitatively or qualitatively) of a biomarker associated with the disease being treated. For example, diagnosis of pancreatitis, as well as the assessment of treatment efficacy for pancreatitis using the subject methods, can be determined by the presence or absence of biomarkers in a biological sample (e.g., blood, stool, etc.) from the patient followed by colonoscopy and/or any other suitable technique for pancreatitis.

Examples of biomarkers that can be used to diagnose and/or determine the severity of pancreatitis can be found, for example, in Momi et al., Minerva Gastroenterol Dietol. 2012, 58(4):283-97; Jin et al., Intern Med. 2011, 50(15):1507-16; Paulo et al., Proteomics Clin Appl. 2011, 5(3-4):109-20; Buxbaum et al., JOP. 2010, 11(6):536-44; Carroll et al., Am Fam Physician. 2007, 75(10):1513-20; Matull et al., J Clin Pathol. 2006, 59(4):340-4; Cavestro et al., JOP. 2005, 6(1 Suppl):53-9; and US patent application publications 20100184662, 20100144850, 20100099615, and 20050166275; contents of all of which are hereby incorporated by reference for their teachings on biomarkers of pancreatitis.

For administration to a subject, the Fc fusion protein described herein can be formulated into pharmaceutically acceptable compositions/formulations.

These pharmaceutically acceptable compositions comprise a Fc fusion protein described herein, formulated together with one or more pharmaceutically acceptable carriers (additives) and/or diluents. As described in detail below, the pharmaceutical compositions described herein can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Examples of solid carriers include starch, sugar, bentonite, silica, and other commonly used carriers. Further non-limiting examples of carriers and diluents which can be used in the formulations comprising a Fc fusion protein described herein include saline, syrup, dextrose, and water.

Pharmaceutically acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

The Fc fusion protein described herein can be formulated in a gelatin capsule, in tablet form, dragee, syrup, suspension, topical cream, suppository, injectable solution, or kits for the preparation of syrups, suspension, topical cream, suppository or injectable solution just prior to use. Also, Fc fusion protein described herein can be included in composites, which facilitate its slow release into the blood stream, e.g., silicon disc, polymer beads.

The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J. Pharm. Sci. Tech. 1997 51:166-171. Methods to make invention formulations include the step of bringing into association or contacting a Fc fusion protein described herein with one or more excipients or carriers. In general, the formulations are prepared by uniformly and intimately bringing into association a Fc fusion protein described herein with liquid excipients or finely divided solid excipients or both, and then, if appropriate, shaping the product.

The preparative procedure may include the sterilization of the pharmaceutical preparations. The Fc fusion protein described herein may be mixed with auxiliary agents such as lubricants, preservatives, stabilizers, salts for influencing osmotic pressure, etc., which do not react deleteriously with the compounds.

Examples of injectable form include solutions, suspensions and emulsions. Injectable forms also include sterile powders for extemporaneous preparation of injectable solutions, suspensions or emulsions. The Fc fusion protein described herein can be injected in association with a pharmaceutical carrier such as normal saline, physiological saline, bacteriostatic water, Cremophor™ EL (BASF, Parsippany, N.J.), phosphate buffered saline (PBS), Ringer's solution, dextrose solution, ethanol, polyol (e.g., glycerol, propylene glycol, and liquid polyethylene glycol), vegetable oils, and suitable mixtures thereof, and other aqueous carriers known in the art. Appropriate non-aqueous carriers may also be used and examples include fixed oils and ethyl oleate. In all cases, the composition must be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin. A suitable carrier is 5% dextrose in saline. Frequently, it is desirable to include additives in the carrier such as buffers and preservatives or other substances to enhance isotonicity and chemical stability.

In some embodiments, Fc fusion protein described herein can be administrated encapsulated within liposomes. The manufacture of such liposomes and insertion of molecules into such liposomes being well known in the art, for example, as described in U.S. Pat. No. 4,522,811. Liposomal suspensions (including liposomes targeted to particular cells, e.g., a pituitary cell) can also be used as pharmaceutically acceptable carriers.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient, e.g., a Fc fusion protein described herein) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185; content of each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif USA)), or a combination thereof to provide the desired release profile in varying proportions.

In some embodiments, the Fc fusion protein described herein is prepared with carriers that will protect the Fc fusion protein against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

In the case of oral ingestion, excipients useful for solid preparations for oral administration are those generally used in the art, and the useful examples are excipients such as lactose, sucrose, sodium chloride, starches, calcium carbonate, kaolin, crystalline cellulose, methyl cellulose, glycerin, sodium alginate, gum arabic and the like, binders such as polyvinyl alcohol, polyvinyl ether, polyvinyl pyrrolidone, ethyl cellulose, gum arabic, shellac, sucrose, water, ethanol, propanol, carboxymethyl cellulose, potassium phosphate and the like, lubricants such as magnesium stearate, talc and the like, and further include additives such as usual known coloring agents, disintegrators such as alginic acid and Primogel™, and the like. The Fc fusion protein described herein can be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, the Fc fusion protein described herein may be incorporated with excipients and used in the form of tablets, capsules, elixirs, suspensions, syrups, and the like. Such compositions and preparations should contain at least 0.1% of compound. The percentage of the agent in these compositions may, of course, be varied and may conveniently be between about 2% to about 60% of the weight of the unit. The amount of the Fc fusion protein described herein in such therapeutically useful compositions is such that a suitable dosage will be obtained. Preferred compositions according to the present invention are prepared so that an oral dosage unit contains between about 100 and 2000 mg of the Fc fusion protein described herein. Examples of bases useful for formulation of suppositories are oleaginous bases such as cacao butter, polyethylene glycol, lanolin, fatty acid triglycerides, WITEPSOL (DYNAMITE NOBEL CO. LTD.) and the like. Liquid preparations may be in the form of aqueous or oleaginous suspension, solution, syrup, elixir and the like, which can be prepared by a conventional way using additives. The compositions can be given as a bolus dose, to maximize the circulating levels for the greatest length of time after the dose. Continuous infusion may also be used after the bolus dose.

The Fc fusion protein described herein can also be administered parenterally. Solutions or suspensions of the Fc fusion protein described herein can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

It may be advantageous to formulate oral or parenteral compositions in dosage unit form for ease of administration and uniformity of dosage. As used herein, “dosage unit” refers to physically discrete units suited as unitary dosages for the subject to be treated; each unit containing a predetermined quantity of the Fc fusion protein described herein calculated to produce the desired therapeutic effect in association with the required pharmaceutical carrier.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

For oral or enteral formulations as disclosed herein for use with the present invention, tablets can be formulated in accordance with conventional procedures employing solid carriers well-known in the art. Capsules employed for oral formulations to be used with the methods of the present invention can be made from any pharmaceutically acceptable material, such as gelatin or cellulose derivatives. Sustained release oral delivery systems and/or enteric coatings for orally administered dosage forms are also contemplated, such as those described in U.S. Pat. No. 4,704,295, “Enteric Film-Coating Compositions,” issued Nov. 3, 1987; U.S. Pat. No. 4,556,552, “Enteric Film-Coating Compositions,” issued Dec. 3, 1985; U.S. Pat. No. 4,309,404, “Sustained Release Pharmaceutical Compositions,” issued Jan. 5, 1982; and U.S. Pat. No. 4,309,406, “Sustained Release Pharmaceutical Compositions,” issued Jan. 5, 1982.

As regards formulations for administering a Fc fusion protein described herein, one particularly useful embodiment is a tablet formulation comprising a compound of Fc fusion protein described herein with an enteric polymer casing. An example of such a preparation can be found in WO2005/021002. The active material in the core can be present in a micronized or solubilized form. In addition to active materials the core can contain additives conventional to the art of compressed tablets. Appropriate additives in such a tablet can comprise diluents such as anhydrous lactose, lactose monohydrate, calcium carbonate, magnesium carbonate, dicalcium phosphate or mixtures thereof; binders such as microcrystalline cellulose, hydroxypropylmethylcellulose, hydroxypropyl-cellulose, polyvinylpyrrolidone, pre-gelatinized starch or gum acacia or mixtures thereof; disintegrants such as microcrystalline cellulose (fulfilling both binder and disintegrant functions) cross-linked polyvinylpyrrolidone, sodium starch glycollate, croscarmellose sodium or mixtures thereof; lubricants, such as magnesium stearate or stearic acid, glidants or flow aids, such as colloidal silica, talc or starch, and stabilizers such as desiccating amorphous silica, coloring agents, flavors etc. Preferably the tablet comprises lactose as diluent. When a binder is present, it is preferably hydroxypropylmethyl cellulose. Preferably, the tablet comprises magnesium stearate as lubricant. Preferably the tablet comprises croscarmellose sodium as disintegrant. Preferably, the tablet comprises microcrystalline cellulose.

The diluent can be present in a range of 10-80% by weight of the core. The lubricant can be present in a range of 0.25-2% by weight of the core. The disintegrant can be present in a range of 1-10% by weight of the core. Microcrystalline cellulose, if present, can be present in a range of 10-80% by weight of the core.

The active ingredient, e.g., a Fc fusion protein described herein preferably comprises between 10 and 50% of the weight of the core, more preferably between 15 and 35% of the weight of the core (calculated as free base equivalent). The core can contain any therapeutically suitable dosage level of the active ingredient, but preferably contains up to 150 mg of the active ingredient. Particularly preferably, the core contains 20, 30, 40, 50, 60, 80 or 100 mg of the active ingredient. The active ingredient can be present as is or as any pharmaceutically acceptable salt. If the active ingredient is present as a salt, the weight is adjusted such that the tablet contains the desired amount of active ingredient, calculated as free base or free acid of the salt.

The core can be made from a compacted mixture of its components. The components can be directly compressed, or can be granulated before compression. Such granules can be formed by a conventional granulating process as known in the art. In an alternative embodiment, the granules can be individually coated with an enteric casing, and then enclosed in a standard capsule casing.

The core is surrounded by a casing which comprises an enteric polymer. Examples of enteric polymers are cellulose acetate phthalate, cellulose acetate succinate, methylcellulose phthalate, ethylhydroxycellulose phthalate, polyvinylacetate pthalate, polyvinylbutyrate acetate, vinyl acetate-maleic anhydride copolymer, styrene-maleic mono-ester copolymer, methyl acrylate-methacrylic acid copolymer or methacrylate-methacrylic acid-octyl acrylate copolymer. These can be used either alone or in combination, or together with other polymers than those mentioned above. The casing can also include insoluble substances which are neither decomposed nor solubilized in living bodies, such as alkyl cellulose derivatives such as ethyl cellulose, cross-linked polymers such as styrene-divinylbenzene copolymer, polysaccharides having hydroxyl groups such as dextran, cellulose derivatives which are treated with bifunctional crosslinking agents such as epichlorohydrin, dichlorohydrin or 1,2-, 3,4-diepoxybutane. The casing can also include starch and/or dextrin.

In some embodiments, an enteric coating materials are the commercially available Eudragit® enteric polymers such as Eudragit® L, Eudragit® S and Eudragit® NE used alone or with a plasticizer. Such coatings are normally applied using a liquid medium, and the nature of the plasticizer depends upon whether the medium is aqueous or non-aqueous. Plasticizers for use with aqueous medium include propylene glycol, triethyl citrate, acetyl triethyl citrate or Citroflex® or Citroflex® A2. Non-aqueous plasticizers include these, and also diethyl and dibutyl phthalate and dibutyl sebacate. A preferred plasticizer is Triethyl citrate. The quantity of plasticizer included will be apparent to those skilled in the art.

The casing can also include an anti-tack agent such as talc, silica or glyceryl monostearate. Preferably the anti-tack agent is glyceryl monostearate. Typically, the casing can include around 5-25 wt % Plasticizers and up to around 50 wt % of anti-tack agent, preferably 1-10 wt % of anti-tack agent.

If desired, a surfactant can be included to aid with forming an aqueous suspension of the polymer. Many examples of possible surfactants are known to the person skilled in the art. Preferred examples of surfactants are polysorbate 80, polysorbate 20, or sodium lauryl sulphate. If present, a surfactant can form 0.1-10% of the casing, preferably 0.2-5% and particularly preferably 0.5-2%.

A seal coat can also be included between the core and the enteric coating. A seal coat is a coating material which can be used to protect the enteric casing from possible chemical attack by any alkaline ingredients in the core. The seal coat can also provide a smoother surface, thereby allowing easier attachment of the enteric casing. A person skilled in the art would be aware of suitable coatings. Preferably the seal coat is made of an OPADRY coating, and particularly preferably it is OPADRY WHITE OY—S-28876. Other enteric-coated preparations of this sort can be prepared by one skilled in the art, using these materials or their equivalents.

The disclosure also provides a polynucleotide encoding a Fc fusion protein described herein. The skilled person will understand that, due to the degeneracy of the genetic code, a given polypeptide can be encoded by different polynucleotides. These “variants” are encompassed herein.

In some embodiments, a nucleic acid encoding a Fc fusion protein described herein is comprised in a vector. In some embodiments, a nucleic acid sequence encoding a Fc fusion protein described herein, or any part thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments, the expression system is an organism other than the source of the native/wild-type sequence (or a cell obtained from such organism). In some embodiments, the vector and/or nucleic acid sequence described herein is codon-optimized for expression in a mammal or mammalian cell, e.g., a mouse, a murine cell, or a human cell. In some embodiments, the vector and/or nucleic acid sequence described herein is codon-optimized for expression in a human cell. In some embodiments, the vector and/or nucleic acid sequence described herein is codon-optimized for expression in a yeast or yeast cell. In some embodiments, the vector and/or nucleic acid sequence described herein is codon-optimized for expression in a bacterial cell. In some embodiments, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

The disclosure also provides a host cell comprising a polynucleotide described herein or a plasmid or vector described herein. As used herein, the term “cell” refers to a single cell as well as to a population of (i.e., more than one) cells. A host cell can be a prokaryotic or eukaryotic host cell. Exemplary host cells include, but are not limited to, bacterial cells, yeast cells, plant cell, animal (including insect) or human cells.

The host cells can be employed in a method of producing a Fc fusion protein described herein. Generally, the method comprises: culturing a host cell comprising a polynucleotide described herein or a plasmid or vector described herein under conditions such that the Fc fusion protein is expressed; and optionally recovering the Fc fusion protein from the culture medium. The Fc fusion protein can be concentrated and purified by a variety of biochemical and chromatographic methods, including methods utilizing differences in size, charge, hydrophobicity, solubility, specific affinity, etc. between the Fc fusion protein and other substances in the cell culture medium. In some embodiments, the Fc fusion protein is secreted from the host cells.

The Fc fusion protein described herein can be produced as recombinant molecules in prokaryotic or eukaryotic host cells, such as bacteria, yeast, plant, animal (including insect) or human cell lines or in transgenic animals. Recombinant methods of producing a polypeptide through the introduction of a vector including nucleic acid encoding the polypeptide into a suitable host cell is well known in the art, such as is described in Sambrook et al., Molecular Cloning: A Laboratory Manual, 2d Ed, Vols 1 to 8, Cold Spring Harbor, N Y (1989); M. W. Pennington and B. M. Dunn, Methods in Molecular Biology: Peptide Synthesis Protocols, Vol 35, Humana Press, Totawa, NJ (1994), contents of both of which are herein incorporated by reference.

The production of Fc fusion proteins at high levels in suitable host cells requires the assembly of the polynucleotides encoding such Fc fusion proteins into efficient transcriptional units together with suitable regulatory elements in a recombinant expression vector that can be propagated in various expression systems according to methods known to those skilled in the art. Efficient transcriptional regulatory elements could be derived from viruses having animal cells as their natural hosts or from the chromosomal DNA of animal cells. For example, promoter-enhancer combinations derived from the Simian Virus 40, adenovirus, BK polyoma virus, human cytomegalovirus, or the long terminal repeat of Rous sarcoma virus, or promoter-enhancer combinations including strongly constitutively transcribed genes in animal cells like beta-actin or GRP78 can be used. In order to achieve stable high levels of mRNA, the transcriptional unit should contain in its 3′-proximal part a DNA region encoding a transcriptional termination-polyadenylation sequence. Generally, this sequence can be derived from the Simian Virus 40 early transcriptional region, the rabbit beta-globin gene, or the human tissue plasminogen activator gene.

The vector is transfected into a suitable host cell line for expression of the Fc fusion protein. Examples of cell lines that can be used to prepare the Fc fusion described herein include, but are not limited to monkey COS-cells, mouse L-cells, mouse C127-cells, hamster BHK-21 cells, human embryonic kidney 293 cells, and hamster CHO-cells.

The expression vector encoding the Fc fusion protein can be introduced in several different ways. For instance, the expression vectors can be created from vectors based on different animal viruses. Examples of these are vectors based on baculovirus, vaccinia virus, adenovirus, and preferably bovine papilloma virus

The transcription units encoding the corresponding DNAs can also be introduced into animal cells together with another recombinant gene, which may function as a dominant selectable marker in these cells in order to facilitate the isolation of specific cell clones, which have integrated the recombinant DNA into their genome. Examples of this type of dominant selectable marker genes are Tn5 amino glycoside phosphotransferase, conferring resistance to geneticin (G418), hygromycin phosphotransferase, conferring resistance to hygromycin, and puromycin acetyl transferase, conferring resistance to puromycin. The recombinant expression vector encoding such a selectable marker can reside either on the same vector as the one encoding the cDNA of the desired protein, or it can be encoded on a separate vector which is simultaneously introduced and integrated to the genome of the host cell, frequently resulting in a tight physical linkage between the different transcription units

Other types of selectable marker genes, which can be used together with the cDNA of the desired protein are based on various transcription units encoding dihydrofolate reductase (dhfr). After introduction of this type of gene into cells lacking endogenous dhfr-activity, preferentially CHO-cells (DUKX-B 11, DG-44) it will enable these to grow in media lacking nucleosides. An example of such a medium is Ham's F12 without hypoxanthine, thymidin, and glycine. These dhfr-genes can be introduced together with the Kazal-type serine protease inhibitors' cDNA transcriptional units into CHO-cells of the above type, either linked on the same vector or on different vectors, thus creating dhfr-positive cell lines producing recombinant protein.

If the above cell lines are grown in the presence of the cytotoxic dhfr-inhibitor methotrexate, new cell lines resistant to methotrexate will emerge. These cell lines may produce recombinant protein at an increased rate due to the amplified number of linked dhfr and the desired protein's transcriptional units. When propagating these cell lines in increasing concentrations of methotrexate (1-10000 nM), new cell lines can be obtained which produce the desired protein at a very high rate.

The above cell lines producing the desired protein can be grown on a large scale, either in suspension culture or on various solid supports. Examples of these supports are micro carriers based on dextran or collagen matrices, or solid supports in the form of hollow fibers or various ceramic materials. When grown in cell suspension culture or on micro carriers the culture of the above cell lines can be performed either as a batch culture or as a perfusion culture with continuous production of conditioned medium over extended periods of time. Thus, according to the present invention, the above cell lines are well suited for the development of an industrial process for the production of the desired recombinant proteins.

An example of such purification is the adsorption of the Fc fusion protein to a monoclonal antibody or a binding peptide, which is immobilized on a solid support. After desorption, the protein can be further purified by a variety of chromatographic techniques based on the above properties.

Exemplary genera of yeast contemplated to be useful in the production of the Fc fusion protein described herein as hosts are Pichia (formerly classified as Hansenula), Saccharomyces, Kluyveromyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopsis, and the like. Genera include those selected from the group consisting of Saccharomyces, Schizosaccharomyces, Kluyveromyces, Pichia and Torulaspora. Examples of Saccharomyces spp. are S. cerevisiae, S. italicus and S. rouxii.

Suitable promoters for S. cerevisiae include those associated with the PGKI gene, GAL1 or GAL10 genes, CYCI, PHO5, TRPI, ADHI, ADH2, the genes for glyceral-dehyde-3-phosphate dehydrogenase, hexokinase, pyruvate decarboxylase, phos-phofructokinase, triose phosphate isomerase, phosphoglucose isomerase, glucokinase, alpha-mating factor pheromone, the PRBI, the GUT2, the GPDI promoter, and hybrid promoters involving hybrids of parts of 5′ regulatory regions with parts of 5′ regulatory regions of other promoters or with upstream activation sites (e.g. the promoter of EP-A-258 067).

Convenient regulatable promoters for use in Schizosaccharomyces pombe are the thiamine-repressible promoter from the nmt gene as described by Maundrell (Maundrell K. 1990. Nmt1 of fission yeast. A highly transcribed gene completely repressed by thiamine. J. Biol. Chem. 265:10857-10864) and the glucose repressible jbpl gene promoter as described by Hoffman and Winston (Hoffman C S and Winston F. 1990. Isolation and characterization of mutants constitutive for expression of the fbp1 gene of Schizosaccharomyces pombe. Genetics 124:807-816).

The transcription termination signal may be the 3′ flanking sequence of a eukaryotic gene which contains proper signals for transcription termination and polyadenylation. Suitable 3′ flanking sequences may, for example, be those of the gene naturally linked to the expression control sequence used, i.e. may correspond to the promoter. Alternatively, they may be different in which case the termination signal of the S. cerevisiae ADHI gene is optionally used.

Exemplary expression systems for the production of the Fc fusion protein described herein in bacteria include Bacillus subtilis, Bacillus brevis, Bacillus megaterium, Caulobacter crescentus, and, most importantly, Escherichia coli BL21 and E. coli K12 and their derivatives. Convenient promoters include but are not limited to trc promoter, tac promoter, lac promoter, lambda phage promoter p_{L}, the L-arabinose inducible araBAD promoter, the L-rhamnose inducible rhaP promoter, and the anhydrotetracycline-inducible tetA promoter/operator.

In some embodiments, the fusion protein, or the polynucleotide encoding the fusion protein, further comprises a signal sequence and/or a leader sequence (see e.g., Example 15). A signal sequence (sometimes referred to as signal peptide, targeting signal, localization signal, localization sequence, or transit peptide) is a short “pre-peptide” (usually 16-30 amino acids long) present at the N-terminus (or occasionally non-classically at the C-terminus or internally) of most newly synthesized secretory proteins. The signal sequence facilitates translocation of the expressed polypeptide to which it is attached into the endoplasmic reticulum. Signal peptide typically comprises a positively charged n-region, a hydrophobic h-region, and a neutral, polar c-region. At the end of the signal sequence, there is typically a stretch of amino acids that is recognized and cleaved by a signal peptidase and therefore named the cleavage site. The signal sequence is normally cleaved off in the course of the secretion process.

In some embodiments, a polynucleotide encoding the Fc fusion protein described herein can be fused to signal sequences which will direct the localization of a protein of the invention to particular compartments of a prokaryotic cell and/or direct the secretion of a protein of the invention from a prokaryotic cell. For example, in E. coli, one may wish to direct the expression of the protein to the periplasmic space. Examples of signal sequences or proteins (or fragments thereof) to which the proteins of the invention may be fused in order to direct the expression of the polypeptide to the periplasmic space of bacteria include, but are not limited to, the pelB signal sequence, the maltose binding protein signal sequence, the ompA signal sequence, the signal sequence of the periplasmic E. coli heat-labile enterotoxin B-subunit, and the signal sequence of alkaline phosphatase. Several vectors are commercially available for the construction of fusion proteins which will direct the localization of a protein, such as the μMAL series of vectors (New England Biolabs).

Leader sequences are polynucleotide regions located between the promoter and the coding region and are involved in the regulation of gene expression. Leader sequences comprise a short open reading frame coding for a leader peptide and a downstream adjacent region with the propensity of forming mutually exclusive secondary structures (stem-loops) by base-pairing of complementary sequences. The signal sequence and/or leader sequence may be heterologous or homologous to the organism used to produce the polypeptide (e.g., Pichia pastoris). Some exemplary sequences comprising leader sequences and/or signal sequences are as follows:

MRQVWFSWIVGLFLCFFNVSSAAPVNTTTEDETAQIPAEAVIGYS
DLEGDFDVAVLPFSNSTNNGLLFINTTIASIAAKEEGVSLEKREA
EA
(SEQ ID NO: 208; pOst1; see e.g.,
SEQ ID NO: 177);
MRFPSIFTAVLFAASSALAAPVNTTTEDETAQIPAEAVIGYSDLE
GDFDVAVLPFSASIAAKEEGVSLEKREAEA
(SEQ ID NO: 209; pro-d57-70; see e.g.,
SEQ ID NO: 179);
or
MRFPSIFTAVLFAASSALAEAEA
(SEQ ID NO: 210; rm_pro;
see e.g., SEQ ID NO: 181).

In some embodiments, the leader sequence and/or signal sequence comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a sequence selected from the group consisting of SEQ ID NO: 208-210. In some embodiments, the leader sequence and/or signal sequence comprises an amino acid sequence having at least 95%, 96%, 97%, 98% or 99% identity to a sequence selected from the group consisting of SEQ ID NO: 208-210. In some embodiments, the leader sequence and/or signal sequence comprises an amino acid sequence having at least 97%, 98% or 99% identity to a sequence selected from the group consisting of SEQ ID NO: 208-210.

In some embodiments, the leader sequence and/or signal sequence are at the N-terminus of the fusion protein. In some embodiments, the leader sequence and/or signal sequence are at the C-terminus of the fusion protein. In some embodiments, the leader sequence and/or signal sequence are at an internal position of the fusion protein. In some embodiments, the leader sequence and/or signal sequence are cleaved from the fusion protein during maturation and/or secretion.

In some embodiments, the fusion protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a sequence selected from the group consisting of SEQ ID NO: 177, 179, and 181. In some embodiments, the fusion protein comprises an amino acid sequence having at least 95%, 96%, 97%, 98% or 99% identity to a sequence selected from the group consisting of SEQ ID NO: 177, 179, and 181. In some embodiments, the fusion protein comprises an amino acid sequence having at least 97%, 98% or 99% identity to a sequence selected from the group consisting of SEQ ID NO: 177, 179, and 181.

In some embodiments, the Fc-SPINK fusion protein is encoded by a polynucleotide comprising one of SEQ ID NOs: 176, 178, or 180 or a nucleic acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to one of SEQ ID NOs: 176, 178, or 180.

Exemplary plant systems for expression of the Fc fusion protein described herein include tobacco, potato, rice, maize, soybean, alfalfa, tomato, lettuce and legume (summarized by Ma J K C et al. 2003. The production of recombinant pharmaceutical proteins in plants. Nat. Rev. Genet. 4:794-805). Expression of recombinant proteins in plant systems may be directed by suitable regulatory elements to specific organs or tissues such as fruits, seeds, leaves or tubers. Alternatively, proteins may be secreted from the roots. Within the cell, proteins may be targeted to particular compartments, e.g. the endoplasmic reticulum, protein bodies or plastids. There the product may accumulate to higher levels or undergo particular forms of posttranslational modification.

Exemplary examples for large-scale transgenic expression systems (for review see Pollock D P. 1999. Transgenic milk as a method for the production of recombinant antibodies. J Immunol Methods 231:147-157) include rabbit (Chrenek P et al. 2007. Expression of recombinant human factor VIII in milk of several generations of transgenic rabbits. Transgenic Res. 2007 Jan. 31), goat (Lazaris A et al. 2006. Transgenesis using nuclear transfer in goats. Methods Mol Biol. 348:213-26), pig and cattle.

A variety of kits and components can be prepared for use in the methods described herein, depending upon the intended use of the kit. Accordingly, in another aspect, provided herein is a kit comprising a Fc fusion described herein or a nucleic acid encoding a Fc fusion described herein. A kit is any manufacture (e.g., a package or container) comprising a Fc fusion protein or a polynucleotide encoding a Fc fusion described herein. The manufacture can be promoted, distributed, or sold as a unit for performing the methods described herein.

The kits described herein can optionally comprise additional components and reagents. As will be appreciated by one of skill in the art, components of the kit can be provided in any desired form, e.g., in a lyophilized form, a liquid form, a solid form, or a concentrated. In some embodiments of the various aspects described herein, the kit can comprise ampoules, syringes, or the like.

In some embodiments, the kit can comprise informational material. The informational material can be descriptive, instructional, marketing or other material that relates to the methods described herein. The informational material of the kits is not limited in its form. In some embodiments, the informational material can include information about production of the reagents, concentration, date of expiration, batch or production site information, and so forth. In one embodiment, the informational material relates to methods for using or administering the components of the kit.

It is notes that the components of a kit can provided singularly or in any combination as a kit. Such a kit includes the components described herein and packaging materials thereof.

In some embodiments, the compositions in a kit can be provided in a watertight or gas tight container which in some embodiments is substantially free of other components of the kit. For example, the reagents described herein can be supplied in more than one container, e.g., it can be supplied in a container having sufficient reagent for a predetermined number of applications, e.g., 1, 2, 3 or greater. One or more components as described herein can be provided in any form, e.g., liquid, dried or lyophilized form. Liquids or components for suspension or solution of the reagents can be provided in sterile form and should not contain microorganisms or other contaminants. When the components described herein are provided in a liquid solution, the liquid solution preferably is an aqueous solution.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of viral infection. A subject can be male or female.

A subject can be one who has been previously diagnosed with or identified as suffering from or having a condition in need of treatment for pancreatitis or one or more complications related to pancreatitis, and optionally, have already undergone treatment for pancreatitis, or the one or more complications related to pancreatitis. Alternatively, a subject can also be one who has not been previously diagnosed as having pancreatitis or one or more complications related pancreatitis. For example, a subject can be one who exhibits one or more risk factors for pancreatitis or one or more complications related to pancreatitis or a subject who does not exhibit risk factors. A “subject in need” of testing for a particular condition can be a subject having that condition, diagnosed as having that condition, or at risk of developing that condition.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

The terms “wild-type” or “wt” or “native” as used herein is meant an amino acid sequence or a nucleotide sequence that is found in nature, including allelic variations. A wild-type protein, polypeptide, antibody, immunoglobulin, IgG, polynucleotide, DNA, RNA, and the like has an amino acid sequence or a nucleotide sequence that has not been intentionally modified.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

The term “amino acid substitution” refers to the replacement of at least one existing amino acid residue in a predetermined or native amino acid sequence with a different “replacement” amino acid. A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested confirm that a desired activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

The term “amino acid insertion” refers to the insertion of one or more additional amino acids into a predetermined or native amino acid sequence. The insertion can be one, two, three, four, five, or up to twenty amino acid residues.

The term “amino acid deletion” refers to removal of at least one amino acid from a predetermined or native amino acid sequence. The deletion can be one, two, three, four, five, or up to twenty amino acid residues.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide's activity according to the assays described herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan to generate and test artificial variants.

The term “nucleic acid” refers to a deoxyribonucleotide or ribonucleotide and polymers thereof in either single strand or double strand form. The term “nucleic acid” is used interchangeably with gene, nucleotide, polynucleotide, cDNA, DNA, and mRNA. The polynucleotides can be in the form of RNA or DNA. Polynucleotides in the form of DNA, cDNA, genomic DNA, nucleic acid analogs, and synthetic DNA are within the scope of the present invention. Unless specifically limited the term encompasses nucleic acids containing known analogues of natural nucleotides that have similar binding propertied as the natural nucleic acid. Unless specifically limited, a particular nucleotide sequence also encompasses conservatively modified variants thereof (for example, those containing degenerate codon substitutions) and complementary sequences as well as the as well as the sequences specifically described.

The polynucleotides can be composed of any polyribonucleotide or polydeoxyribonucleotide, which can be unmodified RNA or DNA or modified RNA or DNA. For example, polynucleotides can be composed of single or double stranded regions, mixed single or double stranded regions. In addition, the polynucleotides can be triple stranded regions containing RNA or DNA or both RNA and DNA. Modified polynucleotides include modified bases, such as tritylated bases or unusual bases such as inosine. A variety of modification can be made to RNA and DNA, thus polynucleotide includes chemically, enzymatically, or metabolically modified forms.

The DNA may be double-stranded or single-stranded, and if single stranded, may be the coding (sense) strand or non-coding (anti-sense) strand. The coding sequence that encodes the polypeptide may be identical to the coding sequence provided herein or may be a different coding sequence, which sequence, as a result of the redundancy or degeneracy of the genetic code, encodes the same polypeptides as the DNA provided herein.

A variant DNA or amino acid sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

In some embodiments of the various aspects described herein, a polypeptide, nucleic acid, or cell as described herein can be engineered. As used herein, “engineered” refers to the aspect of having been manipulated by the hand of man. For example, a polynucleotide is considered to be “engineered” when at least one aspect of the polynucleotide, e.g., its sequence, has been manipulated by the hand of man to differ from the aspect as it exists in nature.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The term “statistically significant” or “significantly” refers to statistical significance and generally means a two standard deviations (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean+1%. In some embodiments of the various aspects described herein, the term “about” when used in connection with percentages can mean±5%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Aspects of the disclosure also can be described with the following Embodiments A-AG:

Embodiment A: An Fc fusion protein comprising a pancreatic trypsin inhibitor (PTI) polypeptide linked to an Fc region, wherein the PTI polypeptide is a serine protease inhibitor Kazal-type 1 (SPINK1) polypeptide or a bovine pancreatic trypsin inhibitor (BPTI) polypeptide, wherein the SPINK1 polypeptide does not comprise a mutation, and wherein the Fc region comprises a hinge region, an IgG CH2 domain and an IgG CH3 domain.

Embodiment B: The Fc fusion protein of any of the preceding paragraphs, wherein the N-terminus of the Fc region is linked to the C-terminus of the PTI polypeptide.

Embodiment C: The Fc fusion protein of any of the preceding paragraphs, wherein the C-terminus of the Fc region is linked to the N-terminus of the PTI polypeptide.

Embodiment D: The Fc fusion protein of any of the preceding paragraphs, wherein the PTI polypeptide is the SPINK polypeptide.

Embodiment E: The Fc fusion protein of any of the preceding paragraphs, wherein the SPINK polypeptide comprises an amino acid sequence having at least 85% identity to an amino acid selected from the group consisting of:

(SEQ ID NO: 14)
DSLGREAKCYNELNGCTKIYDPVCGTDGNTYPNECVLCFENRKRQ
TSILIQKSGPC;
(SEQ ID NO: 15)
DSLGREAKCYNELNGCTKIYDPVCGTDGNTYPNECVLCFENRKRQ
TSILIQKSGPCGPEQKLISEEDLNSALVPRGSRL VDHHHHHH;
(SEQ ID NO: 16)
MKVTGIFLLSALALLSLSGNTGADSLGREAKCYNELNGCTKIYDP
VCGGNTYPNECVLCFENRKRQTSILIQKSGPC;
(SEQ ID NO: 17)
MKVTGIFLLSALALLSLSGNTGADSLGREAKCYNELNGCTKIYNP
VCGGNTYPNECVLCFENRKRQTSILIQKSGPC;
(SEQ ID NO: 18)
MKVTGIFLLSALALLSLSGNTGADSLGREAKCYNELNGCTKIYDP
VCGGDTYPNECVLCFENRKRQTSILIQKSGPC;
(SEQ ID NO: 19)
MKVTGIFLLSALALLSLSGNTGADSLGREAKCYNELNGCTKIYDP
VCGGNTYPNECVLCFEGRKRQTSILIQKSGPC;
(SEQ ID NO: 20)
MKVTGIFLLSAFALLSLSGNTGADSLGREAKCYNELNGCTKIYDP
VCGGNTYPNECVLCFENRKRQTSILIQKSGPC;
(SEQ ID NO: 21)
MKVTGIFLLSALAPLSLSGNTGADSLGREAKCYSELNGCTKIYDP
VCGGNTYPNECVLCFENRKRQTSILIQKSGPC;
(SEQ ID NO: 22)
MKVTGIFLLSALALLSLSGNTGADSLGREAKCYNELNGCTKIYDP
VCGGNTYSNECVLCFENRKHQTSILIQKSGPC;
and
(SEQ ID NO: 186)
AKVTGKEASCHDAVAGCPRIYDPVCGTDGITYANECVLCFENRKR
IEPVLIRKGGPC.

Embodiment F: The Fc fusion protein of any of the preceding paragraphs, wherein the SPINK polypeptide comprises an amino acid sequence having at least 85% identity to SEQ ID NO: 85.

Embodiment G: The Fc fusion protein of any of the preceding paragraphs, wherein the SPINK polypeptide comprises an amino acid sequence having at least 85% identity to an amino acid sequence selected from the group consisting of: SEQ ID NOs: 1, 6-11 and 85-114.

Embodiment H: The Fc fusion protein of any of the preceding paragraphs, wherein the PTI polypeptide is a BPTI polypeptide.

Embodiment I: The Fc fusion protein of any of the preceding paragraphs, wherein

the BPTI polypeptide comprises an amino acid sequence having at least 95% identity to the amino acid sequence:

(SEQ ID NO: 3)
RPDFCLEPPYTGPCKARIIRYFYNAKAGLCQTFVYGGCRAKRNNF
KSAEDCMRTCGGA

Embodiment J: The Fc fusion protein of any of the preceding paragraphs, wherein the BPTI polypeptide comprises an amino acid sequence having at least 99% identity to SEQ ID NO: 3.

Embodiment K: The Fc fusion protein of any of the preceding paragraphs, wherein the BPTI polypeptide comprises a substitution at position 16 of SEQ ID NO: 3.

Embodiment L: The Fc fusion protein of any of the preceding paragraphs, wherein the BPTI comprises an A to H or A to S substitution at position 16.

Embodiment M: The Fc fusion protein of any of the preceding paragraphs, wherein the Fc region is a human IgG Fc region.

Embodiment N: The Fc fusion protein of any of the preceding paragraphs, wherein the Fc region is a IgG1 Fc region.

Embodiment O: The Fc fusion protein of any of the preceding paragraphs, wherein the Fc region is a IgG3 Fc region.

Embodiment P: The Fc fusion protein of any of the preceding paragraphs, wherein the hinge region has a length of about 10 to about 20 amino acids.

Embodiment Q: The Fc fusion protein of any of the preceding paragraphs, wherein the Fc region comprises an amino acid sequence having at least 85% identity to an amino acid sequence selected from the group consisting of:

(SEQ ID NO: 36)
EPKSSDKTHTCPPCPAPELLGGPSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPGA;
(SEQ ID NO: 37)
EPKSSDKTHTCPPCPAPELLGGPSVFLFPPKPKDTLMISRTPEVT
CVVVDVSHEDPEVKFNWYVDGVEVHNAKTKPREEQYDSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPGA;
(SEQ ID NO: 38)
CPAPELLGGPSVFLFPPKPKDTLMISRTPEVTCVVVDVSHEDPEV
QFKWYVDGVEVHNAKTKPREEQYNSTFRVVSVLTVLHQDWLNGKE
YKCKVSNKALPAPIEKTISKTKGQPREPQVYTLPPSREEMTKNQV
SLTCPVKGFYPSDIAVEWESSGQPENNYNTTPPMLDSDGSFFLYS
KLTVDKSRWQQGNIFSCSVMHEALHNhyTQKSLSLSPGA;
(SEQ ID NO: 39)
epkscdkthtcppCPAPELLGGPSVFLFPPKPKDTLMISRTPEVT
CVVVDVSHEDPEVQFKWYVDGVEVHNAKTKPREEQYNSTFRVVSV
LTVLHQDWLNGKEYKCKVSNKALPAPIEKTISKTKGQPREPQVYT
LPPSREEMTKNQVSLTCPVKGFYPSDIAVEWESSGQPENNYNTTP
PMLDSDGSFFLYSKLTVDKSRWQQGNIFSCSVMHEALHNhyTQKS
LSLSPGa;
and
(SEQ ID NO: 40)
EPeeSDKTHTCPPCPAPELLGGPSVFLFPPKPKDTLMISRTPEVT
CVVVDVSHEDPEVKFNWYVDGVEVHNAKTKPREEQYdSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPGe.

Embodiment R: The Fc fusion protein of any of the preceding paragraphs, wherein the Fc region comprises an amino acid sequence having at least 85% identity to an amino acid sequence selected from the group consisting of SEQ ID NOs: 187-204.

Embodiment S: The Fc fusion protein of any of the preceding paragraphs, comprising the amino acid sequence

(X)₁IEPxxSDKTHTCPPCPAPELLGGPSVFLFPPKPKDTL
MISRTPEVTCVVVDVSHEDPEVKFNWYVDGVEVHNAKTKPR
EEQYxSTYRVVSVLTVLHQDWLNGKEYKCKVSNKALPAPIE
KTISKAKGQPREPQVYTLPPSRDELTKNQVSLTCLVKGFYP
SDIAVEWESNGQPENNYKTTPPVLDSDGSFFLYSKLTVDKS
RWQQGNVFSCSVMHEALHNHYTQKSLSLSPG(X)ₘDSLGRE
AKCYNELNGCTKIYDPVCGTDGNTYPNECVLCFENRKRQTS
ILIQKSGPC(X)ₙ,

where each X is independently aspartic acid (D) or glutamic acid (E), and the subscripts ‘ ’1′, ‘m’ and ‘n’ and N independently range from 1 to up to 10 or more.

Embodiment T: The Fc fusion protein of any of the preceding paragraphs, comprising the amino acid sequence

(SEQ ID NO: 9)
EPeeSDKTHTCPPCPAPELLGGPSVFLFPPKPKDTLMISRTPE
VTCVVVDVSHEDPEVKFNWYVDGVEVHNAKTKPREEQYdSTYR
VVSVLTVLHQDWLNGKEYKCKVSNKALPAPIEKTISKAKGQPR
YEPQVYTLPPSRDELTKNQVSLTCLVKGFPSDIAVEWESNGQP
ENNYKTTPPVLDSDGSFFLYSKLTVDKSRWQQGNVFSCSVMHE
ALHNHYTQKSLSLSPGeDSLGREAKCYNELNGCTKIYDPVCGT
DGNTYPNECVLCFENRKRQTSILIQKSGPC.

Embodiment U: The Fc fusion protein of any of the preceding paragraphs, further comprising an inflammatory cytokine inhibitor.

Embodiment V: The Fc fusion protein of any of the preceding paragraphs, wherein the inflammatory cytokine inhibitor comprises an IL-1Ra domain.

Embodiment W: The Fc fusion protein of any of the preceding paragraphs, wherein the IL-1Ra domain comprises an amino acid sequence having at least 85% identity to an amino acid sequence selected from the group consisting of SEQ ID NOs: 205-207.

Embodiment X: The Fc fusion protein of any of the preceding paragraphs, having at least 85% identity to an amino acid sequence selected from the group consisting of SEQ ID NOs: 88, 89, 93-97.

Embodiment Y: The Fc fusion protein of any of the preceding paragraphs, further comprising a signal sequence and/or a leader sequence.

Embodiment Z: The Fc fusion protein of any of the preceding paragraphs, wherein signal sequence and/or leader sequence comprises an amino acid sequence having at least 85% identity to an amino acid sequence selected from the group consisting of SEQ ID NOs: 208-210.

Embodiment AA: A pharmaceutical composition comprising a Fc fusion protein of any of the preceding paragraphs and a pharmaceutically acceptable carrier or excipient.

Embodiment AB: A nucleic acid encoding a Fc fusion protein of any of the preceding paragraphs.

Embodiment AC: A cell comprising a nucleic acid of any of the preceding paragraphs.

Embodiment AD: A kit comprising a nucleic acid of any of the preceding paragraphs or a fusion protein of any of the preceding paragraphs.

Embodiment AE: A method for treating pancreatitis, the method comprising administering a Fc fusion protein of any of the preceding paragraphs to a subject in need thereof.

Embodiment AF: The method of any of the preceding paragraphs, wherein said administering is intravenous (IV) or intraperitoneal (IP) administration.

Embodiment AG: A method for enhancing outcomes of cardiac surgery in a human, the method comprising administering a fusion protein of any of the preceding paragraphs to a subject in need thereof.

The technology described herein is further illustrated by the following examples which in no way should be construed as being further limiting.

### Examples

### Example 1: Mammalian Expression of Novel Fc Fusion Protein Therapeutics for the Treatment of Pancreatitis

### Materials and Methods

Construction of expression plasmids: Wildtype IgG1, IgG3, BPTI and SPINK genetic sequences compiled from natural coding sequences in GenBank. DNA fragments encoding fusion proteins (BPTI-Fc and Fc-BPTI) were chemically synthesized as gBlocks by Integrated DNA Technologies (IDT; Coralville, IA). Sequences were appended with a 10 amino acid C terminus Myc tag (EQKLISEEDL, SEQ ID NO: 84), and a 6 amino acid C terminus His tag (HHHHHH, SEQ ID NO: 83) to facilitate protein identification and purification. Sequences were ordered from Integrated DNA Technologies. Each gene was cloned into a pOptivec mammalian expression vector using Gibson assembly master mix (New England BioLabs Inc.) according to the manufacturer's instruction, for use in both transient transfection and stable pool formation. BPTI mutants were derived from these newly constructed plasmids via PCR based site-directed mutagenesis using the Phusion® Hot Start Flex DNA Polymerase master mix (New England BioLabs Inc.) according to the manufacturer's instruction and again cloned using Gibson assembly. Primers for mutagenesis were ordered from Integrated DNA Technologies. To isolate correctly assembled plasmids, 5 μl of the Gibson reaction was transformed into 50 μl of C2987I E. coli cells (New England BioLabs Inc.) according to the supplier's recommendations. The cells were then plated onto Amp^{R} LB plates and left to grow overnight at 37° C. 3-5 colonies were selected and plasmid purified using the QIAGEN Plasmid Mini Kit (QIAGEN) with a minimum concentration of 50 ng/μl eluted in double distilled H₂O. Results were sequence verified via Sanger Sequencing (GENEWIZ). Following this process, properly formed plasmids were purified for mammalian cell transfection using the QIAGEN Plasmid Maxi Kit (QIAGEN) with a minimum concentration of 300 ng/μl eluted in double distilled H₂O. Glycerol stocks of all clones containing desired, correctly assembled plasmids were made and stored for later use.

Cell culture: Human embryonic kidney suspension cells (FreeStyle 293-F), and dihydrofolate reductase negative Chinese Hamster Ovary suspension cells (FreeStyle CHO-DG44) were obtained from Invitrogen (Carlsbad, CA), and were cultured in FreeStyle 293 Expression medium and complete CD DG44 (Invitrogen) respectively. Prior to use, cryovials of cells were stored in liquid nitrogen.

Protein expression, purification, identification and quantification: Transient expression was done in 293-F, and stable expression was done in CHO DG44 cells using pOptiVEC plasmids. Three to five days prior to transient transfection, 293-F cell cultures were established in disposable, sterile, polycarbonate Erlenmeyer flasks and incubated at 37° C. in 8% CO₂ with shaking at 2.35×g, according to manufacturer instructions. Cell counting and viability was assessed on a ThermoFisher Scientific Countess™ Automated Cell Counter according to manufacturer instruction. Once cell suspension reached greater than 90% viability and 1×10⁶ cells/ml, cells were transfected with the desired expression vector at a concentration of 1 μg/ml using Opti-MEM I Reduced-Serum Medium and 293 fectin following supplier protocol (INVITROGEN). Stably transfected cells were selected by hypoxanthine/thymidine (HT)-deficient CD OptiCHO medium (INVITROGEN), and were subjected to one round of methotrexate (MTX; SIGMA-ALDRICH) genomic amplification as described before.

Following five days of production, cells were spun down at 3000 g for 10 mins, culture supernatant was collected, and the product filtered using a 0.22 μM pore, sterile vacuum filter system (CORNING) at room temperature. After sterile collection, supernatant was concentrated at 4° C. to less than 5 mL over approximately 12 hours using 10 kDa cut-off MACROSEP ADVANCE centrifugal device (PALL). Concentrate was stored overnight at 4° C., and followed by next day assaying for protein expression. Concentrated protein was then purified via either His binding or Protein A binding. His binding consisted of concentrated protein bound to 0.5-1 mL of His60 nickel or HisTalon cobalt resin (TAKARA BIO) for 0.5 h at 4° C. while rotating in a 10-mL Pierce disposable column (THERMO SCIENTIFIC), and then washed and eluted using His60 or HisTalon Buffer Set (TAKARA BIO) according to the manufacturer instructions. Protein A binding consisted of concentrated protein bound to 0.5-1 mL of Protein A agarose beads (EMD Millipore Corp), spin purified through centrifugation in 15 mL falcon tubes per manufacturer instructions. Cell supernatant and purification fractions were analyzed by SDS-PAGE followed by Coomassie Blue staining. Eluted proteins were combined, desalted into endotoxin-free PBS (TEKNOVA: 137 mM NaCl, 1.4 mM KH₂PO₄, 4.3 mM Na₂HPO₄, and 2.7 mM KCl, pH 7.4) using ECONO-PAC 10DG columns (BIO-RAD), and concentrated to <1 mL using MACROSEP ADVANCE centrifugal device. Proteins were stored at 4° C. throughout the described process, ultimately stored as aliquots at −80° C., and thawed once before use. Only endotoxin-free reagents were used. Final protein yield was assessed by SDS page protein gel using serial dilutions of 100 μg/mL Bovine Serum Albumin to set a standard baseline. Gel was run for 1 hour and 20 minutes at 120 amps, then stained using Coomassie Blue and visualized. Stained protein at the appropriate size was quantified via ImageJ and compared to BSA standard to calculate final concentration. Positive protein identification was assayed by Western blotting using anti-His₆-HRP antibody (ABCAM).

Trypsin Inhibition assay: Freshly thawed inhibitor protein was analyzed for trypsin inhibitory efficacy using a Trypsin Activity Colorimetric Assay (BIOVISION). Per manufacturer instructions, a PNa standard for absorbance, alongside kit provided trypsin which had been incubated with serial dilutions of either the thawed inhibitor or the control provided in the kit, were loaded onto a 96 well plate. A color releasing trypsin substrate was then added to each well, and absorbance was immediately measured at 405 nm every 2 minutes per manufacturer instructions, until plateau was reached. Absorbance at 405 nm was read on a BIOTEK SYNERGY NEO HTS microplate reader.

Binding Affinity assay: Lyophilized trypsin was reconstituted in water per supplier instruction, and then biotinylated at a ratio of 1:1 in preparation for biolayer interferometry using the BLITz machine (FORTEBIO) per manufacturer instructions. Biotinylated FcRn was purchased from TODO. Biotinylated target molecule was diluted to a concentration of 30 ng/uL in PBS in preparation for binding to Streptavidin Dip and Read Biosensors (FORTEBIO), as suggested by the manufacturer. Inhibitor protein binding affinity to the desired target molecule was measured using the advanced kinetics assay, using PBS (TEKNOVA: 137 mM NaCl, 1.4 mM KH₂PO₄, 4.3 mM Na₂HPO₄, and 2.7 mM KCl, pH 7.4) serial, 10-fold dilutions per manufacturer protocol. The manufacturer provided BLITz reporting software used to calculate K_{D}, kₐ and k_{d}.^{[13]}

Amylase Inhibition assay: Freshly thawed animal serum was analyzed for amylase activity using an Amylase Activity Colorimetric Assay (BIOVISION). Per manufacturer instructions, a PNa standard for absorbance, alongside 1 μL samples of serum, were loaded onto a 96 well plate. A color releasing amylase substrate was then added to each well, and absorbance was immediately measured at 405 nm every 2 minutes per manufacturer instructions. Absorbance at 405 nm was read on a BIOTEK Synergy Neo HTS microplate reader.

### Results

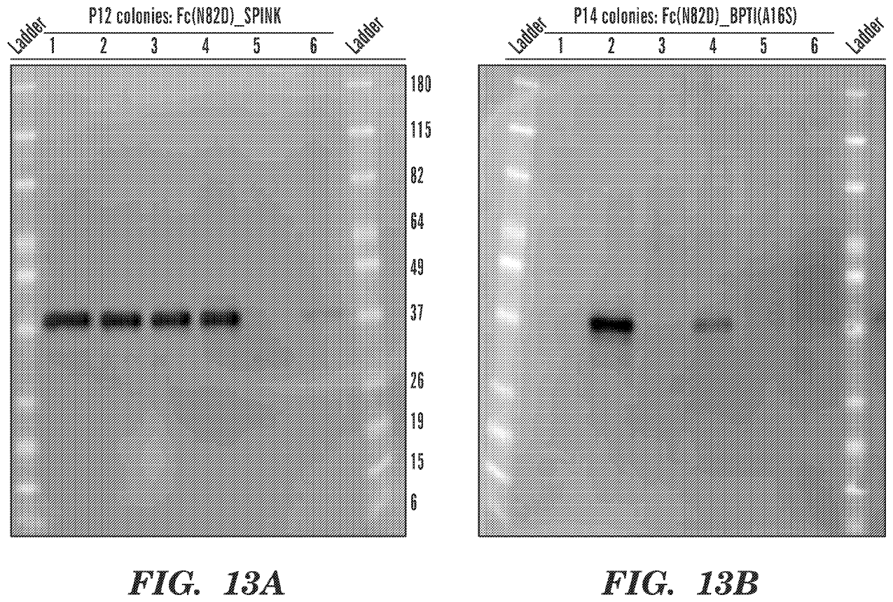

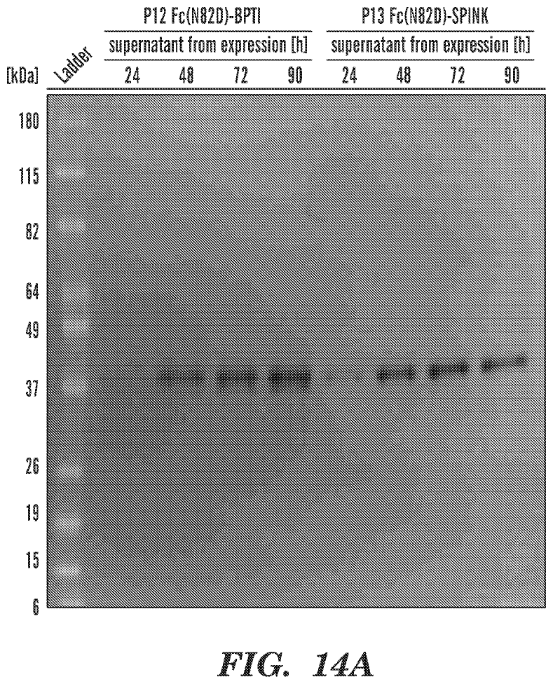

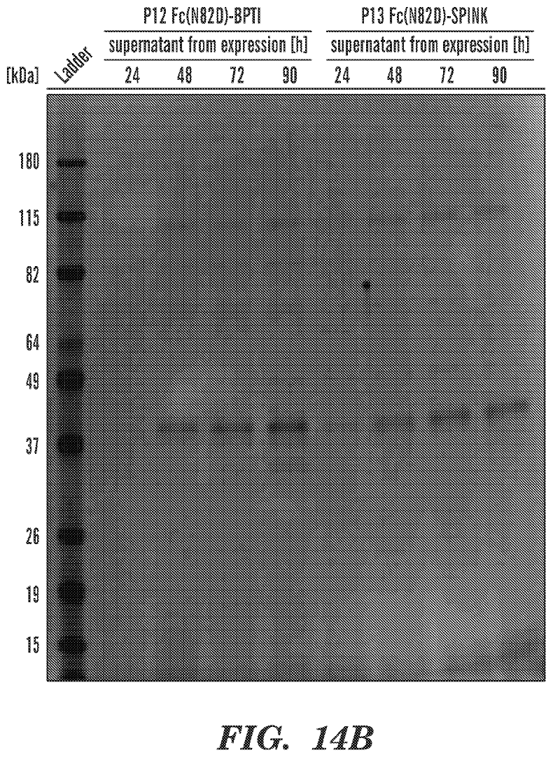

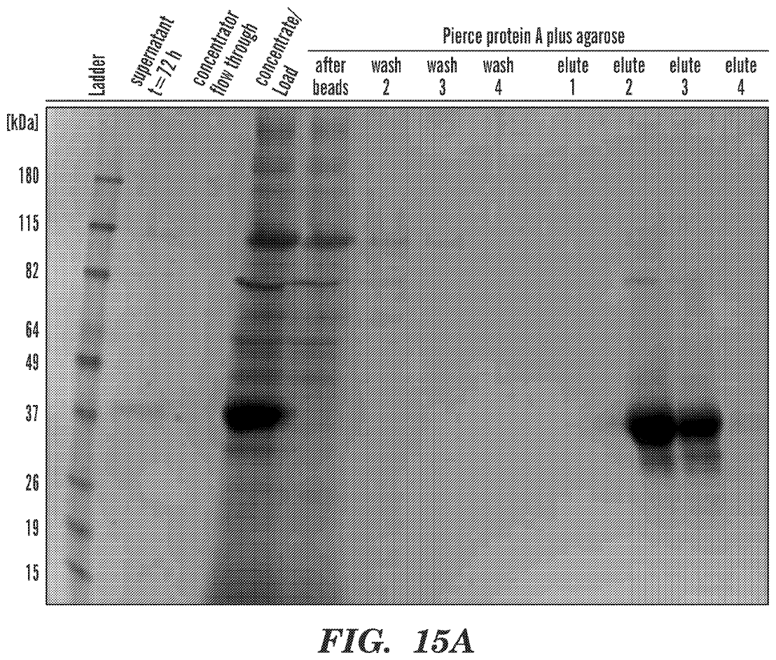

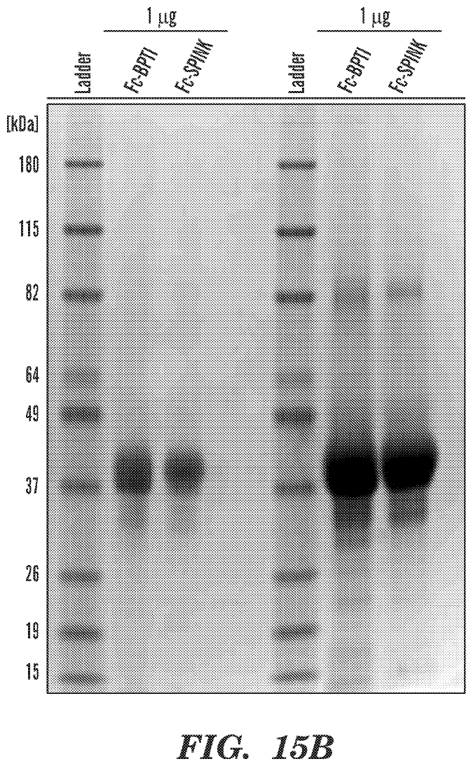

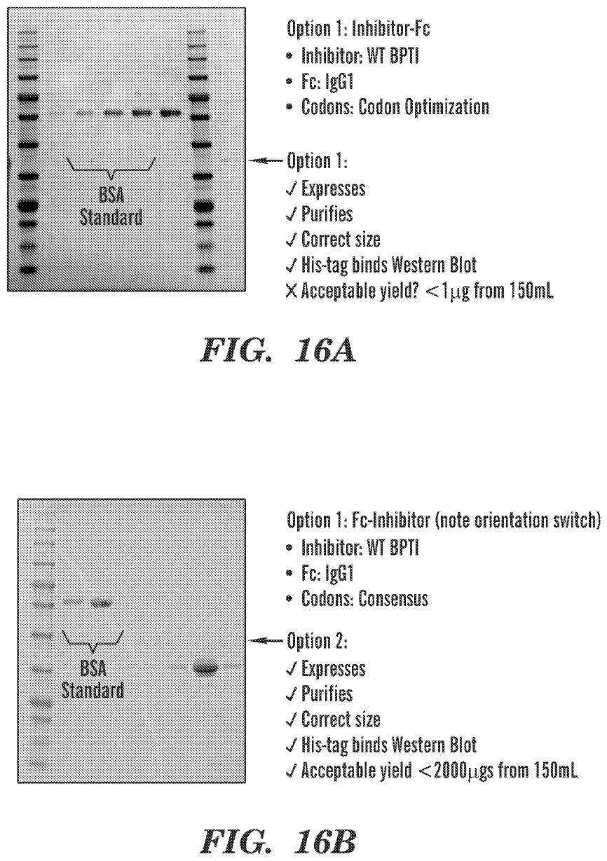

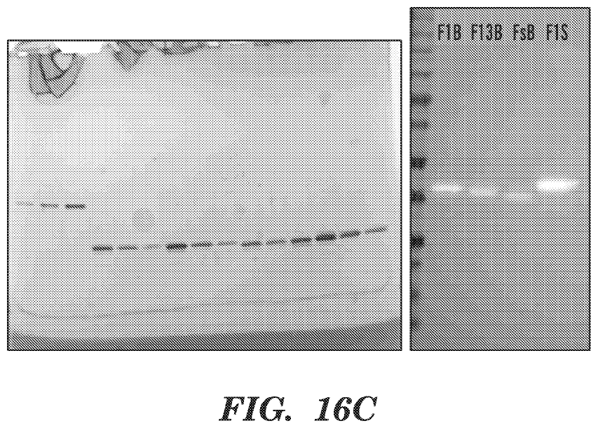

Results of Fc fusion protein production are shown in FIGS. 13-15B. As seen from FIGS. 16A and 16B, orientation of the Fc fusion protein, Fc-Inhibitor versus Inhibitor-Fc, can affect the structural integrity of the Fc fusion protein, exposure of FcRn binding site, steric hindrance of the attached inhibitory element, and access to properly folded elements necessary for protein purification.

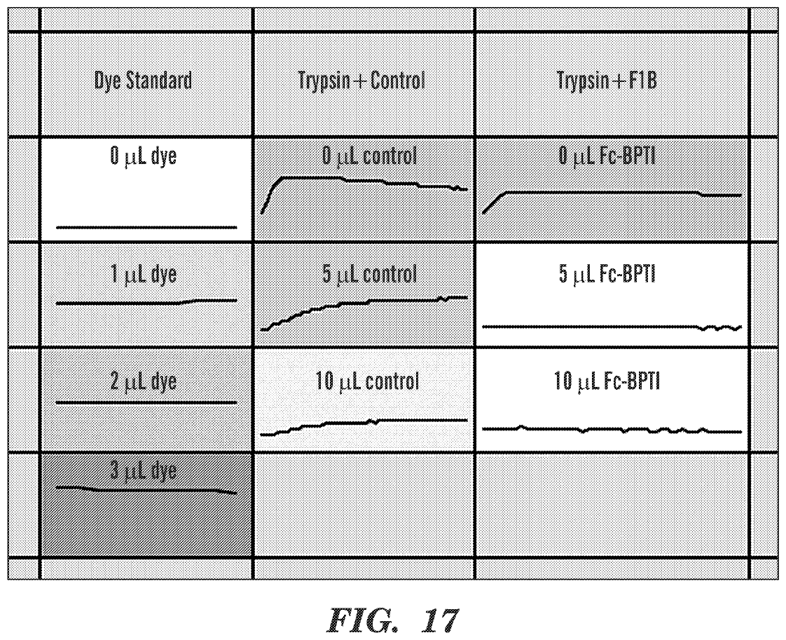

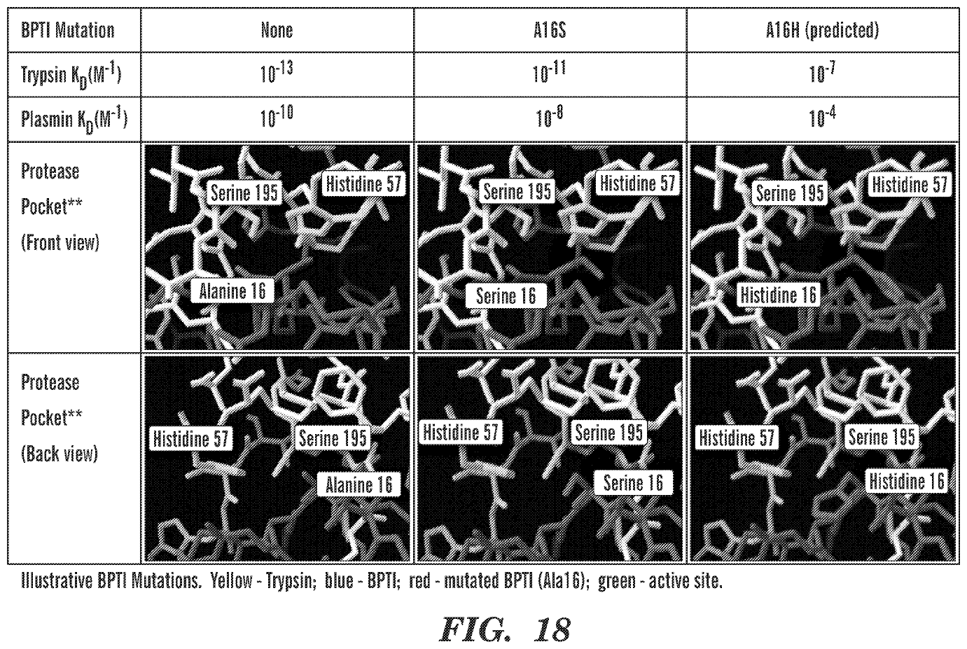

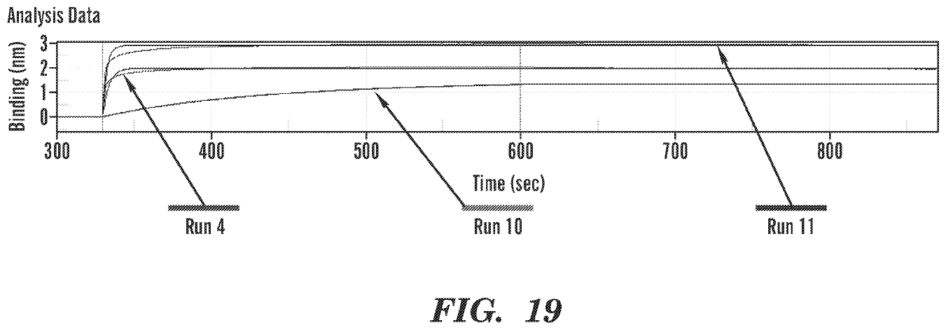

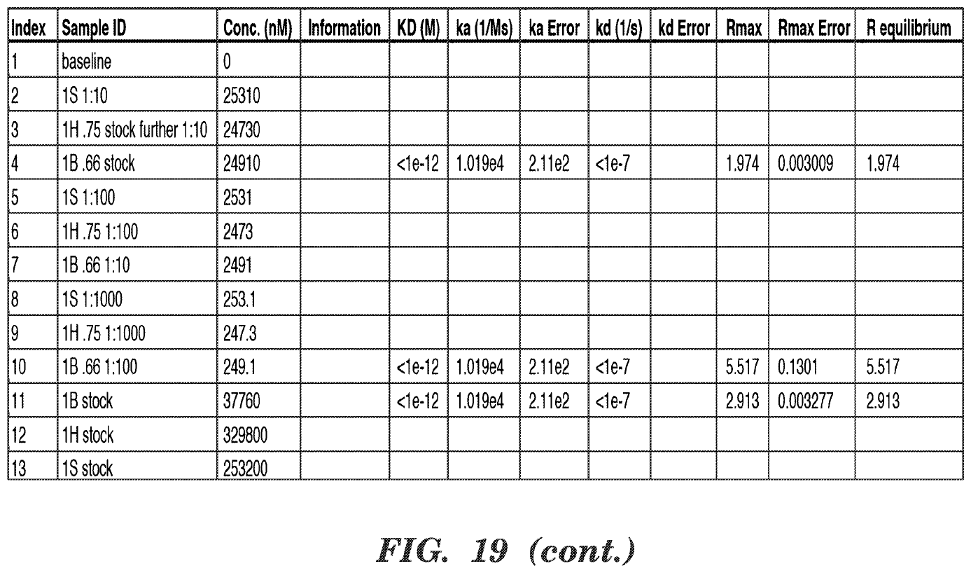

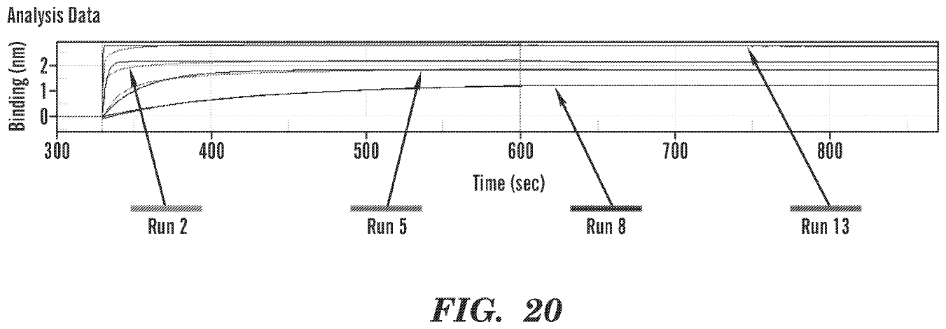

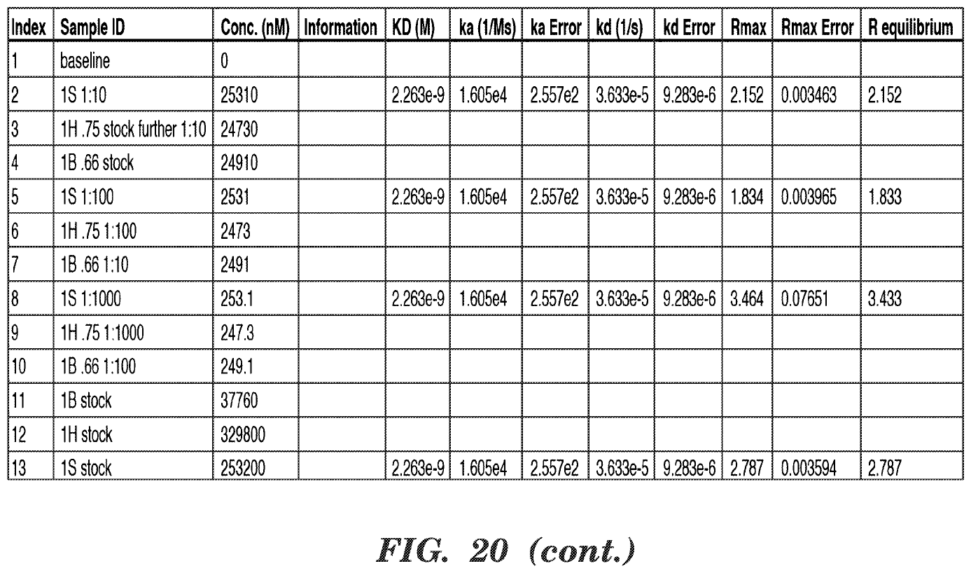

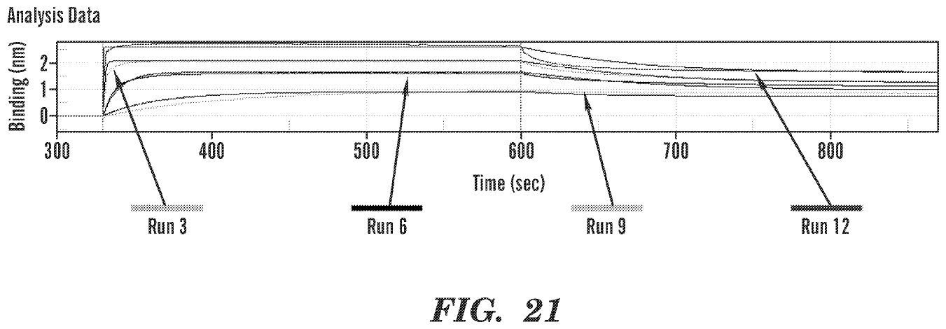

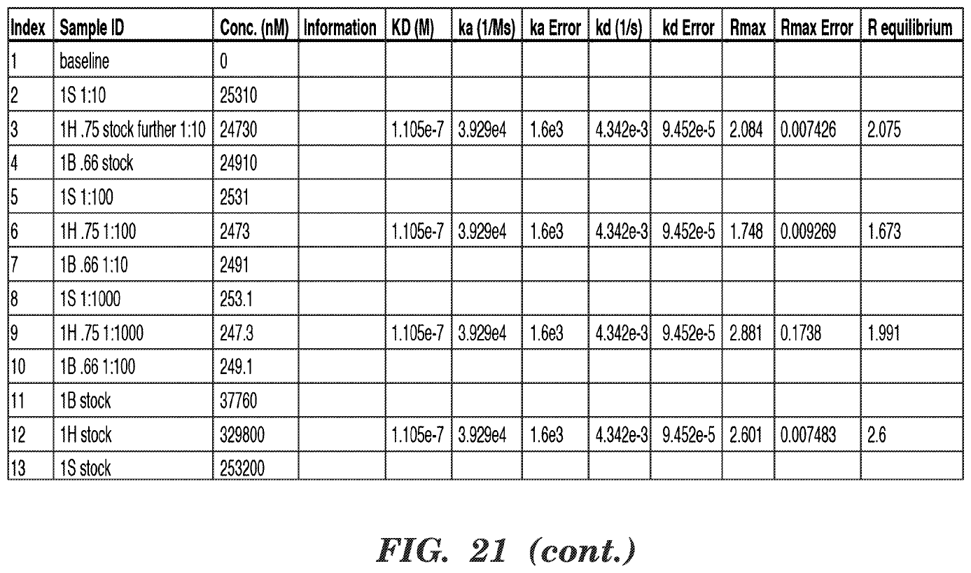

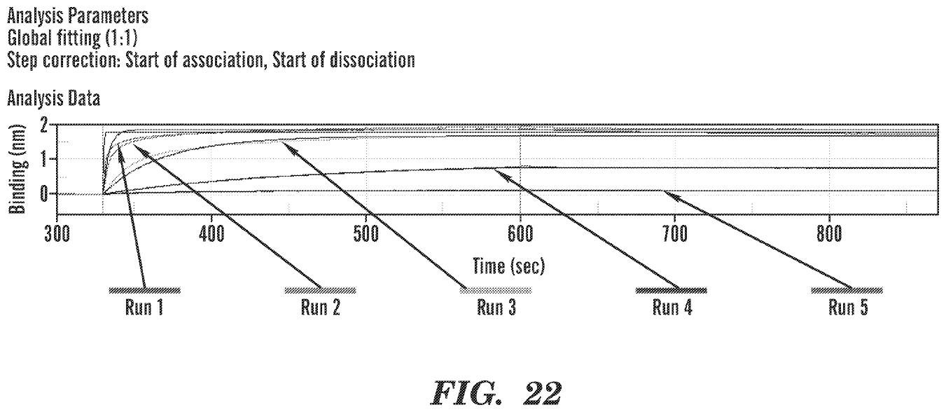

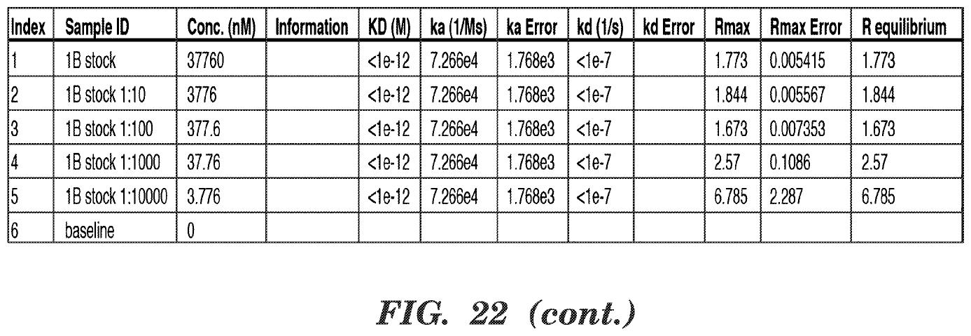

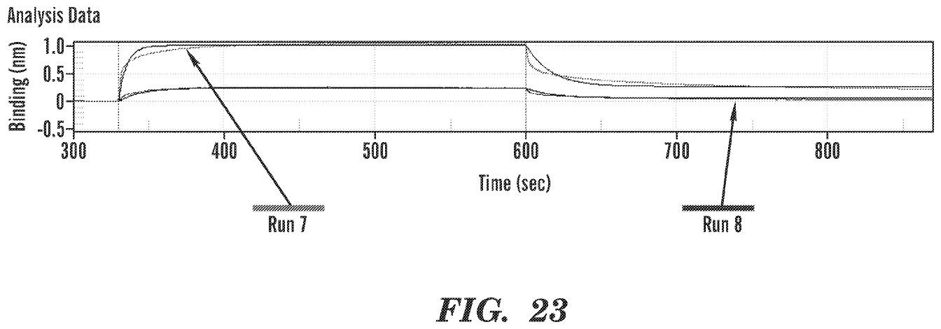

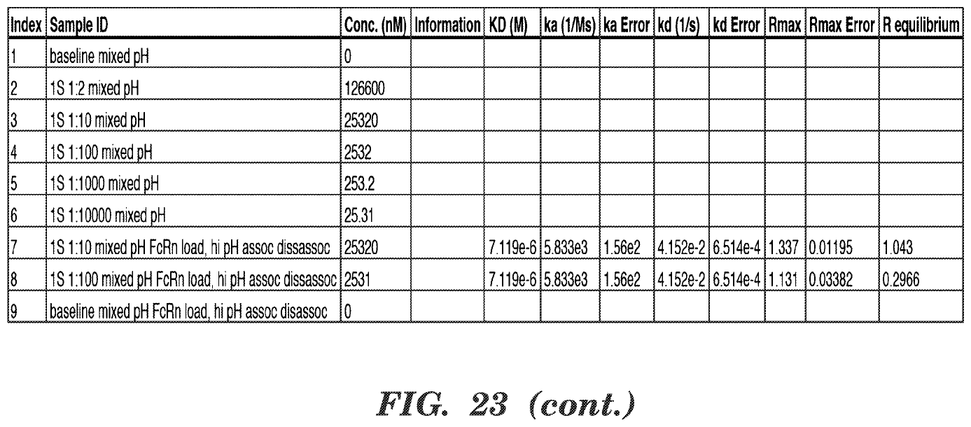

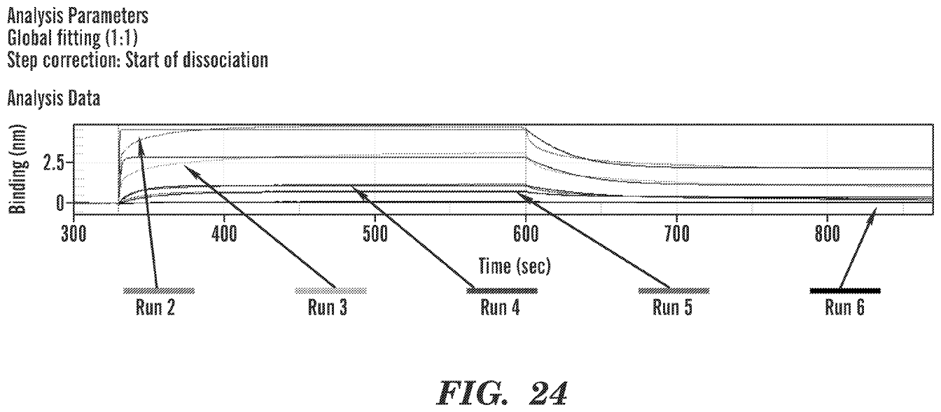

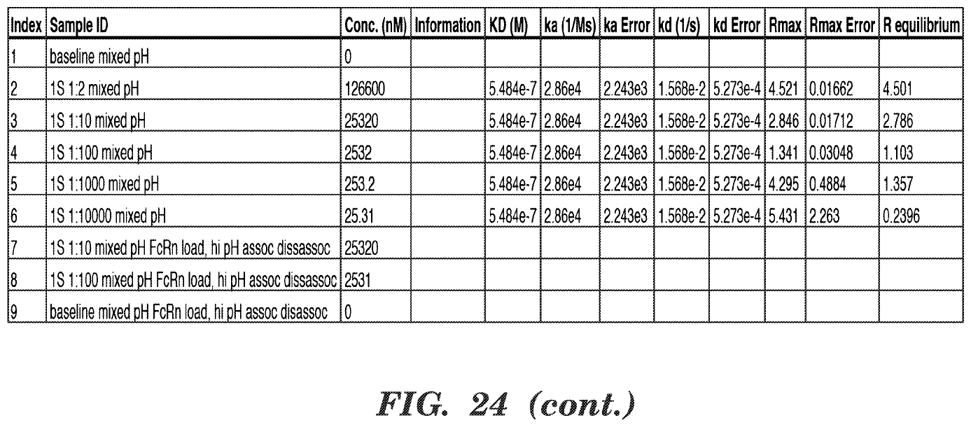

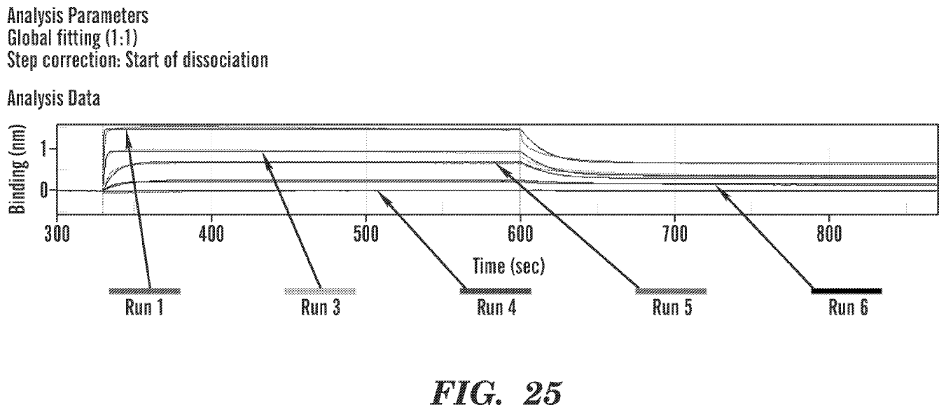

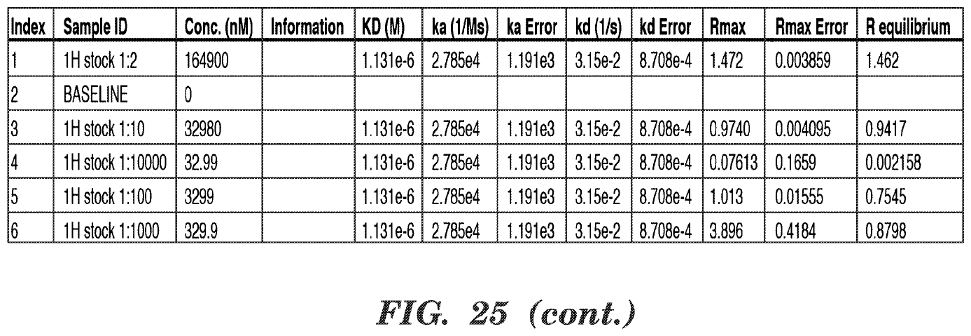

Fc fusion proteins of the invention retain trypsin inhibiting activity (FIG. 17), have improved affinity for trypsin over others (FIGS. 18-21), show unchanged affinity over time (FIG. 22), show 10× improvement in FcRn binding from pH 7.4 to pH 6.5 (FIGS. 23 and 24). As seen from FIG. 25, substitution at position 16 of the BPTI alters kinetics of binding with trypsin (pH 7.4 in first section had K_{D} of 10⁻⁷, now K_{D} of 10⁻⁶ (i.e., 10× worse).

### Example 2: Yeast Expression of Novel Fc Fusion Protein Therapeutics for the Treatment of Pancreatitis

Fc fusion proteins were produced in Pichia pastoris using the EASYSELECT Pichia Expression Kit from INVITROGEN. Yeast codon-optimized sequences for Fc fusion proteins were inserted into the vector pPICZαA by Gibson Assembly. pPICZαA is a shuttle vector for cloning in E. coli and methanol-induced, secreted expression in yeast. After verification of plasmids by Sanger sequencing, plasmids were transformed into P. Pastoris strain X-33 using the Pichia EASYCOMP Transformation Kit (INVITROGEN). After incubation on YPD-Zeocin plates for 3-4 days, 6 clones per construct were inoculated in BMGY medium (10 g/L yeast extract, 20 g/L tryptone, 13.4 g/L yeast nitrogen base, 100 mL/L glycerol, 0.4 mg/L biotin, and 100 mM potassium phosphate buffer pH 6.0) and incubated for 24 h at 30° C. The next day, cells were diluted to a starting OD600 of 1.0 in BMMY (10 g/L yeast extract, 20 g/L tryptone, 13.4 g/L yeast nitrogen base, 5 mL/L methanol, 0.4 mg/L biotin, and 100 mM potassium phosphate buffer pH 6.0) expression medium and incubated for 24 h at 30° C. 5 μl of culture supernatant was then run on a 4-20% SDS gel followed by Coomassie staining (FIGS. 13A and 13B). The clone with the best selection was selected and stored at −80° C.

Expression of target protein was studied over a 4-day time course to identify a suitable expression time for large scale protein expression. The respective protein-expressing yeast strain was inoculated in BMGY medium and incubated for 24 h at 30° C. in a shaking incubator. The next day, cells were diluted to a starting OD600 of 1.0 in BMMY expression medium and incubated for 90 h at 30° C. shaking incubator. 5 μl of culture supernatant was collected after multiple timepoints, then run on a 4-20% SDS gel followed by Coomassie staining. Based on the yield and expression products, the expression times for F-BPTI and Fc-SPINK were set to 48 h and 72 h, respectively.

Fc-trypsin inhibitor fusion proteins were purified from yeast culture supernatant by Protein A affinity chromatography. After protein expression, the culture was centrifuged at 3.000×g for 15 min to separate the protein-containing supernatant from the cells. The supernatant was subsequently cooled to 4° C., sterile-filtered, adjusted to pH 7.5 and concentrated by factor 50-100 using ultrafiltration spin columns with a cut-off molecular weight of 10 kDa (CENTRICON PLUS-70, EMD MILLIPORE). Protease inhibitor (HALT protease inhibitor, THERMO SCIENTIFIC) was added to prevent protein degradation.

Protein A affinity chromatography was performed using Pierce Protein A Plus Agarose (THERMO SCIENTIFIC). In brief, the sample was diluted 1:1 Binding Buffer (THERMO SCIENTIFIC) before adding 1 ml Protein A Plus Agarose per 20 mg IgG Fc fragment containing protein. After incubation for 1 h at 4° C. shaking overhead, the agarose resin was collected by centrifugation for 3 min at 800×g and loaded into a column. Alternatively, the diluted sample was directly applied to a pre-packed and equilibrated column. The column was washed with 15 column volumes Binding Buffer (THERMO SCIENTIFIC) before eluting the target protein with Elution Buffer (THERMO SCIENTIFIC) in 1-4 ml fractions. The collection tubes were prefilled with 100 μl 1M TRIS pH 8.0 per 1 ml of eluate to immediately adjust eluted fractions to physiologic pH. Elution was monitored by measuring the absorbance at 280 nm. Samples from all purification steps were subsequently analyzed by SDS PAGE and Coomassie staining. Protein containing fractions were pooled, dialyzed against PBS and concentrated using ultrafiltration spin columns with a cut-off molecular weight of 10 kDa (AMICON ULTRA-4, EMD MILLIPORE) to a final concentration of 10-20 mg/ml. The final protein was then analyzed on Coomassie gel.

### Example 3: Exemplary Fc Fusion Proteins Enable Potent Trypsin Inhibition In Vitro

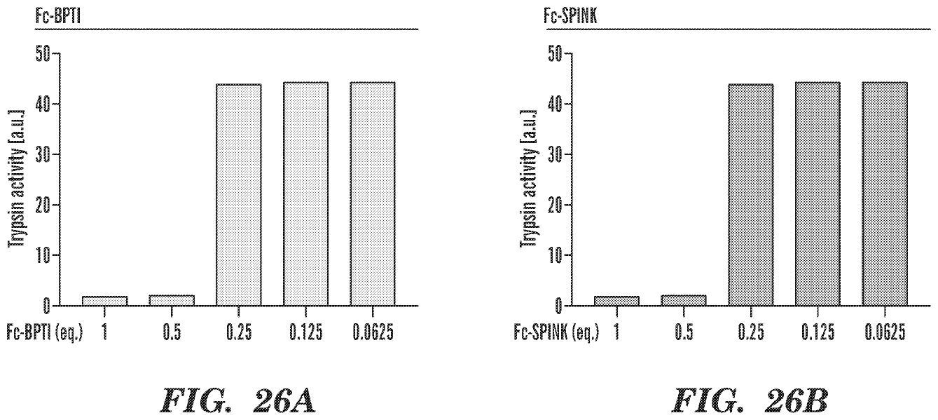

Exemplary Fc-SPINK and Fc-BPTI were incubated in the indicated molar ratios followed by measuring trypsin activity with the Trypsin Activity Colorimetric Assay Kit (BIOVISION, K771). At molar ratios of 1 and 0.5, trypsin activity is highly diminished (FIG. 26).

### Example 4: Pharmacokinetics and Bio-Distribution of Exemplary Fc Fusion Proteins

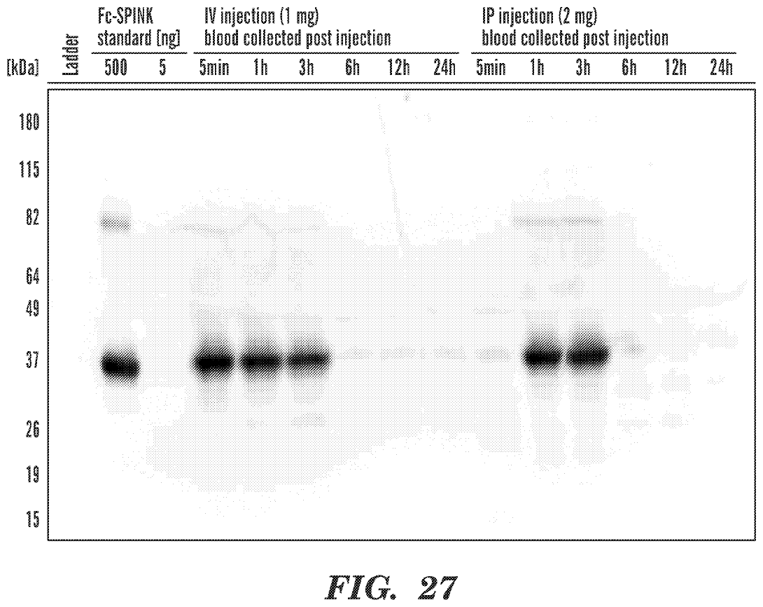

Fc-SPINK was injected intravenously and intraperitoneal and the amount of drug was subsequently analyzed by Western blotting against SPINK in serum from tail vein blood. Samples were analyzed 5 min, 1 h, 3 h, 6 h, 12 h, and 24 h post Fc-SPINK injection. Results are shown in FIG. 27. Note, that the serum concentration in the blood is comparable between IV and IP injection despite the different amounts of administered drug.

Fc-BPTI was injected intravenously and intraperitoneal. 24 hours post injection, organs were harvested, grinded and lysed in RIPA buffer. 50 μg of total protein was then run on a 4-20% SDS gel followed by Western blot using an antibody targeting SPINK1. A majority of the protein ends up on liver and kidney, whereas only a small amount of Fc-SPINK can be detected in the pancreas (FIG. 28).

### Example 5: Caerulein Model of Acute Pancreatitis

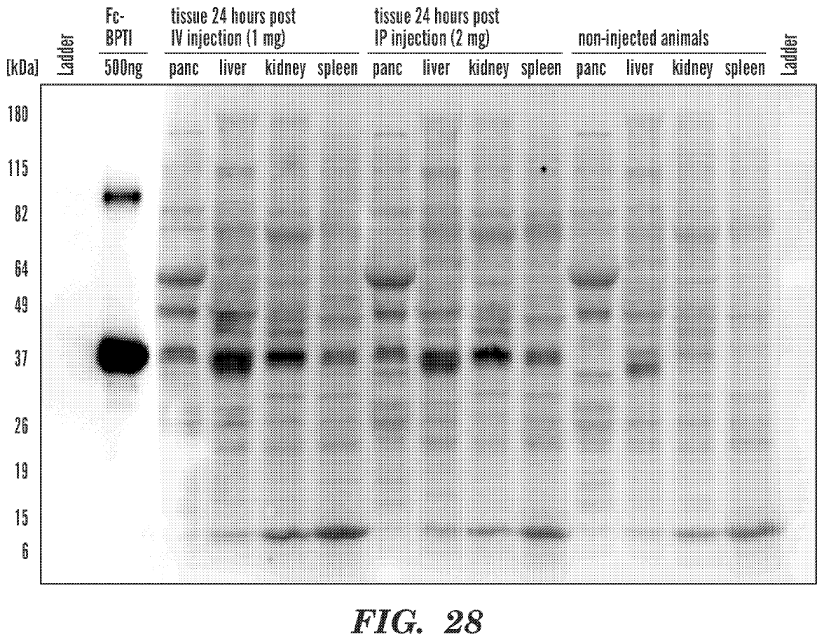

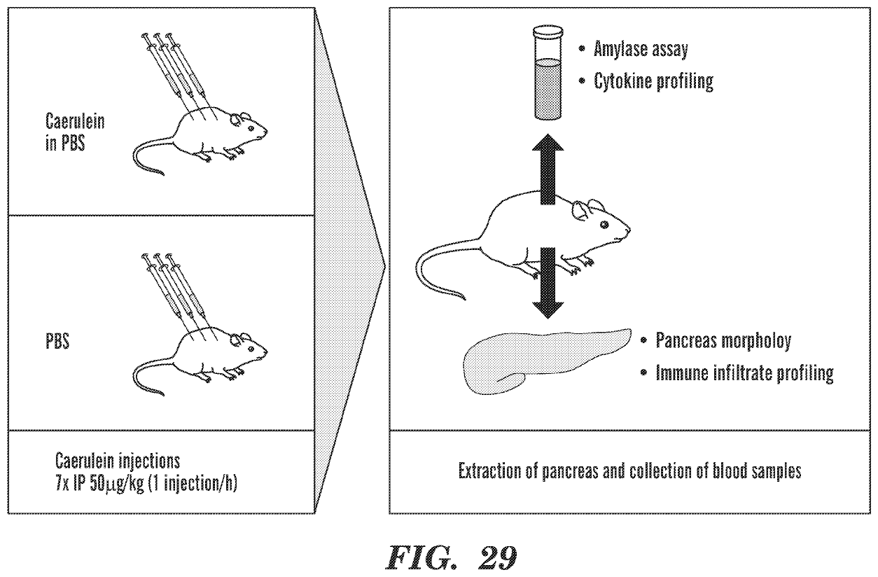

C57BL/6 mice were injected with 50 μg/kg caerulein or PBS seven times, i.e. once per hour over a time-course of 6 h, by intraperitoneal (IP) injection. 2 hours or 24 h after the last injection, mice were sacrificed and the pancreas as well as blood samples were collected followed by downstream analysis (FIG. 29).

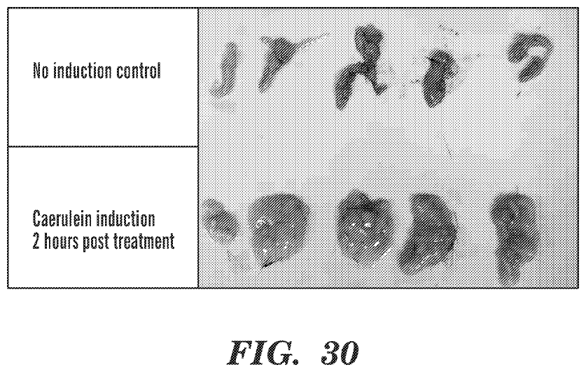

As seen from FIG. 30, The caerulein induction group shows massive swelling indicative of edema, as well as grey coloring suggesting massive immune infiltration. Mice were sacrificed 2 h after the final Caerulein injection.

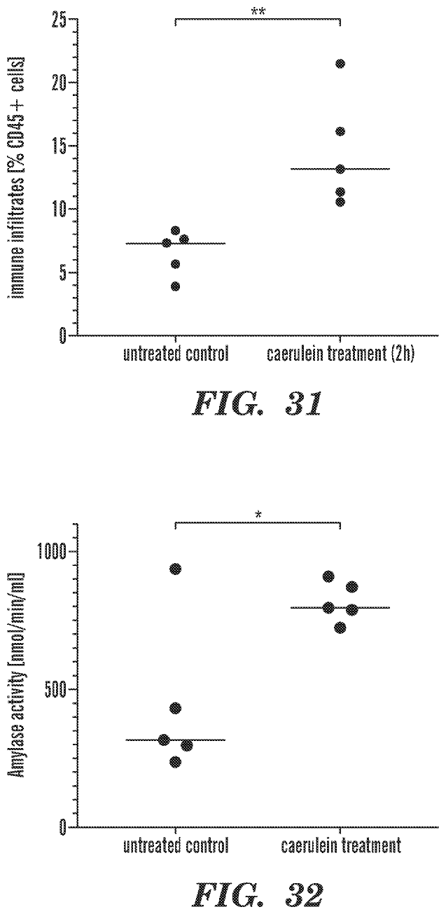

Pancreas tissue was dissociated using collagenase and trypsin inhibitor followed by antibody staining and FACS analysis. As seen from FIG. 31, the proportion of total immune cells (CD45⁺ cells) in the pancreas is elevated as compared to the non-induced group, indicating an inflammatory process.

Amylase activity was measured using the Amylase Activity Colorimetric Assay kit (BIOVISION, #K711). As seen from FIG. 32, amylase activity in the Caerulein induction group was elevated as compared to the non-induced group, indicating induction of acute pancreatitis. Note, that amylase is a pancreatic enzyme which is used as a clinical biomarker for pancreatitis.

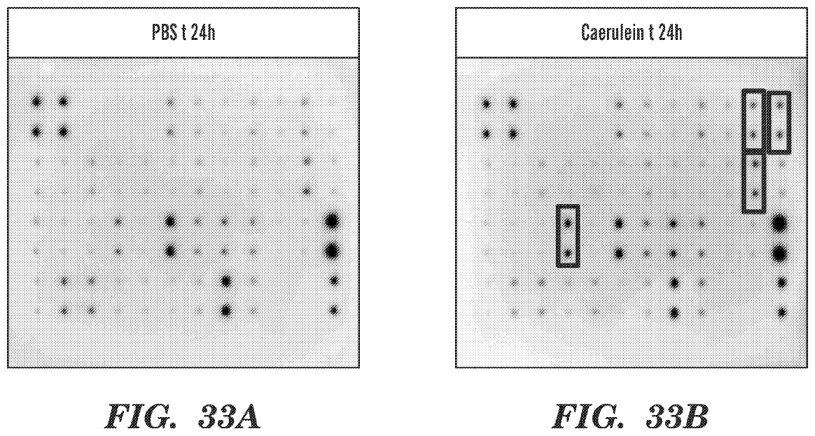

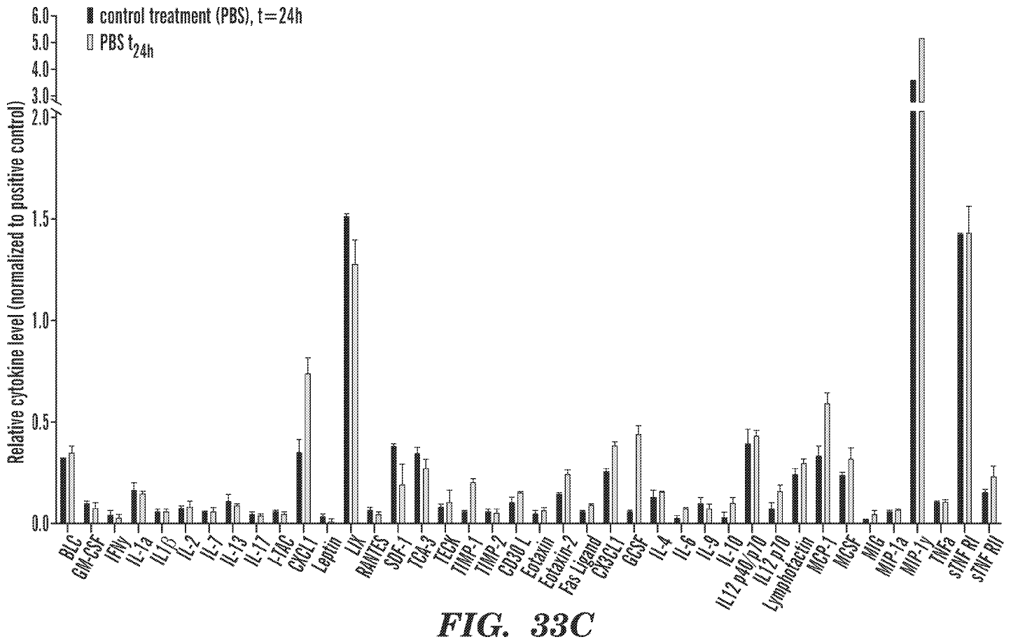

Cytokine array was performed on blood samples from the different mouse groups (FIG. 33A). As seen from FIG. 33B, caerulein-induced pancreatitis is of a generic type characterized by increased levels of chemokines CXCL1 and CX3CL1, as well as the neutrophil proliferation marker GCSF.

### Example 6: Evaluation of Drug Efficacy in a Mouse Model of Acute Pancreatitis

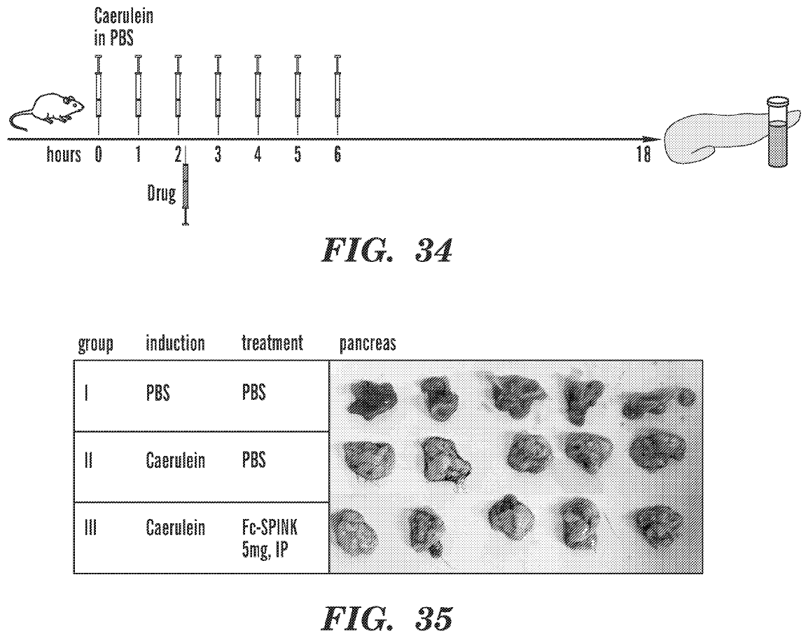

Acute pancreatitis was induced by repeated IP injection of 50 μg/kg caerulein. After the third caerulein injection, 5 mg Fc-SPINK in PBS or PBS only (as control) was injected. Mice were sacrificed 18 h after the first caerulein injection followed by downstream analysis (FIG. 34). Treatment groups are shown in Table 1.

TABLE 1
Treatment groups

| Group | Induction | Treatment |
|---|---|---|
| I | PBS | PBS |
| II | Caerulein | PBS |
| III | Caerulein | Fc-SPINK |

As seen from FIG. 35, the caerulein induction group shows massive swelling indicative of edema, as well as grey coloring suggesting massive immune infiltration. Note, that while the Fc-SPINK treatment group does not show apparent morphological differences as compared to the non-treated caerulein induction group, this is also not to be expected within the short time-frame of this experiment.

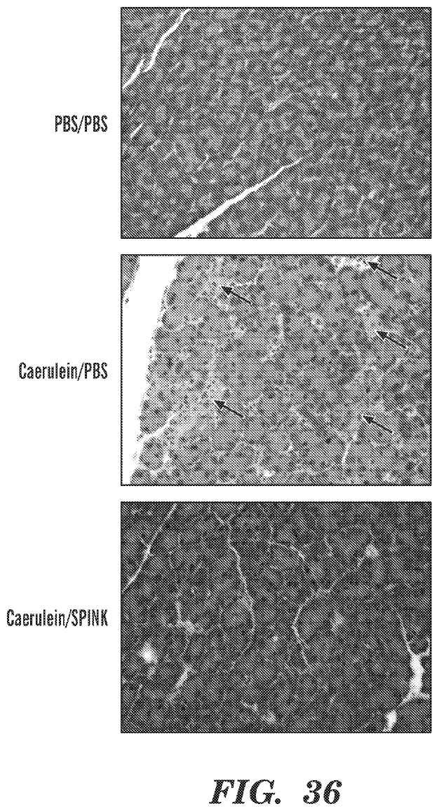

Pancreas tissue was fixed in 10% buffered formalin for 24 hours before paraffin embedding, section cutting and H&E (Haemotoxylin and Eosin) staining. As seen from FIG. 36, the caerulein group shows a large number of necrotic cells (light violet cells, indicated by arrows), infiltration of immune cells, as well as overall less densely packed cells indicating edema. However, the Fc-SPINK treated group does not show necrotic cells, indicating a curative therapeutic effect.

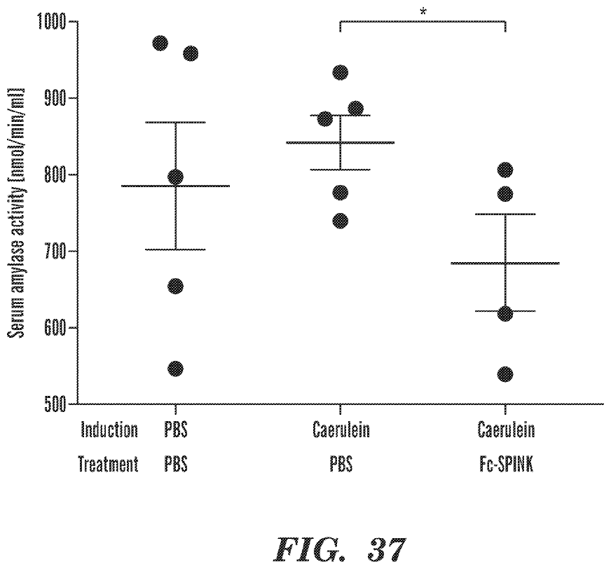

Amylase activity was measured using the Amylase Activity Colorimetric Assay kit (BIOVISION, #K711). As seen from FIG. 37, amylase activity in the caerulein induction group was elevated as compared to the non-induced group, indicating induction of acute pancreatitis. The caerulein induction group treated with Fc-SPINK showed low levels of serum amylase activity, indicating therapeutic efficacy of Fc-SPINK.

### Example 7: Construction of a PK/Distribution Model

Fc fusion proteins of the invention can be expected to avoid the major pharmacodynamics pitfalls of wildtype BPTI as a treatment for pancreatitis. While unaltered BPTI is trapped in binding to off-target molecules and shunted to lysosomes, an ideal Fc-inhibitor fusion would instead be recycled to the plasma repeatedly until reaching its intended binding site to pancreatic trypsin.

A model for the distribution and elimination of Fc fusion proteins is constructed, and populated with: (1) known parameters that are typical of antibodies and Fc fusion proteins, and (2) parameters that one can control, such as the binding constant for trypsin and other proteases, Fc receptor binding at relevant pH values, and possibly endosomal stability of the drug relative to trypsin.

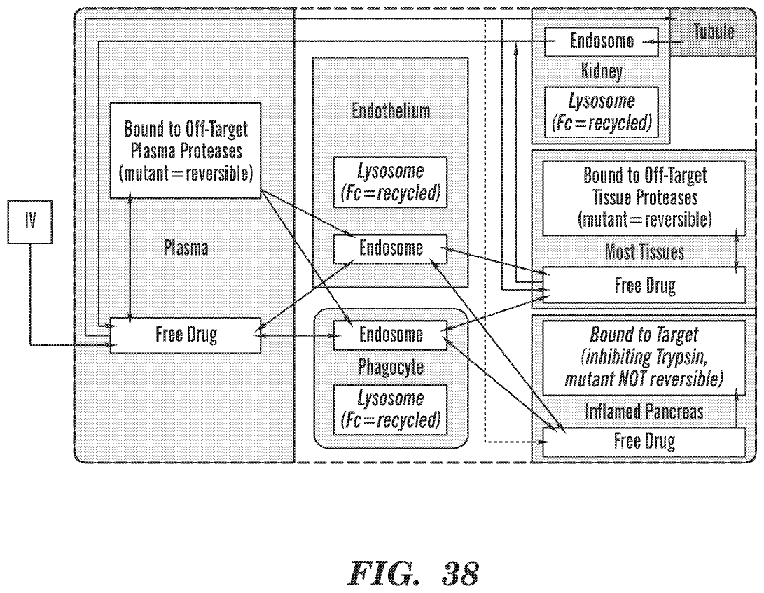

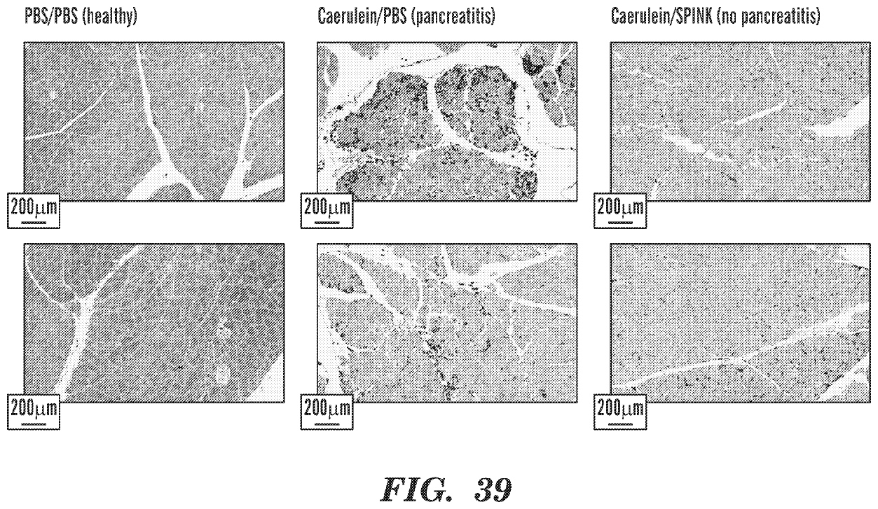

In this PK/distribution model, drug is administered IV into the blood stream. While in the plasma, the drug can inhibit other proteases such as plasmin, potentially leading to side effects. The drug distributes into tissues, at least in part via the endothelial wall and in part via phagocytes with Fc receptors. To exit from such cells, FcRn binding is critical. Upon entering other tissues (e.g. liver), the drug can be bound via Fc receptors (e.g. Kupffer cells in the liver, other leukocytes) and will either be recycled via FcRn or eventually degraded. Such degradation is likely to be the major mode of clearance, especially since the drug molecular weight is greater than the threshold for renal clearance. Thus, binding to FcR can accelerate clearance, but may (or may not) be important for distribution into tissues (FIG. 38).

In the pancreas, the drug can bind to trypsin very rapidly and robustly. The complex can be inert. Ideally, in addition to neutralizing trypsin the drug can carry trypsin into FcR-bearing cells. In these cells, if trypsin affinity is sufficiently reduced at endosomal pH, the trypsin can be released and targeted to the lysosome while the empty Fc fusion protein is recycled back out of the cell, allowing one drug molecule to mediate the degradation of several trypsin molecules. Alternatively, binding can remain strong enough that the neutral complex is either sent back to circulation or sent to the lysosome together, without reusing the drug molecule.

It is important to note that while there are a number of compartments in this model, many of the distribution parameters are repeated used, so that a predictive model can be constructed from just a few numbers, many of which can be controlled by protein engineering. These include the affinity for Fc receptors, FcRn, trypsin plus other proteases, and possibly pH-dependent dissociation of trypsin from the inhibitor (for example using the BPTI Ala16His mutation described above).

Such a model was used to estimate the necessary dose of Fc-SPINK or Fc-BPTI in a human. In an adult, 70-kg human, the level of trypsin produced by the pancreas can be as high as 100 mgs/day, although it is usually less. It is also empirically estimated that about 6% of an injected dose of an antibody accumulates in the pancreas. Since the primary mechanism of inhibition by an inhibitor of the invention is stoichiometric binding, in general the amount of Fc-protease inhibitor must be in molar excess over its target. The molecular weight of trypsin is about 23,000 Daltons, and the molecular weight of a monomeric Fc-BPTI or Fc-SPINK is about 37,000 Daltons, so about 160 mgs of Fc-protease inhibitor must be present in the pancreas for titration. Taken together, these numbers imply a required systemic dose of about 2.5 grams of Fc-protease inhibitor fusion protein to titrate the amount of trypsin that may be produced in a day, if considered in the absence of a multiplier mechanism such as the internalization of Fc-protease inhibitor/trypsin complexes into Fc receptor-bearing cells, specific degradation of trypsin, and recycling of the Fc fusion protein back out of the cell. Indeed, the high dose that is needed to titrate the trypsin in the pancreas was expected to saturate Fc receptors in this tissue, such that Fc receptor binding would play essentially no role in pharmacokinetics or pharmacodynamics at a therapeutic dose.

### Example 8: Treatment of a Human Pancreatitis Patient with a Fusion Protein of the Invention

A patient presenting with pancreatitis is treated with a fusion protein of the invention as follows. First, the underlying cause of the pancreatitis is assessed. In cases with gallstone involvement such as a gallstone blockage at the sphincter of Oddi, one option is to insert a suction device through the esophagus, stomach and small intestine and remove the stone. However, in cases where there is no gallstone involvement, treatment with a fusion protein of the invention is particularly appropriate.

Typically, a patient with pancreatitis is hospitalized, resting in bed, and given intravenous liquids in a continuous manner. Typically, saline without dextrose is administered, because dextrose stimulates the pancreas. No food is given, because food stimulates the pancreas. A fusion protein of the invention that has been formulated in a solution form is added to the saline to be infused into the patient. The infusion may last for one hour, but as a practical matter may take place over a shorter or longer period of time because the patient is bedridden and being given iv fluids in any case. A typical dose of 500 mgs, 1 gram, 2.5 grams or more is used. It is noteworthy that such doses are higher than doses of therapeutic antibodies, but without wishing to be bound by theory this is because of the need to titrate trypsin, which is present in much higher amounts that typical antibody targets such as TNF, IL-1, and so on.

### Example 9: Treatment of a Human Patient with an Fc-BPTI Fusion Protein of the Invention During Cardiac Surgery

The Fc-BPTI fusion protein of the invention is used during cardiac surgery in an adult human as follows. After induction of anaesthesia but prior to cutting the sternum, about 200 to 500 milligrams and more preferably about 320 milligrams of protein is administered to a 70 kg patient. Administration is generally by infusion of a solution with a concentration of 1 to 10 mgs/ml of Fc-BPTI in a pharmaceutically appropriate diluent such as 0.85% NaCl, phosphate-buffered saline, or dextrose for infusion. About 1 milliliter is first administered slowly, over about 10 minutes, to insure that there is no allergic-type reaction. The remainder of the dose is then infused over about 15-20 minutes. In general, Fc-BPTI should be given to patients lying down and should be given slowly (maximum 5-10 mL/min) as an iv injection or infusion.

In general, it is not necessary to continue administration after sternotomy because the plasma half-life of Fc-BPTI is long enough that adequate levels are maintained during a surgery lasting several hours. Moreover, kidney toxicity is avoided because the molecular size of Fc-BPTI is too large to allow passage into the lumen of Bowman's capsule.

### REFERENCES

1) Vos, Theo, et al. “Global, regional, and national incidence, prevalence, and years lived with disability for 310 diseases and injuries, 1990-2015: a systematic analysis for the Global Burden of Disease Study 2015.” The Lancet 388.10053 (2016): 1545-1602.
2) Yadav, Dhiraj, and Albert B. Lowenfels. “The epidemiology of pancreatitis and pancreatic cancer.” Gastroenterology 144.6 (2013): 1252-1261.
3) Fu C Y, Yeh C N, Hsu J T, Jan Y Y, Hwang T L. “Timing of mortality in severe acute pancreatitis: experience from 643 patients.” World J Gastroenterol. 2007; 13(13):1966-9.
4) İlem-Özdemir, Derya, et al. “Effect of microemulsion formulation on biodistribution of 99mTc-Aprotinin in acute pancreatitis models induced rats.” Drug delivery 23.8 (2016): 3055-3062.
5) Ascenzi, Paolo, et al. “The bovine basic pancreatic trypsin inhibitor (Kunitz inhibitor): a milestone protein.” Current Protein and Peptide Science 4.3 (2003): 231-251.
6) Smith, M., H. M. Kocher, and B. J. Hunt. “Aprotinin in severe acute pancreatitis.” International journal of clinical practice 64.1 (2010): 84-92.
7) Fedail, S. S. et al., “Trypsin and lactoferrin in pure pancreatic juice in patients with pancreatic disease.” Gut 20 (1979) 983-986.
8) Grzesiak, A. et al., “Substitutions at the P1′ Position in BPTI Strongly Affect the Association Energy of Serine Proteinases.” Journal of Molecular Biology 301.1 (2000): 205-217.
9) Vidarsson, G. et al. “IgG subclasses and allotypes: from structure to effector functions.” Frontiers in immunology 5 (2014): 520.
10) Glassman, P. M., Abuqayyas, L. and Balthasar, J. P. (2015), Assessments of antibody biodistribution. The Journal of Clinical Pharmacology, 55: S29-S38. doi:10.1002/jcph.365
11) Roland E. Kontermann (2016) Half-life extended biotherapeutics, Expert Opinion on Biological Therapy, 16:7, 903-915, DOI: 10.1517/14712598.2016.1165661
12) Mezo, A. R., McDonnell, K. A., Low, S. C., Song, J., Reidy, T. J., Lu, Q., . . . . Bitonti, A. J. (2012). Atrial Natriuretic Peptide-Fc, ANP-Fc, Fusion Proteins: Semisynthesis, In Vitro Activity and Pharmacokinetics in Rats. Bioconjugate Chemistry, 23(3), 518-526. doi:10.1021/bc200592c
13) Sultana, A., and Lee, J. E. 2015. Measuring Protein-Protein and Protein-Nucleic Acid Interactions by Biolayer Interferometry. Curr. Protoc. Protein Sci. 79:19.25.1-19.25.26. doi: 10.1002/0471140864.psl925s79.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### Example 10: Pancreatitis-Fc-SPINK1 Fusions

Described herein are fusions of the fragment crystalizable (Fc) region of human IgG1/murine IgG2a/b/c and different effector proteins, including human serine peptidase inhibitor Kazal type 1 (SPINK1), murine SPINK-1 analogues and interleukin1 receptor antagonist (IL-1Ra).

The aim in engineering these variants has been to optimize the therapeutic effect for the pathological state of (acute) pancreatitis, inhibiting free proteases, suppressing inflammation of pancreatic tissue, and enhancing the plasma half-life of the compound. Surrogate and therapeutic proteins occur homologous in their design.

The Fc region has been modified in the constructs shown (see e.g., Table 2). Mutations include the removal of a known N-glycosylation site (ND; e.g., N82D, N83D, N89D, N297D), CS in the C-terminal hinge (e.g., C220S), KA at the N-terminus (e.g., K239A, K447A). These mutations enhance folding and stability of the protein and therefore production levels.

Furthermore, some of these constructs inherit mutations of the CH2 and CH3 domains, which alter affinities to Fc gamma receptors and the neonatal Fc receptor (LALA; PG; VE; YTE; LS; DHS). Negative charges have been introduced to prevent entry into the kidney.

The used plasmid is optimized for cloning in E. coli and expression in P. pastoris, including an α-mating factor for secretion of the recombinant protein from the latter organism. This secretion signal has been modified for three of the shown constructs (pre-Ost1; pro(d57-70); pro-sequence removal; see e.g., Example 15).

### Example 11: Additional Forms of Fc-SPINK1 with Extended Plasma Half-Life, Reduced Fc Receptor Binding, and Reduced Entry into the Kidney

Described herein are fusion proteins comprising an antibody Fc region and a SPINK1 moiety, wherein the fusion protein has an extended plasma half-life. Such fusion proteins can be used to treat patients with chronic forms of pancreatitis, such as forms of the disease in which the patient has an underlying genetic propensity to develop pancreatitis. Such patients require long-term treatment, which may proceed for weeks, months, years, or throughout life. In such cases, it is useful to have the dosing be infrequent.

Also described herein are fusion proteins that further comprise an interleukin1 receptor antagonist (IL-1RA) moiety. IL-1 receptor antagonist is a secreted protein that binds to IL-1 receptor and inhibits signaling, by competing with ligands of IL-1 receptor such as IL-1alpha, IL-1beta, and other ligands. The IL-1 signaling system induces inflammation, and plays an important role in ‘sterile inflammation’ in which tissue breakdown and cell lysis occur in the absence of pathogen infection. As described herein, IL-1 signaling and sterile inflammation is particularly relevant to pancreatitis, where damage to cells and cell killing are mediated by endogenously produced proteases, rather than by infectious agents. The proteins “IL1Ra-Fc(N82D)-SPINK_noTag”, “(pOst1)_SPINK-Fc(N82D)-Il1Ra_noTag”, “(rm_pro)_SPINK-Fc(N82D)-IL1Ra_noTag” (see e.g., SEQ ID NOs: 89, 95, 97) are specific embodiments of fusion proteins comprising an IL-1RA moiety, and Fc region, and a SPINK1 moiety. Further examples of fusion proteins comprising an IL-1RA moiety, and Fc region, and a SPINK1 moiety include: “SPINK-Fc(N82D)-Il1Ra_noTag,” “murine_SPINK1-Fc(N83D)_IL-1Ra_noTag,” “murine_SPINK1-Fc(N83D)_IL-1Ra(N84D)_noTag,” and “(pro-d57-70)_SPINK-Fc(N82D)-IllRa_noTag” (see e.g., SEQ ID NOs: 88, 93, 94, 96).

In terms of protein expression, the N-terminal to C-terminal configuration IL-1RA-Fc-SPINK1 was found to be more highly expressed than SPINK1-Fc-IL-1RA, particularly from Pichia pastoris. Thus, the configuration IL-1RA-Fc-SPINK1 is a preferred embodiment. Also in terms of protein expression from Pichia pastoris, the three proteins “(pOst1)_SPINK-Fc(N82D)-Il1Ra_noTag”, “(rm_pro)_SPINK-Fc(N82D)-IL1Ra_noTag” all encode the same mature protein sequence (see e.g., SEQ ID NOs: 95-97), but have different yeast-based leader sequences (see e.g., SEQ ID NOs: 176-181; see e.g., Example 15); no significant differences were found in the expression levels of these three constructs.

In terms of protein expression, it is also important to note that when any yeast or insect-based protein expression system is used, any N-linked oligosaccharide will generally have a non-mammalian structure, and such an oligosaccharide may be recognized by the immune system as foreign. Therefore, the protein constructs that were expressed in Pichia pastoris (see e.g., Example 2) all contained an Fc with a mutation in the N-linked glycosylation site, specifically Asn297Asp. Removal of the N-linked glycosylation site in the Fc has the effect of at least reducing binding to Fc(gamma) receptors and complement factors (see e.g., H Tao and S L Morrison, J Immunol 1989; 143:2595-2601). As used herein, “Fc receptor” or “FcR” refers to Fc(gamma) receptors, which interact with the IgG family of antibodies and with Fc regions derived from IgG antibodies.

Variant proteins comprising an Fc moiety and a SPINK1 moiety with extended plasma half-life were constructed. Some proteins were based on human Fc and SPINK1 sequences, while others were based on mouse-based Fc and SPINK1 sequences. (The murine SPINK1 homologue is sometimes termed SPINK3.) Without wishing to be bound by theory, the Fc region was considered to be the main controlling element in plasma half-life. In one set of embodiments, mutations were introduced that reduce binding to Fc receptor. Binding to Fc receptor may lead to endocytosis into Fc receptor-bearing cells such as macrophages, followed by degradation, thus reducing plasma half-life. As described herein, in a fusion protein with an Fc region at its N-terminus, the Fc receptor binding site is particularly exposed because there are no Fab elements that might sterically occlude an Fc receptor from to its site in the hinge region, so binding may be more efficient than to an intact antibody. As a result, simply mutating the N-linked glycosylation site of the Fc may not be sufficient to block Fc receptor binding.

Mutations converting the sequence “ . . . . TCPPCPAPELLGGPSVFLFPPK . . . . ” (SEQ ID NO: 182) to “ . . . . TCPPCPAPEAAGGPSVFLFPPK . . . . ” (SEQ ID NO: 183) near the N-terminus of the Fc region were introduced into Fc region of the human-based proteins “Fc(N82D;YTE;LALA)SPINK_noTag”, “Fc(N82D;YTE;LALA-PG)SPINK_noTag”, Fc(N82D;LS;LALA-PG)SPINK_noTag, and “Fc(N82D;DHS;LALA-PG)SPINK_noTag” (see e.g., SEQ ID NOs: 111-114). (The mutations of the two adjacent leucines to alanines are referred herein to as “LALA”.) The mutation converting the sequence “CKVSNKALPAPIEKTIS . . . . ” (SEQ ID NO: 184) to “ . . . . CKVSNKALGAPIEKTIS . . . . ” (SEQ ID NO: 185) in the human Fc region (“PG”) was introduced into a subset of these proteins (see e.g., SEQ ID NOs: 112-114); this mutation further reduces Fc receptor binding. The net effect of the LALA mutations and the PG mutations is to improve the plasma half-life of proteins comprising Fc-SPINK1 in mice, humans, and other mammals.

The FcRn receptor (the “neonatal” Fc receptor) plays an important role in extending the plasma half-life of IgG-type antibodies, and also albumin. Binding of FcRn takes place at a different site on Fc than the binding of the canonical Fc(gamma) receptors.

### Example 12: Amelioration of Chronic Pancreatitis by Fc-SPINK1

The Fc-SPINK1 fusion protein tested in Example 5 was also tested in a chronic model of pancreatitis, see e.g., Geisz and Sahin-Toth (Nature Communications (2018)9:5033), and comprises a knock-in mouse with a form of the cationic trypsinogen T7 gene encoding a mutation Asp23Ala in the region of trypsinogen N-terminal to the activating cleavage site. The mutation enhances the rate of trypsinogen/trypsin auto-activation about 50-fold. Mice homozygous for this mutant gene develop a severe pancreatitis that can first be observed about four to five weeks after birth. Relative to normal mice, the pancreas is smaller and has many necrotic cells and infiltrating immune cells. The pancreatitis seen in these mice is much more severe than human forms of chronic pancreatitis.

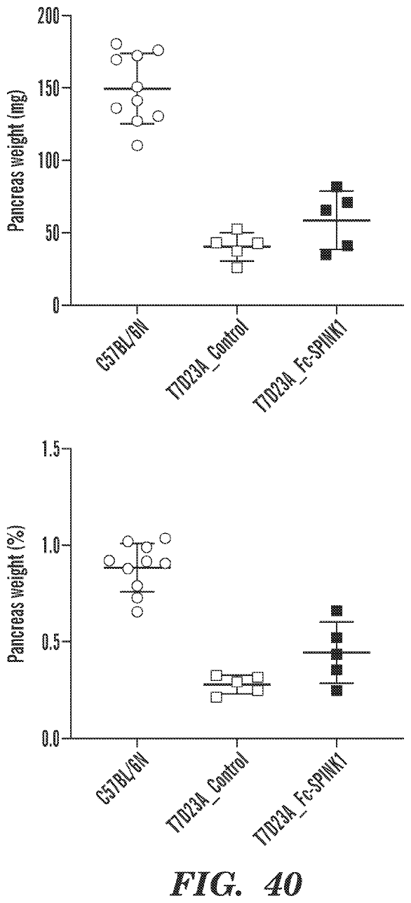

Starting at about three weeks of age, mice were injected with 5 mgs of Fc-SPINK1 on days 1, 3, 5, 8 and 10, and then sacrificed on about day 12. The pancreases were weighed and characterized histologically. Control groups included wild-typec57B⅙ mice and C57B⅙(T7D23A) mice, both untreated with Fc-SPINK1. Weights of the pancreases were calculated as absolute weights and also normalized to the body weight of the mice. As indicated in FIG. 40, the weights of the pancreases in the treated mice were greater than for the untreated mice. This indicates some sparing of the pancreas as a result of drug treatment. Complete sparing of the pancreas in this model was not expected, due to the extreme and non-physiological activation of trypsin.

### Example 13: An Analysis of the Dose Response of Acute Pancreatitis

Using essentially the same procedure and the same form of Fc-SPINK1 as described in Example 5, C57B⅙ mice were injected with cerulein seven times, and injected with either 0, 1, 2.5 or 5 Fc-SPINK1 mgs/mouse after the third cerulein injection. Mice were sacrificed the following day, and the pancreases were characterized by hematoxylin/eosin staining and by staining with an anti-CD11b antibody, which reveals macrophages.

The results indicated that with respect to the presence of necrotic cells and edema, the mice receiving 0 mgs of Fc-SPINK1 showed the greatest edema and generally the most necrotic cells. (It should be noted that sometimes when this is experiment is performed, the presence of necrotic cells is not observed even in mice not treated with Fc-SPINK11, but that edema is reproducibly observed.). These mice showed the highest level of CD11b-positive cells in pancreatic tissue.

The mice treated with 1 or 2.5 mgs of Fc-SPINK1 showed an intermediate level of edema and CD 1b+cell infiltration. The mice treated with 5 milligrams of Fc-SPINK1 showed no detectable edema and very little CD11b+cell infiltration.

Thus, the optimal dose of Fc-SPINK1 in mice was about 5 milligrams per 25 gram mouse. Allometric scaling to humans leads to an estimation that about 1 gram would be the ideal dose for a 75 kilogram adult human with pancreatitis. The exact dose will vary depending on the weight of the person and the type of molecule that is used.

### Example 14: A Form of Fc-SPINK1 with Enhanced FcRn-Based Recycling, Minimized Fc(Gamma) Receptor Binding, and Minimized Passage Across the Kidney Basement Membrane

Three factors are thought to be relevant to the plasma pharmacokinetics of Fc-SPINK1 proteins: recycling out of phagocytic cells mediated by the FcRn system; minimized binding to Fc(gamma) receptors by the Fc element, and minimized entry into the kidney.

The protein “Fc(N82D; YTE;LALA-PG)-SPINK_neg-charge_noTag” was designed based on insights of the invention. This protein is tested in mice, primates and humans, and shows superior plasma half-life compared to other forms of Fc-SPINK1.

TABLE 3
PK-optimized Fc-SPINK1

| | Fusion | | Fusion | |
|---|---|---|---|---|
| | Pro- | | Pro- | |
| Fusion | tein | | tein | |
| Pro- | Amino | | Protein | Fusion |
| tein | Acid | Plasmid | Nucleic | Protein |
| Name | SID | SID | Acid | Sequence |
| Fc | 85 | 115 | 241 | EPEESDKTHTCPPCPA |
| (N82D; | | | | PEaaGGPSVFLFPPK |
| YTE; | | | | PKDTLyItRePEVTC |
| LALA- | | | | VVVDVSHEDPEVKFN |
| PG)- | | | | WYVDGVEVHNAKTKP |
| SPINK_ | | | | REEQYDSTYRVVSVL |
| neg- | | | | TVLHQDWLNGKEYKC |
| charge_ | | | | KVSNKALgAPIEKTI |
| noTag | | | | SKAKGQPREPQVYTL |
| | | | | PPSRDELTKNQVSLT |
| | | | | CLVKGFYPSDIAVEW |
| | | | | ESNGQPENNYKTTPP |
| | | | | VLDSDGSFFLYSKLT |
| | | | | VDKSRWQQGNVFSCS |
| | | | | VMHEALHNHYTQKSL |
| | | | | SLSPGEDSLGREAKC |
| | | | | YNELNGCTKIYDPVC |
| | | | | GTDGNTYPNECVLCF |
| | | | | ENRKRQTSILIQKSG |
| | | | | PC |

SEQ ID NO: 241, a nucleic sequence
encoding the “Fc(N82D; YTE; LALA-PG)-
SPINK_neg-charge_noTag” protein:
GAACCAgaagagTCTGATAAGACTCATACTTGTCCACCATGTCCA
GCTCCAGAAGCTGCTGGTGGTCCATCTGTTTTTTTGTTTCCACCA
AAGCCAAAGGATACTTTGTACATTACTAGAGAGCCAGAAGTTACT
TGTGTTGTTGTTGATGTTTCTCATGAAGATCCAGAAGTTAAGTTT
AACTGGTACGTTGATGGTGTTGAAGTTCATAACGCTAAGACTAAG
CCAAGAGAAGAACAATACGACTCTACTTACAGAGTTGTTTCTGTT
TTGACTGTTTTGCATCAAGATTGGTTGAACGGTAAGGAATACAAG
TGTAAGGTTTCTAACAAGGCTTTGGGTGCTCCAATTGAAAAGACT
ATTTCTAAGGCTAAGGGTCAACCAAGAGAACCACAAGTTTACACT
TTGCCACCATCCAGAGATGAATTGACTAAGAACCAAGTTTCTTTG
ACTTGTTTGGTTAAGGGTTTTTACCCATCTGATATTGCTGTTGAA
TGGGAATCTAACGGTCAACCAGAAAACAACTACAAGACTACTCCA
CCAGTTTTGGATTCTGATGGTTCTTTTTTTTTGTACTCTAAGTTG
ACTGTTGATAAGTCCAGATGGCAACAAGGTAACGTTTTTTCTTGT
TCTGTTATGCATGAAGCTTTGCATAACCATTACACTCAAAAGTCT
TTGTCTTTGTCTCCAGGTgagGATTCTTTGGGTAGAGAAGCTAAG
TGTTACAACGAATTGAACGGTTGTACTAAGATTTACGATCCAGTT
TGTGGTACTGATGGTAACACTTACCCAAACGAATGTGTTTTGTGT
TTTGAAAACAGAAAGAGACAAACTTCTATTTTGATTCAAAAGTCT
GGTCCATGT

### Example 15: Manipulation of the Signal and Leader Sequence of Fc-SPINK1 for Expression in Yeast

It is convenient and cost-effective to express SPINK1-Fc and Fc-SPINK1 proteins in yeast, such as Pichia pastoris. Typically, expression in Pichia pastoris involves the C-terminal attachment of the protein of interest to an N-terminal leader sequence, which comprises a canonical signal sequence followed by a leader peptide that is proteolytically removed during passage of the protein through the secretory apparatus.

Because the SPINK1-Fc-IL1RA tripartite fusion protein (see e.g., SEQ ID NOs: 95-97) has somewhat reduced expression relative to SPINK1-Fc and similar proteins, the leader sequence was varied to see if expression could be improved. Three variant constructions were tested (see e.g., SEQ ID NOs: 176-181). All of them showed similar levels of SPINK1-Fc-IL1RA upon standard methanol induction and characterization of supernatants by SDS-PAGE. These result indicate that fusion proteins comprising Fc and SPINK1 are robust to variations in the signal sequence used for protein expression.

SEQ ID NO: 176,
(pOst1)_SPINK-Fc(N82D)-Il1Ra_noTag
ATGAGGCAGGTTTGGTTCTCTTGGATTGTGGGATTGTTCC
TATGTTTTTTCAACGTGTCTTCTGCTGCTCCAGTCAACAC
TACAACAGAAGATGAAACGGCACAAATTCCGGCTGAAGCT
GTCATCGGTTACTCAGATTTAGAAGGGGATTTCGATGTTG
CTGTTTTGCCATTTTCCAACAGCACAAATAACGGGTTATT
GTTTATAAATACTACTATTGCCAGCATTGCTGCTAAAGAA
GAAGGGGTATCTCTCGAGAAAAGAGAGGCTGAAGCTGATT
CTTTGGGTAGAGAAGCTAAGTGTTACAACGAATTGAACGG
TTGTACTAAGATTTACGATCCAGTTTGTGGTACTGATGGT
AACACTTACCCAAACGAATGTGTTTTGTGTTTTGAAAACA
GAAAGAGACAAACTTCTATTTTGATTCAAAAGTCTGGTCC
ATGTGAACCAAAGTCTTCTGATAAGACACATACTTGTCCA
CCATGTCCAGCTCCAGAATTGTTGGGTGGTCCATCTGTTT
TTTTGTTTCCACCAAAGCCAAAGGATACTTTGATGATTTC
CAGAACTCCAGAAGTTACTTGTGTTGTTGTTGATGTTTCT
CATGAAGATCCAGAAGTTAAGTTTAACTGGTACGTTGATG
GTGTTGAAGTTCATAACGCTAAGACTAAGCCAAGAGAAGA
ACAATACGATTCTACTTACAGAGTTGTTTCTGTTTTGACT
GTTTTGCATCAAGATTGGTTGAACGGTAAGGAATACAAGT
GTAAGGTTTCTAACAAGGCTTTGCCAGCTCCAATTGAAAA
GACTATTTCTAAGGCTAAGGGTCAACCAAGAGAACCACAA
GTTTACACTTTGCCACCATCCAGAGATGAATTGACTAAGA
ACCAAGTTTCTTTGACTTGTTTGGTTAAGGGTTTTTACCC
ATCTGATATTGCTGTTGAATGGGAATCTAACGGTCAACCA
GAAAACAACTACAAGACTACTCCACCAGTTTTGGATTCTG
ATGGTTCTTTTTTTTTGTACTCTAAGTTGACTGTTGATAA
GTCCAGATGGCAACAAGGTAACGTTTTTTCTTGTTCTGTT
ATGCATGAAGCTTTGCATAACCATTACACTCAAAAGTCTT
TGTCTTTGTCTCCAGGTGCTAGGCCATCCGGTAGAAAATC
TTCCAAAATGCAAGCCTTCAGGATCTGGGATGTTAACCAA
AAAACTTTTTACCTGAGGAACAATCAATTGGTGGCTGGAT
ATTTACAAGGACCAAATGTTAACCTCGAAGAGAAAATAGA
TGTAGTCCCTATCGAGCCACATGCCTTGTTTTTGGGAATC
CATGGTGGGAAAATGTGTTTATCGTGTGTCAAGTCTGGAG
ACGAGACGCGTCTCCAATTGGAGGCCGTCGATATTACGGA
CTTATCTGAGAACCGAAAACAAGACAAACGTTTTGCTTTC
ATCAGGTCAGACAGTGGACCCACAACTTCCTTCGAATCAG
CTGCCTGTCCAGGATGGTTTCTATGCACAGCAATGGAAGC
TGATCAGCCTGTTTCGTTGACTAATATGCCCGATGAGGGT
GTCATGGTCACTAAATTCTATTTTCAGGAGGACGAG
SEQ ID NO: 177, Translated (pOst1)_
SPINK-Fc(N82D)-Il1Ra_noTag
(SEQ ID NO: 95 bolded)
MRQVWFSWIVGLFLCFFNVSSAAPVNTTTEDETAQIPAEA
VIGYSDLEGDFDVAVLPFSNSTNNGLLFINTTIASIAAKE
EGVSLEKREAEADSLGREAKCYNELNGCTKIYDPVCGTDG
NTYPNECVLCFENRKRQTSILIQKSGPCEPKSSDKTHTCP
PCPAPELLGGPSVFLFPPKPKDTLMISRTPEVTCVVVDVS
HEDPEVKENWYVDGVEVHNAKTKPREEQYDSTYRVVSVLT
VLHQDWLNGKEYKCKVSNKALPAPIEKTISKAKGQPREPQ
VYTLPPSRDELTKNQVSLTCLVKGFYPSDIAVEWESNGQP
ENNYKTTPPVLDSDGSFFLYSKLTVDKSRWQQGNVFSCSV
MHEALHNHYTQKSLSLSPGARPSGRKSSKMQAFRIWDVNQ
KTFYLRNNQLVAGYLQGPNVNLEEKIDVVPIEPHALFLGI
HGGKMCLSCVKSGDETRLQLEAVDITDLSENRKQDKRFAF
IRSDSGPTTSFESAACPGWFLCTAMEADQPVSLTNMPDEG
VMVTKFYFQEDE
SEQ ID NO: 178, pPICZαA(pro-d57-70)_
SPINK-Fc(N82D)-Il1Ra_noTag
ATGAGATTTCCTTCAATTTTTACTGCTGTTTTATTCGCAG
CATCCTCCGCATTAGCTGCTCCAGTCAACACTACAACAGA
AGATGAAACGGCACAAATTCCGGCTGAAGCTGTCATCGGT
TACTCAGATTTAGAAGGGGATTTCGATGTTGCTGTTTTGC
CATTTTCCGCCAGCATTGCTGCTAAAGAAGAAGGGGTATC
TCTCGAGAAAAGAGAGGCTGAAGCTGATTCTTTGGGTAGA
GAAGCTAAGTGTTACAACGAATTGAACGGTTGTACTAAGA
TTTACGATCCAGTTTGTGGTACTGATGGTAACACTTACCC
AAACGAATGTGTTTTGTGTTTTGAAAACAGAAAGAGACAA
ACTTCTATTTTGATTCAAAAGTCTGGTCCATGTGAACCAA
AGTCTTCTGATAAGACACATACTTGTCCACCATGTCCAGC
TCCAGAATTGTTGGGTGGTCCATCTGTTTTTTTGTTTCCA
CCAAAGCCAAAGGATACTTTGATGATTTCCAGAACTCCAG
AAGTTACTTGTGTTGTTGTTGATGTTTCTCATGAAGATCC
AGAAGTTAAGTTTAACTGGTACGTTGATGGTGTTGAAGTT
CATAACGCTAAGACTAAGCCAAGAGAAGAACAATACGATT
CTACTTACAGAGTTGTTTCTGTTTTGACTGTTTTGCATCA
AGATTGGTTGAACGGTAAGGAATACAAGTGTAAGGTTTCT
AACAAGGCTTTGCCAGCTCCAATTGAAAAGACTATTTCTA
AGGCTAAGGGTCAACCAAGAGAACCACAAGTTTACACTTT
GCCACCATCCAGAGATGAATTGACTAAGAACCAAGTTTCT
TTGACTTGTTTGGTTAAGGGTTTTTACCCATCTGATATTG
CTGTTGAATGGGAATCTAACGGTCAACCAGAAAACAACTA
CAAGACTACTCCACCAGTTTTGGATTCTGATGGTTCTTTT
TTTTTGTACTCTAAGTTGACTGTTGATAAGTCCAGATGGC
AACAAGGTAACGTTTTTTCTTGTTCTGTTATGCATGAAGC
TTTGCATAACCATTACACTCAAAAGTCTTTGTCTTTGTCT
CCAGGTGCTAGGCCATCCGGTAGAAAATCTTCCAAAATGC
AAGCCTTCAGGATCTGGGATGTTAACCAAAAAACTTTTTA
CCTGAGGAACAATCAATTGGTGGCTGGATATTTACAAGGA
CCAAATGTTAACCTCGAAGAGAAAATAGATGTAGTCCCTA
TCGAGCCACATGCCTTGTTTTTGGGAATCCATGGTGGGAA
AATGTGTTTATCGTGTGTCAAGTCTGGAGACGAGACGCGT
CTCCAATTGGAGGCCGTCGATATTACGGACTTATCTGAGA
ACCGAAAACAAGACAAACGTTTTGCTTTCATCAGGTCAGA
CAGTGGACCCACAACTTCCTTCGAATCAGCTGCCTGTCCA
GGATGGTTTCTATGCACAGCAATGGAAGCTGATCAGCCTG
TTTCGTTGACTAATATGCCCGATGAGGGTGTCATGGTCAC
TAAATTCTATTTTCAGGAGGACGAG
SEQ ID NO: 179, pPICZαA(pro-d57-70)_
SPINK-Fc(N82D)-Il1Ra_noTag (SEQ
ID NO: 96 bolded)
MRFPSIFTAVLFAASSALAAPVNTTTEDETAQIPAEAVIG
YSDLEGDFDVAVLPFSASIAAKEEGVSLEKREAEADSLGR
EAKCYNELNGCTKIYDPVCGTDGNTYPNECVLCFENRKRQ
TSILIQKSGPCEPKSSDKTHTCPPCPAPELLGGPSVFLFP
PKPKDTLMISRTPEVTCVVVDVSHEDPEVKFNWYVDGVEV
HNAKTKPREEQYDSTYRVVSVLTVLHQDWLNGKEYKCKVS
NKALPAPIEKTISKAKGQPREPQVYTLPPSRDELTKNQVS
LTCLVKGFYPSDIAVEWESNGQPENNYKTTPPVLDSDGSF
FLYSKLTVDKSRWQQGNVFSCSVMHEALHNHYTQKSLSLS
PGARPSGRKSSKMQAFRIWDVNQKTFYLRNNQLVAGYLQG
PNVNLEEKIDVVPIEPHALFLGIHGGKMCLSCVKSGDETR
LQLEAVDITDLSENRKQDKRFAFIRSDSGPTTSFESAACP
GWFLCTAMEADQPVSLTNMPDEGVMVTKFYFQEDE
SEQ ID NO: 180, pPICZαA(rm_pro)_
SPINK-Fc(N82D)-ILIRa_noTag
ATGAGATTTCCTTCAATTTTTACTGCTGTTTTATTCGCAG
CATCCTCCGCATTAGCTGAGGCTGAAGCTGATTCTTTGGG
TAGAGAAGCTAAGTGTTACAACGAATTGAACGGTTGTACT
AAGATTTACGATCCAGTTTGTGGTACTGATGGTAACACTT
ACCCAAACGAATGTGTTTTGTGTTTTGAAAACAGAAAGAG
ACAAACTTCTATTTTGATTCAAAAGTCTGGTCCATGTGAA
CCAAAGTCTTCTGATAAGACACATACTTGTCCACCATGTC
CAGCTCCAGAATTGTTGGGTGGTCCATCTGTTTTTTTGTT
TCCACCAAAGCCAAAGGATACTTTGATGATTTCCAGAACT
CCAGAAGTTACTTGTGTTGTTGTTGATGTTTCTCATGAAG
ATCCAGAAGTTAAGTTTAACTGGTACGTTGATGGTGTTGA
AGTTCATAACGCTAAGACTAAGCCAAGAGAAGAACAATAC
GATTCTACTTACAGAGTTGTTTCTGTTTTGACTGTTTTGC
ATCAAGATTGGTTGAACGGTAAGGAATACAAGTGTAAGGT
TTCTAACAAGGCTTTGCCAGCTCCAATTGAAAAGACTATT
TCTAAGGCTAAGGGTCAACCAAGAGAACCACAAGTTTACA
CTTTGCCACCATCCAGAGATGAATTGACTAAGAACCAAGT
TTCTTTGACTTGTTTGGTTAAGGGTTTTTACCCATCTGAT
ATTGCTGTTGAATGGGAATCTAACGGTCAACCAGAAAACA
ACTACAAGACTACTCCACCAGTTTTGGATTCTGATGGTTC
TTTTTTTTTGTACTCTAAGTTGACTGTTGATAAGTCCAGA
TGGCAACAAGGTAACGTTTTTTCTTGTTCTGTTATGCATG
AAGCTTTGCATAACCATTACACTCAAAAGTCTTTGTCTTT
GTCTCCAGGTGCTAGGCCATCCGGTAGAAAATCTTCCAAA
ATGCAAGCCTTCAGGATCTGGGATGTTAACCAAAAAACTT
TTTACCTGAGGAACAATCAATTGGTGGCTGGATATTTACA
AGGACCAAATGTTAACCTCGAAGAGAAAATAGATGTAGTC
CCTATCGAGCCACATGCCTTGTTTTTGGGAATCCATGGTG
GGAAAATGTGTTTATCGTGTGTCAAGTCTGGAGACGAGAC
GCGTCTCCAATTGGAGGCCGTCGATATTACGGACTTATCT
GAGAACCGAAAACAAGACAAACGTTTTGCTTTCATCAGGT
CAGACAGTGGACCCACAACTTCCTTCGAATCAGCTGCCTG
TCCAGGATGGTTTCTATGCACAGCAATGGAAGCTGATCAG
CCTGTTTCGTTGACTAATATGCCCGATGAGGGTGTCATGG
TCACTAAATTCTATTTTCAGGAGGACGAG
SEQ ID NO: 181, Translated pPICZαA(rm_pro)_
SPINK-Fc(N82D)-IL1Ra_noTag
(SEQ ID NO: 97 bolded)
MRFPSIFTAVLFAASSALAEAEADSLGREAKCYNELNGC
TKIYDPVCGTDGNTYPNECVLCFENRKRQTSILIQKSGPC
EPKSSDKTHTCPPCPAPELLGGPSVFLFPPKPKDTLMISR
TPEVTCVVVDVSHEDPEVKFNWYVDGVEVHNAKTKPREEQ
YDSTYRVVSVLTVLHQDWLNGKEYKCKVSNKALPAPIEKT
ISKAKGQPREPQVYTLPPSRDELTKNQVSLTCLVKGFYPS
DIAVEWESNGQPENNYKTTPPVLDSDGSFFLYSKLTVDKS
RWQQGNVFSCSVMHEALHNHYTQKSLSLSPGARPSGRKSS
KMQAFRIWDVNQKTFYLRNNQLVAGYLQGPNVNLEEKIDV
VPIEPHALFLGIHGGKMCLSCVKSGDETRLQLEAVDITDL
SENRKQDKRFAFIRSDSGPTTSFESAACPGWFLCTAMEAD
QPVSLTNMPDEGVMVTKFYFQEDE

What is claimed is:

1. An Fc fusion protein comprising a pancreatic trypsin inhibitor (PTI) polypeptide linked to an Fc region, wherein the PTI polypeptide is a serine protease inhibitor Kazal-type 1 (SPINK1) polypeptide or a bovine pancreatic trypsin inhibitor (BPTI) polypeptide, wherein the SPINK1 polypeptide does not comprise a mutation, and wherein the Fc region comprises a hinge region, an IgG CH2 domain and an IgG CH3 domain.

2. The Fc fusion protein of claim 1, wherein the N-terminus of the Fc region is linked to the C-terminus of the PTI polypeptide.

3. The Fc fusion protein of claim 1, wherein the C-terminus of the Fc region is linked to the N-terminus of the PTI polypeptide.

4. The Fc fusion protein of claim 1, wherein the PTI polypeptide is the SPINK polypeptide.

5. The Fc fusion protein of claim 4, wherein the SPINK polypeptide comprises an amino acid sequence having at least 85% identity to an amino acid selected from the group consisting of:
(SEQ ID NO: 14)
DSLGREAKCYNELNGCTKIYDPVCGTDGNTYPNECVLCFENRKRQ
TSILIQKSGPC;
(SEQ ID NO: 15)
DSLGREAKCYNELNGCTKIYDPVCGTDGNTYPNECVLCFENRKRQ
TSILIQKSGPCGPEQKLISEEDLNSALVPRGSRL VDHHHHHH;
(SEQ ID NO: 16)
MKVTGIFLLSALALLSLSGNTGADSLGREAKCYNELNGCTKIYDP
VCGGNTYPNECVLCFENRKRQTSILIQKSGPC;
(SEQ ID NO: 17)
MKVTGIFLLSALALLSLSGNTGADSLGREAKCYNELNGCTKIYNP
VCGGNTYPNECVLCFENRKRQTSILIQKSGPC;
(SEQ ID NO: 18)
MKVTGIFLLSALALLSLSGNTGADSLGREAKCYNELNGCTKIYDP
VCGGDTYPNECVLCFENRKRQTSILIQKSGPC;
(SEQ ID NO: 19)
MKVTGIFLLSALALLSLSGNTGADSLGREAKCYNELNGCTKIYDP
VCGGNTYPNECVLCFEGRKRQTSILIQKSGPC;
(SEQ ID NO: 20)
MKVTGIFLLSAFALLSLSGNTGADSLGREAKCYNELNGCTKIYDP
VCGGNTYPNECVLCFENRKRQTSILIQKSGPC;
(SEQ ID NO: 21)
MKVTGIFLLSALAPLSLSGNTGADSLGREAKCYSELNGCTKIYDP
VCGGNTYPNECVLCFENRKRQTSILIQKSGPC;
(SEQ ID NO: 22)
MKVTGIFLLSALALLSLSGNTGADSLGREAKCYNELNGCTKIYDP
VCGGNTYSNECVLCFENRKHQTSILIQKSGPC;
and
(SEQ ID NO: 186)
AKVTGKEASCHDAVAGCPRIYDPVCGTDGITYANECVLCFENRKR
IEPVLIRKGGPC.

6. The Fc fusion protein of claim 4, wherein the SPINK polypeptide comprises an amino acid sequence having at least 85% identity to SEQ ID NO: 85.

7. The Fc fusion protein of claim 4, wherein the SPINK polypeptide comprises an amino acid sequence having at least 85% identity to an amino acid sequence selected from the group consisting of: SEQ ID NOs: 1, 6-11 and 85-114.

8. The Fc fusion protein of claim 1, wherein the PTI polypeptide is a BPTI polypeptide.

9. The Fc fusion protein of claim 8, wherein the BPTI polypeptide comprises an amino acid sequence having at least 95% identity to the amino acid sequence:
(SEQ ID NO: 3)
RPDFCLEPPYTGPCKARIIRYFYNAKAGLCQTFVY
GGCRAKRNNFKSAEDCMRTCGGA.

10. The Fc fusion protein of claim 9, wherein the BPTI polypeptide comprises an amino acid sequence having at least 99% identity to SEQ ID NO: 3.

11. The Fc fusion protein of claim 9, wherein the BPTI polypeptide comprises a substitution at position 16 of SEQ ID NO: 3.

12. The Fc fusion protein of claim 10, wherein the BPTI comprises an A to H or A to S substitution at position 16.

13. The Fc fusion protein of claim 1, wherein the Fc region is a human IgG Fc region.

14. The Fc fusion protein of claim 1, wherein the Fc region is a IgG1 Fc region.

15. The Fc fusion protein of claim 1, wherein the Fc region is a IgG3 Fc region.

16. The Fc fusion protein of claim 1, wherein the hinge region has a length of about 10 to about 20 amino acids.

17. The Fc fusion protein of claim 1, wherein the Fc region comprises an amino acid sequence having at least 85% identity to an amino acid sequence selected from the group consisting of:
(SEQ ID NO: 36)
EPKSSDKTHTCPPCPAPELLGGPSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPGA;
(SEQ ID NO: 37)
EPKSSDKTHTCPPCPAPELLGGPSVFLFPPKPKDTLMISRTPEVT
CVVVDVSHEDPEVKFNWYVDGVEVHNAKTKPREEQYDSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPGA;
(SEQ ID NO: 38)
CPAPELLGGPSVFLFPPKPKDTLMISRTPEVTCVVVDVSHEDPEV
QFKWYVDGVEVHNAKTKPREEQYNSTFRVVSVLTVLHQDWLNGKE
YKCKVSNKALPAPIEKTISKTKGQPREPQVYTLPPSREEMTKNQV
SLTCPVKGFYPSDIAVEWESSGQPENNYNTTPPMLDSDGSFFLYS
KLTVDKSRWQQGNIFSCSVMHEALHNhyTQKSLSLSPGA;
(SEQ ID NO: 39)
epkscdkthtcppCPAPELLGGPSVFLFPPKPKDTLMISRTPEVT
CVVVDVSHEDPEVQFKWYVDGVEVHNAKTKPREEQYNSTFRVVSV
LTVLHQDWLNGKEYKCKVSNKALPAPIEKTISKTKGQPREPQVYT
LPPSREEMTKNQVSLTCPVKGFYPSDIAVEWESSGQPENNYNTTP
PMLDSDGSFFLYSKLTVDKSRWQQGNIFSCSVMHEALHNhyTQKS
LSLSPGa;
and
(SEQ ID NO: 40)
EPeeSDKTHTCPPCPAPELLGGPSVFLFPPKPKDTLMISRTPEVT
CVVVDVSHEDPEVKFNWYVDGVEVHNAKTKPREEQYdSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPGe.

18. The Fc fusion protein of claim 1, wherein the Fc region comprises an amino acid sequence having at least 85% identity to an amino acid sequence selected from the group consisting of SEQ ID NOs: 187-204.

19. The Fc fusion protein of claim 1, comprising the amino acid sequence
(X)₁EPxxSDKTHTCPPCPAPELLGGPSVFLFPPKPKDTLMISRT
PEVTCVVVDVSHEDPEVKFNWYVDGVEVHNAKTKPREEQYxSTYR
VVSVLTVLHQDWLNGKEYKCKVSNKALPAPIEKTISKAKGQPREP
QVYTLPPSRDELTKNQVSLTCLVKGFYPSDIAVEWESNGQPENNY
KTTPPVLDSDGSFFLYSKLTVDKSRWQQGNVFSCSVMHEALHNHY
TQKSLSLSPG(X)ₘDSLGREAKCYNELNGCTKIYDPVCGTDGNTY
PNECVLCFENRKRQTSILIQKSGPC(X)ₙ,
where each X is independently aspartic acid (D) or glutamic acid (E), and the subscripts ‘ ’1′, ‘m’ and ‘n’ and N independently range from 1 to up to 10 or more.

20. The Fc fusion protein of claim 1, comprising the amino acid sequence
(SEQ ID NO: 9)
EPeeSDKTHTCPPCPAPELLGGPSVFLFPPKPKDTLMISRTPEVT
CVVVDVSHEDPEVKFNWYVDGVEVHNAKTKPREEQYdSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPGeDSLGREAKCYNELNGCTKIYDPVCGTDGNTYPNECVLC
FENRKRQTSILIQKSGPC.

21. The Fc fusion protein of claim 1, further comprising an inflammatory cytokine inhibitor.

22. The Fc fusion protein of claim 21, wherein the inflammatory cytokine inhibitor comprises an IL-1Ra domain.

23. The Fc fusion protein of claim 22, wherein the IL-1Ra domain comprises an amino acid sequence having at least 85% identity to an amino acid sequence selected from the group consisting of SEQ ID NOs: 205-207.

24. The Fc fusion protein of claim 23, having at least 85% identity to an amino acid sequence selected from the group consisting of SEQ ID NOs: 88, 89, 93-97.

25. The Fc fusion protein of claim 1, further comprising a signal sequence and/or a leader sequence.

26. The Fc fusion protein of claim 25, wherein signal sequence and/or leader sequence comprises an amino acid sequence having at least 85% identity to an amino acid sequence selected from the group consisting of SEQ ID NOs: 208-210.

27. A pharmaceutical composition comprising a Fc fusion protein of claim 1 and a pharmaceutically acceptable carrier or excipient.

28. A nucleic acid encoding a Fc fusion protein of claim 1.

29. A cell comprising a nucleic acid of claim 28.

30. A kit comprising a nucleic acid of claim 28 or a fusion protein of claim 1.

31. A method for treating pancreatitis, the method comprising administering a Fc fusion protein of claim 1 to a subject in need thereof.

32. The method of claim 31, wherein said administering is intravenous (IV) or intraperitoneal (IP) administration.

33. A method for enhancing outcomes of cardiac surgery in a human, the method comprising administering a fusion protein of claim 1 to a subject in need thereof.
